(19) United States
(12) Patent Application Publication
(10) Pub. No.: US 2024/0197794 A1
(43) Pub. Date: Jun. 20, 2024

(54) PROBIOTIC BACTERIAL STRAINS PRODUCING ANTIMICROBIAL PROTEINS AND COMPOSITIONS COMPRISING THESE FOR USE IN THE TREATMENT OF DIARRHEAL AND OTHER MICROBIAL DISEASES

(71) Applicant: CRIGASSENI AG, Beckenried (NW) (CH)

(72) Inventor: Federico GRAF, Beckenried (NW) (CH)

(21) Appl. No.: 18/533,453

(22) Filed: Dec. 8, 2023

Related U.S. Application Data
(62) Division of application No. 17/052,139, filed on Oct. 30, 2020, now Pat. No. 11,918,611.

(30) Foreign Application Priority Data
May 3, 2018 (EP) 18170567.4

(51) Int. Cl. A61K 35/741; A61K 47/02; A61K 47/18; A61K 47/26; A61P 31/04; C12N 9/14

(52) U.S. Cl. CPC A61K 35/741; A61P 31/04; C12N 9/14; A61K 47/02; A61K 47/183; A61K 47/26

(57) ABSTRACT

The present invention is directed to a bacterial, preferably probiotic bacterial peptidoglycan hydrolase (PGH), a peptidoglycan hydrolase (PGH)-including bacterial, preferably probiotic bacterial strain or a peptidoglycan hydrolase (PGH)-including composition for use in the therapeutic or prophylactic treatment of a bacterial infection, preferably for the treatment of a bacterial infection resulting in diarrhea. Further aspects of the present invention relate to corresponding methods for preparing a medicament and to a corresponding method of treatment.

### CROSS-REFERENCE TO RELATED APPLICATIONS

This application is a divisional application of U.S. patent application Ser. No. 17/052,139, filed 30 Oct. 2020, which is a National Stage application of PCT/EP2019/061259, filed 2 May 2019, titled PROBIOTIC BACTERIAL STRAINS PRODUCING ANTIMICROBIAL PROTEINS AND COMPOSITIONS COMPRISING THESE FOR USE IN THE TREATMENT OF DIARRHEAL AND OTHER MICROBIAL DISEASES, which claims the benefit and priority to European Patent Application No. 18170567.4, filed on 3 May 2018, both of which are incorporated herein by reference in their entirety for all purposes.

### INCORPORATION BY REFERENCE

In compliance with 37 C.F.R. 1.52(e), the sequence information contained in electronic file name: TSW0002USD Sequence Listing ST26.xml; size 342 KB; created on: 7 Mar. 2024, is incorporated herein by reference in its entirety.

### BACKGROUND

The present invention is directed to a bacterial, preferably probiotic bacterial peptidoglycan hydrolase (PGH), a peptidoglycan hydrolase (PGH)-comprising bacterial, preferably probiotic bacterial strain or a peptidoglycan hydrolase (PGH)-comprising composition for use in the therapeutic or prophylactic treatment of a bacterial infection, preferably for the treatment of a bacterial infection resulting in diarrhea. Further aspects of the present invention relate to corresponding methods for preparing a medicament and to a corresponding method of treatment.

Treatment of diarrhea by administering living (e.g. yogurt) or tyndallized (e.g. “Lactdol®”) dried bacteria to restore a disturbed intestinal microflora was introduced in the twenties of the last century and is still practiced. Additionally, in recent decades, many strains of probiotics have been used in the attempt to prevent or cure diarrheal diseases. Different types of diarrhea were investigated clinically (Ref. de Vrese, M. et al., J. Nutr. 137, 803S-811S, 2007) including antibiotic-associated diarrhea (AAD), Clostridium difficile gastroenteritis, traveler's diarrhea, chemo- or radio-induced diarrhea. Even though many studies yielded inconsistent results it appears ascertained that L. rhamnosus LGG may be effective in preventing C. difficile infections, whereas S. boulardii may be useful in the treatment of C. difficile-associated diarrhea in combination with antibiotics.

S. Guandalini (J Clin Gastroeneterol, 2011 November; 45 Suppl: 5149-53) concluded the same: “The vast majority of published trials show a statistically significant benefit and moderate clinical benefit of a few, well identified probiotic strains—mostly Lactobacillus GG and S. boulardii—in the treatment of acute, watery diarrhea . . . ”. However, the achieved improvements seem rather modest: “Such a beneficial effect results, on average, in a reduction of diarrhea duration of approximately one day”.

The mechanisms of action of the above-mentioned two leading strains used in general practice and in hospital for diarrheal conditions have been studied and published in detail, main findings are reported in De Keersmaecker S C et al. (FEMS Microbiol Lett. 2006 June; 259(1):89-96) who report that “Lactobacillus rhamnosus GG produced a low-molecular weight, heat-stable, non-proteinaceous bactericidal substance, active at acidic pH against a wide range of bacterial species. SCS of L. rhamnosus GG grown in MRS medium contained five compounds that could meet the above description, if present at the appropriate concentration. Therefore, the antimicrobial activity of L. rhamnosus GG against S. typhimurium is not due the production of a bacteriocin”.

With respect to peptidoglycan hydrolases the enzyme spectrum of LGG was studied and published as early as 1994 (W. H. Ling et al., Microbial Ecology in Health and Disease: Vol. 7:99-104 (1994)). The aforementioned article teaches the enzyme profile of Lactobacillus GG, and the value for producing enzyme E18, i.e. glucosaminidase, is zero. In other words, no NAGase or other glycosidase production was detected. Hence, no defined type of antimicrobial low- or high-molecular peptide has been associated with the observed antimicrobial activity of LGG.

For S. boulardii many different mechanisms were postulated for its activity alone or as adjuvant for use in C. difficile infections (Therap Adv Gastrenterol. 2012 March: 5(2):111-125). For nosocomial infections a special protease is postulated that cleaves the C. difficile Toxin A and, thus, inhibits its action. No bacteriocin-like substance produced by S. boulardii has been reported so far.

With respect to peptidoglycan hydrolases, the APIZYM profiles of a number of Saccharomyces cerevisiae have been studied (C. Pennacchia et al., Journal of Applied Microbiology, 105:1919-1928 (2008)). All studied strains of S. cerevisae do not secrete N-acetyl-β-glucosaminidase or equivalent PGH.

In conclusion, none of the two leading probiotic strains used in the treatment or prevention of CDI/CDAD were found to produce bacteriocins or antimicrobial enzymes active against C. diff.

The bacterial cell wall consists of glycan strands which are cross-linked by flexible peptide side chains, providing strength and rigidity to the bacterial cell wall. The peptidoglycan of both the Gram-positive and Gram-negative bacteria features repeating units of N-acetylglucosamine (NAG) and β-(1-4)-N-acetylmuramic acid (NAM) cross-linked by peptide stem chains attached to NAM residues. The so-called peptidoglycan hydrolases (PGHs) are the enzymes responsible for cleaving the bonds within the peptidoglycan chains and side-chains branches. PGHs maintain the overall cell wall peptidoglycan turnover. Furthermore, PGH are involved in many essential processes of the bacteria, e.g. the separation of daughter cells during cell division and autolysis (Waldemar Vollmer Bernard Joris Paulette Charlier Simon Foster, FEMS Microbiology Reviews, Volume 32, Issue 2, 1 Mar. 2008, Pages 259-286).

Peptidoglycan hydrolases originate from different sources including animals, plants, bacteria and phages. According to their sources PGHs can be classified into lysozymes, bacteriocins, autolysins and endolysins (Current Research, Technology and Education Topics in Applied Microbiology and Microbial Biotechnology, Chapter: Peptidoglycan hydrolases, Editors: Mendez-Vilas, pp. 463-472).

It is noted that there is a substantial difference between the single lysozymes of animal origin (human, chicken, a.s.o.), discovered by Fleming in 1921, and the class of muramidases/PGHs (also sometimes referred to as lysozymes) of bacterial origin, i.e. bacterial PGHs. The lysozymes of animal origin (human, chicken, a.s.o.) are abundant in animal secretions as tears, milk, mucus a.s.o., are part of the innate immune system and exhibit only a weak antimicrobial activity against Gram-positive bacteria. The bacterial muramidases/PGHs possess the properties listed above and differ in size, function and structure from the former of animal origin. Egg-white derived lysozyme has, e.g. a molecular weight of 14.3 kDa, which corresponds to 1′166 bp DNA, whereas the bacterial PGHs found and reported in this patent application are molecular sequences varying from e.g. as little as 201 to 999 and to 1542 bp DNA.

Alternatively, peptidoglycan hydrolases are also classified in 3 groups according to their substrate specificity:
Glycosidases: N-acetylglucosaminidases (NAGases) and N-acetylmuramidases,
Amidases: N-acetylmuramoyl-L-alanine amidase, and
Peptidases: carboxypeptidases and endopeptidases.

Besides NAGases, that cleave NAG units from the peptidoglycan, also more broadly active N-acetylhexosaminidases as well as other specific glycosidases like N-acetylgalactosaminidase and N-acetylmannosidase have been described/published and will be referred to herein. NAGases and muramidases both cleave the same glycosidic bond between the N-acetylglucosamin and N-acetylmuramic acid units of the glycan chains and are fuctionally and structurally closely related.

Another type of peptides, gene encoded and ribosomally produced by bacteria is categorized as bacteriocins (see: Rodney H Perez, Takeshi Zendo, and Kenji Sonomoto, Microb Cell Fact. 2014; 13(Suppl 1): S3 for a recent review.). Mainly, these bacteriocins (polypeptides) can kill or inhibit taxonomically closely related bacteria within their narrow spectrum of activity, but do not have an essential role in bacterial meta- and catabolism as do PGH. Those bacteriocins produced by safe (GRAS/QPS) bacteria like probiotics are mostly used in the food and dairy industry as preservatives (e.g. nisin from Lactococcus lactis under E-number E234, a representative of the Class I of bacteriocins, the so-called lantibiotics,). It is thus important to clearly separate between bacterial PGHs and bacteriocins, bacterial PGHs being, among other things, much larger in size than bacteriocins. For example, the bacteriocin nisin A has a molecular weight of 3′354.07 Dalton, whereas a typical NAGase, e.g. isolated from Xanthomonas manihotis (New England BioLabs, Catalog #P0732) has a MW of 71 kDalton.

In summary, consistent and significant results in diarrhea, in particular in C. diff-associated diarrhea (CDAD) and/or the C. diff-infection (CDI) are reported only for Lactobacillus rhamnosus LGG and for Saccharomyces boulardii. With regard to the direct antimicrobial activity against gastro-intestinal pathogens causing infections and diarrhea, in particular against C. difficile, neither of the above-mentioned two probiotics produces peptidoglycan hydrolases, e.g. of the type of N-acetylglucosaminidase or glycosidase. Furthermore, it appears that they do not produce other antimicrobial peptides of the type of bacteriocins.

Clostridium difficile (CD or C. diff.) is an enterotoxin-producing (enterotoxin A and cytotoxin B), anaerobic, motile, spore-forming Gram-positive bacterium that is ubiquitous in nature, especially prevalent in soil and which is present in 2 to 5% of the adult human population. C. diff-infection (CDI) is one of the most prevalent nosocomial infections with a dramatic increase in incidence and severity in the past decade.

C. diff-associated diarrhea (CDAD) and C. diff-infection (CDI) are a major burden for Western health systems. Next to antibiotic therapy, many attempts for treating CDAD or CDI focus on different probiotics. The efficacy of probiotics in the prevention of CDAD is by the majority positive, as it typically reduces the occurrence of diarrhea by around 50%. Lau and Chamberlain (Int J Gen Med. 2016 Feb. 22; 9:27-37) concluded that Lactobacillus GG and Saccharomyces probiotics significantly reduce the risk of CDAD in both adults and children, especially among hospitalized patients. However, they also point out that these preliminary results will require additional research for determining the optimum probiotic strain.

The topic of Clostridium difficile colitis is regularly updated in Medscape by Faten N Aberra (http://emededicine.medscape.com/article/186458-overview, Update: Apr. 6, 2017) and the author emphasizes that presently “Probiotics are not recommended as a single agent for the treatment of active CDI owing to limited data supporting their benefit and a potential risk for septicemia. Use of Saccharomyces boulardii and Lactobacillus species have shown mixed results in reducing the risk of CDI relapse. However, S. boulardii has generated interest for the treatment of CDI, because it seems to inhibit the effects of toxins A and B on the human colonic mucosa.”

Allen et al. (The Lancet, Volume 382, No. 9900, p 1249-1257, 12.10.2013) observed that older inpatients suffering from CDI did not benefit from a probiotic combined Lactobacilli and Bifidobacteria supplementation against antibiotic-associated diarrhoea (AAD) and CDAD. The investigators admitted that they “ . . . identified no evidence that a multi-strain preparation of lactobacilli and bifidobacteria was effective in prevention of AAD or CDD”. So far, neither bifidobacterium nor Lactobacillus have shown a significant clinical efficacy in CDI.

It is generally assumed that 4 different mechanisms may play a role in the treatment with CDI/CDADby probiotics (Corr, Hill & Gahan, Adv Food Nutr Res. 2009; 56:1-15):
1. direct antimicrobial activity through production of lactic acid, hydrogen peroxide and bacteriocins;
2. competitive exclusion at binding sites or stimulation of the epithelial barrier function;
3. stimulation of immune responses via increase of sIgA and influence on the production of cytokines;
4. and inhibition of virulence genes or protein expression in gastrointestinal pathogens.

Mechanisms 2 and 3 involve functions of the host and/or of the entire gut microbiome, which are extremely complex to investigate. Only mechanism 1, the direct inhibition of the pathogen by metabolites of lactic acid bacteria such as lactic acid, hydrogen peroxide bacteriocins and the like can be verified relatively easily. This approach has been tried before in different clinical studies that mainly failed.

In the search for probiotic strains against C. difficile P. Naaber (Journal of Medical Microbiology (2004) 53, 551-554) selected strains of different species such as L. plantarum and L. paracasei, and the observed antimicrobial effects were attributed to lactic acid and H₂O₂ production.

More recently, M Ratsep (J. Prob. Health 2014, 2:1) reported in vitro results on the effect of L. plantarum on clinical isolates of C. diff Although several L. plantarum strains inhibited different clinical isolates of C. diff (supposedly on account of the bacteriocin plantaricin), some C. diff strains were not inhibited by any of the probiotic L. plantarum strains. And a clinical study with the commercially successful probiotic L. plantarum strain 299v failed to show any significant positive effect on the production of C. diff toxin in AAD patients (Lönnermark et al., J Clin Gastroenterol. 2010; 44(2):106-12).

Schoster et al. (Anaerobe, vol. 20, April 2013, Pages 36-41) assessed the inhibitory effects of 16 Lactobacillus and one Bifidobacterium commercial strains on reference strains of C. diff and C. perfringens by an agar well diffusion assay and by a broth culture inhibition assay using cell-free supernatant harvested at different growth phases, with and without pH neutralization. The cell-free supernatant (CFSN) of the probiotics (mainly L. rhamnosus, L. casei and L. plantarum) inhibited most of the C. diff and C. perfringens strains when applied after 6, 12 or 24 h (at a pH of 4 or lower). However, when neutralized (to pH 6.9), no one of the CFSN of the probiotics significantly inhibited C. difficile. The effect of other possible agents against C. difficile produced by these lactic acid bacteria (LAB) was in the end masked by the dominating antimicrobial effect of lactic acid at low pH.

More sophisticated experiments were performed by Lee et al. (Toxicol Res. 2013 June; 29(2): 99-106) in order to investigate the activity of 4 different lactic acid bacteria and bifidobacteria (Lactobacillus rhamnosus, Lactococcus lactis, Bifidobacterium breve, and Bifidobacterium lactis) against C. difficile. These probiotics inhibited C. diff in different experimental set-ups. However, the authors eventually admit that the effect of putative antimicrobial substances against C. diff produced by the lactic acid bacteria is masked by the same effect caused by lactic or acetic acid. The strongest anti-C. diff activity exerted by a Lactococcus lactis strain was supposed likely to be mediated through the action of a bacteriocin similar to nisin.

In fact, nisin is presently almost the sole bacteriocin of any probiotic strain that has proven in vitro to inhibit C. difficile.

In summary, the present literature indicates that certain species of lactic acid bacteria, in particular Bifidobacteria and Lactococci, might possess the ability to produce substances that inhibit C. diff. However, in the experiments this inhibition was masked by lactic and other acids produced by these bacteria which eventually lower the pH of the medium and cause an unspecific antimicrobial activity also towards C. diff And none of these antimicrobial substances were identified, except for Lactococcus which is known to produce nisin, an already FDA- and EU-approved substance, widely used in the dairy and food industry against food spoilage.

The therapeutic use of nisin as an antimicrobial against C. difficile has 2 main drawbacks because it is rapidly degraded by proteolytic enzymes in the upper part of the intestine (Ahmad et al., The American Journal of Gastroenterology 108, 625, April 2013), and because it exhibits inhibitory activity against many strains of protective gut bacteria such as Bifidobacteria and Lactobacilli, which activity could be detrimental upon administration to patients (Le Blay et al., Letters in Applied Microbiology 45, 2007, 252-257).

Nisin is bactericidal on the germinated cells of C. difficile as well as on its spores (Avila et al., Int. J Food Microbio. 2014 Feb. 17; Z172:70-5), but it cannot be administered as pure substance (see Ahmad et al., above).

Last but not least, nisin activity is pH-dependent. A pH-value around 4 (Schoster et al. (Anaerobe, vol. 20, April 2013, 36-41) is only relevant for a vaginal environment, whereas in the intestine the pH is higher than, or equal to 5, and depends on the position in this organ.

Next to C. difficile, the most important causative bacterial pathogens for diarrheas include: entero-pathogenic Escherichia coli, diarrhoeagenic Salmonella typhimurium, Shigella flexneri or sonnei, Listeria monocytogenes, Campylobacter jejuni and Clostridium perfringens.

It is the objective of the present invention to provide improved compounds, bacterial strains, compositions and methods for use in the treatment and prophylaxis of bacterial, preferably diarrheal diseases, preferably without the significant drawbacks and side-effects of current antibiotic-based treatments.

### SUMMARY

Disclosed herein are a bacterial peptidoglycan hydrolase (PGH), preferably a probiotic bacterial PGH, a peptidoglycan hydrolase (PGH)-secreting bacterial strain, preferably a PGH-secreting probiotic bacterial strain and a bacterial peptidoglycan hydrolase (PGH)-comprising composition, preferably a probiotic bacterial PGH-comprising composition for use in the therapeutic or prophylactic treatment of a bacterial infection.

In a first aspect, the present invention is directed to a peptidoglycan hydrolase (PGH)-secreting probiotic bacterial strain selected from the group consisting of

Lactococcus lactis, preferably subsp. lactis and subsp. cremoris, Lactobacillus gasseri, Lactobacillus crispatus, Lactobacillus jensenii, Lactobacillus plantarum CCOS 893 (DSM 32352), Lactobacillus fermentum, Lactobacillus johnsonii, Lactobacillus rhamnosus, Lactobacillus paracasei, Lactobacillus casei, Bifidobacterium animalis subsp. lactis, Bifidobacterium longum, Bifidobacterium breve, Bifidobacterium pseudocatenulatum and Bifidobacterium bifidum, for use in the therapeutic or prophylactic treatment of a bacterial infection.

It was surprisingly found that bacterial and probiotic bacterial strains producing and secreting N-acetylglucosaminidase (NAGase), or more generally, producing and secreting suitable peptidoglycan hydrolases (PGHs) such as glycosidases, have an antimicrobial effect on pathogenic, including enteropathogenic, bacteria and in particular on Clostridium difficile strains.

The bacterial, preferably probiotic bacterial peptidoglycan hydrolases (PGH), peptidoglycan hydrolase (PGH)-secreting bacterial strains, preferably probiotic bacterial strains, or bacterial, preferably probiotic bacterial peptidoglycan hydrolase (PGH)-comprising compositions disclosed herein can disrupt any pathogen featuring a cell-wall consisting of a peptidoglycan layer, such as Gram-positives, Gram-negatives and mycobacteria.

The term “PGH-secreting” for a bacterial and/or probiotic bacterial strain means that the strain for use according to the present invention contains the corresponding gene and is capable to secrete the PGH into the extracellular environment.

The term “bacterial, preferably probiotic bacterial PGH-comprising” as used herein, means that a composition contains the bacterial, preferably probiotic bacterial PGH next to at least one further component.

The term “bacterial, preferably probiotic bacterial PGH” refers to any PGH that is preferably produced by bacteria, preferably probiotic bacteria, and therefore preferably is of bacterial origin, more preferably of probiotic bacterial origin. Optionally, the term “bacterial PGH” can exclude PGHs from animals such as humans, chicken or dogs. For example, the term “probiotic bacterial PGH” can exclude PGHs from non-probiotic bacteria, e.g. from pathogenic bacteria. Optionally, the bacterial and probiotic bacterial PGH disclosed herein also exclude any non-bacterial and/or non-probiotic bacterial lysozyme.

It is further preferred that the bacterial PGH is a PGH of probiotic bacteria. It is further preferred that the bacterial and/or probiotic bacterial PGH is a PGH from non-pathogenic bacteria. It is also preferred that the PGH disclosed herein is a PGH that is genotypically encoded and/or expressed and/or secreted by a probiotic strain for use in the present invention, preferably a probiotic strain selected from the group consisting of Lactococcus lactis (preferably subsp. lactis and subsp. cremoris), Lactobacillus gasseri, Lactobacillus crispatus, Lactobacillus jensenii, Lactobacillus plantarum, Lactobacillus fermentum, Lactobacillus johnsonii, Lactobacillus rhamnosus, Lactobacillus paracasei, Lactobacillus casei, Bifidobacterium animalis subsp. lactis, Bifidobacterium longum, Bifidobacterium breve, Bifidobacterium pseudocatenulatum and Bifidobacterium bifidum; more preferably a probiotic strain selected from the group consisting of Lactococcus lactis CCOS 949 (DSM 32294), Lactobacillus gasseri CCOS 960 (DSM 32296); Lactobacillus crispatus CCOS 961 (CCOS 961); Lactobacillus plantarum CCOS 893 (DSM 32352); Lactobacillus johnsonii CCOS 824 (CCOS 824); Lactobacillus paracasei subsp. paracasei CCOS 1205 (CCOS 1205); Lactobacillus paracasei subsp. paracasei CCOS 1201 (CCOS 1201); Lactobacillus fermentum CCOS 1030 (CCOS 1030); Lactobacillus jensenii CCOS 962 (CCOS 962); Lactobacillus rhamnosus CCOS 965 (CCOS 965); Bifidobacterium bifidum CCOS 571 (CCOS 571); Bifidobacterium longum CCOS 974 (CCOS 974); Bifidobacterium breve CCOS 971 (CCOS 971) and Bifidobacterium breve CCOS 586 (CCOS 586), most preferably, the bacterial, preferably probiotic bacterial strain is Lactococcus lactis CCOS 949 (DSM 32294).

The term “a bacterial infection”, as used herein, is meant to define any bacterial invasion into a host, preferably into an animal host, more preferably into a mammalian host, most preferably into a human host, which invasion is undesired, i.e. results in negative health impact(s) and/or is pathological. More preferably the bacterial infection is by Gram-positives, Gram-negatives and/or mycobacteria.

The terms “probiotics” or “probiotic bacterial strains” are commonly defined as “live microorganisms (here: bacteria) that, when administered in adequate amounts, confer a health benefit on the host” (Hill et al., (2014) “Expert consensus document. The International Scientific Association for Probiotics and Prebiotics consensus statement on the scope and appropriate use of the term probiotic” Nat Rev Gastroenterol Hepatol 11: 506-514).

In the context of the present invention, the terms “probiotic bacteria” or “probiotic bacterial strains” are preferably understood as defined by the European Food Safety Administration (EFSA) which grants the label QPS (Qualified Presumption of Safety) to those bacterial strains that are considered “probiotic” in the present application. The EFSA publishes a list of such probiotic strains or family of strains periodically and reference is made to the EFSA Journal, Volume 14, issue 7, published in July 2016, Appendix B, Table B1. Therefore, in the context of the present invention and in addition to the general understanding in the art, the term “probiotic” or “probiotic strain” refers to all the bacterial microorganisms contained in the mentioned list of approved QPS-labelled phyla, classes, families or specific strains of bacterial microorganisms. More preferably, “probiotic bacteria” in the context of the present invention are those of the families Lactobacillaceae, Bifidobacteriaceae and Lactococcaceae; more preferably of the genus Lactobacillus, Bifidobacterium and Lactococcus; most preferably of the species Bifidobacterium bifidum, Bifidobacterium longum, Bifidobacterium breve, Lactobacillus crispatus, Lactobacillus fermentum, Lactobacillus gasseri, Lactobacillus rhamnosus, Lactobacillus jensenii, Lactobacillus johnsonii, Lactobacillus paracasei, Lactobacillus plantarum and Lactococcus lactis.

Additionally, and in line with the QPS label, the label GRAS (Generally Recognized As Safe) issued in the USA is preferably used to characterize “probiotics” or “probiotic strain” as used herein. Generally and as understood by those skilled in the art, the term “probiotic” excludes pathogenic bacteria, in particular bacteria that are pathogenic to humans.

Disclosed herein are the bacterial, preferably probiotic bacterial PGH, the bacterial, preferably probiotic bacterial strain or the composition for use in the present invention are for use in therapeutic or prophylactic treatment of a bacterial infection, which infection results in diarrhea. In a preferred embodiment, the peptidoglycan hydrolase (PGH)-secreting probiotic bacterial strain is for use in the treatment of a bacterial infection that results in diarrhea or is a bacterial urogenital, preferably vaginal infection. The present disclosure also relates to the bacterial, preferably probiotic bacterial PGH or the composition for use in the present invention are for use in therapeutic or prophylactic treatment of a bacterial urogenital, preferably vaginal infection.

As used herein and with reference to the factsheet “Diarrhoeal disease” issued by the World Health Organization (WHO) in May 2017, the term “diarrhea” is defined as the passage of three or more loose or liquid stools per day (or more frequent passage than is normal for the individual). Frequent passing of formed stools is not diarrhoea, nor is the passing of loose, “pasty” stools by breastfed babies. The three clinical types of diarrhoea are: acute watery diarrhoea—lasts several hours or days and includes cholera; acute bloody diarrhoea—also called dysentery; and persistent diarrhoea—lasts 14 days or longer.

It was surprisingly found that probiotic bacteria strains featuring the production of one or more PGH class enzymes, preferably one or more glycosidases such as N-acetylglucosaminidases (NAGase) or hexosaminidases or galactosaminidase exhibit high activity against pathogenic, preferably enteropathogenic bacteria, in particular against Clostridium difficile (C. diff). The data reported in the Examples below demonstrate that the probiotic PGH-secreting strains, in particular the glycosidases- and NAGase-secreting strains, exert a strong inhibitory activity against hospital isolates of C. diff Preferred non-limiting examples of such PGHs-secreting strains are L. lactis CCOS 949, L. plantarum CCOS 893, L. gasseri CCOS 960 and L. crispatus CCOS 961 (see below for deposition details). These strains in particular L. lactis CCOS 949 are preferably also active against spores of C. diff.

It is preferred that probiotic bacteria for use in the present invention are administered in a concentration of between about 1×10\hat{ }6 and 1×10\hat{ }11 cfu/dose, preferably between about 1×10\hat{ }9 and about 1×10\hat{ }10 cfu/dose. The total bacterial dose can, e.g., vary between about 1×10\hat{ }9 and about 5×10\hat{ }11 for a total weight between 0.5 and 20 g per dose, for example 1 g, 5 g, or 10 g.

In a further preferred embodiment, the present invention is directed to a peptidoglycan hydrolase (PGH)-secreting probiotic bacterial strain for use in the therapeutic or prophylactic treatment of a bacterial infection, preferably a bacterial diarrhoe or a bacterial urogenital, preferably vaginal infection, wherein the bacterial infection is caused by a pathogenic microorganism selected from the group consisting of enteroinvasive Escherichia coli, enterohaemorragic Escherichia coli, diarrheagenic Escherichia coli, Helicobacter pylori, Salmonella enterica, Shigella flexneri and sonnei, Campylobacter jejuni, Listeria monocytogenes, Bacillus cereus, Vibrio cholerae, Staphylococcus aureus, Gardnerella vaginalis, Clostridium perfringens and Clostridium difficile, preferably the pathogenic microorganism is Clostridium difficile. Also disclosed herein is a bacterial, preferably probiotic bacterial peptidoglycan hydrolase (PGH) for the use described above.

If the diarrhoeal disease of bacterial origin is caused by, or associated with, an infection by C. difficile, this disease is termed C. difficile-associated diarrhea (CDAD).

The bacterial, preferably probiotic bacterial PGH, PGH-secreting bacterial, preferably probiotic bacterial strain or bacterial, preferably probiotic bacterial PGH-comprising composition is active against the peptidoglycan-comprising cell-wall of a bacterial pathogen. For example, the bacterial, preferably probiotic bacterial PGH as such, produced by the strain or forming part of the composition can be, but is not limited to, a bacterial, preferably probiotic bacterial PGH selected from the group consisting of N-acetylglucosaminidase (NAGase), N-acetylhexosaminidase, N-acetylgalactosaminidase, N-acetylmuramidase, endopeptidase, carboxypeptidase, and N-acetylmuramoyl-L-alanine amidase.

Further disclosed herein is the bacterial, preferably probiotic bacterial PGH, the bacterial, preferably probiotic bacterial strain or the composition for use in the present invention, wherein the bacterial, preferably probiotic bacterial PGH is selected from the group consisting of:
alpha- and beta-N-acetylglucosaminidase (NAGase), preferably beta-N-acetylglucosaminidase, more preferably exo- or endo-beta-N-acetylglucosaminidase;
N-acetylmuramidase;
alpha- and beta-N-acetylhexosaminidase, preferably beta-N-acetylhexosaminidase;
alpha- and beta-N-acetylgalactosaminidase, preferably beta-N-acetylgalactosaminidase;
mannosyl-glycoprotein endo-beta-N-acetylglucosaminidase;
beta-hexosaminidase;
alpha-mannosidase;
N-acetylmuramoyl-L-alanine amidase;
glycoside hydrolase;
endopeptidase;
carboxy-petidase;
bacterial surface protein AcmA; and
neuraminidase.

In a preferred embodiment, the peptidoglycan hydrolase (PGH)-secreting probiotic bacterial strain for use according to the present invention is one, wherein the peptidoglycan hydrolase (PGH) is selected from the group described above.

The bacterial enzymes listed above with peptidoglycan-hydrolase activity are systematically described and listed in the Brenda-Database of enzyme classification (https://www.brenda-enzymes.org). This database is referred to for all synonyms of the above-listed PGHs. Preferably, the bacterial, preferably probiotic bacterial PGHs as such, secreted by the bacterial, preferably probiotic bacterial strain or comprised by the composition for use in the present invention are members of the Enzyme Class E.C. 3.2.1., with, e.g., 3.2.1.52 denoting beta-D-acetylglucosaminidase.

It is further preferred that the bacterial, preferably probiotic bacterial peptidoglycan hydrolase (PGH) described herein, preferably a glycosidase, more preferably an N-acetylglucosaminidase (NAGase) or hexosaminidase or muramidase is combined with nisin or another bacteriocin, and/or that the bacterial, preferably probiotic bacterial strains for use according to the present invention also secrete nisin or another bacteriocin, e.g., for enhancing antimicrobial activity. Such other bacteriocins can, e.g., be found in the genomes of the probiotic strains mentioned in this invention and/or are preferably selected from the group consisting of: Pediocin; Gassericin; Enterolysisn; Helveticin; Penocin; Lactoccin; and Colicin.

Optionally, the bacterial, preferably probiotic bacterial peptidoglycan hydrolase (PGH), bacterial, preferably probiotic bacterial strain or composition for use according to the present invention is one, wherein the PGH-secreting bacterial, preferably probiotic bacterial strain is selected from the group consisting of Lactococcus lactis, preferably subsp. lactis and subsp. cremoris, Lactobacillus gasseri, Lactobacillus crispatus, Lactobacillus jensenii, Lactobacillus plantarum, Lactobacillus fermentum, Lactobacillus johnsonii, Lactobacillus rhamnosus, Lactobacillus paracasei, Lactobacillus casei, Bifidobacterium animalis subsp. lactis, Bifidobacterium longum, Bifidobacterium breve, Bifidobacterium pseudocatenulatum and Bifidobacterium bifidum.

The present disclosure also encompasses a combination of two or more, preferably 3 to 7, bacterial, preferably probiotic bacterial peptidoglycan hydrolases (PGH) or a composition comprising two or more, preferably 3 to 7, bacterial, preferably probiotic bacterial peptidoglycan hydrolases (PGH) for the use described above.

In a preferred embodiment, the present invention is also directed to a combination of two or more, preferably 3 to 7, bacterial, preferably probiotic bacterial strains for use according to the embodiments of the present invention. Preferably, and as an example, a strain for use according to the present invention, preferably L. lactis, more preferably L. lactis CCOS 949, which produces bacteriocins, preferably nisin and/or lactococcin, is combined with other PGH-producing, preferably glycosidase-producing strains as described herein, preferably L. gasseri CCOS 960, L. plantarum CCOS 893, B. breve CCOS 571 and/or B. breve CCOS 971, to potentiate, preferably synergistically potentiate the antipathogen activity against C. diff.

Preferably the bacterial, preferably probiotic bacterial PGH as such and when comprised in the composition for use as described herein is a PGH expressed and secreted in one or more of the herein-identified PGH-comprising bacterial, preferably probiotic bacterial strains.

The finding that bacterial, preferably probiotic bacterial strains secreting PGHs have an antimicrobial effect on bacterial pathogens, in particular enteropathogenic species, and in particular on C. diff strains, was verified for the representative organism Lactococcus and subsequently for Lactobacillus strains, as well as for another large family of (probiotic) bacteria, the Bifidobacteria (see Examples below).

The bacterial, preferably probiotic bacterial strains for use in the present invention demonstrate a significant inhibitory activity against bacterial pathogens including C. diff and preferably up to an intermediate pH-range of 5-6. Therefore, the bacterial, preferably probiotic bacterial strains for use in the present invention have the advantage that their activity is extended over the entire length of the intestine while damages to the healthy part of the gut microbiota are avoided or at least reduced.

Lactococcus lactis is a Gram-positive, aerotolerant, non-motile and non-sporulating bacterial species belonging to the Streptococcaceae family. L. lactis includes four subspecies, cremoris, hordniae, lactis and tructae, and one biovar, L. lactis subsp. lactis by. diacetylactis. In accordance with their traditional use in food, members of the L. lactis species has been listed in the “inventory of microbial food cultures” with documented use in food fermentations and are most commonly classed as Generally Recognized as Safe (GRAS) by the Food and Drug Administration (FDA); Salminen et al. (1998) “Demonstration of safety of probiotics—a review” Int J Food Microbiol 44: 93-106).

Furthermore, L. lactis received from the European Food Safety Authority (EFSA) the status of Qualified Presumption of Safety (QPS), which is granted to those taxonomic groups deliberately introduced into the food chain that do not raise safety concerns and are considered “probiotic” herein (doi:10.2903/j.efsa.2007.587).

Although the intestine is not its primary habitat, L. lactis can survive the gastrointestinal transit and may remain metabolically active in various compartments of the alimentary canal (Drouault et al., (1999) Appl Environ Microbiol 65: 4881-4886; Kimoto et al., (2003) Can J Microbiol 49: 707-711), demonstrating that L. lactis is a suitable microorganism for probiotic applications.

L. lactis is particularly preferred for use in the present invention, e.g. because of its various habitats. It possesses wide genetic variability, has a variety of phenotypes and is potentially beneficial for the host's health.

CDI occurs preferably in the colon, but also in the small intestine. Consequently, a transient probiotic colonization with anti-C. difficile probiotic strains should reach both parts of the gut, which can, for example, be implemented by the parallel administration of Lactobacillus, more strongly present in the small intestine, and Bifidobacteria, more strongly present in the large intestine.

In a preferred embodiment the peptidoglycan hydrolase (PGH)-secreting, bacterial, preferably probiotic bacterial strain or composition for use according to the present invention is one, wherein the bacterial, preferably probiotic bacterial strain is selected from the group consisting of Lactococcus lactis CCOS 949 (DSM 32294), Lactobacillus gasseri CCOS 960 (DSM 32296); Lactobacillus crispatus CCOS 961 (CCOS 961); Lactobacillus plantarum CCOS 893 (DSM 32352); Lactobacillus johnsonii CCOS 824 (CCOS 824); Lactobacillus paracasei subsp. paracasei CCOS 1205 (CCOS 1205); Lactobacillus paracasei subsp. paracasei CCOS 1201 (CCOS 1201); Lactobacillus fermentum CCOS 1030 (CCOS 1030); Lactobacillus rhamnosus CCOS 965 (CCOS 965), Lactobcillus jensenii CCOS 962 (CCOS 962), Bifidobacterium bifidum CCOS 571 (CCOS 571); Bifidobacterium longum CCOS 974 (CCOS 974); Bifidobacterium breve CCOS 971 (CCOS 971) and Bifidobacterium breve CCOS 586 (CCOS 586), preferably, the bacterial strain is Lactococcus lactis CCOS 949 (DSM 32294).

Also disclosed herein is a bacterial, preferably probiotic bacterial peptidoglycan hydrolase (PGH) secreted by one or more of the above-listed strains.

It is noted that the strain Lactobacillus paracasei subsp. paracasei CCOS 1205 (CCOS 1205) is identical to the strain Lactobacillus paracasei subsp. paracasei CCOS 1201 (CCOS 1201) and this strain was deposited twice.

It is noted that Lactococcus lactis CCOS 949 produces the bacteriocin nisin on one hand and, on the other hand, several peptidoglycan hydrolases (PGH) of the glycosidase type. A preferred embodiment of the present invention is directed to the use of a bacterial, preferably probiotic bacterial strain that expresses bacteriocins, preferably nisin, and a PGH, preferably of the gylcosidase type. Without wishing to be bound by theory, it is noted that a bacteriocin, preferably nisin, and a PGH, preferably of the gylcosidase type act together synergistically, thus exhibiting a potent, unexpected inhibitory activity against C. diff.

Lactococcus lactis CCOS 949 was deposited under the Budapest Treaty on 21 Apr. 2016 under accession number DSM 32294 by Probioswiss GmbH, Technoparkstrasse 1, Zurich, Switzerland with the DSMZ (Deutsche Sammlung von Mikroorganismen und Zellkulturen, Leibniz-Institut, InhoffenstraBe 7B, 38124 Braunschweig, Germany).

Lactobacillus gasseri CCOS 960 was deposited under the Budapest Treaty on 21 Apr. 2016 under accession number DSM 32296 by Probioswiss GmbH, Technoparkstrasse 1, Zurich, Switzerland with the DSMZ (Deutsche Sammlung von Mikroorganismen und Zellkulturen, Leibniz-Institut, InhoffenstraBe 7B, 38124 Braunschweig, Germany).

Lactobacillus crispatus CCOS 961 was deposited under the Budapest Treaty on 10 Dec. 2017 under deposit accession number CCOS 961 by Crigasseni AG, Ledergasse 34, Beckenried, Switzerland with the Culture Collection of Switzerland AG (CCOS, Einsiedlerstr. 34, 8820 Wadenswil, Switzerland).

Lactobacillus plantarum CCOS 893 was deposited under the Budapest Treaty on 27 Jul. 2016 under accession number DSM 32352 by Probioswiss GmbH, Technoparkstrasse 1, Zurich, Switzerland with the DSMZ (Deutsche Sammlung von Mikroorganismen und Zellkulturen, Leibniz-Institut, InhoffenstraBe 7B, 38124 Braunschweig, Germany).

Lactobacillus johnsonii CCOS 824 was deposited under the Budapest Treaty on 16 Apr. 2018 under deposit accession number CCOS 824 by Crigasseni AG, Ledergasse 34, Beckenried, Switzerland with the Culture Collection of Switzerland AG (CCOS, Wadenswil, Switzerland).

Lactobacillus paracasei subsp. paracasei CCOS 1205 was deposited under the Budapest Treaty on 21 Apr. 2018 under deposit number CCOS 1205 by the Culture Collection of Switzerland AG (CCOS, Einsiedlerstr. 34, 8820 Wadenswil, Switzerland) at the same institution.

Lactobacillus paracasei subsp. paracasei CCOS 1201 was deposited under the Budapest Treaty on 17 Apr. 2019 under deposit accession number CCOS 1201 by Crigasseni AG, Ledergasse 34, Beckenried, Switzerland with the Culture Collection of Switzerland AG (CCOS, Einsiedlerstr. 34, 8820 Wadenswil, Switzerland).

Lactobacillus fermentum CCOS 1030 was deposited under the Budapest Treaty on 10 Apr. 2018 under deposit accession number CCOS 1030 by Crigasseni AG, Ledergasse 34, Beckenried, Switzerland with the Culture Collection of Switzerland AG (CCOS, Einsiedlerstr. 34, 8820 Wadenswil, Switzerland).

Lactobacillus jensenii CCOS 962 was deposited under the Budapest Treaty on 174 April 2019. under deposit accession number CCOS 962 by Crigasseni AG, Ledergasse 34, Beckenried, Switzerland with the Culture Collection of Switzerland AG (CCOS, Einsiedlerstr. 34, 8820 Wadenswil, Switzerland).

Lactobacillus rhamnosus CCOS 965 was deposited under the Budapest Treaty on 17 Apr. 2019 under deposit accession number CCOS 965 by Crigasseni AG, Ledergasse 34, Beckenried, Switzerland with the Culture Collection of Switzerland AG (CCOS, Einsiedlerstr. 34, 8820 Wadenswil, Switzerland).

Bifidobacterium bifidum CCOS 571 was deposited under the Budapest Treaty on 6 Nov. 2017 under deposit accession number CCOS 571 by Martin Sievers of the Culture Collection of Switzerland AG, Wadenswil, Switzerland, with the Culture Collection of Switzerland AG (CCOS, Einsiedlerstr. 34, 8820 Wadenswil, Switzerland).

Bifidobacterium longum CCOS 974 was deposited under the Budapest Treaty on 10 Apr. 2018 under deposit accession number CCOS 974 by Crigasseni AG, Ledergasse 34, Beckenried, Switzerland with the Culture Collection of Switzerland AG (CCOS, Einsiedlerstr. 34, 8820 Wadenswil, Switzerland).

Bifidobacterium breve CCOS 971 was deposited under the Budapest Treaty on 20 Apr. 2018 under deposit accession number CCOS 971 by Crigasseni AG, Ledergasse 34, Beckenried, Switzerland with the Culture Collection of Switzerland AG (CCOS, Einsiedlerstr. 34, 8820 Wadenswil, Switzerland).

Bifidobacterium breve CCOS 586 was deposited under the Budapest Treaty on 6 Nov. 2017 under deposit accession number CCOS 586 by Martin Sievers of the Culture Collection of Switzerland AG, Wadenswil, Switzerland, with the Culture Collection of Switzerland AG (CCOS, Einsiedlerstr. 34, 8820 Wadenswil, Switzerland).

All deposits were made on the same terms as those laid down in the Budapest Treaty on the International Recognition of the Deposit of Microorganisms for the purpose of Patent Procedure. Probioswiss GmbH, Zurich Switzerland, the Culture Collection of Switzerland AG and Martin Sievers of the Culture Collection of Switzerland AG have assigned the deposited strains to Crigasseni AG, Beckenried, Switzerland and have given their unreserved and irrevocable consent to the deposited material being made available to the public as evidenced by the Statements of Authorization and Consent filed with the European Patent Office (as the Receiving Office).

For some probiotic strains for use in the present invention it can be of advantage for stable growth and maintenance of viability to administer these in combination with strain-specific nutritional compounds, which compounds might not be, e.g. naturally, available in sufficient amounts in the gut.

In a further preferred embodiment, the composition, preferably a composition comprising the bacterial, preferably probiotic bacterial strain, more preferably Lactococcus lactis, most preferably subspecies lactis or subspecies cremoris, for use as described above, further comprises
at least one N-acetylated monosaccharide;
at least one prebiotic carbon source;
at least one nitrogen source;
and/or at least a sulfur source.

Lowering the pH in the intestine is a further preferred option when administering the (probiotic) bacteria or (probiotic) bacteria-comprising composition for use in the present invention.

In some instances, bacterial, preferably probiotic bacterial strains capable of inhibiting pathogenic bacteria, e.g. C. diff, can incur a partial loss of inhibition, which is sometimes observed at a pH higher than 5.5. To minimize or avoid such loss, a dose of at least one prebiotic carbon source (e.g. between 2 and 15 g per dosage, preferably between 5 and 10 g, more preferably between 5 and 6 g) can preferably be provided that is sufficient to lower the pH of the intestinal milieu. The lowering of the pH in the intestinal milieu can be achieved by fermentation of the at least one prebiotic carbon source by the bacterial, preferably probiotic bacterial strain, thus leading to the production of lactic acid and/or short chain fatty acids (SCFA) like acetic or butyric acid.

In this context, a prebiotic carbon source is a compound that can be, preferably selectively, fermented by bacterial, preferably probiotic and eubiotic bacterial strains for use in the present invention and preferably leads to fermentation products that lower the pH in the intestinal milieu. Examples of preferred prebiotic carbon sources are provided further below.

In many cases the production of PGHs by (probiotic) bacterial strains for use in the present invention, e.g. of NAGase, is stimulated by the presence of the corresponding simple sugar, e.g. N-acetyl-glucosamine (NAG). In a preferred embodiment, the composition, preferably the bacterial, preferably probiotic bacterial strain-comprising composition, for use in the present invention further comprises an N-acetylated monosaccharide selected from the group consisting of N-acetylglucosamine, N-acetylgalactosamine, N-acetyl-hexosamine, N-acetylmannosamine, and mixtures thereof.

Prebiotic carbon sources for use in combination with the bacterial, preferably probiotic bacterial strains and composition for use in the present invention are preferably selective carbon sources specific for the (probiotic) bacterial strains which sources preferably cannot be utilized by competing pathogens. Kondepudi et al. (Anaerobe 18 (5), 489-497. 2012 August 24.) investigated the ability of C. diff strains to ferment a selection of different prebiotic non-digestible oligosaccharides (NDOs). Whereas the tested Bifidobacterium (breve, longum, lactis, etc.) fermented all the NDOs to different extents, C. diff strains could not. Feeding bacterial, preferably probiotic bacterial strains with such selective prebiotics (e.g. fructooligosac-charides (FOS), galacto-oligosaccharides (GOS), glycooligosaccharides, lactulose, xylooligosaccharides (XOS) and/or isomaltooligosaccharides (IMOS)) can warrant that the benefit is limited to the (probiotic) bacterial strains.

In a further preferred embodiment, the composition for use in the present invention is one, wherein the prebiotic carbon source is selected from the group consisting of a fructooligosaccharide, galactooligosaccharide, glyco-oligosaccharide, lactulose, xylooligosaccharide, isomaltooligosaccharide, and mixtures thereof.

Preferably, a prebiotic carbon source or carbon source mix should weigh in the range of about 0.5 to 10 grams per dosis bacterial, preferably probiotic bacterial formulation(s), or about 2% to 50% by weight of a composition for use in the present invention comprising bacterial, preferably probiotic bacterial strain(s), prebiotic carbon source(s) and other galenical ingredients.

Nitrogen sources for use in combination with the bacterial, preferably probiotic bacterial strains and composition for use in the present invention are, e.g., ammonium salts (e.g. ammonium chloride or citrate), urea, amino acids, preferably nitrogen-rich amino acids such as, e.g., glutamic acid, arginine, aspartic acid and/or alanine, as well as mixtures thereof, preferably capable of supporting the growth of intestinal and vaginal lactobacilli.

Because nisin, as well as further lantibiotic, sulfur-containing bacteriocins, is an effective agent against pathogenic bacteria, in particular against C. diff., it is important to optimize its production in the intestine. Nisin production can be increased by adding physiologically effective sulfur sources (L. de Vuyst, 1995, Journal of Applied Bacteriology, 78:28-33) to bacterial, preferably probiotic bacterial strains for use in the present invention. For example, suitable sources may be the amino acids methionine, serine or cysteine, which can be administered to the intestine in amounts of about 0.1 to 5 g of cysteine/cystine, N-acetylcysteine, methionine and/or serine), preferably about 0.2 to 2.0 g per dosage form.

In another preferred embodiment, the composition for use in the present invention comprises at least one sulfur source selected from the group consisting of methionine, cysteine, cystine, cystathionine, a sulfur-containing inorganic salt, preferably magnesium sulfate, sodium or potassium thiosulfate, and mixtures thereof.

A preferred proportion of the sulfur source(s) for preparing a composition for use in the present invention may vary between about 1% and 10%, preferably about 3% to 8% by weight of the total weight of the composition.

In another aspect, the present invention is directed to a peptidoglycan hydrolase (PGH) for use in the therapeutic or prophylactic treatment of a bacterial infection, preferably a bacterial infection that results in diarrhea or a bacterial urogenital, preferably vaginal infection, wherein the PGH is a polypeptide selected from the group consisting of:
(a) polypeptides comprising, preferably having an amino acid sequence selected from the group consisting of SEQ ID NOs: 89 to 151 and 158 to 163;
(b) polypeptides comprising an amino acid sequence having an amino acid sequence identity of at least 70 or 80%, preferably at least 90 or 95% with an amino acid sequence selected from the group consisting of SEQ ID NOs: 89 to 151 and 158 to 163, preferably over the whole length of the sequence; and
(c) functional fragments and/or functional derivatives of (a) or (b).

The identity of related amino acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two amino acid sequences comprise, but are not limited to, TBLASTN, BLASTP, BLASTX, TBLASTX (Altschul et al., (1990) J. Mol. Biol., 215, 403-410), ClustalW (Larkin M A et al., Bioinformatics, 23, 2947-2948, 2007) or PHYRE2 (Kelley L A et al., (2015) Nature Protocols 10, 845-858). The BLAST programs can be obtained from the National Center for Biotechnology Information (NCBI) and from other sources (BLAST handbook, Altschul et al., NCB NLM NIH Bethesda, MD 20894). The ClustalW program can be obtained from http://www.clustal.org and the PHYRE2 program from http://www.sbg.bio.ic.ac.uk/phyre2/html/page.cgi?id=index.

The term “functional derivative” of a polypeptide for use in the present invention is meant to include any polypeptide or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative still has at least some PGH activity to a measurable extent, e.g. of at least about 1 to 10% or more than 50% PGH activity of the original unmodified polypeptide for use in the invention, e.g. SEQ ID NOs: 89 to 151 and 158 to 163. Functional derivatives of a polypeptide for use in the present invention include non-natural polypeptides and glycosylated, phosphorylated, PEGylated, etc. derivatives.

In this context a “functional fragment” for use in the invention is one that forms part of a polypeptide or derivative for use in the invention and still has at least some PGH activity to a measurable extent, e.g. of at least about 1 to 10% or more than 50% PGH activity of the original unmodified polypeptide for use in the invention, e.g. SEQ ID NOs: 89 to 151 and 158 to 163.

In another aspect, the present invention is directed to a peptidoglycan hydrolase (PGH) for use in the therapeutic or prophylactic treatment of a bacterial infection, preferably a bacterial infection that results in diarrhea or a bacterial urogenital, preferably vaginal infection, wherein the PGH is encoded by a nucleic acid comprising or consisting of a nucleic acid sequence selected from the group consisting of:
(a) nucleic acid sequences selected from the group consisting of SEQ ID NOs: 1 to 11, 24 to 53, 65 to 81, 84 to 88 and 152 to 157;
(b) nucleic acid sequences having at least 80 or 90% identity, preferably at least 95% identity, more preferred at least 98% identity with a nucleic acid sequence listed in SEQ ID NOs: 1 to 11, 24 to 53, 65 to 81, 84 to 88 and 152 to 157, preferably over the whole sequence;
(c) nucleic acid sequences that hybridize to a nucleic acid sequence of (a) or (b) under stringent conditions;
(d) fragments of any of the nucleic acid sequences (a) to (c), that hybridize to a nucleic acid sequence of (a) or (b) under stringent conditions; and
(e) a nucleic acid sequence, wherein said nucleic acid sequence is derivable by substitution, addition and/or deletion of one of the nucleic acids of (a) to (d) that hybridizes to a nucleic acid sequence of (a) or (b) under stringent conditions.

The term “% (percent) identity” as known to the skilled artisan and used herein in the context of nucleic acids indicates the degree of relatedness among two or more nucleic acid molecules that is determined by agreement among the sequences. The percentage of “identity” is the result of the percentage of identical regions in two or more sequences while taking into consideration the gaps and other sequence peculiarities.

The identity of related nucleic acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two nucleic acid sequences comprise, but are not limited to, BLASTN (Altschul et al., (1990) J. Mol. Biol., 215:403-410) and LALIGN (Huang and Miller, (1991) Adv. Appl. Math., 12:337-357). The BLAST programs can be obtained from the National Center for Biotechnology Information (NCBI) and from other sources (BLAST handbook, Altschul et al., NCB NLM NIH Bethesda, MD 20894).

The nucleic acid molecules encoding the PGH for use according to the invention may be prepared synthetically by methods well-known to the skilled person, but also may be isolated from suitable DNA libraries and other publicly available sources of nucleic acids and subsequently may optionally be mutated. The preparation of such libraries or mutations is well-known to the person skilled in the art.

In some instances, the present invention also provides PGHs for use as described herein which are encoded by novel nucleic acids characterized in that they have the ability to hybridize to a specifically referenced nucleic acid sequence, preferably under stringent conditions. Next to common and/or standard protocols in the prior art for determining the ability to hybridize to a specifically referenced nucleic acid sequence under stringent conditions (e.g. Sambrook and Russell, (2001) Molecular cloning: A laboratory manual (3 volumes)), it is preferred to analyze and determine the ability to hybridize to a specifically referenced nucleic acid sequence under stringent conditions by comparing the nucleotide sequences, which may be found in gene databases (e.g. http://www.ncbi.nlm.nih.gov/entrez/query.fcgi?db=nucleotide and http://genome.jgi.doe.gov/programs/fungi/index.jsf) with alignment tools, such as e.g. the above-mentioned BLASTN (Altschul et al., (1990) J. Mol. Biol., 215:403-410), LALIGN alignment tools and multiple alignment tools such as e.g. CLUSTALW (Sievers F et al., (2011) Mol. Sys. Bio. 7: 539), MUSCLE (Edgar., (2004) Nucl. Acids Res. 32:1792-7) or T-COFFEE (Notredame et al., (2000) J of Mol. Bio 302 1: 205-17).

Most preferably, the ability of a nucleic acid of the present invention to hybridize to a nucleic acid, e.g. those listed in any of SEQ ID NOs: 1 to 11, 24 to 53, 65 to 81, 84 to 88 and 152 to 157, is confirmed in a Southern blot assay under the following conditions: 6× sodium chloride/sodium citrate (SSC) at 45° C. followed by a wash in 0.2×SSC, 0.1% SDS at 65° C.

The term “PGH encoded by a nucleic acid” as used in the context of the present invention is meant to include allelic variations and redundancies in the genetic code.

The nucleic acid encoding the PGH for use in the present invention is preferably operably linked to a promoter that governs expression in suitable vectors and/or host cells producing the PGH of the present invention in vitro or in vivo.

Suitable promoters for operable linkage to the isolated and purified nucleic acid are known in the art. In a preferred embodiment the nucleic acid encoding the PGH for use in the present invention is one that is operably linked to a promoter selected from the group consisting of the Pichia pastoris GAP promoter, AUG1 promoter, FLD1 promoter and AOX1 promoter (see for example Pichia Expression Kit Instruction Manual, Invitrogen Corporation, Carlsbad, Calif.), the Saccharomyces cerevisiae GAL1, ADH1, GAP, ADH2, MET25, GPD, CUP1 or TEF promoter (see for example Methods in Enzymology, 350, 248, 2002), the Baculovirus polyhedrin p10 or ie1 promoter (see for example Bac-to-Bac Expression Kit Handbook, Invitrogen Corporation, Carlsbad, Calif., and Novagen Insect Cell Expression Manual, Merck Chemicals Ltd., Nottingham, UK), the Lentivirus CMV, UbC, EF1α, or MSCV promoter (see for example System Biosciences, Mountain View, CA, USA), the Adenovirus CMV promoter (see for example ViraPower Adenoviral Expression System, Life Technologies, Carlsbad, CA, USA), the Simian virus 40 promoter SV40, the E. coli T7, araBAD, rhaP BAD, tetA, lac, trc, tac or pL promoter (see Applied Microbiology and Biotechnology, 72, 211, 2006), the B. subtilis, vegI, vegII, σA, P_{grac}, P_{glv}, manP or P43 promoter (see Applied Microbiology and Biotechnology, 72, 211, 2006), the plant CaMV35S, ocs, nos, Adh-1, Tet promoters (see e.g. Lau and Sun, Biotechnol Adv. 2009, 27, 1015-22) or inducible promoters for mammalian cells as described in Sambrook and Russell (2001).

In a further preferred embodiment, the present invention is directed to a recombinant vector for producing a PGH for use as described herein, comprising a nucleic acid as described above, preferably a viral or episomal vector, preferably a baculovirus vector, lentivirus vector, adenovirus vector, vaccinia or retroviral vector, yeast or bacterial episomal vector.

The selection of a suitable vector and expression control sequences as well as vector construction are within the ordinary skill in the art. Preferably, the viral vector is a lentivirus vector (see for example System Biosciences, Mountain View, CA, USA), adenovirus vector (see for example ViraPower Adenoviral Expression System, Life Technologies, Carlsbad, CA, USA), baculovirus vector such as bacmid (or see for example Bac-to-Bac Expression Kit Handbook, Invitrogen Corporation, Carlsbad, Calif.), the pcDNA, pVITRO, pSV and pCMV series of plasmid vectors, vaccinia and retroviral vectors (see for example Hruby, D. E. (1990). Vaccinia virus vectors: new strategies for producing recombinant vaccines. Clinical Microbiology Reviews, 3(2), 153-170.), bacterial vector pGEX and pET (or see for example Novagen, Darmstadt, Germany)) or yeast vector pPIC (or see for example ATCC Manassas, Virginia). Vector construction, including the operable linkage of a coding sequence with a promoter and other expression control sequences, is within the ordinary skill in the art.

In another preferred embodiment, the present invention is directed to a host cell producing and/or secreting a PGH for use as described herein, comprising a nucleic acid or a vector as described above.

Preferred host cells for producing the polypeptide of the invention are selected from the group consisting of yeast cells preferably Saccharomyces cerevisiae (see for example Methods in Enzmology, 350, 248, 2002), Pichia pastoris cells (see for example Pichia Expression Kit Instruction Manual, Invitrogen Corporation, Carlsbad, Calif.)], bacterial cells preferably E. coli cells (BL21(DE3), K-12 and derivatives) (see for example Applied Microbiology and Biotechnology, 72, 211, 2006) or B. subtilis cells (1012 wild type, 168 Marburg or WB800N)(see for example Westers et al., (2004) Mol. Cell. Res. Volume 1694, Issues 1-3 P: 299-310), plant cells, preferably Nicotiana tabacum or Physcomitrella patens (see e.g. Lau and Sun, Biotechnol Adv. 2009 May 18. [electronic publication ahead of print]), NIH-3T3 mammalian cells (see for example Sambrook and Russell, 2001), Human Embryonic Kidney 293 cells (HEK 293, adherent or in suspension, also large T antigen transformed HEK 293T cells), Chinese hamster ovary (CHO) cells, COS cells, and insect cells, preferably sf9 insect cells (see for example Bac-to-Bac Expression Kit Handbook, Invitrogen Corporation, Carlsbad, Calif.).

More preferably, the host cell for use in the present invention is selected from the group consisting of yeast cells, preferably Saccharomyces cerevisiae, Pichia pastoris cells, bacterial E. coli, Vibrio natriegens or B. subtilis cells, plant cells, preferably Nicotiana tabacum or Physcomirella patens cells, NIH-3T3, HEK293, HEK293T, CHO and COS mammalian cells, and insect cells, preferably sf9 insect cells

In another aspect, the present invention is directed to the use of a a peptidoglycan hydrolase (PGH)-secreting bacterial, preferably probiotic bacterial strain, a composition comprising the PGH-secreting strain, a PGH comprising an amino acid sequence as defined herein and/or encoded by the nucleic acids described herein, a recombinant vector or a host cell as described herein for the manufacture of a medicament for the therapeutic or prophylactic treatment of a bacterial infection, preferably a bacterial infection resulting in diarrhoe or a bacterial urogenital, preferably vaginal infection.

Also disclosed herein is the use of a bacterial, preferably probiotic bacterial peptidoglycan hydrolase (PGH) or a bacterial, preferably probiotic peptidoglycan hydrolase (PGH)-comprising composition for the manufacture of a medicament for the therapeutic or prophylactic application described above.

In another aspect, the present invention is directed to a pharmaceutical composition comprising a peptidoglycan hydrolase (PGH)-secreting bacterial, preferably probiotic bacterial strain or a bacterial, preferably probiotic bacterial peptidoglycan hydrolase (PGH)-secreting strain-comprising composition, as defined herein, a PGH comprising an amino acid sequence as defined herein an/or encoded by the nucleic acids described herein, a recombinant vector or a host cell as described herein and at least one physiologically acceptable excipient.

Also disclosed herein is a pharmaceutical composition comprising a bacterial, preferably probiotic bacterial peptidoglycan hydrolase (PGH) or a bacterial, preferably probiotic peptidoglycan hydrolase (PGH)-comprising composition and at least one physiologically acceptable excipient.

In a further aspect, the present invention encompasses corresponding food- or feed additive-compositions or dietary compositions, which, next to food, feed, and/or other dietary components, comprise a peptidoglycan hydrolase (PGH)-secreting bacterial, preferably probiotic bacterial strain, a PGH that comprises the amino acid sequences defined herein or that is encoded by the nucleic acids, a recombinant vector or a host cell for use in the present invention. Also disclosed are food- or feed additive-compositions or dietary compositions, which, next to food, feed, and/or other dietary components, comprise a bacterial, preferably probiotic bacterial peptidoglycan hydrolase (PGH). These compositions have particular utility for the prophylactic treatment/prevention of bacterial infections, preferably bacterial diarrhoe, preferably in humans and animals, more preferably in humans.

It is preferred that the bacteria, preferably probiotic bacteria as such or, e.g., in the form of their lyophilisates, tyndallisates, sonicates, cell-walls and cell extracts are present in the pharmaceutical, food additive, feed additive or dietary composition for use in the present invention in a quantity of about 0.5% to 50%, preferably about 5% to 20% by weight, relative to the total weight of the composition.

For therapeutic or prophylactic use the pharmaceutical composition of the invention may be administered in any conventional dosage form in any conventional manner. Suitable routes of administration include oral, rectal or intravenous, intramuscular and subcutaneous injections. The preferred modes of administration are oral and rectal for bacterial lyophilisates and derived forms or injectable (intravenous or subcutaneous) for the pure (probiotic) bacterial PGH enzymes and their mixtures. In general, the galenical form of the pharmaceutical composition for use in the present invention can be a tablet, a capsule, a sachet, a vial or a ready-to-use liquid preparation.

The bacterial, preferably probiotic bacterial PGH, the bacterial, preferably probiotic bacterial strain, the composition, the PGH comprising the amino acid sequences or encoded by the nucleic acids, the recombinant vector or the host cell described herein may be administered alone or in combination with adjuvants that enhance stability of the PGH, nucleic acids, vectors, host cell and/or strains, facilitate administration of pharmaceutical compositions containing them in certain embodiments, provide increased dissolution or dispersion, increase activity, provide adjunct therapy (e.g. corticosteroids, beta-adrenoceptor antagonists, anti-inflammatory drugs, immune-suppressants), and the like, including other active ingredients, in particular antiobiotics. Advantageously such combination therapies utilize lower dosages of the conventional therapeutics, in particular antibiotics, thus avoiding possible toxicity and adverse side effects incurred when those agents are used as monotherapies. The herein-described (probiotic) bacterial PGH, (probiotic) bacterial strains, PGH comprising the amino acid sequences or encoded by the nucleic acids, recombinant vectors or host cells as described herein may be physically combined with conventional therapeutics or other adjuvants into a single pharmaceutical composition. Reference in this regard may be made to Cappola et al.: U.S. patent application Ser. No. 09/902,822, PCT/US 01/21860 und U.S. provisional application No. 60/313,527, each incorporated by reference herein in their entirety. Advantageously, the bacterial, preferably probiotic bacterial PGH and the bacterial, preferably probiotic bacterial strain may be administered together in a single dosage form, thus forming a composition for use in the present invention. The optimum percentage (w/w) of the (probiotic) bacterial PGH, the (probiotic) bacterial strain, PGH comprising the amino acid sequence or encoded by the nucleic acids, recombinant vector or host cell in a composition for use in the invention may vary and is within the purview of those skilled in the art. Alternatively, the (probiotic) bacterial PGH, the (probiotic) bacterial strain, the composition, the PGH comprising the amino acid sequence or encoded by the nucleic acids, the recombinant vector or host cell for use in the present invention may be administered separately (either serially or in parallel). Separate dosing allows for greater flexibility in the dosing regime.

As mentioned above, dosage forms of the bacterial, preferably probiotic bacterial PGH, the bacterial, preferably probiotic bacterial strain, the composition, the PGH comprising the amino acid sequence or encoded by the nucleic acids, the recombinant vector or host cell for the use described herein optionally include pharmaceutically acceptable carriers and adjuvants known to those of ordinary skill in the art. Methods for preparing such dosage forms are known (see, for example, Ansel and Popovish, Pharmaceutical Dosage Forms and Drug Delivery Systems, 5^{th} ed., Lea and Febiger (1990)). Dosage levels and requirements are well-recognized in the art and may be selected by those of ordinary skill in the art from available methods and techniques suitable for a particular patient. Dosages for the bacterial, preferably probiotic bacterial peptidoglycan hydrolase (PGH), the peptidoglycan hydrolase (PGH)-comprising composition, the PGH comprising the amino acid sequence or encoded by the nucleic acids, the recombinant vector or host cell for the use described herein optionally range from 10-200 mg/dose for a 70 kg patient (Masaro KAJI et al., Journal of the Japanese Association for Infectious Diseases, Volume 40 (1966-1967) Issue 8 Pages 295-309).

Although one dose per day may be sufficient, optionally up to 5 doses per day may be given. In total, 1′000 mg/day may be required. In some embodiments, dosages for the peptidoglycan hydrolase (PGH)-comprising (probiotic) bacterial strain for the use described herein may range between 1×10\hat{ }6 and 1×1\hat{ }11 cfu/dose, preferably between about 1×10\hat{ }9 and about 1×10\hat{ }10 cfu/dose. The total bacterial dose can, e.g., vary between about 1×10\hat{ }9 and about 5×10\hat{ }11 for a total weight between 0.5 and 20 g per dose, for example 1 g, 5 g, or 10 g.

Reference in this regard may also be made to U.S. provisional application No. 60/339,249. As the skilled artisan will appreciate, lower or higher doses may be required depending on particular factors. For instance, specific doses and treatment regimens will depend on factors such as the patient's general health profile, the severity and course of the patient's disorder or disposition thereto, and the judgment of the treating physician.

In preferred embodiments, the present invention also encompasses bacterial, preferably probiotic bacterial PGH and nisin-secreting strains, preferably Lactococcus cells, more preferably live Lactococcus cells that may transiently colonize the gut and deliver PGH and nisin in situ. For example, a PGH and nisin-secreting Lactococcus strain such as L. lactis CCOS 949 is strongly active against C. diff and therefore advantageous for use as effective oral probiotic formulation directed against this pathogen. In another preferred embodiment, the present invention also encompasses bacterial, preferably probiotic bacterial PGH secreting strains such as L. gasseri CCOS 960 producing bacteriocins like pediocin, gassericin and helveticin. In another preferred embodiment the invention contains as well bacterial, preferably probiotic bacterial PGH producers and L. crispatus CCOS 961 capable of producing enterolysin A and penocin. For more evenly distributing a bacterial, preferably probiotic bacterial strain, more preferably a PGH and nisin-secreting (probiotic) bacterial strain, e.g. a Lactococcus strain such as L. lactis CCOS 949, along the intestine, it is preferred to prepare retard/slow release formulations, e.g. capsules, so that the slow release-formulated bacteria, preferably probiotic bacteria will also reach the large intestine. Alternatively, lyophlisates of very active, i.e. preferred strains, such as L. lactis CCOS 949 or L. plantarum CCOS 893 can be microencapsulated to reach the same retard/slow release effect (A. Kumar, Trends in Food Science and Technology, Vol. 18, Issue 5, May 2007, pg. 240-251).

It is further preferred to formulate retard/slow release formulations of Bifidobacteria strains, such as for example B. longum CCOS974 and/or B. breve CCOS 971, suitable for use in the present invention, e.g. capsules that protect the (probiotic) bacterial strain(s) from acidity and bile salts during the gastric and duodenal passage, e.g. by microencapsulation.

In a further aspect, the present invention is directed to a method for the prophylaxis and/or treatment of a bacterial infection, preferably a bacterial infection resulting in diarrhoe or a bacterial urogenital, preferably vaginal infection, in a patient in need of such treatment or prophylaxis, comprising the steps of:
(i) administering an effective amount of a peptidoglycan hydrolase (PGH)-secreting bacterial, preferably probiotic bacterial strain, a peptidoglycan hydrolase (PGH)-secreting strain-comprising composition, a PGH comprising an amino acid sequence as described herein and/or encoded by the nucleic acids described herein, a recombinant vector or a host cell as defined above, preferably a pharmaceutical composition as described above to the patient, preferably an animal selected from the group consisting of mammalian animals, preferably agricultural farm animals, or humans, and
(ii) repeating said administration if needed.

Also disclosed herein is a method as described above, wherein a bacterial, preferably probiotic bacterial peptidoglycan hydrolase (PGH) or a bacterial, preferably probiotic bacterial peptidoglycan hydrolase (PGH)-comprising composition are administered instead of or in addition to the peptidoglycan hydrolase (PGH)-secreting bacterial, preferably probiotic bacterial strain.

In a preferred embodiment, the method according to the present invention is one, wherein the effective amount of the peptidoglycan hydrolase (PGH)-secreting bacterial, preferably probiotic bacterial strain, the PGH comprising an amino acid sequence as described herein and/or encoded by the nucleic acids described herein, the recombinant vector or the host cell is administered orally or rectally.

Also disclosed herein is a method as described above, wherein the effective amount of the bacterial, preferably probiotic bacterial peptidoglycan hydrolase (PGH), the bacterial, preferably probiotic bacterial peptidoglycan hydrolase (PGH)-comprising composition, the PGH comprising an amino acid sequence as described herein and/or encoded by the nucleic acids described herein, the recombinant vector or the host cell is administered orally or by intravenous or subcutaneous injection.

Unless specified otherwise, within the scope of the present invention the percentages and amounts of a component in a mixture are intended to refer to the weight of that component relative to the total weight of the mixture.

Unless specified otherwise, within the scope of the present invention, in relation to ranges of numerical values for a certain feature, the indication “from X to Y” comprises the extremes, i.e. X and Y, as well as all the possible intermediate numerical values.

In the context of the present invention, the term “composition(s)” is meant to include a pharmaceutical composition, a composition for a food or feed supplement, or a composition for a food/dietary product.

### BRIEF DESCRIPTION OF THE DRAWINGS

The following Figures and Examples serve to illustrate the invention and are not intended to limit the scope of the invention as described in the appended claims.

FIG. 1: shows inhibitory effects of Lactococcus lactis on C. difficile. The bars represent the ratio between the colony forming units determined at beginning and after 25 hours of incubation in the co-cultivation experiments.

FIG. 2: shows the data of the inhibition of C. difficile C3 through L1 and L2 as outlined in Example 7 (Table 8b).

FIG. 3: shows the data of the inhibition of C. difficile C1 through L1 and L2 as outlined in Example 7 (Table 8c).

FIG. 4: shows the data of the inhibition of C. difficile C2 by Lactobacilli L1 and L2 as outlined in Example 7 (Table 8d).

FIG. 5: shows the data of the inhibition of C. difficile C4 by Lactobacillus L1 and L2 as outlined in Example 7 (Table 8e).

FIG. 6: shows the data of the inhibition of C. difficile C5 by Lactobacillus L1 and L2 as outlined in Example 7 (Table 8f).

FIG. 7A-E: shows the data of the inhibition of C. difficile C1, C2, C3, C4 and C5 by L. plantarum CCOS 893, respectively as outlined in Example 8 (Tables 9a-e).

FIG. 8: shows examples of inhibitory effects of 3 Bifidobacteria against 2 different C. difficile strains. 1: B. lactis BB12, 2: B. bifidum CCOS 571, 3: B. breve CCOS 586. (−): no zone of inhibition, (+): inhibition detected, (++): strong inhibitory effect.

FIG. 9: shows a phase contrast image of L. lactis CCOS 949.

FIG. 10: shows a phase contrast image of Lactobacillus plantarum CCOS 893.

FIG. 11: shows a phase contrast image of Lactobacillus crispatus CCOS 961.

FIG. 12: shows a phase contrast image of Lactobacillus gasseri CCOS 960.

FIG. 13: shows a phase contrast image of Lactobacillus jensenii CCOS 962.

FIG. 14: shows a phase contrast image of Lactobacillus rhamnosus CCOS 965.

FIG. 15: shows a phase contrast image of Lactobacillus paracasei CCOS 1201.

FIG. 16: shows the WesternBlot analysis of purified NAGase. Lanes 1&2: Protein fractions from expression at 37° C., lanes:3 to 6: expression at 20° C.

### DETAILED DESCRIPTION

### Example 1: Spot Experiments: Comparative Inhibition of C. difficile by Different Strains of Lactococci

a. Introduction

In order to have a more comprehensive basis for a comparison between the killing activities of different probiotic strains against C. diff, 3 different Lactococcus strains were tested against 5 hospital isolates of C. diff

TABLE 1

| Nr. | Species | Working Code | Access Code/Provider |
|---|---|---|---|
| L1 | Lactococcus lactis | FG 03 | CCOS 949 |
| L2 | Lactococcus lactis | SP 38 | DSM 26868 |
| L3 | Lactococcus lactis | SP 47 | Sacco S.r.l., 22071 |
| | | | Cadorago (IT) |
| C1 | Clostridium difficile | 2014 11678 | CCOS 937 |
| C2 | Clostridium difficile | 2014 11648 | CCOS 938 |
| C3 | Clostridium difficile | 2014 11698 | CCOS 939 |
| C4 | Clostridium difficile | 2015 11285 | CCOS 940 |
| C5 | Clostridium difficile | Test-strain Lab | CCOS 941 |

The Clostridium difficile strains are available from the public collection of the Culture Collection of Switzerland CCOS, Einsiedlerstrasse 34, 8820 Wadenswil, Switzerland, www.ccos.ch. Lyophilisates of strains L2 (SP38) and L3 (SP47) were purchased from Sacco S.r.l. 22071 Cadorago (IT).

b. Methods

Media and growth conditions of Lactococcus sp.: the preparatory cultures were incubated for 48 h at 37° C. (with CO₂7.5%) on COS sheep blood plates (aerobically, without CO₂). Afterwards fresh cultures were inoculated in MRS broth and incubated over night at 37° C. (aerobically, without CO₂).

c. Test Procedure

The overnight cultures of the C. diff strains were then evenly scratched out on the COS plates (for each one C1-C5 one plate). Then 10 microliters of the suspensions of the overnight cultures of the Lactococcus lactis strains were pipetted on the plates that had been before inoculated with C. difficile. In order to observe the effect of pH value each suspension was additionally neutralized (with NaOH 4%) at a pH-value of 5-6 and likewise pipetted onto the inoculated plates. Finally, the plates were incubated at 37° C. for 48 hours and the inhibition of C. difficile assessed.

d. Results

TABLE 1a
Inhibition of Clostridium difficile by Lactococci in spot experiments

| | | C. difficile | | | | |
|---|---|---|---|---|---|---|
| | pH- | CCOS 937 | CCOS 938 | CCOS 939 | CCOS 940 | CCOS 941 |
| Lactococcus | value | (C1) | (C2) | (C3) | (C4) | (C5) |
| CCOS 949 | 5 | ++ | ++ | ++ | ++ | ++ |
| CCOS 949 n | 6 | ++ | ++ | ++ | ++ | ++ |
| DSM 26868 | 4.5 | + | + | +/− | +/− | +/− |
| DSM 26868 n | 5-5.5 | + | + | − | − | +/− |
| SP 47 | 4.5 | +/− | +/− | − | − | +/− |
| SP 47 n | 5.5 | +/− | +/− | − | − | +/− |
| MRS (neg. control) | >6 | − | − | − | − | − |

Legend:
++: very good −;
+: good −;
+/−: weak −;
−: no inhibition;
n = neutralized

e. Conclusion

For the Lactococcus lactis DSM 26868 and SP 47, both nisin producers, the usual pattern was observed: The activity is good at pH 4.5, but then clearly diminishes when the pH is raised to pH 5.5.

However, the Lactococcus lactis strain CCOS 949 (DSM 32294, also abbreviated as LCL 949) shows a different behavior: Not only it is strongly active against all of the tested Clostridium difficile at pH<=5, but this activity remains strong even after raising the pH to 6. From Table 1a, one preliminary conclusion can be drawn: although DSM 26868 and SP 47 are also nisin producer, they are less active against C. difficile (C1-C5) than LCL 949. Hence, there must be another anti-microbial factor beside nisin that confers to LCL 949 its particular efficacy (see below).

### Example 2: Determination of the Inhibitory Properties Lactococcus lactis CCOS 949 Against Clostridium difficile in Co-Cultivation Experiments in Liquid Medium

TABLE 2
Tested bacterial cultures and cultivation conditions

| | | Pre-cultue | |
|---|---|---|---|
| Test strains | Strain code | medium | Co-culture medium |
| Clostridium | CCOS 941 | Thioglycollate | Thioglycollate-MRS |
| difficile | | | (9:1) broth |
| Lactococcus | CCOS 949 | MRS | Thioglycollate-MRS |
| lactis | | | (9:1) broth |

a. Methods

All cultures were re-activated from being cryopreserved (−80° C.) working stocks by cultivation on suitable agar medium at 37° C. under anaerobic conditions. For the co-cultivation experiments, 10 mL of the pre-cultivation medium were inoculated with a single colony of each strain and incubated (overnight for Lactococcus lactis, 4-5 hours for C. difficile). These cultures were used as inocula for the co-cultivation medium (a 9:1 vol/vol mixture of Thioglycollate and MRS broth). The ratio of Lactococcus lactis to C. difficile was set to approx. 100:1 based on optical density measurements at 590 nm of the inocula. In addition, all strains were inoculated separately and run as controls in parallel. The growth of the cultures was monitored anaerobically for 25 hours at 37° C., with shaking (120 rpm) by measuring pH, OD and colony forming units (cfu) at 3 to 4 time points. Colony numbers for Lactococcus lactis were determined by plating on MRS agar with aerobic incubation at 37° C. for 1-2 days, the aerobic incubation inhibited the growth of C. difficile. The number of C. difficile was determined by plating on Thioglycollate agar and anaerobe incubation at 37° C. for 1 day. Both C. difficile and Lactococcus lactis were able to grow on this medium, however a discrimination between Lactococcus lactis and C. difficile was possible by colony morphology. For the quantification of the inhibitory capacity of Lactococcus lactis against C. difficile the ratio of the number of colony forming units at the beginning and the end of the experiment for both Lactococcus lactis and C. difficile was calculated. The lower the ratio the stronger the inhibitory effect.

To determine the effect of pH on C. difficile survival due to lactic acid formation by Lactococcus lactis, additional experiments with buffered co-cultivation medium (0.2 M sodium phosphate, pH 5.5) were performed. All anaerobic work was carried out in an anaerobic chamber (Coy Laboratory Products, USA).

b. Results

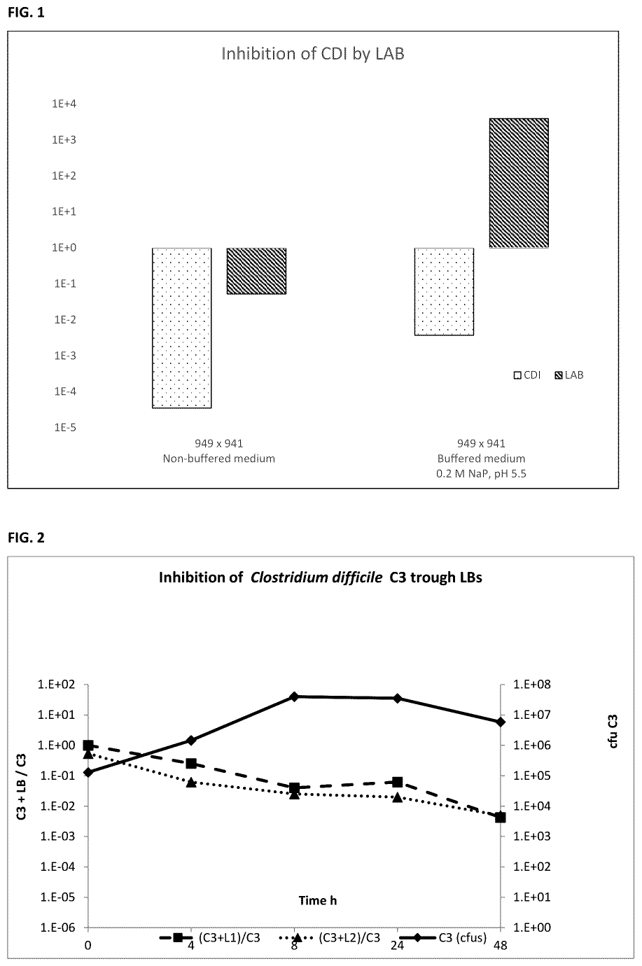

Results are illustrated in FIG. 1. Table 3 shows the results of co-cultivation experiments, development of pH and cell numbers (cfu/mL).

TABLE 3

| | | t = 0 h | | | t = 25 h | | |
|---|---|---|---|---|---|---|---|
| | | cfu/mL | cfu/mL | | cfu/mL | cfu/mL | |
| Cultures | Code | Lc. lactis | CDI | pH | Lc. lactis | CDI | pH |

Thioglycollate-MRS (1:9) medium, not buffered

| | | | | | | | |
|---|---|---|---|---|---|---|---|
| Lc. lactis | 949 | 9.0E+6 | — | 6.98 | 2.6E+5 | — | 4.55 |
| C. difficile | 941 | — | 2.9E+7 | 6.98 | — | 1.2E+9 | 5.74 |
| Lc. lactis × CDI | 949 × 941 | 9.0E+6 | 2.9E+7 | 6.98 | 4.7E+5 | <100 | 4.70 |

Thioglycollate-MRS (1:9) medium, buffered: 2 m M NaP, pH 5.5

| | | | | | | | |
|---|---|---|---|---|---|---|---|
| Lc. lactis | 949 | 1.9E+6 | — | 5.55 | 4.2E+9 | — | 4.90 |
| C. difficile | 941 | — | 6.7E+5 | 5.59 | — | 2.5E+8 | 5.60 |
| Lc. lactis × CDI | 949 × 941 | 2.5E+6 | 2.7E+5 | 5.54 | 9.8E+9 | <1000 | 5.16 |

× = co-cultivation

c. Conclusion
a) Non-buffered medium, individual strains: this apparently gives a preference to C. difficile as compared to Lactococcus lactis CCOS 949. The latter decreases after 25 h by one log, the former increases by 3 log. In the co-culture to Lactococcus lactis CCOS 949 decreases by 1 log, whereas C. difficile decreases by 5-6 logs. Alternatively, the Lactococcus lactis culture is already exhausted after 25 h.
b) Buffered medium: surprisingly Lactococcus lactis CCOS 949 has a better growth by 3 logs, about the same as for C. difficile. In the co-culture Lactococcus lactis CCOS 949 increases by 3 logs, whereas C. difficile decreases about 3 logs.
c) The inhibitory effect of Lactococcus lactis CCOS 949 on C. difficile is maintained in the buffered co-culture even at pH over 5 (pH=5.16) as compared to the unbuffered one with pH=4.70.

The co-culture experiments confirm the spot experiments in as much as the Lactococcus lactis CCOS 949 has an anti-C. difficile activity, which is less dependent on pH as compared with other strain of the species Lactococcus.

Screening for enzymes was carried out, with the aim of identifying other, different anti-microbial products contributing besides nisin to the anti-microbial activity of LCL 949. Besides lactic (mainly L-) and acetic acids, and nisin in certain cases, Lactococcus lactis is not known to produce other kind of antimicrobial metabolites like e.g. hydrogen peroxide, that only certain Lactobacillus, especially those of vaginal origin, produce. The enzyme spectrum of Lactococcus lactis CCOS 949 was therefore investigated, looking for proteins which could potentially have anti-microbial activities and contribute to the remarkable antibacterial activity of Lactococcus lactis CCOS 949. As potential candidates for such active proteins the different peptidases, esterases, galactosidases a.s.o. were considered, but eventually the peptidoglycan hydrolases were more closely investigated.

The different functions of the peptidoglycan hydrolases in the cell meta- and catabolism were reviewed in detail by W. Vollmer et al. (FEMS Microbiol Rev. 2008 March; 32(2):259-86. doi: 10.1111/j.1574-6976.2007.00099.x. Epub 2008 February 11. Bacterial peptidoglycan (murein) hydrolases, Vollmer W, Joris B, Charlier P, Foster S.).

However, the prior art does not mention the production of glycosidases (e.g. N-acetyl-glucosaminidase (NAGase) or N-acetylmuramidases or N-acetylhexosaminidases) or more generally a peptidoglycan hydrolase by some lactic acid bacteria, or more specifically some bacteria or probiotic bacteria, for the purpose of inhibiting or killing competing, pathogenic bacteria.

### Example 3: NAGase Production Among Lactic Acid Bacteria (LAB)

The screening experiments conducted by the inventors showed that the NAGase activity as revealed by established enzymatic tests kits varies between species and strains of lactic acid bacteria.

TABLE 4
Assay with whole cells

| | Score (0-5) | | | Concentration (nmol) | | |
|---|---|---|---|---|---|---|
| Strain | 12 h | 18 h | 24 h | 12 h | 18 h | 24 h |
| Lactobacillus jensenii | 2 | 1 | 0 | 10 | 5 | 0 |
| KS 119.1 | | | | | | |
| Lactococcus lactis | 4 | 2 | 0 | 30 | 10 | 0 |
| CCOS 949 | | | | | | |
| Lactobacillus gasseri | 5 | 2 | 3 | >40 | 20 | 30 |
| KS 120.1 | | | | | | |

a. Method

Lactic acid bacteria strains were tested for their enzyme activity using APIzym test from BioMérieux, reference no. 25200. All test cultures were first checked for identity and purity and preserved at −80° C. After reactivation the lactic acid bacteria were grown on MRS agar for 24 to 48 hours under anaerobe conditions. Bacterial cells were then harvested from the agar plates and the enzyme activities were determined according to the manufacturer's instructions. The tests were performed in duplicate and measurements were done at 12, 18 and 24 hours after incubation, interpretation of the results was done according to the guidelines of the manufacturer by attributing values from 0 to 5 to the observed colour reactions.

b. Results

Comparing the detection of NAGase of the 3 strains it was observed observed that L. jensenii, used as the negative control, secretes only low levels of this enzyme whereas L. gasseri KS 120.1, the positive control, does it at high levels. Lactococcus lactis CCOS 949 also significantly produces NAGase.

c. Conclusion

Hence it was concluded that NAGase (and hexosaminidase or muramidase) contributes, besides nisin, to the significant anti-C. difficile activity of Lactococcus lactis CCOS 949. If NAGase/muramidase/hexosaminidase (that is: glycosidase) is an independent contributor to the activity of Lactococcus lactis. CCOS 949 against C. difficile, then in bacteria lacking nisin, but producing/expressing NAGase/muramidase, a correlation should be found between the degree of glycosidase production and the antimicrobial activity. Furthermore, the genome analysis of LCL 949 should then reveal, besides genes for nisin-type bacteriocins also genes corresponding to peptidoglycan hydrolases, in particular glycosidases.

### Example 4: Genome Sequencing and Analysis of Lactococcus lactis CCOS 949

### 4.1 Whole Genome Sequencing, Assembly and Annotation.

Whole genomic high molecular weight DNA extraction of Lactococcus lactis CCOS 949 —Lactococcus lactis was grown in 10 ml of liquid MRS (Man, Rogosa and Sharpe medium) at 37° C. without shaking in a 50 ml conical tube for 48 h (cell density 1×10⁹ cells/ml). A 1 ml aliquot was centrifuged for 5 min at 8,000×g. The supernatant was removed and resuspended in 500 μl of 5M LiCl. The cells were placed on ice and vigorously vortexed every 2 minutes for 15 minutes. The cells were then centrifuged at 8,000×g for 5 min. A total of 500 μl of saturated phenol was added to the cell pellet, vortexed and centrifuged at 8,000×g. The cell pellet was washed with PBS and resuspended in 500 μl of PBS. Whole genomic DNA was extracted using MasterPure™ DNA Purification Kit (Epicentre, Cat. No. MCD85201). A total of 10 μg of DNA was used to construct a large insert (5-20 kb) SMRTbell™ sequencing library that was sequenced on a PacificBiosciences RSII instruments according to the manufacturer recommendations at the Institute for Genome Sciences, University of Maryland School of Medicine, Baltimore, MD USA.

Two SMRT cells were sequenced (P6 polymerase and C4 chemistry) which generated a total of 467,794 sequence reads with a mean read length of 6,038 bp totaling 2.8 billion bp and a maximum read length of 47,345 bp. The genome of Lactococcus lactis CCOS 949 was assembled using the MinHash Alignment Process (MHAP) (Berlin K, Koren S, Chin C-S, Drake J P, Landolin J M, Phillippy A M. “Assembling large genomes with single-molecule sequencing and locality-sensitive hashing” Nat Biotechnol. 2015; 33:623-30).

### 4.2 Analysis of N-Acetyl-Glucosaminidase Encoding Genes

The genome of Lactococcus lactis CCOS 949 encodes different genes for PGHs and in particular four genes annotated as encoding for N-acetylglucosaminidase.

| | SEQ ID NO | SEQ ID NO | |
|---|---|---|---|
| Gene ID | nucleotide | amino acid | Annotation |
| Llactis_02290 | 65 | 130 | Muramidase-2 precursor |
| Llactis_03700 | 66 | 131 | N-acetylmuramoyl-L-alanine amidase domain containing |
| | | | protein precursor |
| Llactis_04950 | 67 | 132 | Exopolysaccharide biosynthesis protein related |
| | | | to Nacetylglucosamine-1-phosphodiester |
| | | | alpha-N-acetylglucosaminidase |
| Llactis_11300 | 68 | 133 | N-acetylmuramoyl-L-alanine amidase domain containing |
| | | | protein precursor |
| Llactis_14060 | 69 | 134 | Bacteriophage peptidoglycan hydrolase |
| Llactis_14990 | 70 | 135 | Lyzozyme M1 (1,4-beta-N-acetylmuramidase) |
| Llactis_15230 | 71 | 136 | N-acetylmuramoyl-L-alanine amidase domain containing |
| | | | protein precursor |
| Llactis_18610 | 72 | 137 | Beta-N-acetylhexosaminidase precursor |
| Llactis_18640 | 73 | 138 | Endo-beta-N-acetylglucosaminidase D |
| Llactis_19470 | 74 | 139 | Exo-glucosaminidase LytG precursor |
| Llactis_22080 | 75 | 140 | Bacteriophage peptidoglycan hydrolase |
| Llactis_22440 | 76 | 141 | Bacteriophage peptidoglycan hydrolase |
| Llactis_22630 | 77 | 142 | Beta-hexosaminidase |
| Llactis_23080 | 78 | 143 | Muramidase-2 precursor |
| Llactis_25570 | 79 | 144 | N-acetylmuramoyl-L-alanine amidase domain containing |
| | | | protein precursor |
| Llactis_18530 | 80 | 145 | putative alpha-1,2-mannosidase |
| Llactis_18650 | 81 | 146 | Alpha-mannosidase |

Endo-β-N-acetylglucosaminidase (as well as N-acetylmuramoyl-L-alanine amidase, 1,4-beta-N-acetylmuramidase, Beta-hexosaminidase a.s.o.) are a class of proteins with activity against bacterial peptidoglycan and as such have potential antibacterial activities.

4.3 Bacteriocin Analysis of L. lactis CCOS 949

Using the BAGEL3 Algorithm (de Jong A, van Heel A J, Kok J, Kuipers O P. “BAGEL2: mining for bacteriocins in genomic data” Nucleic Acids Res. 2010; 38:W647-51) and BLAST, two bacteriocin biosynthetic gene clusters were identified: nisin and lactococcin.

### 4.4 Nisin A

This biosynthetic gene cluster encodes for the biosynthesis and modification of Nisin A. The gene cluster highly similar to that found in Lactococcus lactis M78 [Trmčić A, Samelis J, Monnet C, Rogelj I, Matijašić BB. “Complete nisin A gene clusterfrom Lactococcus lactis M78 (HM219853)—obtaining the nucleic acid sequence and comparing it to other published nisin sequences” Genes Genom. 2011; 33:217-21]. The 16.3 kb pathway is located on contig tig00000001 and comprises 11 genes and is flanked by insertion sequence IS904.

The gene nisA (SEQ ID NO: 54) encodes for NisA, a 57-amino acid peptide that contains a leader peptide of 23 aa. The sequence of NisA is (\hat{ } indicates the activation site):

(SEQ ID NO 82)
MSTKDFNLDLVSVSKKDSGASPR\hat{ }ITSISLCTPGCKTGALMGCNMKTAT
CHCSIHVSK

The sequence of each the 11 core genes is as follows:

| Gene | Gene length | SEQ ID NO | Annotation |
|---|---|---|---|
| nisA | 174 bp | 54 | Nisin A |
| nisB | 2,982 bp | 55 | Dehydratase |
| nisT | 1,803 bp | 56 | Transporter-Translocation protein |
| nisC | 1,113 bp | 57 | Thioether-forming enzyme |
| | | | (Lanthionine formation) |
| nisl | 738 bp | 58 | Lipoprotein-Immunity |
| nisP | 2,049 bp | 59 | Subtilisin-like serine protease |
| nisR | 687 bp | 60 | Regulator |
| nisK | 1344 bp | 61 | Regulator |
| nisF | 678 bp | 62 | Transporter-Immunity |
| nisE | 729 bp | 63 | Transporter-Immunity |
| nisG | 645 bp | 64 | Transporter-Immunity |

### 4.6 Lactococcin

The gene cluster putatively associated with the biosynthesis of a lactococcin bacteriocin is not encoded on a plasmid. This gene was originally annotated as coding for a hypothetical protein, but BAGEL3 identified it as a Lactococcin. Further, a Conserved Domain search shows similarity to members of PFAM04639, defined as Lactococcin-like family (family of bacteriocins from lactic acid bacteria).

Genomic analysis thus confirmed that Lactococcus lactis CCOS 949's exceptional activity against C. difficile may be linked to the production of peptogylcan hydrolases (PGHs). The one and same strain carries genes for, on one side, peptogylcan hydrolases (PGHs), specifically N-acetylglucosaminidases (4 in number), muramidases (2 in number) and, on the other side, for 3 bacteriocins, specifically 1 of the Nisin type. This combination is of particular relevance to the present invention as it allows to postulate that it is the presence of peptidoglycan hydrolases such as gylcosidases and amidases that contributes in this particular strain to the said unexpected activity.

### 4.7 Conclusion of Example 4

Both the Nisin genes and the gylcosidase-type genes (e.g. Nagase) expected on the basis of the enzyme and the inhibitory activity experiments could be indeed found. These genomic data support the concept that the exceptional activity of this particular strain is also due to the combined action of two types of antimicrobial peptides, bacteriocins (nisin and lactococcin) and PGHs (NAGases and muramidases).

In other words, LAB strains lacking nisin, but expressing PGHs, can be expected to possess an inhibitory activity against C. difficile, too. The data hereafter and those relating to further tests with Lactobacillus strains reported further below confirm this finding.

### Example 5: Spot Experiments Correlating NAGase Production and Anti-C. difficile Inhibition

Spot-experiment Series 1 was performed in order to qualitatively assess the inhibitory effect of cultures of Lactobacillus on C. difficile. To this end 2 Lactobacillus strains and 5 C. difficile hospital isolates were used. In a semi-quantitative set-up, the sediments of the cultured Lactobacillus were pipetted on sheep blood agar plates that had been previously inoculated with C. difficile. In order to boost the Lactobacillus counts the sampled cultures were suspended in MRS broth.

The Lactobacillus strains were enriched overnight with MRS broth. From these cultures 1-2 drops of the sediment were retrieved and filled in a tube. Out of this tube one drop was pipetted onto the agar plates previously spread over with C. difficile (McF 0.5-1). The plates prepared in the said way were incubated overnight (24 h) and results were read off the next day. Results are shown in Table 5.

TABLE 5
Reference Lactobacillus strains tested in spot-experiments against 5 C. diff in Series 1

| Lactobacillus | | | | | | | | |
|---|---|---|---|---|---|---|---|---|
| | | | “NAGase” | C. difficile Access number | | | | |
| | | Access | activity in | CCOS | CCOS | CCOS | CCOS | CCOS |
| Species | Code | number | APIZYM test | 958 | 957 | 938 | 937 | 939 |
| L. gasseri | KS | CNCM | high | (+) | ++ | + | + | + |
| | 120.1 | I-3218 | | | | | | |
| L. jensenii | KS | CNCM | Low or | − | (+) | (+) | − | + |
| | 119.1 | I-3217 | absent | | | | | |

Legend to Table 5:
Antimicrobial activity:
++: strong;
+: medium;
(+): weak:
−: no activity
Enzymatic activity for N-acetyl-glucosaminidase (NAGase) as determined by APYzym kit of Biomérieux:
Score range: 0 to 5.
Score >= 3: high activity;
Score = 1-2: low activity;
Score = 0 : absent activity.

The finding that the production of glycosidase is related to an anti-C. difficile activity in vitro was thus confirmed at the phenotypic level. Further experiments were conducted on Lactobacillus strains belonging to different species to verify the hypothesis that the presence glycosidases (NAGases or muramidases) is related to the anti-C. difficile activity.

After having found a remarkably active, nisin-producing Lactococcus lactis (CCOS 949) and based on the assumption, in view of experimental evidence, that such activity might be due to the contribution of the activity of Peptoglycan Hydrolases, in particular NAGase, investigation was extended to include a number of probiotic Lactobacillus species to see whether this hypothesis can be confirmed.

Like the other members of the Lactobacillus acidophilus group A, Lactobacillus gasseri is an anaerobic, gram-positive bacterium that falls into the category of lactic acid bacteria. It is also a rod shaped and of the non-spore-forming type. It was first described by Lauer and Kandler as a novel species in 1980 (Ref Lauer, Eckhard, and Otto Kandler. “Lactobacillus gasseri sp. nov., a new species of the subgenus Thermobacterium.” Zentralblatt fir Bakteriologie: I. Abt. Originale C: Allgemeine, angewandte und ökologische Mikrobiologie 1.1 (1980): 75-78). It is typically found in the gastrointestinal tracts of humans and animals due to its largely fermentative function (Alatossova, T., Munro, K., Ng, J., Tannock, G. W., & Tilsala-Timisjarvi, A. (1999) Identification of Lactobacillus Isolates from the Gastrointestinal Tract, Silage, and Yoghurt by 16S-23S rRNA Gene Intergenic Spacer Region Sequence Comparisons. Applied and Enviromental Microbiology. 65(9). 4364-4267; Falsen, E, Pascual, C, Sjoden, B, Ohlen, M, & Collins, MD. (1999) Phenotypic and phylogenetic characterization of a novel Lactobacillus species from human sources: description of Lactobacillus iners sp. Nov. Int J Syst Bacteriol. 49. 217-221; Mitsuoka, T. (1992) “The human gastrointestinal tract. In, The Lactic Acid Bacteria: Volume 1, The Lactic Acid Bacteria in Health and Disease” B.J.B. Wood (ed), pp69-114. Elsevier Science Publishers, Ltd. Essex, England).

Although mainly found in the GI tract, it can also be found in many other locations as well, in particular in the lower genital tract of healthy females.

Isolation of this bacterium was achieved by taking a sample from the gastrointestinal tract and was discovered to be part of what is today known as the Lactobacillus acidophilus complex (Kullen, M. J., R. B. Sanozky_Dawes, D. C. Crowell and T. R. Klaenhammer. (2000) Use of DNA sequence of variable regions of the 16SrRNA gene for rapid and accurate identification of bacteria in the Lactobacillus acidophilus complex. J. Appl. Microbiol. 89:511-518.). One of its roles, as described above, is fermentation in the GI tract. Recently, its function as a probiotic has been the area of most interest (Alatossova et al, see above). The complete genome of the type strain, L. gasseri ATCC 33323, has been sequenced by a combination of efforts from the Joint Genome Institute, Fidelity Systems Inc., and the North Carolina State University. The final draft was accepted on Oct. 13, 2006. (see DOE Joint Genome Institute http://genome.jgi-psf.org/finished_microbes/lacga/lacga.home.html). L. gasseri is an anaerobe so it lives mostly in body sites where no oxygen is present. It also participates in fermentative actions, which produce lactic acid as well as the energy required for growth.

Lactobacillus plantarum is a Gram positive, catalase negative bacterium, belonging to the heterogenous Lactobacillus genus, which includes more than 200 species. It is a lactic acid bacterium with a facultative heterofermentative metabolism. Unlike other members of the Lactobacillus genus, the distribution of L. plantarum in nature is rather wide, ranging from dairy products, vegetables, meat, silage, wine, as well as oral, gastrointestinal, vaginal and urogenital tracts (Seddik H A, Bendali F, Gancel F, Fliss I, Spano G, Drider D. “Lactobacillus plantarum and Its Probiotic and Food Potentialities” Probiotics Antimicrob Proteins. 2017; 9(2):111-22). The species L. plantarum is characterized by genomic and phenotypic variability, as around 120 genes (within more than 2000 constituting the core genome of L. plantarum) were found to be unique for this species, and 50 genes to be unique for the reference strain WCFS1 (Siezen, R.J., Bayjanov, J., Renckens, B., Wels, M., van Hijum, S.A., Molenaar, D., and van Hylckama Vlieg, J. E. (2010) “Complete genome sequence of Lactococcus lactis subsp. lactis KF147, a plant-associated lactic acid bacterium”. J Bacteriol 192: 2649-2650). The genus Lactobacillus includes Gram positive rods with fastidious growth requirement, most of which are commensals of gut and vaginal ecosystem, where, in particular, their preponderance is a sign of health (Petrova M I, Lievens E, Malik S, Imholz N, Lebeer S. “Lactobacillus species as biomarkers and agents that can promote various aspects of vaginal health” Front Physiol. 2015 Mar. 25; 6:81). Inside the genus, Lactobacillus crispatus includes strains colonizing the gastro-intestinal tract (Ojala T, Kuparinen V, Koskinen J P, Alatalo E, Holm L, Auvinen P, Edelman S, Westerlund-Wikström B, Korhonen T K, Paulin L, Kankainen M. “Genome sequence of Lactobacillus crispatus STi” J Bacteriol. 2010 July; 192(13):3547-8) and, typically, is one of the dominant species of the human vagina, together with L. gasseri, L. jensenii and L. iners (Ravel J, Gajer P, Abdo Z, Schneider G M, Koenig S S, McCulle S L, Karlebach S, Gorle R, Russell J, Tacket C O, Brotman R M, Davis C C, Ault K, Peralta L, Forney L J. “Vaginal microbiome of reproductive-age women” Proc Natl Acad Sci USA. 2011 Mar. 15; 108 Suppl 1:4680-7).

In the perspective of its use in food, notably, L. crispatus received in 2013 the qualified presumption of safety (QPS) status (https://www.efsa.europa.eu/it/efsajoumal/pub/4522), which allows its deliberate use in food and feed in Europe. Although L. crispatus does not ferment milk efficiently, it can survive in milk up to one month at 4° C., suggesting the use of this bacterium as adjunctive culture in dairy products with probiotic potential (Siroli L, Patrignani F, Serrazanetti D I, Parolin C, Ñahui Palomino R A, Vitali B, Lanciotti R. Determination of Antibacterial and Technological Properties of Vaginal Lactobacilli for Their Potential Application in Dairy Products. Front Microbiol. 2017 Feb. 7; 8:166). The spot-experiment Series 1 described below was performed in order to assess qualitatively the inhibitory effect of cultures of Lactobacillus on C. difficile. To this end, 12 Lactobacillus strains typical of the urogenital or the intestinal tract and 5 C. difficile hospital isolates were used. In a semi-quantitative set-up the sediment of the cultured Lactobacillus was pipetted on sheep blood agar plates that had been previously inoculated with C. difficile. In order to boost the Lactobacillus counts the sampled cultures were suspended in MRS broth.

a. Method

Lactobacillus strains were enriched over night with MRS broth. From these cultures 1-2 drops of the sediment were retrieved and filled in a tube. Out of this tube 1 drop was pipetted onto the agar plates previously spread over with C. difficile (McF 0.5-1). The plates prepared in the said way were incubated overnight (24 h) and results were read off the next day.

b. Results

Results are shown in Table 6.

TABLE 6
Inhibitory activity of reference Lactobacillus strains
in spot-experiments against 5 C. difficile in Series 1

| Lactobacillus | | | | C. difficile Access number | | | | |
|---|---|---|---|---|---|---|---|---|
| | | Access | NAGase | CCOS | CCOS | CCOS | CCOS | CCOS |
| Species | Code | number | activity | 957 | 958 | 938 | 937 | 939 |
| L. gasseri | CCOS | LMG | 1 | ++ | + | − | − | − |
| Type strain | 696 | 9203T | | | | | | |
| L. gasseri | KS | CNCM | 3 | ++ | + | ++ | + | + |
| | 120.1 | I-3218 | | | | | | |
| L. gasseri | KS | CNCM | 2 | ++ | (+) | + | (+) | + |
| | 124.3 | I-3220 | | | | | | |
| L. gasseri | 123.1 | CNCM | 0 | − | + | − | (+) | − |
| | | I-3485 | | | | | | |
| L. crispatus | KS | CNCM | 0 | (+) | − | + | + | (+) |
| | 116.1 | I-3483 | | | | | | |
| L. jensenii | KS | CNCM | 1 | (+) | − | (+) | − | + |
| | 119.1 | I-3217 | | | | | | |
| L. jensenii | KS | CNCM | 0 | + | − | − | − | − |
| | 121.1 | I-3219 | | | | | | |
| L. helveticus | KS | CNCM | 0 | ++ | (+) | − | (+) | − |
| | 300 | I-3360 | | | | | | |
| L. johnsonii | 824 | CCOS | 3 | + | ++ | + | + | + |
| | | 824 | | | | | | |
| L. reuteri | RC | ATCC | 0 | − | − | (+) | (+) | − |
| | 14 | 55845 | | | | | | |
| L. rhamnosus | GR-1 | ATCC | 0 | + | − | − | − | − |
| | | 55826 | | | | | | |
| L. rhamnosus | LGG | ATCC | 0 | + | − | (+) | − | + |
| | | 53103 | | | | | | |
| L. rhamnosus | | DSM | 2 | + | (+) | + | (+) | + |
| type strain | | 20021 | | | | | | |
| L. acidophilus | | ATCC | 0 | (+) | − | − | − | (+) |
| type strain | | 4356 | | | | | | |
| L. paracasei | DG ® | CNCM | 0 | − | + | − | (+) | − |
| subsp. paracasei | | I-1572 | | | | | | |
| L. paracasei | | CCOS | 4 | + | ++ | + | (+) | + |
| subsp. paracasei | | 1205/1201 | | | | | | |

Legend to Table 6:
Antimicrobial activity:
++: strong;
+: medium;
(+): weak:
−: no activity
Enzymatic activity for N-acetyl-glucosaminidase (NAGase) as determined by APYZym kit of Biomérieux:
Score range: 0 to 5.
Score >= 3: high activity;
Score = 1-2: medium-low activity;
Score = 0: not detected activity

c. Conclusion

From Series 1 it can be seen that the stronger and more consistent activity against C. difficile is exhibited by those strains with medium to high NAGase activity, e.g. L. gasseri KS 120.1, L. johnsonii CCOS 824, L. paracasei subsp. paracasei CCOS 1205 (identical to CCOS 1201). For most species strains can be found with no or low NAGase production, which are in the majority, as well as strains with high activity, actually a minority. It was found that L. paracasei subsp. paracasei DG has a low NAGase production and a low anti-Clostridium difficile activity, whereas the strain of the same species CCOS 824 hasa a significant NAGase production and anti-C. difficile activity. For the gasseri species it was found that examples with high, medium and low/no Nagase activity correlated to the C. difficile inhibition. The well-documented L. rhamnosus LGG, albeit reported active against numerous pathogens, does not strongly perform against C. difficile inhibition and lacks NAGase production.

A consistent inhibitory activity exhibited by L. johnsonii CCOS 824 was noted against different biotypes of C. difficile and its correlation with a high NAGase production. This combination—high NAGase activity with high inhibition of C. difficile—makes it a good probiotic strain for anti-infective applications, in particular for CDAD/CDI. This strain was also tested against different C. difficile strains in co-culture experiments yielding positive results above average thus confirming its inhibitory activity against this pathogen (data not shown).

Noticeable is also the good performace of L. paracasei strains producing NAGase e.g. CCOS 1205 (identical to CCOS 1201), as generally speaking L. paracasei exhibit numerous interesting probiotic properties and are suitable for industrial production.

In this line of reasoning, said correlation was tested with further Lactobacillus strains against the same C. difficile hospital strains used in the preceding experiment, with the perspective extend the knowledge of new strains with clear anti-C. difficile activity. In the same experiment, the pH-dependence of the activity was also evaluated, a point already considered in the co-culture experiment concerning Lactococcus lactis CCOS 949.

### Example 6: Series 2 Spot Experiments: Comparative Inhibition of C. difficile Strains by Different Species of Lactobacillus

New test strains belonging to the species L. plantarum, L. gasseri and L. reuteri were investigated. The dependency on the pH value and the correlation with the NAGase production of the strains was measured. As a negative control, L. reuteri RC-14 was used, as in the previous Examples.

TABLE 7
Lactobacillus strains tested in spot experiments
against 5 C. difficile in Example 6 (Series 2)

| Species/Strain | Access Code |
|---|---|
| Lactobacillus plantarum | CCOS 893 |
| Lactobacillus plantarum BG 112 | LMG P-20353 |
| Lactobacillus gasseri | CCOS 960 |
| Lactobacillus crispatus | CCOS 961 |
| Lactobacillus reuteri RC14 | ATCC 55845 |
| Clostridium difficile | CCOS 937* |
| Clostridium difficile | CCOS 938* |
| Clostridium difficile | CCOS 939* |
| Clostridium difficile | CCOS 940* |
| Clostridium difficile | CCOS 941* |

*Strains available from CCOS, Wädenswil, Switzerland

a. Methods

Media and growth conditions Lactobacillus spp.: the preparatory cultures were incubated for 48 h at 37° C. (with CO₂ 7.5%) on COS sheep blood plates (aerobically, without CO₂). Afterwards fresh cultures were inoculated in MRS broth and incubated overnight at 37° C. (aerobically, without CO₂). The overnight cultures of the C. difficile strains were then evenly scratched out on the COS plates (for each one C1-C5 one plate). Then 10 microliters of the suspensions of the overnight cultures of the Lactobacilli were pipetted on the plates that had been before inoculated with C. difficile. In order to observe the effect of pH value each suspension was additionally neutralized (with NaOH 4%) at a pH-value of 5-6 and likewise pipetted onto the inoculated plates. Finally the plates were incubated at 37° C. over the weekend and the inhibition of C. difficile assessed.

b. Results

TABLE 7a
Inhibition of Clostridium difficile by selected Lactobacilli in spot experiment

| | | | C. difficile | | | | |
|---|---|---|---|---|---|---|---|
| | pH-value | NAGase | CCOS | CCOS | CCOS | CCOS | CCOS |
| Lactobacillus | suspension | activity | 937 | 938 | 939 | 940 | 941 |
| RC 14 | 4.5 | low | +/− | +/− | − | − | − |
| RC 14 n | 5.5 | | +/− | − | − | − | − |
| CCOS 960 | 4 | high | + | ++ | + | + | + |
| CCOS 960 n | 5.5 | | + | +/− | − | − | +/− |
| BG 112 | 4 | medium | + | +/− | + | + | + |
| BG 112 n | 5.5 | | +/− | − | − | − | +/− |
| CCOS 893 | 4 | high | + | +/− | + | + | + |
| CCOS 893 n | 5.5 | | +/− | − | +/− | +/− | +/− |
| CCOS 961 | 4.5 | medium | + | +/− | + | +/− | +/− |
| CCOS 961n | 5.0-5.5 | | +/− | +/− | − | − | − |
| MRS | >6 | — | − | − | − | − | − |

Legend to Table 7a:
n: neutralized;
Antimicrobial activity:
++: strong;
+: medium;
+/−: weak:
−: no activity;
Enzymatic activity for N-acetyl-glucosaminidase (NAGase) as determined by APYZym kit of Biomérieux:
Score range: 0 to 5.
Score >= 3: high activity;
Score = 1-2: medium activity;
Score = 0: low or undetected activity

c. Conclusion

The strain L. reuteri RC 14 shows only a feeble activity at low pH, which disappears completely at higher pH., whereas L. plantarum BG 112 is quite active especially at low pH. The 2 strains L. gasseri CCOS 960 and L. plantarum CCOS 893 exhibit a consistent antimicrobial activity at lower pH that is partially maintained at higher pH. Clearly, L. plantarum CCOS 893 shows the best, and L. gasseri CCOS 960 the second-best performance. It is also worth noticing that the data Table 7a, showing a positive correlation between NAGase production and anti-C. difficile activity, confirm this link already reported for other species/strains in the experiments in Tables 1-6. On this basis, these 2 last strains are thus considered useful for being used in a probiotic anti-C. difficile or more generally in an anti-diarrhoeal formulation according to the present invention. Co-culture experiments of C. difficile with selected Lactobacillus test strains (L. gasseri and L. plantarum) were then carried out.

### Example 7: Series 3 Experiments: Test Trains Versus Strain Established in the Gastro-Intestinal Tract

In order to confirm the above results on a more quantitative basis the inhibition of C. difficile by Lactobacillus was studied by means of a co-culture technique. Three Lactobacillus strains known for their marked activity were investigated against a selection of C. difficile isolates in a similar way as in the previous Examples.

TABLE 8
Test and Reference Lactobacillus Strains tested in co-culture
experiments against 5 C. difficile in Serie 3

| Nr. | Species | Working Code | Access Code |
|---|---|---|---|
| L1 | Lactobacillus gasseri | KS 120.1 | CNCM I-3218 |
| L2 | Lactobacillus gasseri | CCOS 960 | DSM 32296 |
| L6 | Lactobacillus plantarum | CCOS 893 | DSM 32352 |
| C1 | Clostridium difficile | 2014 11678 | CCOS 937 |
| C2 | Clostridium difficile | 2014 11648 | CCOS 938 |
| C3 | Clostridium difficile | 2014 11698 | CCOS 939 |
| C4 | Clostridium difficile | 2015 110285 | CCOS 940 |
| C5 | Clostridium difficile | Test-strain Lab | CCOS 941 |

a. Method:

Media and growth conditions: Lactobacillus sp. pre-cultures were inoculated in MRS-broth and incubated over the weekend at 37° C. (without CO₂). Out of this broth fresh cultures were subsequently inoculated in MRS-broth and aerobically incubated overnight at 37° C. (without CO₂). C. difficile pre-cultures were inoculated in CMC under gas flow and incubated anaerobically over the weekend at 37° C. (without CO₂). Out of this broth fresh cultures were subsequently inoculated in CMC-broth under gas-flow and incubated overnight at 37° C. (without CO₂).

Experimental procedure: The overnight cultures of the Lactobacillus strains were diluted to a McFarland standard 4 with MRS broth. The overnight cultures of the C. difficile strains were diluted in a 1:100 ratio in CMC. Co-cultures were made by inoculating 3 ml of the diluted C. difficile strains with 600 mcl of the Lactobacillus strain suspension diluted to McFarland 4 in small Pyrex-tubes (with air-tight locked screw cap).In that way co-cultures resulted with a CMC-MRS ratio of 5:1. For the growth control of the pathogens 600 μl MRS broth were added to a diluted C. difficile culture and carried along in the incubation. At time of mixing, Lactobacillus outnumber C. difficile by about 2 powers of 10. Shortly after the assemblage the first test-sample was withdrawn (t=0 h) and the co-cultures were incubated for 48 h, whereby at any one time additional samples were withdrawn after 4 h, 8 h, 24 h and 48 h. At each sample taking 100 μl of the co-cultures were pipetted in 900 microliter NaCl-solution and out of it a dilution series of 1:10, 1:100 and 1:1000 was made. Subsequently, out of each dilution 10 microliter were pipetted onto a sheep blood agar plate and evenly smoothed over the same. These plates were finally incubated for 2 d and the respective colonies forming unit (cfu) counted. As the C. difficile colonies and those of the Lactobacillus are morphologically different it was feasible to distinguish on the same plate. C. difficile colonies were assessed quantitatively, those of the Lactobacillus semi-quantitatively. The media used as well as the co-culture medium were such as to guarantee the growth of both Lactobacillus and C. difficile.

The average values measured were as follows:
CMC not inoculated: 7
MRS not inoculated: 6.5
Mixed growth medium
Precultures Lactobacillus: 4
Precultures C. difficile: 6

| pH | Co-culture medium | C. difficile-only in medium |
|---|---|---|
| t = 0 h | 6.5 | 7 |
| t = 24 h | 5 | 6 |
| t = 48 h | 5 | 6 |

b. Results

TABLE 8a
In a first experiment Lactobacillus L1 and L2 were tested
against each one of the reference C. difficile strains C1 to C5.
The raw data are shown in detail for e.g. C. difficile C3

| Point in time | Dilution | Control | L1 | | L2 | |
|---|---|---|---|---|---|---|
| t = 0 h | 1:10 | 123 | 91 | +++ | 42 | ++ |
| | 1:100 | 13 | 13 | ++ | 7 | 1 |
| | 1:1000 | 0 | 0 | + | 0 | +/− |
| t = 4 h | 1:10 | nc | 200* | +++ | 150 | +++ |
| | 1:100 | 145 | 37 | +++ | 9 | ++ |
| | 1:1000 | 18 | 4 | ++ | 0 | + |
| t = 8 h | 1:10 | nc | nc | +++ | nc | +++ |
| | 1:100 | nc | 86 | +++ | 84 | ++ |
| | 1:1000 | 400* | 16 | ++ | 10 | + |
| t = 24 h | 1:10 | nc | nc | +++ | nc | +++ |
| | 1:100 | nc | 148* | ++ | 105 | ++ |
| | 1:1000 | 356* | 22 | ++ | 7 | + |
| t = 48 h | 1:10 | nc | 25 | +++ | 29 | +++ |
| | 1:100 | nc | 1 | ++ | 0 | ++ |
| | 1:1000 | 59 | 0 | ++ | 0 | + |

Quantitation C. difficile
Number: cfus on plate
*number extrapolated
nc: not countable (>>100 cfus)
Estimation Lactobacillus
+++: bacterial lawn on plate
++: bacterial lawn with single colonies delimitable
+: >500 cfu
+/−: 100-500 cfus

In order to represent graphically the data C. difficile concentrations were processed as follows:
Control concentrations: in rounded-up cfus on a logarithmic scale
Co-cultures: as a rounded-up ratio between the cfus in the presence of Lactobacillus and the control cfus at the same point in time

The corresponding Table 8b for the same C. difficile C3 looks then as follows

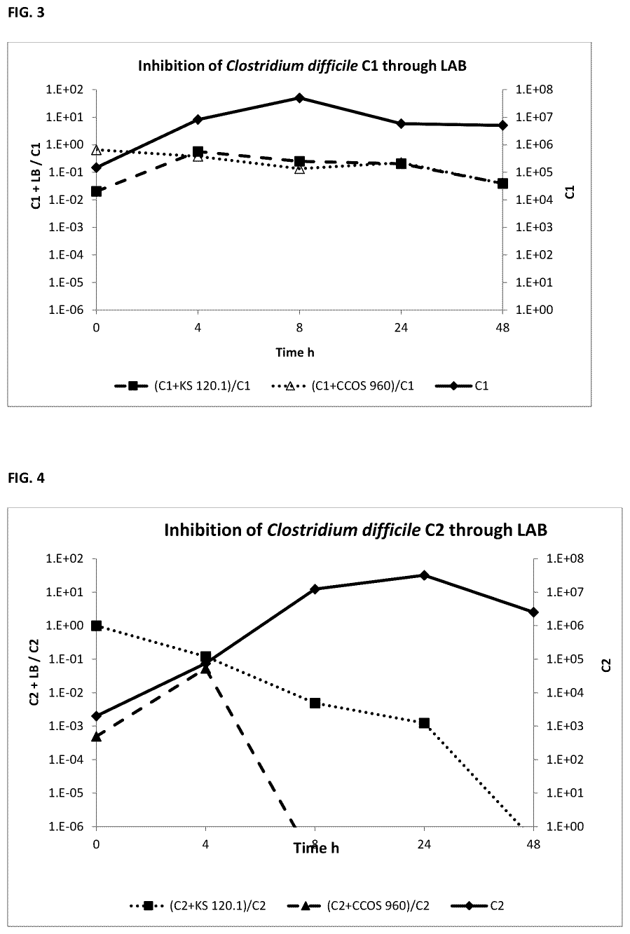

TABLE 8b
Inhibition of C. difficile C3 through L1 and L2.
This data is shown graphically in FIG. 2.

| Hours | C3 (cfus) | (C3 + L1)/C3 | (C3 + L2)/C3 |
|---|---|---|---|
| 0 | 1.E+05 | 1.E+00 | 5.E−01 |
| 4 | 1.E+06 | 3.E−01 | 6.E−02 |
| 8 | 4.E+07 | 4.E−02 | 3.E−02 |
| 24 | 4.E+07 | 6.E−02 | 2.E−02 |
| 48 | 6.E+06 | 4.E−03 | 5.E−03 |

From the diagram in FIG. 2, it can be concluded that, the concentration of C3 on the average increases over time, indicating that the medium is suitable for the vitality and growth of C. difficile and that there is therefore no medium-related bias. In the following, the results for the other 4 C. difficile strains are shown in compact form.

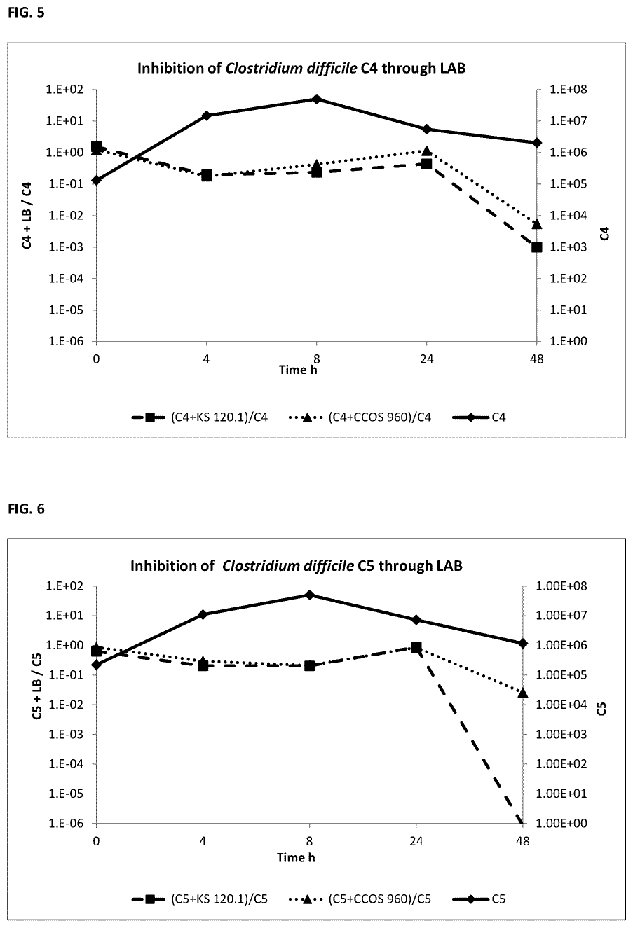

Table 8c (left) Inhibition of C. difficile C1 through L1 and L2. These data are reported in the graphic of FIG. 3 and Table 8d (right): Inhibition of C. difficile C2 by Lactobacilli L1 and L2. These data are reported in the graphic of FIG. 4.

| Hours | Cl | (C1 + L1)/C1 | (C1 + L2)/C1 |
|---|---|---|---|
| 0 | 1.E+05 | 2.E−02 | 7.E−01 |
| 4 | 8.E+06 | 6.E−01 | 4.E−01 |
| 8 | 5.E+07 | 3.E−01 | 1.E−01 |
| 24 | 6.E+06 | 2.E−01 | 2.E−01 |
| 48 | 5.E+06 | 4.E−02 | 4.E−02 |

| Hours | C2 | (C2 + L1)/C2 | (C2 + L2)/C2 |
|---|---|---|---|
| 0 | 2.E+03 | 1.E+00 | 5.E−04 |
| 4 | 8.E+04 | 1.E−01 | 5.E−02 |
| 8 | 1.E+07 | 5.E−03 | 8.E−08 |
| 24 | 3.E+07 | 1.E−03 | 3.E−08 |
| 48 | 3.E+06 | 4.E−07 | 4.E−07 |

The two L. gasseri achieve a reduction of almost 2 logs in the viability of C. difficile C1 after 48 h. This shows that the NAGase-producing strains L1 (Lactobacillus gasseri KS 120.1, CNCM I-3218) and L2 (Lactobacillus gasseri CCOS 960, deposit number DSM 32296) are able to efficiently kill C. difficile C2.

Table 8e (left): Inhibition of C. difficile C4 by Lactobacillus L1 and L2. These data are reported in the graphic of FIG. 5. Table 8f (right): Inhibition of C. difficile C5 by Lactobacillus L1 and L2. These data are reported in the graphic of FIG. 6.

| | C4 | (C4 + L1)/C4 | (C4 + L2)/C4 |
|---|---|---|---|
| 0 | 1.E+05 | 2.E+00 | 1.E+00 |
| 4 | 1.E+07 | 2.E−01 | 2.E−01 |
| 8 | 5.E+07 | 2.E−01 | 4.E−01 |
| 24 | 6.E+06 | 4.E−01 | 1.E+00 |
| 48 | 2.E+06 | 1.E−03 | 5.E−03 |

| Hours | C5 | (C5 + L1)/C5 | (C5 + L2)/C5 |
|---|---|---|---|
| 0 | 2.20E+05 | 6.E−01 | 9.E−01 |
| 4 | 1.09E+07 | 2.E−01 | 3.E−01 |
| 8 | 5.00E+07 | 2.E−01 | 2.E−01 |
| 24 | 7.20E+06 | 9.E−01 | 8.E−01 |
| 48 | 1.17E+06 | 9.E−07 | 3.E−02 |

L. gasseri KS 120.1 shows the best activity of the 3 Lactobacillus with a reduction of 3 log after 48 h.

c. Conclusion

The C. difficile Test-strain Lab CCOS 941 (C5) is a strain sensitive to specific probiotics: after 48 h L. gasseri KS 120.1 eliminates the pathogen in the co-culture. L1 (L. gasseri KS 120.1, CNCM I-3218) and L2 (L. gasseri CCOS 960, deposit number DSM 32296) show activity against C. difficile. In order to gather more information, it was then decided to add to the test another relevant species investigated before for its reported consistent activity against C. difficile, L. plantarum.

### Example 8: Experiments Series 4

The same methods and experimental procedures were used as described in Example 7.

TABLE 9
strains involved in Experiments Series 4

| Codes | Species | Working Code | Access Code |
|---|---|---|---|
| L6 | Lactobacillus plantarum | CCOS 893 | DSM 32352 |
| Cl | Clostridium difficile | 2014 11678 | CCOS 937 |
| C2 | Clostridium difficile | 2014 11648 | CCOS 938 |
| C3 | Clostridium difficile | 2014 11698 | CCOS 939 |
| C4 | Clostridium difficile | 2015 110285 | CCOS 940 |
| C5 | Clostridium difficile | Test-strain Lab | CCOS 941 |

a. Results Series 4: Tables 9a-e and FIGS. 1-5

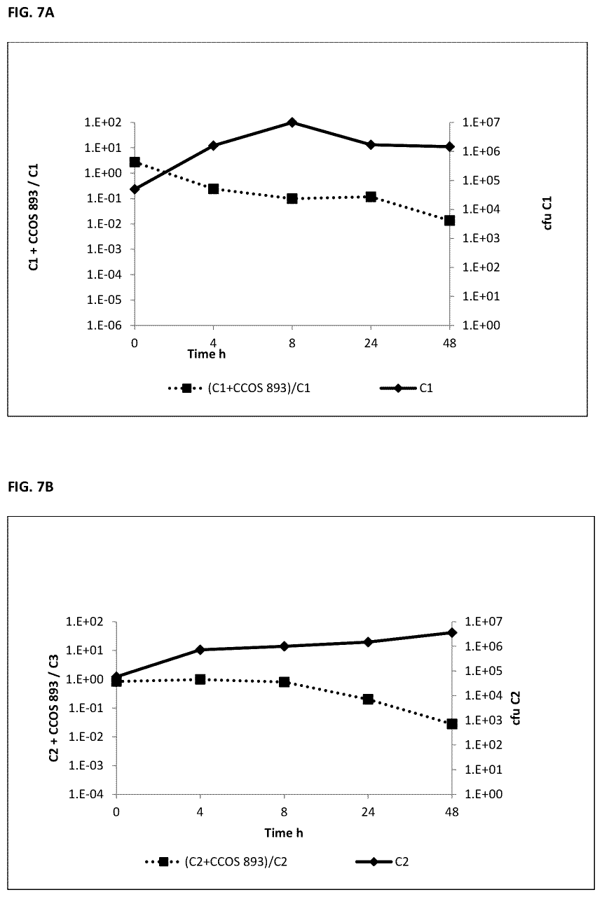

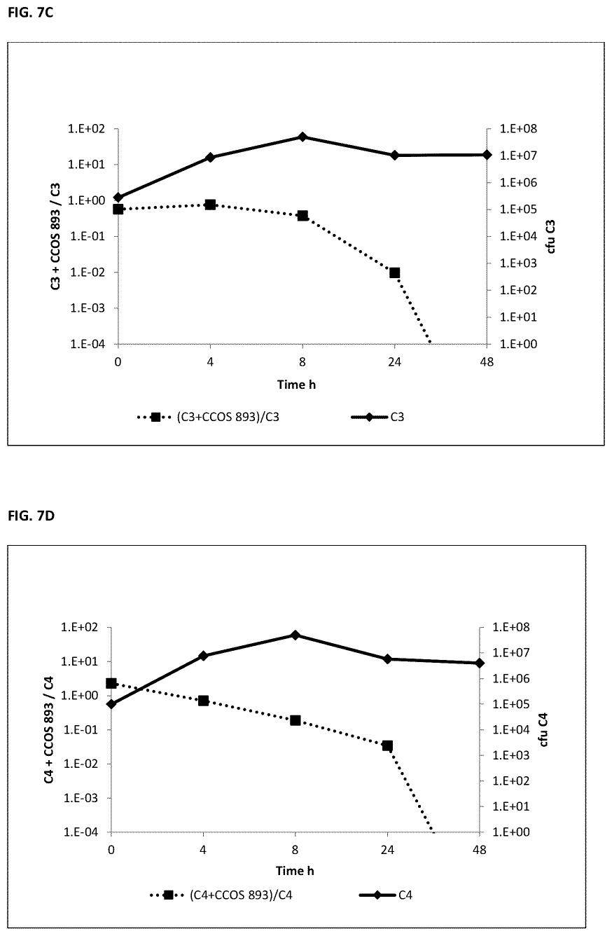

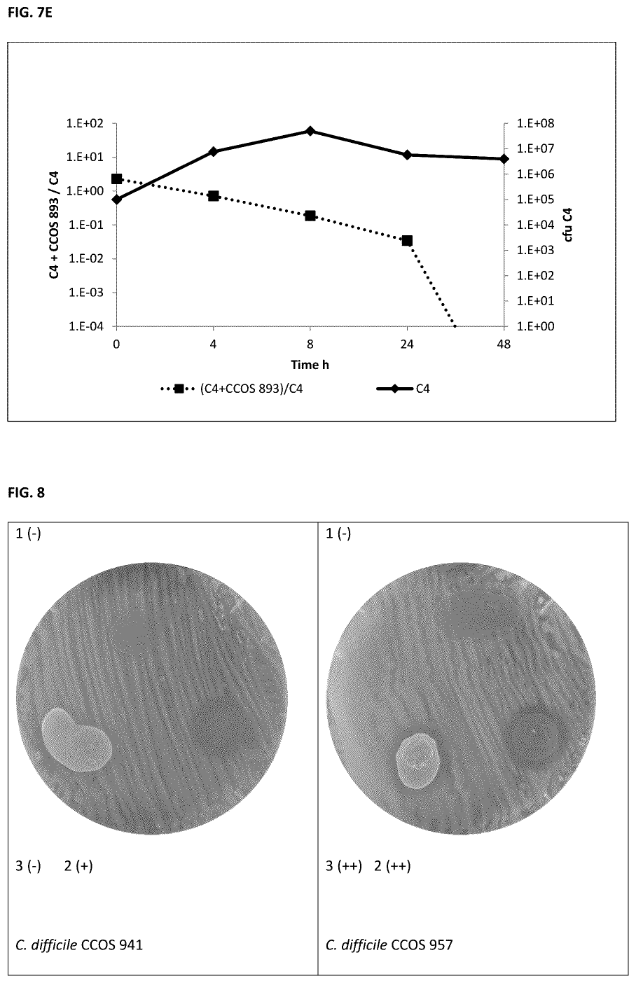

Tables 9a-e regarding inhibition of C. difficile C1-C5 by L. plantarum CCOS 893 are reported hereunder (The same data are reported in the Graphics in FIGS. 7A-7E). The absolute concentration of the C. difficile alone (cfu) as well as the relative reduction of the concentration of C. difficile in the presence of a Lactobacillus are shown as a function of time

TABLE 9a
Inhibition of C. difficile Cl by L. plantarum CCOS 893

| time h | C1 | (C1 + CCOS 893)/C1 |
|---|---|---|
| 0 | 5.E+04 | 3.E+00 |
| 4 | 2.E+06 | 2.E−01 |
| 8 | 1.E+07 | 1.E−01 |
| 24 | 2.E+06 | 1.E−01 |
| 48 | 1.E+06 | 1.E−02 |

TABLE 9b
Inhibition of C. difficile C2 by L. plantarum CCOS 893

| time h | C2 | (C2 + CCOS 893)/C2 |
|---|---|---|
| 0 | 6.E+04 | 8.E−01 |
| 4 | 7.E+05 | 1.E+00 |
| 8 | 1.E+06 | 8.E−01 |
| 24 | 2.E+06 | 2.E−01 |
| 48 | 4.E+06 | 3.E−02 |

TABLE 9c
Inhibition of C. difficile C3 by L. plantarum CCOS 893

| time h | C3 | (C3 + CCOS 893)/C3 |
|---|---|---|
| 0 | 3.E+05 | 6.E−01 |
| 4 | 9.E+06 | 8.E−01 |
| 8 | 5.E+07 | 4.E−01 |
| 24 | 1.E+07 | 1.E−02 |
| 48 | 1.E+07 | 9.E−08 |

TABLE 9d
Inhibition of C. difficile C4 by L. plantarum CCOS 893

| time h | C4 | (C4 + CCOS 893)/C4 |
|---|---|---|
| 0 | 1.E+05 | 2.E+00 |
| 4 | 8.E+06 | 7.E−01 |
| 8 | 5.E+07 | 2.E−01 |
| 24 | 6.E+06 | 3.E−02 |
| 48 | 4.E+06 | 3.E−07 |

TABLE 9e
Inhibition of C. difficile C5 by L. plantarum CCOS 893

| time h | C5 | (C5 + CCOS 893)/C5 |
|---|---|---|
| 0 | 1.E+05 | 9.E−01 |
| 4 | 1.E+07 | 5.E−01 |
| 8 | 5.E+07 | 2.E−01 |
| 24 | 3.E+06 | 1.E−01 |
| 48 | 1.E+06 | 2.E−02 |

b. Conclusion

Against all C. difficile, the more resistant C1 and C5 as well as the less resistant C2, C3 and C4, L. plantarum CCOS 893 achieves to gradually reduce over time the concentration of all the tested C. difficile strains between 2 and 6 logs. This is even better than the results of the strains in Series 3. The results of the investigations show that a correlation between the production of NAGase and the antimicrobial activity against C. difficile can be assessed for different strains of probiotic species comprising: L. gasseri, L. plantarum and L. crispatus. This could be verified in spot and/or co-culture experiments even at pH=or >5.0.

### Example 9: Genome Sequences of Lactobacillus Gasseri CCOS 960, Lactobacillus plantarum CCOS 893, Lactobacillus Crispatus CCOS 961 and Lactobacillus jensenii CCOS 962

Whole genomic high molecular weight DNA extraction of each strain was grown in 10 ml of liquid MRS (Man, Rogosa and Sharpe medium) at 37° C. without shaking in a 50 ml conical tubes for 48 h (cell density 1×10⁹ cells/ml). A 1 ml aliquot was centrifuged for 5 min at 8,000×g. The supernatant was removed and resuspended in 500 μl of 5M LiCl. The cells were placed on ice and vigorously vortexed every 2 minutes for 15 minutes. The cells were then centrifuged at 8,000×g for 5 min. A total of 500 μl of saturated phenol was added to the cell pellet, vortexed and centrifuged at 8,000×g. The cell pellet was washed with PBS and resuspended in 500 μl of PBS. Whole genomic DNA was extracted using MasterPure™ DNA Purification Kit (Epicentre, Cat. No. MCD85201). A total of 10 μg of DNA was used to construct a large insert (5-20 kb) SMRTbell™ sequencing library that was sequenced on a PacificBiosciences RSII instruments according to the manufacturer recommendations at the Institute for Genome Sciences, University of Maryland School of Medicine, Baltimore, MD USA. The genomes were assembled using the MinHash Alignment Process (MHAP)(Berlin K, Koren S, Chin C-S, Drake J P, Landolin J M, Phillippy A M. “Assembling large genomes with single-molecule sequencing and locality-sensitive hashing” Nat Biotechnol. 2015; 33:623-630 The estimated genome sizes, number of contigs, gene and coding sequence counts are shown in Table 10a. Annotation was generated using the Prodigal annotation pipeline (Hyatt D, Chen G-L, LoCascio P F, Land M L, Larimer F W, Hauser L J. “Prodigal: prokaryotic gene recognition and translation initiation site identification” BMC Bioinformatics. 2010; 11:119).

Glucosaminidase in these Lactobacillus strains are mostly mannosyl-glycoprotein endo-beta-N-acetylglucosaminidase and muramidase (cell-wall hydrolases). This large family of protein is involved in peptidoglycan catabolic processes. Of note is the identification of homologues to AcmA, a modular autolysin, consisting of a N-terminal Nacetylglucosaminidase and C-terminal LysM domains (peptidoglycan binding domain). The enzyme has a muramidase activity. Other muramidases were identified (Table 10a).

TABLE 10a
beta-D-N-acetylglucosaminidase and further PGHs
encoded on the genomes of Lactobacillus gasseri CCOS 960,
Lactobacillus plantarum CCOS 893, Lactobacillus crispatus CCOS 961.

| | | SEQ ID NO | SEQ ID NO | |
|---|---|---|---|---|
| Strain | Gene name | nucleotide | amino acid | Annotation/function |
| L. gasseri | LG_M960_06890 | 2 | 90 | Mannosyl-glycoprotein endo-beta-N- |
| CCOS 960 | | | | acetylglucosaminidase |
| | LG_M960_00270 | 1 | 89 | Mannosyl-glycoprotein endo-beta-N- |
| | | | | acetylglucosaminidase |
| | LG_M960_22860 | 3 | 91 | Muramidase-2 |
| | LG_M960_22870 | 4 | 92 | Muramidase (Glucosaminidase |
| | | | | Superfamily) |
| L. plantarum | Lplant_09370 | 5 | 93 | Mannosyl-glycoprotein endo-beta-N- |
| CCOS 893 | | | | acetylglucosaminidase |
| | Lplant_29740 | 6 | 94 | Mannosyl-glycoprotein endo-beta-N |
| | | | | acetylglucosaminidase |
| | Lplant_14230 | 7 | 95 | Mannosyl-glycoprotein endo-beta-N |
| | | | | acetylglucosaminidase |
| | Lplant_20460 | 8 | 96 | mannosyl-glycoprotein endo-beta-N |
| | | | | acetylglucosaminidase |
| | Lplant_20470 | 9 | 97 | mannosyl-glycoprotein endo-beta-N |
| | | | | acetylglucosaminidase |
| L. crispatus | LC119_17710 | 11 | 99 | Mannosyl-glycoprotein endo-beta-N- |
| CCOS 961 | | | | acetylglucosaminidase |
| | LC119_09570 | 10 | 98 | Bacterial surface layer protein acmA |

Using the BAGEL3 Algorithm (de Jong A, van Heel A J, Kok J, Kuipers O P. “BAGEL2: mining for bacteriocins in genomic data” Nucleic Acids Res. 2010; 38:W647-51) and BLAST these bacteriocin biosynthetic gene clusters were identified encoded in the genomes of L. gasseri CCOS 960 and L. crispatus CCOS 961 (Table 10b). No bacteriocins were found encoded in the genome of L. plantarum CCOS 893.

TABLE 10b

| | | SEQ | |
|---|---|---|---|
| Strain | Gene name | ID NO | Annotation |
| L. gasseri | LG_M960_18060 | 12 | Pediocin [1e-09] |
| CCOS 960 | | | |
| | LG_M960_18120 | 13 | Acidocin_LF221B(GassericinK7B) [3e-27] |
| | | | Acidocin_LF221B(GassericinK7B) [1e-11] |
| | LG_M960_18130 | 14 | Gassericin_T [3e-40] |
| | LG_M960_19200 | 15 | Bacteriocin_helveticin J [5e-60] |
| L. crispatus | LC119_03500 | 16 | enterolysin_A [9e-22] |
| CCOS 961 | | | |
| | LC119_04790 | 17 | Bacteriocin_helveticin_J [3e-171] |
| | LC119_12230 | 18 | Helveticin-J [3e-124] |
| | LC119_14630 | 19 | bacteriocin_LS2chaina [1e-09] |
| | LC119_14940 | 20 | Penocin_A [1e-08] |
| | LC119_15020 | 21 | Penocin_A [2e-12] |
| | LC119_27080 | 22 | enterolysin_A [1e-59] |
| | LC119_27330 | 23 | enterolysin_A [3e-94] |

| | SEQ ID NO | SEQ ID NO | | |
|---|---|---|---|---|
| locus tag | nucleotide | amino acid | Annotation | Site of action |
| CCOS-96249 | 84 | 147 | Urocanate reductase | Glycan-Peptide bond |
| 00293 | | | | |
| CCOS-96249 | 85 | 148 | Exo-glucosaminidase | Glycan-Peptide bond |
| 01072 | | | LytG | |
| CCOS-96249 | 86 | 149 | Penicillin-binding | Glycan-Peptide bond |
| 01421 | | | protein 1A | |
| CCOS-96249 | 87 | 150 | C protein alpha- | Glycan-Peptide bond |
| 01513 | | | antigen | |
| CCOS-96249 | 88 | 151 | Exo-qlucosaminidase | Glycan strand |
| 00553 | | | LvtG | |

Genomic analysis thus confirms that all 4 new Lactobacillus with clear inhibitory activity against C. difficile possess genes for different PGHs (mainly of the type Mannosyl-glycoprotein endo-beta-N-acetylglucosaminidase, but also e.g. muramidases or bacterial surface layer protein AcmA) that enable the carrier bacterium to attack, modify and/or disrupt bacterial cell walls. For instance, L. plantarum CCOS 893 is endowed with as much as 5 such genes.

On the side of the bacteriocins mainly such belonging to Class II were found (e.g. Gassericin, Helveticin, Penocin a.s.o.) with L. crispatus CCOS 961 having a total of 10 of the corresponding genes.

These data as presented above make it plausible that in these probiotic Lactobacillus the presence of PGHs targeted against the typical cell wall of bacterial pathogens, i.e. exhibiting NAGases or Muramidases a.s.o., possibly associated with Class II bacteriocins, enables these probiotic strains to exert an anti-microbial activity against C. difficile Of course, the presence of the genes does not per se warrant the production of the PGHs, for our probiotic Lactobacillus this was indeed confirmed by in vitro enzyme tests.

Of note, L. plantarum CCOS 893 does not possess bacteriocins genes (and therefore no bacteriocin activity) even though such are known for this species and have been described since the 1990's (e.g. plantaricins). Thus, this example proves that PGHs may not only contribute to the anti-microbial activity of Lactococcus and Lactobacillus, as discussed above, but even, as in this particular case of L. plantarum CCOS 893, constitute the main if not only component for the pronounced anti-microbial activity against C. difficile (as lactic acid alone or hydrogen peroxide are not sufficient to generally inhibit C. difficile). This proves that PGHs are a so far unrecognized, independent class of anti-microbial peptides of probiotic bacteria, a discovery of significant practical value.

### Example 10: Comparative Activity of Selected Bifidobacteria Against C. difficile in Spot Experiments and Correlation with PGHs

Bifidobacterium longum subsp. longum strain CCOS 974 is a gram-positive, obligate anaerobic, non-motile and non-spore forming bacterium, belonging to the Bifidobacterium genus, phylum Actinobacteria. Bifidobacteria are strict anaerobic microorganisms with a fermentative metabolism, particularly adapted to gut environments (Sun, Z. et al. “Comparative genomic analysis of 45 type strains of the genus Bifidobacterium: a snapshot of its genetic diversity and evolution” PLoS One 10(2), e0117912, 2015).

In the review by Christine S M Lau and Ronald S Chamberlain (“Probiotics are effective at preventing Clostridium difficile-associated diarrhea: a systematic review and meta-analysis” Int J Gen Med. 2016; 9: 27-37. doi: 10.2147/IJGM.S98280 26 RCT) involving 7,957 patients were analysed and some effect in CDAD found. None of these studies showed, however any positive clinical effect related to Bifidobacteria on CDAD.

There are only scarce papers in the literature dealing with the antimicrobial activity of Bifidobacteria against C. difficile. One of the reasons, is that bifidobacteria do not produce bacteriocins as do Lactobacillus. Lacking these substances, which are potent weapons against similar competing species as well as against pathogens, the Bifidobacteria were up to now not deemed as performant as the Lactobacillus w.r.t. antimicrobial activity.

Even at the laboratory level no successful results referring to an inhibition of C. difficile have been so far reported. For instance, in the systematic investigation reported by P. Hutt et al. (Journal of Applied Microbiology 100 (2006) 1324-1332, “038) 5 best-in-class Lactobacillus and 2 such Bifidobacteria were systematically tested for their anti-microbial activity against the leading uro- and enteropathogens. With respect to C. difficile the result for the best-in-class Bifidobacteria (Bifidobacterium lactis Bb12, Bifidobacterium longum 46) was clear: “No efficient antagonist against C. difficile was found”. Nevertheless, the inventors attempted to investigate the possible use of bifidobacteria against enteropathogens in particular Clostridium difficile.

a. Method

In a first step, detection of NAGase activity of selected Bifidobacteria was investigated by means of the APIZym test kit of Biomdrieux as summarized in Table 11.a. Bifidobacteria were first grown anaerobically in liquid MRS and then incubated under anaerobic conditions for 24 h at 37° C. APIZYM tests were prepared and analysed according the manufacturers instructions. Bifidobacteria were grown on MRS medium supplemented with cystein (0.5 g/l) under anaerobe conditions. For the test, a fresh overnight culture grown was resuspended in sterile physiological saline solution at an optical density corresponding to a McFarland standard of 5 to 6. This solution was dispensed into the wells of the strips of the test system. The tests strips were incubated ar 37° C. for 20 hours. After addition of the test reagents the strips were exposed to light and then read visually and interpreted according to the test instructions resulting as a value from 0 (negative) to 5 (strong positive) for each test. Each test was repeated at least twice.

TABLE 11a
List of test and reference Bifidobacteria and their NAGase score

| Bifidobacterium | | | Commercial resp. | NAGase |
|---|---|---|---|---|
| species | Strain code | Origin | Deposit code | score |
| B. breve | CCOS 586 | Infant faeces | CCOS 586 | 4 |
| B. bifidum | CCOS 571 | Infant faeces | CCOS 571 | 5 |
| B. lactis BB12 | CCOS 973 | Probiotic Isolate | DSM 15954 | 1 |
| B. longum | CCOS 974 | Probiotic isolate | CCOS 974 | 4 |

b. Conclusion:

From these experiments we infer that Bifidobacteria are able, like Lactobacillus spp., to produce, depending on the species and the strain, different amounts of N-acetylglucosaminidase (or glycosidase). For instance B. breve CCOS 586 and B. bifidum CCOS 571 produce high amounts (>=40 nmol), whereas the B. longum CCOS 974 produces about 30 mmol and BB12 only 10 nmol of this particular PGH.

### Example 11: Series 3 Experiments: Inhibition of C. difficile by Selected Bifidobacteria

TABLE 11c
List of tested C. difficile strains

| Strain code | Origin | Access # |
|---|---|---|
| CCOS 871 | Type strain | LMG 21717 |
| CCOS 876 | Clinical isolate | CCOS 876, public collection |
| CCOS 877 | Clinical isolate | CCOS 877, public collection |
| CCOS 937 | Clinical isolate | CCOS 937, public collection |
| CCOS 938 | Clinical isolate | CCOS 938, public collection |
| CCOS 939 | Clinical isolate | CCOS 939, public collection |
| CCOS 940 | Clinical isolate | CCOS 940, public collection |
| CCOS 941 | Clinical isolate | CCOS 941, public collection |
| CCOS 957 | Clinical isolate | CCOS 957, public collection |
| CCOS 958 | Clinical isolate | CCOS 958, public collection |

TABLE 11.d
Number of times a Bifidobacterium strain showed inhibitory activity aganst a C. difficile
strain. The number in parenthesis refer to the number of experiments. The table is vertically sorted
according to the number of C. difficile strains inhibited by a certain Bifidobacterium

| | | Clostridium difficile strains | | | | | | | | | | |
|---|---|---|---|---|---|---|---|---|---|---|---|---|
| | | | | | | | | | | | | Number of |
| | | | | | | | | | | | | inhibited |
| | Strain | CCOS | CCOS | CCOS | CCOS | CCOS | CCOS | CCOS | CCOS | CCOS | CCOS | C. difficile |
| Bifido-bacteria | code | 871 | 876 | 877 | 937 | 938 | 939 | 940 | 941 | 957 | 958 | strains |
| B. bifidum | CCOS 571 | 0 (2) | 3 (3) | 1 (3) | 1 (7) | 0 (4) | 0 (1) | 1 (2) | 1 (6) | 2 (4) | 1 (4) | 7 |
| B. longum | CCOS 974 | 0 (2) | 1 (2) | 0 (2) | 5 (6) | 3 (3) | 1 (1) | 0 (2) | 3 (5) | 1 (3) | 1 (3) | 7 |
| B. breve | CCOS 586 | 0 (2) | 0 (3) | 0 (3) | 1 (7) | 0 (4) | 0 (1) | 0 (2) | 0 (6) | 1 (4) | 1 (4) | 3 |
| B. lactis | BB12 | 0 (0) | 0 (0) | 0 (0) | 0 (3) | 0 (3) | 0 (1) | 0 (0) | 1 (3) | 0 (0) | 0 (0) | 1 |

| | | | | | | | | | | |
|---|---|---|---|---|---|---|---|---|---|---|
| Number of Bifidobacteria | 0 | 2 | 1 | 3 | 1 | 1 | 1 | 3 | 3 | 3 |
| inhibiting a strain certain | | | | | | | | | | |
| C. difficile | | | | | | | | | | |

The Clostridium difficile strains are available from the public collection of the Culture Collection of Switzerland CCOS, Einsiedlerstrasse 34, 8820 Wadenswil, Switzerland, www.ccos.ch.

Examples of inhibitory effects out of this experimental series of 3 Bifidobacteria against 2 different C. difficile strains are shown in FIG. 8. 1: B. lactis BB12, 2: B. bifidum CCOS 571, 3: B. breve CCOS 586. (−): no zone of inhibition, (+): inhibition detected, (++): strong inhibitory effect. The activity against C. difficile strains correlates also for Bifidobacteria with the NAGase activity. The strain B. longum CCOS 974, which ranks highest together with B. bifidum CCOS 571 in the inhibitory activity, produces a medium level of NAGase. It can be anticipated at this point that the genome of B. longum CCOS 974 contains indeed also an N-acetylgalactosaminidase gene (see below) and that C. diff. possesses also a glycan layer containing galactosamin

In looking for Bifidobacteria to be used against CDI/CDAD by acting as inhibitors of the pathogen C. difficile, strains showing a high production of NAGase, or more generally specific PGHs, are preferred. Suitable candidates are e.g. B. bifidum CCOS 571, B. longum CCOS 974 and B. breve CCOS 586. Also another B. breve, CCOS 971, which produces high concentrations (>40 nmol) of NAGase according to the APIZYM test showed strong inhibitory activity against the tested C. difficile isolates (data not shown).

### Example 12: Genome Sequences of Bifidobacterium bifidum CCOS 571, Bifidobacterium longum CCOS 974 and Bifidobacterium breve CCOS 586

Whole genomic high molecular weight DNA extraction of each strain was grown in 10 ml of liquid MRS (Man, Rogosa and Sharpe medium) at 37° C. without shaking in a 50 ml conical tubes for 48 h (cell density 1×10⁹ cells/ml). A 1 ml aliquot was centrifuged for 5 min at 8,000×g. The supernatant was removed and resuspended in 500 μl of 5M LiCl. The cells were placed on ice and vigorously vortexed every 2 minutes for 15 minutes. The cells were then centrifuged at 8,000×g for 5 min. A total of 500 μl of saturated phenol was added to the cell pellet, vortexed and centrifuged at 8,000×g. The cell pellet was washed with PBS and resuspended in 500 μl of PBS. Whole genomic DNA was extracted using MasterPure™ DNA Purification Kit (Epicentre, Cat. No. MCD85201). A total of 10 μg of DNA was used to construct a large insert (5-20 kb) SMRTbell™ sequencing library that was sequenced on a Pacific Biosciences RSII instruments according to the manufacturer recommendations at the Institute for Genome Sciences, University of Maryland School of Medicine, Baltimore, MD USA. The genomes were assembled using the MinHash Alignment Process (MHAP) (Berlin K, Koren S, Chin C-S, Drake J P, Landolin J M, Phillippy A M. Assembling large genomes with single-molecule sequencing and locality-sensitive hashing. Nat Biotechnol. 2015; 33:623-630). The estimated genome sizes, number of contigs, gene and coding sequence counts are shown in Table 12.a. Annotation was generated using the Prodigal annotation pipeline (Hyatt D, Chen G-L, LoCascio P F, Land M L, Larimer F W, Hauser L J. “Prodigal: prokaryotic gene recognition and translation initiation site identification” BMC Bioinformatics. 2010; 11:119).

Bifidobacterium strains sequenced are not so much rich in glucosaminidase, at least in enzymes with similarities to those carried by Lactobacillus spp. Interestingly, these strains carry galactosaminidases (Table 12.a). B. bifidum CCOS 571 furthermore has a large panoply of glycoside hydrolases compared to the other two species/strains.

TABLE 12a
N-acetylglucosaminidases and N-acetylgalactosaminidases encoded on the genomes
of Bifidobacterium bifidum CCOS 571, Bifidobacterium longum CCOS 974 and
Bifidobacterium bifidum CCOS 586

| | | SEQ ID NO | SEQ ID NO | |
|---|---|---|---|---|
| Strain | Gene name | nucleotide | amino acid | Annotation/function |
| B. | b_bifidum_ccos_571_07770 | 24 | 100 | beta-N-acetylhexosaminidase |
| bifidum | | | | |
| CCOS | | | | |
| 571 | | | | |
| | b_bifidum_ccos_571_09950 | 25 | 101 | endo-alpha-N |
| | | | | acetylgalactosaminidase |
| | b_bifidum_ccos_571_10670 | 26 | 102 | hexosaminidase |
| | b_bifidum_ccos_571_10680 | 27 | 103 | hexosaminidase |
| | b_bifidum_ccos_571_10690 | 28 | 104 | hexosaminidase |
| | b_bifidum_ccos_571_12320 | 29 | 105 | endo-alpha-N |
| | | | | acetylgalactosaminidase |
| | b_bifidum_ccos_571_14230 | 30 | 106 | Lysozyme M1 (1,4-beta-N |
| | | | | acetylmuramidase) |
| | b_bifidum_ccos_571_14480 | 31 | 107 | beta hexosamidase or lacto-N |
| | | | | biosidase or beta N |
| | | | | acetylglucosamidase |
| | b_bifidum_ccos_571_14630 | 32 | 108 | glycoside hydrolase |
| | b_bifidum_ccos_571_16210 | 33 | 109 | glycoside hydrolase- |
| | | | | Endo-alpha-N |
| | | | | acetylgalactosaminidase |
| | b_bifidum_ccos_571_16870 | 34 | 110 | Hyaluronoglucosaminidase |
| | b_bifidum_ccos_571_17230 | 35 | 111 | neuraminidase |
| | b_bifidum_ccos_571_17880 | 36 | 112 | hexosaminidase |
| | b_bifidum_ccos_571_18050 | 37 | 113 | Hyaluronoglucosaminidase |
| | b_bifidum_ccos_571_18170 | 38 | 114 | glycoside hydrolase- |
| | | | | alpha_N_acetylglucosaminidase |
| | b_bifidum_ccos_571_18860 | 39 | 115 | hexosaminidase |
| B. | | | | |
| breve | | | | |
| CCOS | | | | |
| 586 | | | | |
| | b_breve_ccos_586_09940 | 43 | 119 | Endo-alpha-N |
| | | | | acetylgalactosaminidase |
| | | | | glycoside hydrolase |
| | b_breve_ccos_586_21940 | 46 | 122 | glycoside hydrolase |
| | b_breve_ccos_586_07460 | 42 | 118 | mannosyl-glycoprotein endo |
| | | | | beta-N-acetylglucosaminidase |
| | b_breve_ccos_586_12590 | 44 | 120 | beta-N-acetylhexosaminidase |
| | b_breve_ccos_586_19890 | 45 | 121 | neuraminidase |
| | b_breve_ccos_586_02180 | 40 | 116 | beta hexosamidase or lacto-N |
| | | | | biosidase or beta N |
| | | | | acetylglucosamidase |
| | b_breve_ccos_586_02560 | 41 | 117 | N-acetylmuramoyl-L-alanine |
| | | | | amidase |
| | b_breve_ccos_586_23580 | 47 | 123 | Lyzozyme M1 (1,4-beta-N |
| | | | | acetylmuramidase) |
| B. | | | | |
| longum | | | | |
| CCOS | | | | |
| 974 | | | | |
| | Blong_03460 | 48 | 124 | beta-galactosidase/beta |
| | | | | glucuronidase |
| | Blong_06280 | 50 | 126 | Endo-alpha-N |
| | | | | acetylgalactosaminidase |
| | | | | glycoside hydrolase |
| | Blong_03970 | 49 | 125 | glycoside hydrolase |
| | Blong_09530 | 51 | 127 | glycoside hydrolase |
| | Blong_17510 | 53 | 129 | glycoside hydrolase |
| | Blong_12820 | 52 | 128 | glycoside hydrolase |

In the context of the present invention, it was investigated whether bifidobacteria, which were active against Clostridium difficile, would produce PGH or equivalent substances. As described above, it was indeed found that the investigated Bifidobacteria are capable of producing a few N-acetyl-beta-glucosaminidase but more often e.g. endo-alpha-N-acetylgalactosaminidases, beta-hexosaminidases and glycoside hydrolases. Bifidobacterium bifidum CCOS 571 and B. breve CCOS 586 strains as described herein produce significant signals of amounts of N-acetyl-glucosaminidase in the enzymatic testing, that is they have at the phenotypic level a proven equivalent NAGase activity. In conclusion, a clear correlation was found between the in vitro anti-C. difficile activity and the NAGase enzymatic activity in vitro (with the presence of PGH-genes in the genome constituting the final proof).

In conclusion it is the production specific PGHs the main reason for the up-to now unexplained antimicrobial activity of the bifidobacteria and for the differences in anti-microbial activity among Bifidobacterium strains themselves.

Considering more generally the structure of cell walls in the Gram-positive bacteria a single lipid membrane is present, surrounded by a cell wall composed of a thick layer of peptidoglycan and lipoteichoic acid, which is anchored to the cell membrane by diacylglycerol. Gram-negative bacteria exhibit additionally an outer membrane carrying lipopolysaccharides. Cell walls of mycobacteria consist of thin layers of peptidoglycan and arabinogalactan, and, as the most outer layer, a thick layer of mycolic acids. In summary, without wishing to be bound by theory, it can be considered that the probiotic strains of the present invention, able to produce the different peptidoglycan hydrolases, possess a weapon with which they can attack and disrupt basically the cell wall of every pathogen as it contains a peptidoglycan layer.

For the therapeutic practice this means that the use of such PGH-producing probiotics as described in this invention is justified whenever the cell wall of a pathogenic microorganism of any of the 3 types: Gram-positives, Gram-negatives and mycobacteria is to be tackled in a probio-therapeutic application. Besides lactic acid (D and L), hydrogen peroxide, bacteriocins and other up to now unknown metabolites, the PGHs are an important, basically novel agent for this use, of selected probiotic bacteria in their fight for nutrition and against pathogens. PGHs of viruses, phages or multicellular organisms have been described including their antimicrobial action, but not of probiotic strains.

The scope of the present invention includes not only nosocomial CDI/CDAD, but also other types of infectious diarrhoeal disorders. Besides C. difficile, the most important causative pathogens for such nosocomial diarrhoeas, there are also: entero-pathogenic E. coli (EPEC), diarrhoeagenic Salmonella typhimurium, Shigella flexneri or sonnei, Campylobacter jejuni, Listeria monocytogenes and Clostridium perfringens.

### Example 13: Selected Representatives of Pathogen Species Tested within Spot Experiments with Respect to their Inhibition by the Probiotic Strains of the Present Invention

a. Method

All cultures were passaged 2 times under respective optimal growth conditions prior to the test. All suspensions were prepared in sterile peptone-salt solution (0.1% peptone, 0.9% NaCl). A suspension of each pathogen, corresponding to a McFarland Standard of 0.5 was spread on the surface of Muller-Hinton agar plates. On these plates, 10 μl (corresponding to a McFarland standard of 4 to 5) of each probiotic strain was spotted. After drying, the agar plates were incubated for 24 hours and the zones of inhibition were valued visually.)

b. Results

TABLE 13

| | | Species | | | | | | | |
|---|---|---|---|---|---|---|---|---|---|
| | | L. jensenii | L. gasseri | L. crispatus | L. lactis | L. plantar. | L. paracasei | L. fermentum | L. rhamnosus |
| | Strain | KS | CCOS | CCOS | CCOS | CCOS | CCOS | CCOS | CCOS |
| | code | 119.1 | 960 | 961 | 949 | 893 | 1201 | 1030 | 965 |
| Campylob. | CCOS | (+) | + | (+) | ++ | ++ | + | (+) | + |
| jejuni | 1192 | | | | | | | | |
| Salmonella | CCOS | ++ | + | ++ | + | (+) | + | − | ++ |
| enterica | 505 | | | | | | | | |
| Shigella | CCOS | + | − | ++ | + | + | ++ | − | ++ |
| flexneri | 471 | | | | | | | | |
| Listeria | CCOS | (+) | ++ | + | ++ | (+) | + | ++ | − |
| monocytog. | 468 | | | | | | | | |
| Escherichia | CCOS | + | + | ++ | + | ++ | ++ | (+) | ++ |
| coli | 492 | | | | | | | | |
| Staph. | CCOS | ++ | ++ | − | ++ | + | + | ++ | − |
| aureus | 666 | | | | | | | | |

Legend:
(−): no inhibition,
(+): slight inhibition,
+: inhibition:
++: strong inhibition

c. Conclusion

Against the most common pathogens involved in infectious diarrhoea the probiotic strains found active against C. difficile show, over a row of the above matrix, a medium to good activity against these pathogens.

Combining a strain of L. lactis (e.g. L. lactis CCOS 949) which produces the bacteriocins nisin and lactococcin, with other probiotic strains as e.g. L. gasseri CCOS 960, L. plantarum CCOS 893 or B. breve 571 or 971, which are strong producers of glycosidases, would allow to syergistically potentiate the antipathogen activity against C. diff..

A multi-strain formulation containing Lactococcus lactis 949 and 1 or more of Lactobacillus plantarum CCOS 893, Lactobacillus gasseri CCOS 960, Lactobacillus jensenii KS 119.1, Lactobacillus crispatus CCOS 961, L. paracasei CCOS 1205 (identical to CCOS 1201) is able to inhibit the most common pathogens causing infectious diarrhoeas comprising, but not limited to those caused by entero-pathogenic E. coli, diarrhoeagenic Salmonella enterica, Shigella flexneri, Campylobacter jejuni, and Clostridium perfringens. A multi-strain formulation in the above sense is thus suitable for the prevention and treatment of general diarrhoeal diseases of bacterial origin.

### Example 14: The Lactobacillus casei Group (LCG)

The Lactobacillus casei group (LCG) is composed of the closely related Lactobacillus casei, Lactobacillus paracasei, and Lactobacillus rhamnosus.

The following selected PGHs were identified in L. paracasei CCOS 1201 (identical to CCOS 1205) and L. rhamnosus CCOS 965.

TABLE 14a
Selected PGHs identified in L. paracasei CCOS 1201 (1205) and L. rhamnosus (CCOS 965)

| | | SEQ ID NO | SEQ ID NO | | |
|---|---|---|---|---|---|
| Strain | Gene name | nucleotide | amino acid | Annotation | Site of action |
| L. paracasei | CCOS-1201- | 155 | 161 | N-acetylmuramoyl-L- | Glycan- |
| CCOS 1201 | 19_00127 | | | alanine amidase LytC | Peptide bond |
| (1205) | | | | | |
| | CCOS1201- | 156 | 162 | Autolytic lysozyme | Glycan- |
| | 19_01355 | | | | Peptide bond |
| | CCOS1201- | 157 | 163 | Lysozyme M1 | Glycan strand |
| | 19_01655 | | | | |
| L. rhamnosus | CCOS-965- | 152 | 158 | Sporulation-specific N- | Glycan- |
| CCOS 965 | 19_00255 | | | acetylmuramoyl-L- | Peptide bond |
| | | | | alanine amidase | |
| | CCOS-965- | 153 | 159 | Exo-glucosaminidase | Glycan strand |
| | 19_00690 | | | LytG | |
| | CCOS-965- | 154 | 160 | Autolytic lysozyme | Glycan- |
| | 19_01009 | | | | Peptide strand |

Of Lactobacillus rhamnosus GG we already knew, as disclosed above on page 1, that it does not produce NAGase. Therefore, Lactobacillus casei Shirota YT9029 was used as a reference and was compared with members of the LCG.

The production of NAGase by the strains was correlated with the antimicrobial activity against the representative gastro-intestinal pathogen Clostridium difficile.

Investigated strains in the tests below:

TABLE 14b
Investigated strains

| Species | Code | Reference or Test |
|---|---|---|
| L. paracasei | CCOS 1201 | Test |
| L. paracasei Shirota | CCOS 1226 | Reference |
| L. rhamnosus | CCOS 967 | Test |
| L. rhamnosus | CCOS 965 | Test |
| L. plantarum | CCOS 893 | Reference |
| L. rhamnosus LGG | CCOS 832 | Reference |

TABLE 14c
Results of the APIZYM test

| | APIZYM | | Corresp. | | Calculated Conc, | |
|---|---|---|---|---|---|---|
| | Score | | nanomoles | | (nmol/ul) | |
| Lactobacillus Strain | 18 h | 24 h | 18 h | 24 h | 18 h | 24 h |
| L. paracasei CCOS 1201 | 3 | 5 | 20 | >40 | 0.31 | 0.62 |
| L. paracasei CCOS 1243 | 1 | 3 | 5 | 20 | 0.08 | 0.31 |
| L. rhamnosus CCOS 967 | 1 | 1 | 5 | 5 | 0.08 | 0.08 |
| L. rhamnosus CCOS 965 | 2 | 5 | 10 | >40 | 0.15 | 0.62 |
| L. plantarum CCOS 893 | 1 | 3 | 5 | 20 | 0.08 | 0.31 |

The strain L. plantarum CCOS 893 described above (see Example 13) confirmed the significant production of NAGase with 0.31 nmol/ul after 24 hour incubation. The Shirota strains also showed a significant production of NAGase at 24 h.

The results confirm that NAGase production, and more generally PGH production, is an important antimicrobial mechanism of probiotic strains. One rhamnosus strain, CCOS 967, showed a detectable, but low production of NAGase, whereas the rhamnosus strain 965 had a very high production of this enzyme amounting to calculated 0.62 nmol/ul. The test strain L. paracasei CCOS 1201 reaches the same high level of calculated 0.62 nmol/ul after 24 h.

Proof of this correlation was provided by the experimental assessment of antimicrobial activity. To this end, the same strains underwent the same inhibition experiments against Clostridium difficile strains as described above.

a. Method

The pH of the growth plate was adjusted to pH 5 with a phosphate buffer to limit the effect of lactic acid on inhibition.

b. Results

TABLE 14d
Inhibition of C. difficile

| Genus | Species | | 937 | 940 | 941 | 948 |
|---|---|---|---|---|---|---|
| Lactobacillus | paracasei | 1201 | +++ | + | ++ | ++ |
| Lactobacillus | paracasei | 1243 | ++ | ++ | ++ | ++ |
| | Shirota | | | | | |
| Lactobacillus | rhamnosus | 967 | + | + | ++ | + |
| Lactobacillus | rhamnosus | 965 | ++ | + | ++ | ++ |
| Lactobacillus | rhamnosus | 832 | ++ | + | ++ | + |
| | LGG | | | | | |

Legend:
CCOS 937, 940, 941 and 958 are C. difficile strains of the CCOS collection.
+++, ++, +, (+), (−) stand for: strong, medium, weak, very weak, missing inhibition.

L. paracasei Shirota and CCOS 1201 show about the same pattern of consistent strong to medium inhibition as also L. rhamnosus CCOS 965, whereas the CCOS 967 of this species is slightly less active. L. rhamnosus LGG exhibits a medium to weak activity depending on the indicator strain.

To more completely reduce the effect of the lactic acid TSB/MRS plates were used with a phosphate buffer at pH=6.2 with the following results.

TABLE 14e
Inhibition of C. difficile on TSB/MRS plates

| Genus | Species | CCOS-Nr. | 937 | 941 | 958 |
|---|---|---|---|---|---|
| Lactobacillus | paracasei | 1201 | − | + | + |
| Lactobacillus | paracasei | 1243 | − | + | (+) |
| | Shirota | | | | |
| Lactobacillus | rhamnosus | 967 | − | + | (+) |
| Lactobacillus | rhamnosus | 965 | + | + | + |
| Lactobacillus | rhamnosus LGG | 832 | − | − | − |

It is concluded that, within the important LCG Group, with respect to the activity against C. difficile, L. paracasei CCOS 1201 is about equivalent to L. paracasei Shirota and the L. rhamnosus CCOS 965. L. rhamnosus 967 is less active. That the observed activity is not due to undissociated lactic acid is confirmed by the fact that L. rhamnosus LGG, which is a huge producer of L-lactic acid (but does not produce NAGase) is about inactive at this pH, where the lactic acid is almost completely dissociated.

### Example 15: Selected Representatives of Pathogen Species in Urogenital Infections Tested within Spot Experiments with Respect to their Inhibition by the Probiotic Strains of the Present Invention

The most common vaginal infection among women of childbearing age is bacterial vaginosis (BV). This condition is characterized by replacement of vaginal lactobacilli with predominantly anaerobic microorganisms such as Gardnerella vaginalis and Prevotella, Peptostreptococcus and Bacteroides spp. Historically; G. vaginalis is thought to have the leading role in the infection, making the niche suitable for colonization by strict anaerobes that are largely responsible for the clinical symptoms of BV (Swidsinski et al. Am J Obstet Gynecol. 2008 January; 198(1):97.el-6. Epub 2007 Nov. 19)

Treatment of this condition using recommended antibiotics is often associated with failure and high rates of recurrence. This is due, among other reasons, to the fact that an adherent Gardnerella vaginalis biofilm persists on the vaginal epithelium after standard therapy with oral metronidazole.

The present invention shows that the probiotic strains described herein, which produce NAGase or other PGHs, are capable of inhibiting and/or killing Gardnerella vaginalis, the leading germ of BV. Gardnerella is a genus of Gram-variable-staining (mostly Gram positive) facultative anaerobic bacteria of which Gardnerella vaginalis is the only species. The organisms are small non-spore-forming, non-motile coccobacilli.

A number of lactic acid bacteria mostly of vaginal origin or at least of species reported to have been isolated from vagina was selected as test or reference strains. They are summarized in the following table.

TABLE 15a
Strain selection for Gardnerella vaginalis inhibition experiments

| Species | Code | Comment |
|---|---|---|
| L. rhamnosus | CCOS 965 | Test |
| L. fermentum | CCOS 1030 | Test |
| L. jensenii | KS 119.1 | Reference |
| L. jensenii | CCOS 962 | Test |
| L. crispatus | CCOS 961 | Test |
| L. gasseri | CCOS 960 | Test |
| Lactococcus lactis | CCOS 949 | Test |
| L. plantarum | CCOS 893 | Test |
| Gardnerella vaginalis Type strain | CCOS 807 | Indicator strain |

a. Agar Spot Assay:

20 μl of probiotic culture (McFarland Standard of 6) were spotted onto Muller Hinton Agar, incubated anaerobically at 37° C. for 2 days. 10 ml of liquid soft agar (Tryptic soy broth with 0.7% agar) were inocculated with 200 μL of a suspension of Gardnerella vaginalis (McFarland Std. 0.5) and poured on top of the plates with the grown probiotics. After incubation (37° C., anaerob, 2 days) the zones of inhibition (no growth of G. vaginalis) around each spot were compared visually.

b. Cross Streaking Assay:

10 μL of probiotic culture were streaked in a horizontal line on BHI agar (supplemented with 10% fetal calf serum, 1% yeast extract, 0.1% maltose, 0.1% glucose), incubated anaerobically at 37° C. for 2 days. 10 μL of a suspension of Gardnerella vaginalis (McFarland Std. 0.5) were streaked in a vertical line as close to the probiotic line as possible. After incubation (37° C., anaerob, 2 days) the distances of inhibition (no growth of G. vaginalis) between the lines were compared visually.

c. Results:

TABLE 15b
inhibition of C. difficile

| | | <<Spot | <<Cross- | NAGase |
|---|---|---|---|---|
| Species | Code | Assay>> | Streaking Assay>> | activity |
| L. rhamnosus | CCOS 965 | + | + | ++ |
| L. fermentum | CCOS 1030 | − | + | + |
| L. jensenii | KS 119.1 | + | + | + |
| L. jensenii | CCOS 962 | − | (+) | + |
| L. crispatus | CCOS 961 | − | − | − |
| L. gasseri | CCOS 960 | + | + | ++ |
| Lactococ. lactis | CCOS 949 | +++ | +++ | ++ |
| L. plantarum | CCOS 893 | + | not tested | ++ |

There is a significative correlation between the NAGase activity as measured by APIZYM and the inhibition activity of the strains. Similarly as for Clostridium difficile inhibition the strongest activity is exhibited by Lactococcus lactis strain CCOS 949.

### Example 16: Stimulating PGH Production with Sugars

For the probiotic strains showing an inhibitory effect against C. difficile and producing NAGase in the enzymatic tests the corresponding genes could be found in their respective genomes. However, the quantity of the produced PGHs and the rate of production can vary according to the environment. In this respect, methods to stimulate the production of the PGHs are helpful in increasing the efficacy of the anti-C. difficile formulation. The present inventors found that in most cases the production of NAGase or more generally of the PGHs is stimulated by the presence of the corresponding simple sugar, e.g. N-acetyl-glucosamine (NAG), in the reaction solution.

The following results were obtained with the VITEK 2 System (bioMdrieux, France) following the manufacturer's instructions, utilizing cards GP respectively ANC (c.f. Instrument User Manual). Lactic acid bacteria were grown aerobically on MRS medium and Bifidobacteria on MRS medium supplemented with cystein (0.5 g/l) under anaerobic conditions. For the test, a fresh overnight culture grown was resuspended in sterile physiological saline solution at an optical density corresponding to a McFarland standard of 5 to 6. This solution was dispensed into the wells of the strips of the test system. The tests strips were incubated at 37° C. for 20 hours. After addition of the test reagents the strips were exposed to light and then read visually and interpreted according to the test instructions resulting in a value from 0 (negative) to 5 (strong positive) for each test. Each test was repeated at least twice.

TABLE 16
Fermentation of N-acetylglusosamine (NAG) by selected probiotic
bacteria suitable for an anti-C. difficile multi-strain formulation.

| Strain | Card GP | Card ANC |
|---|---|---|
| Lactococcus lactis CCOS 949 | | |
| L. gasseri CCOS 960 | neg | pos |
| L. gasseri KS 120.1 | neg | n.a. |
| L. gasseri 124.3 | pos | pos |
| L. crispatus CCOS 961 | pos | pos |
| L. jensenii KS 119.1 | pos | pos |
| L. plantarum CCOS 893 | pos | pos |
| B. bifidum CCOS 571 | n.a. | pos |
| B. breve CCOS 586 | n.a. | neg |
| B. longum CCOS 974 | n.a | n.a |
| B. bifidum CCOS 975 | pos | pos |

Legend: pos: positive, neg: negative, n.a.: not available

Among the candidates for the multi-strain anti-C. difficile symbiotic composition there is a majority of probiotic strains fermenting N-acetyl-D-glucosamine. The bacterial PGHs of the probiotic bacteria selectively attack the cell walls of pathogens. The addition of simple N-acetylated sugars like N-acetyl-D-glucosamine, N-acetylgalactosamine, N-acetylhexosamine, and the like was found by the present inventors to boost the production of the corresponding lytic enzymes. Typically, they can be added to the formulation in amounts varying between 0.5 and 5 g per dosage.

### Example 17: Recombinant production of β-N-Acetylglucaminidase (NAGase) in E. coli

An E. coli codon optimised DNA sequence was determined based on the DNA sequence of the β-N-Acetyglucamindase (GenBank Acc. No. AB025100) from Lactobacillus paracasei CCOS 1201. This DNA sequence was commercially synthesised and cloned into the E. coli expression vector pRSET A (Thermo Fisher) (using the restriction sites BamHI and HindIII and T4-DNA ligase).

This construct (pRsetA_Nagase_Ic) was first transformed into E. coli TOP10F′ (Thermo Fisher), 5 clones were selected and the inserted plasmids were verified by restriction digest and DNA-sequencing and one plasmid was then used to transform the expression strain E. coli Lemo21 (DE3) (NEB).

The expression strain was cultivated in LB-broth supplemented with 50 μg/mL ampicillin at 30° C. in an orbital shaker (120 rpm). After an OD₆₀₀ᵢₘ of 0.6 was reached, protein expression was induced with IPTG (400 μM final conc.).

In a first step, a small scale expression experiment with 10 mL culture volume, different L-Rhamnose concentrations and expression temperatures as shown as in Error!Reference source not found.7 was performed.

TABLE 17
Expression conditions

| | L-rhamnose | Temperature/ |
|---|---|---|
| | concentration | Expression |
| Sample | (μM) | time |
| 1 | 0 | 18° C., 16 hours |
| 2 | 100 | 18° C., 16 hours |
| 3 | 250 | 18° C., 16 hours |
| 4 | 500 | 18° C., 16 hours |
| 5 | 750 | 18° C., 16 hours |
| 6 | 1000 | 18° C., 16 hours |
| 7 | 2000 | 18° C., 16 hours |
| 8 | 0 | 37° C., 16 hours |
| 9 | 100 | 37° C., 16 hours |
| 10 | 250 | 37° C., 16 hours |
| 11 | 500 | 37° C., 16 hours |
| 12 | 750 | 37° C., 16 hours |
| 13 | 1000 | 37° C., 16 hours |
| 14 | 2000 | 37° C., 16 hours |

After expression, the cells were harvested by centrifugation. The pellets and the supernatants were then analyzed by SDS PAGE and Western Blot (anti-His6, Novagen). On SDS-PAGE gels, no expression was detectable. In the corresponding Western Blot, weak signals could be detected, and no difference in signal intensities was observed.

Two larger scale expression experiment with 100 ml culture volume was performed (no Rhamnose, 20° C. and 37° C. induction temperature, for 16 hours).

After expression the cells were harvested by centrifugation, the cell pellets were resuspended in binding buffer (containing 6 M Urea) and lysed by sonication. After centrifugation the supernatant was purified by FPLC (Akta FPLC system, with HisTrap FF Crude, GE Lifes Sciences), several fractions were collected for further analysis.

After a buffer exchange by dialysis (Amicon Ultra-4, PLGC Ultracel-PL Membran, 10 kDa) the protein fractions were analysed by SDS PAGE and Western Blot. Signals were detected in the Western Blot in 3 fractions (see FIG. 16).

### Example 18: Characterization of of Lactococcus lactis CCOS 949

Lactococcus lactis CCOS 949 was deposited on 3 May 2016 under number DSM 32294 with the DSMZ (Deutsche Sammlung von Mikroorganismen und Zelikulturen, Leibniz-Institut, Braunschweig, Germany).

Specifications

| | |
|---|---|
| Organism: | Lactococcus lactis |
| Strain code: | CCOS 949 |

Patent Deposit

| | |
|---|---|
| Deposited at: | Leibniz-lnstitut, DSMZ-Deutsche Sammlung |
| | von Mikroorganismen und Zellkulturen GmbH, |
| | Inhoffenstra8e 7 B, 38124 Braunschweig, |
| | Germany |
| Depositon date: | 21 Apr. 2016 |
| Deposition code: | DSM 32294 |

Taxonomy

| | |
|---|---|
| Kingdom: | Bacteria |
| Phylum: | Firmicutes |
| Class: | Bacilli |
| Order: | Lactobacillales |
| Family: | Streptococcaceae |
| Species reference: | Schleifer et al. 1986 |

Depositor

| | |
|---|---|
| Name | Probioswiss GmbH, Zurich |
| Country: | Switzerland |

Origin

| | |
|---|---|
| Source: | Food |
| Source material: | Cheese |
| Country: | Switzerland |
| Strain history: | <- F. Graf, Crigasseni AG |

Biosafety

| | |
|---|---|
| Biosafety level: | 1 |

Growth conditions

| | |
|---|---|
| Recommended growth | MRS Agar or MRS Broth |
| medium: | |
| Alternative growth | TSA (Tryptic Soy Agar) |
| media: | BLA (Tryptic Soy Agar with 5% Sheep blood) |
| Temperature: | 30° C. |
| Oxygen requirements: | facultative aerobe, anaerobic growth conditions |
| | recommended |

Rapid tests

| | |
|---|---|
| Gram type: | positive |
| KOH test: | negative |
| Catalase test: | negative |

Preservation

| | |
|---|---|
| Method: | Cryopreservation |
| Temperature: | −80° C. |
| Antifreeze: | 50% Glycerol |

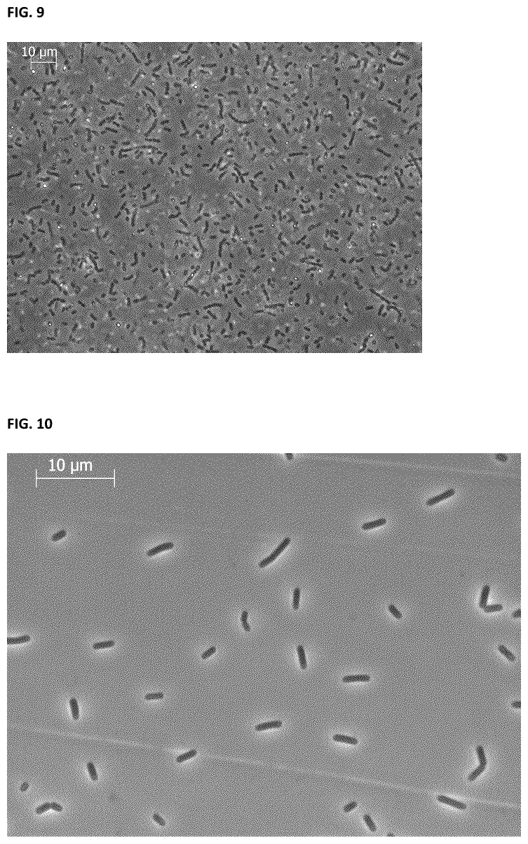

Morphology: cocci, in chains up to 8, non motile (Phase contrast image of L. lactis CCOS 949 in FIG. 9). Identification tests: MALDI-TOF, Lactococcus lactis subsp. lactis, score: 2.37 (+++) (Bruker Biotyper); 16S rDNA sequencing: Lactococcus lactis, 99% ID (NCBI Genbank); DNA Sequencing: GenePartial 16S rDNA; Method: Sanger Sequencing; Sequence: SEQ ID NO: 83.

### Example 19: Characterization of Lactobacillus plantarum CCOS 893

Lactobacillus plantarum CCOS 893 was deposited on 27 Jul. 2016 under number DSM 32352 with the DSMZ (Deutsche Sammlung von Mikroorganismen und Zellkulturen, Leibniz-Institut, Braunschweig, Germany).

Specifications

| | |
|---|---|
| Organism: | Lactobacillus plantarum |
| Strain code: | CCOS 893 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | (Orla-Jensen 1919) Bergey et al. 1923 |

Depositor

| | |
|---|---|
| Name | Probioswiss GmbH, Zürich, Switzerland |
| | on 27 Jul. 2016 |
| Country: | Switzerland |

Origin

| | |
|---|---|
| Source: | Plant |
| Source material: | Silage |
| Country: | Switzerland |

Biosafety

| | |
|---|---|
| Biosafety level: | 1 |

Growth conditions

| | |
|---|---|
| Growth medium: | MRS |
| Alternative growth | BLA (Tryptic Soy Agar with 5% Sheep blood) |
| media: | |
| Temperature: | 37° C. |
| Oxygen requirements: | facultative aerobe, anaerobic growth conditions |
| | recommended |
| Microscopy | Morphology: rods, not motile |
| | (Phase contrast image shown in FIG. 10) |

Rapid tests

| | |
|---|---|
| Gram type: | positive |
| KOH test: | negative |

Identification tests

| | |
|---|---|
| MALDI-TOF | Lactobacillus plantarum, score: 2.463 (+++) |
| | (Bruker Biotyper) |
| 16S rDNA sequencing | Lactobacillus plantarum, 99% ID (NCBI |
| | Genbank) |
| VITEK ANC-Card | not determined |

Preservation

| | |
|---|---|
| Method: | Cryopreservation |
| Temperature: | −80° C. |
| Antifreeze: | 50% Glycerol |

### Example 20: Characterization of Lactobacillus Crispatus CCOS 961

Lactobacillus crispatus CCOS 961 was deposited on 10.12.2017 under deposit number CCOS 961 with the Culture Collection of Switzerland AG (CCOS, Wadenswil, Switzerland).

Specifications

| | |
|---|---|
| Organism: | Lactobacillus crispatus |
| Strain code: | CCOS 961 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | (Brygoo and Aladame 1953) Moore and |
| | Holdeman 1970 |

Depositor

| | |
|---|---|
| Name | Crigasseni AG, Beckenried, Switzerland |
| | on 10 Dec. 2017 |
| Country: | Switzerland |

Origin

| | |
|---|---|
| Source: | Healthy Human |
| Source material: | Vaginal swab |
| Country: | Switzerland |
| Strain history: | <- F. Graf Crigasseni |

Biosafety

| | |
|---|---|
| Biosafety level: | 1 |

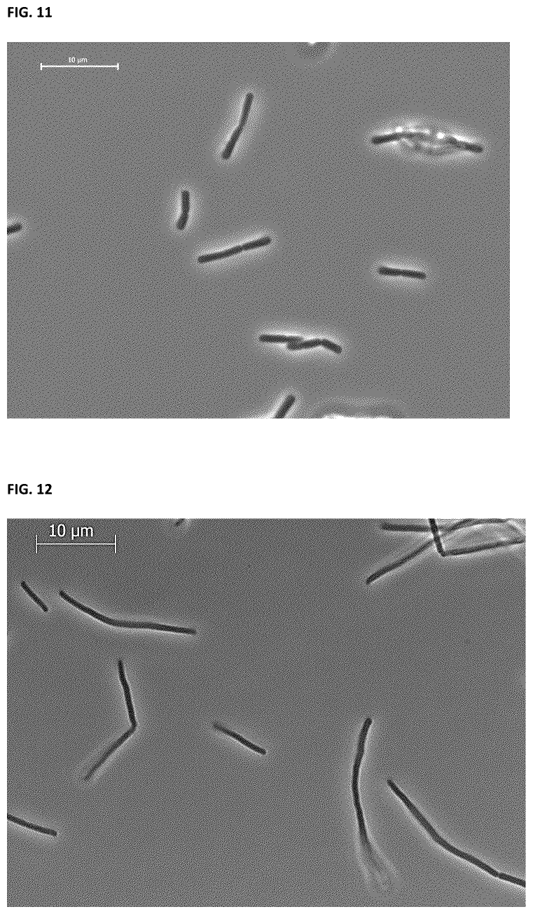

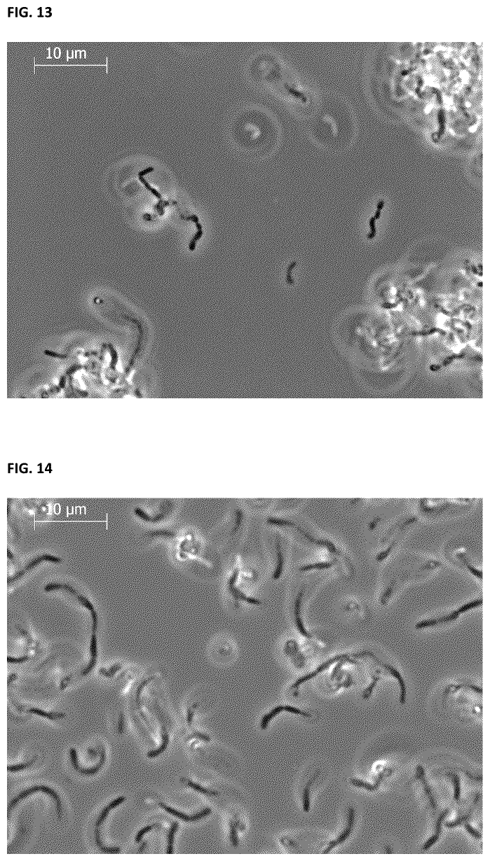

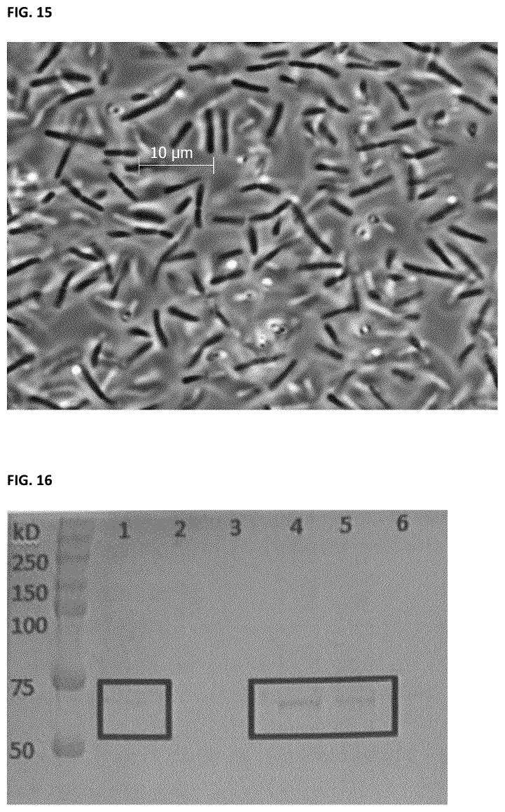

Growth conditions

| | |
|---|---|
| Growth medium: | MRS |
| Alternative growth | BLA (Tryptic Soy Agar with 5% Sheep blood) |
| media: | |
| Temperature: | 37° C. |
| Oxygen requirements: | facultative aerobe, anaerobic growth conditions |
| | recommended |
| Microscopy: | Morphology: rods, non motile (Phase contrast |
| | image shown in FIG. 11). |

Rapid tests

| | |
|---|---|
| Gram type: | positive |
| KOH test: | negative |

Identification tests

| | |
|---|---|
| MALDI-TOF | Lactobacillus crispatus, score: 2.273 (++) |
| | (Bruker Biotyper) |
| 16S rDNA sequencing | not determined |
| VITEK ANC-Card | not determined |

Preservation

| | |
|---|---|
| Method: | Cryopreservation |
| Temperature: | −80° C. |
| Antifreeze: | 50% Glycerol |

### Example 21: Characterization of Lactobacillus gasseri CCOS 960

Lactobacillus gasseri identified as Lactobacillus gasseri CCOS 960 was deposited on 21 Apr. 2016 under number DSM 32296 with the DSMZ (Deutsche Sammlung von Mikroorganismen und Zellkulturen, Leibniz-Institut, Braunschweig, Germany).

Specifications

| | |
|---|---|
| Organism: | Lactobacillus gasseri |
| Strain code: | CCOS 960 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | Lauer and Kandler 1980 |

Depositor

| | |
|---|---|
| Name | Probioswiss GmbH, Zürich, Switzerland |
| | on 21 Apr. 2016 |
| Country: | Switzerland |

Biosafety

| | |
|---|---|
| Biosafety level: | 1 |

Growth conditions

| | |
|---|---|
| Growth medium: | MRS |
| Alternative growth | BLA (Tryptic Soy Agar with 5% Sheep blood) |
| media: | TSA (Tryptic Soy Agar) |
| Temperature: | 37° C. |
| Oxygen requirements: | facultative aerobe, anaerobic growth conditions |
| | recommended |
| Microscopy | Morphology: Very long rods, non-motile |
| | (Phase contrast image in FIG. 12) |

Rapid tests

| | |
|---|---|
| Gram type: | positive |
| KOH test: | negative |

Identification tests

| | |
|---|---|
| MALDI-TOF | Lactobacillus gasseri, score: 2.425 (+++) |
| | (Bruker Biotyper) |
| 16S rDNA sequencing | not determined |
| VITEK ANC-Card | no identification, Bionumber 2173100020001 |

Preservation

| | |
|---|---|
| Method: | Cryopreservation |
| Temperature: | −80° C. |
| Antifreeze: | 50% Glycerol |

### Example 22: Characterization of Lactobacillus jensenii CCOS 962

Lactobacillus jensenii identified as Lactobacillus jensenii CCOS 962 was deposited on 17 Apr. 2019 under number CCOS 962 with the CCOS (Culture Collection of Switzerland AG, Wddenswil, Switzerland).

Specifications

| | |
|---|---|
| Organism: | Lactobacillus jensenii |
| Strain code: | CCOS 962 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | Gasser et al. 1970 |

Depositor

| | |
|---|---|
| Name | Crigasseni AG, Ledergasse 34, 6375 |
| | Beckenried, Switzerland, 30 May 2016 |
| Country: | Switzerland |

Biosafety

| | |
|---|---|
| Biosafety level: | 1 |

Growth conditions

| | |
|---|---|
| Growth medium: | MRS |
| Alternative growth | BLA (Tryptic Soy Agar with 5% Sheep blood) |
| media: | TSA (Tryptic Soy Agar) |
| Temperature: | 37° C. |
| Oxygen requirements: | facultative aerobe, anaerobic growth conditions |
| | recommended |
| Microscopy | Morphology: short rods, in chains up to 4, |
| | non-motile (Phase contrast image in |
| | FIG. xx) |

Rapid tests

| | |
|---|---|
| Gram type: | positive |
| KOH test: | negative |

Identification tests

| | |
|---|---|
| MALDI-TOF | Lactobacillus jensenii, score: 2.251 (+++) |
| | (Bruker Biotyper) |
| 16S rDNA sequencing | Lactobacillus jensenii, 99% ID (16S rRNA, |
| | NCBI Genbank) |
| VITEK | ANC-Card not determined |

Preservation

| | |
|---|---|
| Method: | Cryopreservation |
| Temperature: | −80° C. |
| Antifreeze: | 50% Glycerol |

### Example 23: Characterization of Lactobacillus rhamnosus CCOS 965

Lactobacillus rhamnosus identified as Lactobacillus rhamnosus CCOS 965 was deposited on 17 Apr. 2019 under number CCOS 965 with the CCOS (Culture Collection of Switzerland AG, Wädenswil, Switzerland).

Specifications

| | |
|---|---|
| Organism: | Lactobacillus rhamnosus |
| Strain code: | CCOS 965 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | (Hansen 1968) Collins et al. 1989 |

Depositor

| | |
|---|---|
| Name | Crigasseni AG, Ledergasse 34, 6375 |
| | Beckenried, Switzerland, 30 May 2016 |
| Country: | Switzerland |

Biosafety

| | |
|---|---|
| Biosafety level: | 1 |
| Growth conditions | |
| Growth medium: | MRS |
| Alternative growth | BLA (Tryptic Soy Agar with 5% Sheep blood) |
| media: | TSA (Tryptic Soy Agar) |
| Temperature: | 37° C. |
| Oxygen requirements: | facultative aerobe, anaerobic growth conditions |
| | recommended |
| Microscopy | Morphology: Very long rods, non-motile |
| | (Phase contrast image in FIG. xx) |

Rapid tests

| | |
|---|---|
| Gram type: | positive |
| KOH test: | negative |

Identification tests

| | |
|---|---|
| MALDI-TOF | Lactobacillus rhamnosus, score: 2.177 (++) |
| | (Bruker Biotyper) |
| 16S rDNA sequencing | Lactobacillus rhamnosus, 99% ID (16S rRNA, |
| | NCBI Genbank) |
| VITEK ANC-Card | not determined |

Preservation

| | |
|---|---|
| Method: | Cryopreservation |
| Temperature: | −80° C. |
| Antifreeze: | 50% Glycerol |

### Example 24: Characterization of Lactobacillus paracasei CCOS 1201

Lactobacillus paracasei identified as Lactobacillus paracasei CCOS 1201 (identical to CCOS 1205) was deposited on 17 Apr. 2019 under number CCOS 1201 with the CCOS (Culture Collection of Switzerland AG, Wädenswil, Switzerland).

Specifications

| | |
|---|---|
| Organism: | Lactobacillus paracasei |
| Strain code: | CCOS 1201 |

Taxonomy

| | |
|---|---|
| Kingdom: | Bacteria |
| Phylum: | Firmicutes |
| Class: | Bacilli |
| Order: | Lactobacillales |
| Family: | Lactobacillaceae |
| Species reference: | Collins et al. 1989 |

Depositor

| | |
|---|---|
| Name | Crigasseni AG, Ledergasse 34, 6375 |
| | Beckenried, Switzerland, 30 May 2016 |
| Country: | Switzerland |

Biosafety

| | |
|---|---|
| Biosafety level: | 1 |

Growth conditions

| | |
|---|---|
| Growth | medium: MRS |
| Alternative growth | BLA (Tryptic Soy Agar with 5% Sheep blood) |
| media: | TSA (Tryptic Soy Agar) |
| Temperature: | 37° C. |
| Oxygen requirements: | facultative aerobe, anaerobic growth conditions |
| | recommended |
| Microscopy | Morphology: rods, non-motile (Phase contrast |
| | image in FIG. xx) |

Rapid tests

| | |
|---|---|
| Gram type: | positive |
| KOH test: | negative |

Identification tests

| | |
|---|---|
| MALDI-TOF | Lactobacillus paracasei, score: 2.35(+++) |
| | (Bruker Biotyper) |
| 16S rDNA sequencing | Lactobacillus paracasei, 99% ID (16S rRNA, |
| | NCBI Genbank) |
| VITEK ANC-Card | not determined |

Preservation

| | |
|---|---|
| Method: | Cryopreservation |
| Temperature: | −80° C. |
| Antifreeze: | 50% Glycerol |

What is claimed is:

1. A method for the prophylaxis and/or treatment of a bacterial infection in a patient in need of such treatment or prophylaxis, the method comprising administering an effective amount of a peptidoglycan hydrolase (PGH), wherein the PGH is a polypeptide selected from:
(a) polypeptides comprising an amino acid sequence selected from the group consisting of SEQ ID NOs: 89 to 151 and 158 to 163;
(b) polypeptides comprising an amino acid sequence having an amino acid sequence identity of at least 70 or 80% with an amino acid sequence selected from the group consisting of SEQ ID NOs: 89 to 151 and 158 to 163; and
(c) functional fragments or functional derivatives of (a) or (b),
wherein the method effectuates the prophylaxis and/or treatment of a bacterial infection in the patient.

2. The method of claim 1, further comprising repeating administration.

3. The method according to claim 1, wherein the bacterial infection results in diarrhea, is a bacterial urogenital infection or is a bacterial vaginal infection.

4. The method according to claim 1, wherein the peptidoglycan hydrolase (PGH) is in the form of a pharmaceutical composition further comprising at least one physiologically acceptable excipient.

5. The method according to claim 1, wherein the peptidoglycan hydrolase (PGH) is administered orally, rectally, by intravenous injection or by subcutaneous injection.

6. A method for the prophylaxis and/or treatment of a bacterial infection in a patient in need of such treatment or prophylaxis, the method comprising administering an effective amount of a peptidoglycan hydrolase (PGH), wherein the PGH is encoded by a nucleic acid comprising or consisting of a nucleic acid sequence selected from:
(a) nucleic acid sequences selected from the group consisting of SEQ ID NOs: 1 to 11, 24 to 53, 65 to 81, 84 to 88 and 152 to 157;
(b) nucleic acid sequences having at least 80 or 90% identity with a nucleic acid sequence listed in SEQ ID NOs: 1 to 11, 24 to 53, 65 to 81, 84 to 88 and 152 to 157;
(c) nucleic acid sequences that hybridize to a nucleic acid sequence of (a) or (b) under stringent conditions;
(d) fragments of any of the nucleic acid sequences (a) to (c), that hybridize to a nucleic acid sequence of (a) or (b) under stringent conditions; and
(e) a nucleic acid sequence, wherein said nucleic acid sequence is derivable by substitution, addition and/or deletion of one of the nucleic acids of (a) to (d) that hybridizes to a nucleic acid sequence of (a) or (b) under stringent conditions,
wherein the method effectuates the prophylaxis and/or treatment of a bacterial infection in the patient.

7. The method of claim 6, further comprising repeating said administration.

8. The method according to claim 6, wherein the bacterial infection results in diarrhea, is a bacterial urogenital infection or is a bacterial vaginal infection.

9. The method according to claim 6, wherein the peptidoglycan hydrolase (PGH) is in the form of a pharmaceutical composition further comprising at least one physiologically acceptable excipient.

10. The method according to claim 6, wherein the peptidoglycan hydrolase (PGH) is administered orally, rectally, by intravenous injection or by subcutaneous injection.

11. The method according to claim 6, wherein the nucleic acid for producing a PGH is comprised in a recombinant vector.

12. The method according to claim 11, wherein the recombinant vector is a viral vector, an episomal vector, a baculovirus vector, a lentivirus vector, an adenovirus vector, a vaccinia vector, a retroviral vector, a yeast vector or a bacterial episomal vector.

13. The method according to claim 6, wherein the PGH is produced or secreted by a host cell comprising the nucleic acid or comprising a vector comprising said nucleic acid sequence.

14. The method according to claim 13, wherein the host cell is selected from the group consisting of yeast cells, Saccharomyces cerevisiae cells, Pichia pastoris cells, bacterial E. coli, Vibrio natriegens cells, B. subtilis cells, plant cells, Nicotiana tabacum cells, Physcomirella patens cells, NIH-3T3 mammalian cells, HEK293 mammalian cells, HEK293T mammalian cells, CHO mammalian cells, COS mammalian cells, insect cells, and sf9 insect cells.


SEQUENCE LISTING (163 sequences)

SEQ ID NO: 1 - DNA, 1677 bp, Lactobacillus gasseri
  source 1..1677: mol_type = genomic DNA; strain = CCOS 960
atgcttgctcaagcagcggtcgaatcagcttggggtcaatcaggtttggcacaatcgcctaataataacttattcggtattaagggctcttacaatgggcaatcagtaaatatgaatactggtgagtacggcagcaatggttattacactactaacgctggatttagaaagtatccatcatatactgaatcatttgaagataatggctctttattacgtaaccaaatgggtaactattactctggtacgtgggtagaaaattcaaagaactacgctcaagcaactcaaaatggtttacaaggcaagtatgcaacagcgccaaactatgctcagacacttaatagtgttattgcagctaatggatttgataagtacgatcccgttacccaagttgttaacgaaaatcgtacagtcgcacaaaccacaccaattatgagtgccccagttgatgctagcgttggtactcaagttggtactgcaagaactggtcaaaacgtaaatgttactaagtacatcacttataacaatggtgttaagcgtgcatatattggtactggctggattaacgcacttgcatttagtccaattactactaatacaactactaagcaaaatactgccgctaatactaataatcaagcaagtcaagcagttaagacgccagtcgcacaaactcaacaagttaagagtcaagcacctgcagctccagtaaaagctgcaactgtaaaagtaaagagtgccgctgaagttaaaactccagttcaaactaccactttaaacgttaaaactgaaaataaagcagcacaacctgtaaagcaattagctgtttcattagctgttgcacctaagaaggttgaagttaagaagaacgttgtgaaagctgaaccagctaagacaactccagtaaaaactgaaactactaaaactgaaaacaaagaagttaagactgcaactccagtagttaaaactactgatacagttaaaaaagttgaaactccagcagttaaaccagttgaatctgtaaagaaggaatcaactccagttgttaagactactccagttactgtaactaaagaagctgcaaagaccaccgaagctccagtagttaaacctaagaaggttgaagttaaggaaattaagactactccagtaactactagtgctaaaactgttgtaaagcctgttcaaagttaccaaagtgcagtaactactgctgcaaacaataactatggtactcaatggattagtactaatactgctcctaaggctgcatctactgttttaattaaggttactaagactgttgatgttttatcagcaccagatggtcaaaaattagaccaacaagttgaagctggctcagaatttgttgttgttgcttctaagtactacaatggcaacttatattacgaaattagtaatggtaaatggattatggcaaaatatactactcaagaagcacaaattactgcaaaatctggtgtattaactattaattctaagccagactatggtgtaccagtatggcgtgttccaggtcaagatcaaattgctggtaagttcttgaaagacggctcaagctggagatacttccgtgtagcaaacgttcaaggacaaacttggtatgaccttggcggaaatcaatgggtatctgccaagtctgtactagttcgttaa

SEQ ID NO: 2 - DNA, 627 bp, Lactobacillus gasseri
  source 1..627: mol_type = genomic DNA; strain = CCOS 960
atgccgcgtagacgccgtcaaaaaaatattcaatatgtaattgcaagagcctttgctatttgctttactttggtagtaattttatcaggctttttgtactggcgtcacgaggtcgcggttaatgaacaattaaggcaagcgcagttagaaaaggaacgcgctcttcaaagtaaagaaaggtttattaaagtagtagctccaattgctcaaagagcagataagccttatggtttatttccaagtgttaccattgcgcaagcatgcttagaaagtaattttggtcaaagtgagctttcaaaaaaatattataatttgttcggtgtcaagggaaccgatcctaatactagccgagagctgacgacttcggaatttgttaatgatcactgggaaactgtaactggtcgttttcaaatttataattcttatgaagaatctattcaggcacatacgcggttatttgtgaatggaactagctggaataaagatcaatatcagcatgttttagctgcaaaagattatgcaagtcaagcgcaagctttagagacggatggttatgcgactgatccaggttatgctaaaaagctaattgatttaattaaagagttcaatttaacgcaatatgattaa

SEQ ID NO: 3 - DNA, 1542 bp, Lactobacillus gasseri
  source 1..1542: mol_type = genomic DNA; strain = CCOS 960
atggttattacactactaacgctggatttagagaagtatccatcatatactgaatcatttgaagataatggctctttattacgtaaccaaatgggtaactattactctggtacgtgggtagaaaattcaaagaactacgctcaagcaactcaaaatggtttacaaggcaagtatgcaacagcgccaaactatgctcagacacttaatagtgttattgcagctaatggatttgataagtacgatcccgttacccaagttgttaacgaaaatcgtacagtcgcacaaaccacaccaattatgagtgccccagttgatgctagcgttggtactcaagttggtactgcaagaactggtcaaaacgtaaatgttactaagtacatcacttataacaatggtgttaagcgtgcatatattggtactggctggattaacgcacttgcatttagtccaattactactaatacaactactaagcaaaatactgccgctaatactaataatcaagcaagtcaagcagttaagacgccagtcgcacaaactcaacaagttaagagtcaagcacctgcagctccagtaaaagctgcaactgtaaaagtaaagagtgccgctgaagttaaaactccagttcaaactaccactttaaacgttaaaactgaaaataaagcagcacaacctgtaaagcaattagctgtttcattagctgttgcacctaagaaggttgaagttaagaagaacgttgtgaaagctgaaccagctaagacaactccagtaaaaactgaaactactaaaactgaaaacaaagaagttaagactgcaactccagtagttaaaactactgatacagttaaaaaagttgaaactccagcagttaaaccagttgaatctgtaaagaaggaatcaactccagttgttaagactactccagttactgtaactaaagaagctgcaaagaccaccgaagctccagtagttaaacctaagaaggttgaagttaaggaaattaagactactccagtaactactagtgctaaaactgttgtaaagcctgttcaaagttaccaaagtgcagtaactactgctgcaaacaataactatggtactcaatggattagtactaatactgctcctaaggctgcatctactgttttaattaaggttactaagactgttgatgttttatcagcaccagatggtcaaaaattagaccaacaagttgaagctggctcagaatttgttgttgttgcttctaagtactacaatggcaacttatattacgaaattagtaatggtaaatggattatggcaaaatatactactcaagaagcacaaattactgcaaaatctggtgtattaactattaattctaagccagactatggtgtaccagtatggcgtgttccaggtcaagatcaaattgctggtaagttcttgaaagacggctcaagctggagatacttccgtgtagcaaacgttcaaggacaaacttggtatgaccttggcggaaatcaatgggtatctgccaagtctgtactagttcgttaa

SEQ ID NO: 4 - DNA, 315 bp, Lactobacillus gasseri
  source 1..315: mol_type = genomic DNA; strain = CCOS 960
atgaaaaaaagaacattcactggtattgctaccgcggcacttatcacaactgccggtatatcagttaccaacaaccttaaaccagaaaatccattaaaaactggtgaaggtactgttcaagctgcaacatatcaacaagaatttttaaacaaagctattccagctgctacaactgcatcatctaagtatggtacttatacttcagttatgcttgctcaagcagcggtcgaatcagcttgggtcaatcaggtttggcacaatcgcctaataataacttattcggtattaagggctcttacaatgggcaatcagtaa

SEQ ID NO: 5 - DNA, 2319 bp, Lactobacillus plantarum
  source 1..2319: mol_type = genomic DNA; strain = CCOS 893
ttgctaagtaccgctttgctgccgatgttgagtggtaaagctgatacggcaagtacaaatcaaaaaccggcagctgcgactaagggaaacagcgctgcttcagctgcaagccaacaagtgaccttaagtgctggcagtcagacggaaacgacagcggccggtgcgactgaccaatcagttgccagtgacggtgccaagactgacgatcaagctgaatcgaccagcacgacgacggctactaccagtgcgacaagtcgtgtcacagtgcgggcagccagtcaagtcgctaaagctgacagtacagaactgcaaagtcagtcctccgccagtgaagctgccaaggataacgcggctgctagtgctacggcggactccacaactagtgctgttgatcaacttgataaaacggccaaagctagtgcagccacgagccaagccagtcacagtacgactaatgagacggctaaagccagcgcggccgcaagtcaggatagtcatgtcactacggaccaatccagcgtgactgtgacgagtgaagtagccaaatcagccgcatcgtcagccgcacctaagcaagcgactgagcaggcggtagccgcgaaaatctcaccaaagattgagacggctgtggcggctgatgcggtccagtcatcagcgatgatggcacggtccacgcgggcaatgactagtcaggaaatcttcctgagtcagattaaagcgggggcaatctctggttggaacaagtatcaagtcttgccatcagtcacggccgcacaggcaattctggaaagtggttggggacaatcacaacttgcaactcagggaaacaacttattcggtatcaagggaagttatcatgggcaatcaatctacttcccaacccaggaatggaatggctcacaatacattacgattcaagacgcttttcggaagtacccgaattggtcagctagtgttgaagatcatggcgcctttttggttgtcaatccgcgttacagcaatttaattggggttaccgattatcggcgcgttgccagtctcttgcaacaagacggctacgcaaccgcgccaacgtacgcgagttcgctgatcagcatcattgaatataacaagctacacgagtgggatcaagaagcgctcagtggtcaggcaagtggcggaaatgataataaccaagttcagcctgatcaagacgtcaccccaaccagtggtacacataagtttactaagacaacgacaattcacaatgcgccggacgccacgagtgccgtagtgggtacttacaacgctggtgaaacagtcaattacaatggtaagttaacggtcggtaatgcaacttggttacgctatcagtcctattctggcgtatcacggtatgtgatgattagtcaaacaacaactaatgataataataaccaggcgacagttacgccagctagtgggtcgtataagtttaccgcaaagaccaacattcgatcagctgcaagcaaaacggctcaagtggtcggcacttataatgctggtgagacggtttactataatggcaagattaccacgggtgggacaacttggttacggtatttatcctattcaggcgctcagcattacgtggctatgagtggtgatgaggttggttctgtggctaaaccggacgtggttgcaaccagtgggtcgtatcgtttcacgaaaacgacagcaatcaagagttcacctgcaactagtgcgacaacggttgggtcttataatgctggcgatacggtctattacaatggtaaggtgacgacaaatggtcagacttggttacgttacatgtcttactcaggtgcccagcattatgtgcaaattagtggtgagagtacgtcgaccaatgttgataaaccgcaagtaacgcctcaaagtggtagttatcgctttacgcaaacaacggcgattaagaatacaccggccggaaatgccccaagtgtcggtacctatagtgctggtgatacagtctactacaacgctaaggtaacggctaacggccagacttggttacggtatttgtcttattcgggcgcccaacattacgtagcgattagtggtaatgccgctaccggaaatacgacgtctaagccggtaacaaacagtcaaggggcgttccgcttcgtaacgacgactaatattcgtacggcgccaagtacgcgcgcaagcgttgtcggtgaatataacccaggtgaaacggtttattacaatgggacggttcaagctgagggttacacgtggctccgatatctgagtcgctccggtgcaacgcactatgttgctaagcttgaaggctag

SEQ ID NO: 6 - DNA, 639 bp, Lactobacillus plantarum
  source 1..639: mol_type = genomic DNA; strain = CCOS 893
atggcgggtaagcggcgaaaatcgcgccggaagcaacggacaaagacacaattatttgtgaagaagggccgccttcagtgggtcaatatcttattagtgttggcggcgatggtggggatggtttggtatattcagcataattgggcggttaagtcgcgggttacggcgacagcacctacaacaacgcatgcggcatttattaaaaagctagtaccagctgcgcaacagcttgatcagcaataccacgtgctggctagtatcacgctaagtcaggcgattttggaatcggattggggacaaagtaccaatgcgacagagaataataatttatttggggttaagtccacgtcgggacggctcatgacgacgcaggagtattatgacggggcctatcatacggtcaaacggcgcttcgccgtttatgatagctggcacgcttcgctggtcgatcatgccaagaaattggcgtacggaacgacttgggattctcagcactacgctgcggtgattaaggcaactgattatcagacagctgcgcaagcattgcagacggctggatatgcgactgatccaagttatgcacaaaaattaattaatattattcaaaaatatgacttacaacggtatgatagaaagtaa

SEQ ID NO: 7 - DNA, 1266 bp, Lactobacillus plantarum
  source 1..1266: mol_type = genomic DNA; strain = CCOS 893
atgagcagtgttttaggaattacattagcaaagcctgtacaagggaaagctgatcaaacatcaagtcctagtaccactaaagttaaagccgcaacttcaggtgctacctcagaagtatcatcaattagtacaattacggctaacagtgctactaacgtgtcttcagcaacccatcaattttcaacagcaactactactagccagcaggccagttcagccactagtaatatttccacaactacagcaagcaaacaaatatctaaatccactactggtaagctaaattcaaatgagacagcaactacaagtaatagcgcggctacctccgtagacagtacggcaccatatataccagcaaaatccaatgtcagtttcaccgacaacatcaagtcaactattgcaaatactccgacaacggtagcagcaccagctattcctagtcatagcacgcaatttattgatgattcagcccctatcacatccccgacacccgttacaactaattctatccatgttcgaccatttacagtacatagttcattttcgtttaaaccaggtcaatttttacgcttcacacttcctaatgttgctttacttcgcgatgacgttgaacaaggtgttccaaattacgtaaaaaactttttcattgcaattaaacctggtgcaatgattgggtggtcacaatatcatattctaccatccatttctggagcacaagcattactcgaaagtggttggggaaaatccacattatcagttcaaggtcataatttatttggaatcaaaggctcatatcatggtcattctattgaaatgccaacaacagaatatttaaatggcgaagacgtaacgattgaggctacctttcgaaaatacccagactgggcaactagtattgttgatcatggcgcattccttaatcaaaattctcgttaccgtaatttgcttggagtaaaaaattattcgaccgttgcatgggatttgcaaaacgatggctacgcaacggcacctaactatgccacttcattaattaatgccattcaagattacgatctgcaggaatgggatcaagaagcctttacgggcaatactggttcgacaactacaaccaccggtaaccacaggtccggcacttatacctttattcagaattcgaatatccggacagaaccatccttatctgcacctatcattggtgtttattatcctggggacgtggttaattacacaggtcagattaaagccgaaggatatacttggttataa

SEQ ID NO: 8 - DNA, 777 bp, Lactobacillus plantarum
  source 1..777: mol_type = genomic DNA; strain = CCOS 893
atggtcgttccagttgatatttggaaccaacgtggcgatgcttctgaccaagttaccttaaaatggccggataatcgtaaacctaaagcggcatttacggttaaccgcaccctggcagctcccggtaacacgatcaagtttacaaatgcctcatccaaaaacgcaaccagctataaatgggaatttgacggtgccacaaagacgaccagcaccgccaaaaaccccacggtcacttaccgtaaggctggcacttataacgtcacattaaccgctaaaaataaggatggccaacggcacgttacaatgaaaaagctcattacgattacaccgaaagctcaaggcgctttaaccttactatccaagaacgccaagaccagtgcctccggatacacgaatagtagtgaagcacctaaaatggcggtggatgggaaactcgatactaaatggtgtgcaaccggtaaggcaccgcataccttgaggctggatttaggccgtcaaacgactgtgagtgcggttaaattagcgcatgccaaagctggtggcgaaggtgctgatatgaacacccgcgcttggaccattcaggtcagcactgatggcaaacgctacacggacgttgcccggacctacaataacacgcaagccactagtctcaatactttcgcggctacttcggcgcgctacgtccggttagtagtcgataaaccaacccaagttgctgacaccgccgttcggatttacgaaatggacgtgctaggattgacgcaaacactcaaataa

SEQ ID NO: 9 - DNA, 285 bp, Lactobacillus plantarum
  source 1..285: mol_type = genomic DNA; strain = CCOS 893
atgcgtaaggacaccgctagcacaatcaaactctacaccacggacctagacgtcactgccgatacccaggttagtttaaccgctaaggcatctggtaaatctacagctaaactagtggtgaccttaaaggacggccgcactaagaccatcgcgggtgaccggacactcagcaagcactggacgaccgttagctatgacgttagtcagttaacaaaaaaacaatcaaaggccttagcctcaaaatcagtgccgccgagactgatgctagctacagcgttcaattag

SEQ ID NO: 10 - DNA, 1227 bp, Lactobacillus crispatus
  source 1..1227: mol_type = genomic DNA; strain = CCOS 961
atgaagaagagacttttgaccagttttgctgctgccgcaatgcttacttctgtagctgtgccagcagtaaatactacgatgatgaatcaagcctcaagccaacgagtttctgccgctaccgctgatcaaactgcatttttaaacaaggctgccaagcaggcagttaaagcagctaaaaaatatggaactttgccatcagtgatgattgcccaggcaataactgagtcaggctggggaaagtcaggattagccgtgaatgctaataacctttttgggatgaaggctgatgattcttggacaggtgagacttatactgccaagactagagaagaagataaaaacggtaagagctactatattactgctaagtttagaaaatacccatcttttgaacaatcatttgaagataatgggagtaagttgagaaatggtgtttcttgggatccgttgcgttataagggcacttggattgaaaatgcatcaacttatgcagctgcaaccaaggctttgaccggtacgtatgctacagactctaagtatgataaggcattaaatagtcatattactagttctaatttgactaagtatgatccagtaaccgttaatactactagaacttatactgctggcaaggatagttcaacttataattggccaactgctccatctgttgccagtgttattggctctgttaaagctggtgaaaaagtagtagttaccaagactattactttccatgatggttcaagcagaatgtatattgatggtcgcggttgggttaatggctcagttttggacaagagcagttccgcaactaaggaaccagtaactcaggcacctaagaatgtgccagctgtttctaagaacttgatgcataatgcctatgtttatgaccaaaatggtaagaagctaaagggcaagatgtacaagaccagtgatgaaaacggtggtaagtggatcaatacttatggtaccaagaccatcaagggtaagacctactaccgtgtcggtgaaaatgaatacattgcagctggtaatattgatggtagcctcagattcttgaagaagaatgcttatgtttacaatcaatatggcaatcgcgataacaatttgaagcacaagaagaatagccaaattgctacttacggcagtgctataactatcaatggtaagaaatactataaggtaggtattcgtcaatacgttaagaaatcgaacttcatgtaa

SEQ ID NO: 11 - DNA, 648 bp, Lactobacillus crispatus
  source 1..648: mol_type = genomic DNA; strain = CCOS 961
atggcaagaaaacgtaaatcaagagtaccaaaaagtgtcaaaaccgttgtgcgtgtattcgtaattctatttattctgatggttgcttttgtcggctttagatattataggcgttacgcgattcaatcagagcagattcaacaagcacagctgcaaaaagaacaggaagcagcaaaactattaaaacagaaaaaagactttatcaaaaaaattggtccgattgcgcgcgaagtggacaagtcgtatgacttattgccaagcatcacgattgctcaagcctgcctagaaagtaattatggacaaagtgatttgtcgcaaaaatacaacaatctttttggtgttaaaggatccaacccgaacacttcggcagtgatgacaactaaggaatatgtaaagaataagtgggttaccgtgaaggcgcgtttccaaatatatgactcatatgaggcttctattcgggctcacgctagattattccagaatgggacaacgtggaaccacgatcagtataagcatgttctagcatccaaagattataagacacaagctaaagccttggttaccgacggctatgcgactgatccggactatgccgataagctgattaatttgattgaacagtttgatcttgaaaaatatgataagtaa

SEQ ID NO: 12 - DNA, 315 bp, Lactobacillus gasseri
  source 1..315: mol_type = genomic DNA; strain = CCOS 960
atgaaaaatgaattaaaacaagtgttgaatgactttatctcacttgctaagactcattatgatagaaaaggcaacgaatacttattagaacaacttgaagttgctttagataagttagaacataatgttcaagatgaagtagatgaagcccgtgcaacttatcaaaatataaatactatttgtcttactaaccatcttcacttggaaacagatgaagaagcattacttgaaaagattaaagaattttcaatgagtaaaggctggcttggtggcttaaacagttggaatacaactaatacttggcctggaagataa

SEQ ID NO: 13 - DNA, 141 bp, Lactobacillus gasseri
  source 1..141: mol_type = genomic DNA; strain = CCOS 960
gtggggaatgctgtaataggagctgctacgggagctactcgcggagtaagttggtgcagaggattcggaccatggggaatgactgcctgtggcttaggaggtgctgcaattggaggatatctgggatataagagtaattaa

SEQ ID NO: 14 - DNA, 228 bp, Lactobacillus gasseri
  source 1..228: mol_type = genomic DNA; strain = CCOS 960
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 15 - DNA, 996 bp, Lactobacillus gasseri
  source 1..996: mol_type = genomic DNA; strain = CCOS 960
atgattggaaaagaaactcaaatacgtttagtaaataaattagaaaatatacaccacgttgttgttcaagcttcagcactcgatggaagtaatgtatttgtcctacaattacttcatagacagagcgatgttattgtttatcaaactccaaatgatagcgaaactgtgacctttaatgaggatcatccaattttatacttgaaaggacccaattcagctggtacagcgggtggacatactcaaacttgggtacaaagtggagaagacaataaatggttcgttggaactaaacctaaaaagcatggtaatacttattggacaacgcaaattgcgcgagtaacagttcctggctatcaaactcaaatttttaccagtaatacggagttgccaagactttcttatcttaatcgcgcaggttcaggctatggcgatggaagtgtagcttatcctggcaaagatttagttagagtagaagcagctgtttcaccgaataaacaatatttcttaattgcaagtattgacatcaatcacacaggtcattttgctatctataatcttgatgaagttaataaaaaactagatcaagcagaagaaaaagctgaagatgttaatattcagagcttgaattgtttaggtgcatttaatatcccatattttaatgatcaaaagattatttcgattcaaggctatggaattgatgataataaggacatttatatttccagtcagcccagcccgcatacaacgttcttaggatttccaaaacaaggcaaaccacgtgaaattgttaaaattccctggggaatatccgatctaagtaaatggtcagtggtaaatttagacaacagtttaaaattagatgcactaaacttttgcactgaatttgaaggaattcaagtaactggtgattgtctttacttaaccgttgcctatcaccaacgaaacagtgatttgactactttgatgaatcgaatatatcaggttgaaaaattttag

SEQ ID NO: 16 - DNA, 1461 bp, Lactobacillus crispatus
  source 1..1461: mol_type = genomic DNA; strain = CCOS 961
atgataacaggtattaagcggttaagacagacaaaaagatgggctggattagccgcttttttattaggaattttagctgttttatcagttatatattttattgtaattttaattgcagcaatgggagctgctacagatgatgaatgtgatacaggaggagaacaaggaggagtagtagaaggacgaattagtgtcattgaatcacctaagtatggacagaccgctatgatgcatatagcggatgctgtgcatgagaaaactgggattagtgctagattactttttgctcaaatgggacaggaaacaggtaatggtgatagttcagttgctaaacatgatcataactttggtggtatgacttattcagaaggaagcactatcggtaccaaaggtgaaagccgtggtagtgaaggtggtaattacgttcactacaagaacttaagtgattttgcgaccgagtgggctgtgacggttcagaatggttttaaaaaagctggattaggaaaagatgctacagttgctcaatatgctcatgcaatgaaaaaagcaggatactataccgcagctgaaagtgattaccaagcaggtatggaagctcaagctaaaaaatatgatgccttgaaaggcaataaatcactagctaaaggtgatgggtctagtggctcagataattcagctgaggaagattgtaagccaataagcggcaaatgggggtggccattcaagtcaattcctaaaaaaggaccaggtaatacaatctcaggtgagcaattatttggtaagtcatctacaagaacaggtggctttcatgatggtgttgactttggtactgttccatatggtggacaagatattttggcaattcatggtggaaccgttaagaaaattgcatttcaaggacatactcaagatggtctaggaatgtacgtttgggtagaaggctccgacggttggaatgtcatttatcaggaattcggctttgatgaagcagatttgaaatacgtcaaggtagaggtaggcgacaaagtttcagtaggagataaaataggtcacttagcaagtaatgcacatggtattactcacgttcacattggtgcaactgaaaaagattttgctaccgcagagctctctgcgtacaaagatgatggtacttggaaagacccaattaaattaattaaagatggtttgaacaatgatgcagaatctgaagaagatgatggcttatcaaaaactgaatctgaagcacgtgactggattgttcagagagaatctggaggtagatgggacgcagttaacccagctaacccaagtgtttatggacgttatcaattaaagagagaatatcttaatggtgattacagtcataagaatcaaacaagggtagcaaataaatatgtaaaaggcagatatggttcatggcgtaatgcacaaaaattctggatgaaacatacctggtattaa

SEQ ID NO: 17 - DNA, 981 bp, Lactobacillus crispatus
  source 1..981: mol_type = genomic DNA; strain = CCOS 961
atggttaaaagtattacacctcatttgttttatcgcttgaatgggatgcaccatgttgtagcccaagttggtgtagtaaatggtgatcatgtttttgccttgcaacttcttcacagtgcacatgatgtgttggtttatagaaaacatgaaggtttaactaagaacatcgattatactgatccacacttagtaatgatgggctttggccacacgcaaacctgggtagcagctaatgacaaggatgaatatttcgtaggtgctaaaccaaattcaggcaactggactactcaaattgcacgagtaaagtacccaagacttttaccagaaagatatacttcaaatacacaacttccacgtttgtcacacttgaatcacgtaaccgacgttccttatgatggtcatgatcacttgcacagagtagaagcttcagtttcaccaaatggcaagtacttcatgattgcttcaatttgggatgatggttcaggtcactttggtttgtttgacttaaatgaagtaaaccaaaagttgaatgaaaatggcactaagaacacgccaattactgatttgcattgccttagtgcttttcacattgataactttgatcatccaagcgtggcaccaagtgaagaagctccacaaatgattgattctgttcagggctacgccattgatgatgacaagaatatttatatttctaaccaattgtcacccaagattgatcatgcgaatggtgaagttactacttggtcacgtaagatcgttaagtttccatggggtgaaactaatccagagaactggcaagtagcaatgattgatggtattgatttgcctgatcgctacagtgaagtagaaagtattcatgttcaggcgccagatgacatttatctaactgtggcttaccaccaaaaatatgtcaaagatggcgagtttaagttaagaactttggagaatcaaattttccacattagtgatttaggctaa

SEQ ID NO: 18 - DNA, 936 bp, Lactobacillus crispatus
  source 1..936: mol_type = genomic DNA; strain = CCOS 961
atgttagatgcaacaaatattagaattttagatagattcgatatgaatacaggttacagagctgtagttcaaaaaggtaatgtaggatcaaagtatgtttatggtttacagttaagaaataaccagacagaaacacatgtattgcgtggcttccgtggcaacgtgacaacccctgtgcttactttagtaggtgcagcagctggacacacccaaacttgggaatattcaggtagatctggtaaatggttcgttggtactaaaaacaagaataaatgggcttctcaaattgctcgagcagatattagatacaaatcatatgcttcatcaaatactgaattcccacgtttagcatacttgaaccgcgcaggtaatcctgaagatcaatgctctggtgatgaaatggaacgcgctgaagtagcagtttcaccagattatagtatgcttttaattgctactattgagaataatggaacaggccacttttcaatttatgacctaaatgtcattaataatgcattagatgaggctggaaataatggtttcgtaaatatgggaaattatcaatgtgaaactagttttacagtatatggtttgtatggatctgttttaaactctgtacaaggttatgatcttgataattctggtaatatttatattactagtcaaaaatcaccaagtttaagtaatggttcatggtcatactatcacaaggaaattgtaaaaattccgttttatgcacgtaatgatcaatctcaatgggaaaatgtaaacttaagtgcatttggtggattagatatttcaggtgaacatagtgaagttgaaagtatccaaattattgatgaaaatcacggttacttaactgttgcataccatgctaatgttaacggtaaaaataaaactgtttcaaataaggtttatgaattagcttgggactaa

SEQ ID NO: 19 - DNA, 252 bp, Lactobacillus crispatus
  source 1..252: mol_type = genomic DNA; strain = CCOS 961
atgaacaaatttgaaactttaacttatgaagaactgtcagctgttttcggtggtaacggtggtaaagcaagacgccgtagaaaaatcacaaattgtgctaaagctattggaatgggagcgttgaaggatggactaaaatatggcattgcaggcactgcatttggtactccaattggtacagtaggtggagctatatttggtgctaatgtgggtattattagcggatcagtttcttgtgtatcacatttgtaa

SEQ ID NO: 20 - DNA, 198 bp, Lactobacillus crispatus
  source 1..198: mol_type = genomic DNA; strain = CCOS 961
atgaagaattttaaggtagtaaataatattgagctaaatagagttgttggtggtaaaattattcgattaactccatatatgttgtataatactaagacacataaaactatcccagattacggtgcaatttggggtaaagcgggtcaaaccgtagccaatggatggttacaatatggaccatggggcagtagaggttaa

SEQ ID NO: 21 - DNA, 219 bp, Lactobacillus crispatus
  source 1..219: mol_type = genomic DNA; strain = CCOS 961
atggtaaagtttcaagaactaaaagaaaatcaattaagtcaagttttaggcggtacacatcacaagcgcggaggcggaaagtaccattactatggtaatggtgtatattgcaaccgatattattgtcatgataatttagcacaaatgtgggatagcgttggtcgtattatgtatactggatggcagaaggatggaccatttgcgcatcctttggtgtaa

SEQ ID NO: 22 - DNA, 675 bp, Lactobacillus crispatus
  source 1..675: mol_type = genomic DNA; strain = CCOS 961
atgaaaaaaactgaaagtaagtttgcacttttagctgctttaattgctattttagcttttgcctctattcccttatggcaaaacaatctgaatagtttacgaccacaaactcacacggttaagaaaaagaagactgctaaaaagaaaaaagtcgttcatcttacttggggttacccttttaagaagctctatgaaaagaaaatcaaatttaaatctggacaaaagtttggtgaaactgacgtcattcgtcgagtctatcctagcaagagttattttcatgatggctatgacttcggatttagtgaagttggacattcttccgtatatgctgtgcatgcaggaactgtccatcgtgttaaatacgccccaggtttaggactttatatttggattatttccgatgatggctatgttgaagtttaccaggaaggcttcctcagtattactgatatttatgttaaaaaaggtcaaaaagtcaagttgggacaaaaaattggtaaactgaccggctctcacattcatttaggagttactaagacagataaagattacattgataaaaagcatgacaatacgccatgcaaatactattggaaagataatggcacttggttaaatccaatgaaaatcattgaagataatctaagggcagcaggtaaagatccagtccaataa

SEQ ID NO: 23 - DNA, 624 bp, Lactobacillus crispatus
  source 1..624: mol_type = genomic DNA; strain = CCOS 961
atgaaatttcgtaaattaataatttctttgttaggtactgcattattaacttcaagcgttggtttatcaaccaccactgcttctgctgatacgcttgatgactcacaaaatacaactgaagttcaacccaagaacctcaagtgggcttatccgtttaaagccaacaagaaaaatggtgttcgtccaatgtataatgcacaaacttttggcataactaactatatgcgttctactacaccaccttcctactttcatgatggttgggattttggtttttcagaagttgggcattctaacgtatatgcaattcatcaaggtactgttaaaaaggttgcttatggcaacggccttggctggttcatctgggttattagtcctgataattacgttgaagtttaccaagaagggtttaataagaaaaaagacatttatgttaagactggtcaaaagattaaattagatcaaaagatcggtaagctaactggctcacacttacaccttggcgttactcaaacaaataaagattacataaacaaatatggttttccatgtaagaattggaacgttaacaacggaacttggctcaatccaatcgaggttatcaaaagcaacttaaagaaatag

SEQ ID NO: 24 - DNA, 1293 bp, Bifidobacterium bifidum
  source 1..1293: mol_type = genomic DNA; strain = CCOS 571
atgaggatgcagcagatcaaacacggcggcaggaggccgcgccccagttcagcccccaccatcatcgcggccatgctctgcgtggtggcccttgtggcggcaggcacggcatggtggatgctgcgcccccagcaaaaagacacgctcgaacatctcgcgcagccacagtcctcctcttcgacgctgaaccgcgacaaggcccccacaccgaaaccgcagaaatctcaggagcggcagaaggcagccggtgactcccccgcggccaaggcgagccgcgccatcgccacgatgagtctcgatgaacgggccgggcagctcatcatggcaccgatgttcgcaggcggcaatccggccgatctcagcgccctcatcagcacacgccatgtcggttccgtggtgctcatcggcaactggaacaacggcacagccgcagccaagaccgccgctgacgcgctgcaaagctatgctccgagcggcaaccagctgatcgtctccaccgatcaggaaggcggccaggtgcagcatctcaagggcagtggattcgacacgatgccgtccgcggtggcgcagggccagatgagcgctgacacgctgcgctcctccgcaaagacctggggcggccagctcaagcaagccggcatcaacgtcgacctggccccggtattggggacggtgcaggtcaagcgttcctcgaacgctccgattggcgcgctgaaccgcgatttcggtttggattccaacggaaacgcgcagcacggcatcgcgttcgtcgaaggcatgcgcgacgcgggcgtcggcgcgaccgtcaagcactaccccggtttgggcgcggtgaccggcaataccgatttcacgaccgaaggcatcctcgacaccacgaccacgcttgatggcgaggagataggcgcgttcaacaccacgatcaagcaggcgaagccggctatggtgatgatgtcgctggcgacctaccagcgcatcgactcgtcggcgcccgccgcgttctccagcaagatcatcgacggcacgctgcgcggcagcgtcggctatgacggcgtggtgatctcagactccctttcggccgcggcggtcagcggcatagcgaccaaggacctgggcgtgcgtctggtggacgccggcggcgatttggcgtgcataggcgatacgtcatatgtgacgccgattcttgacggtatcatcgcccgtgcgcagtccgacccggccttcgcgaagaaggtgacggcctccgcaacgcgcgtaatgaccctgaaataccagatgggactggcgaagtga

SEQ ID NO: 25 - DNA, 5808 bp, Bifidobacterium bifidum
  source 1..5808: mol_type = genomic DNA; strain = CCOS 571
atggcagttcgcagacttggtggccgcatcgtggctttcgccgccacagtggccttgtcaataccgttagggttgttaacaaattcagcgtgggcggtcgaggacgccacccgatccgactccaccacgcagatgagctccacgccggaggtggtctactccagcgccgtggattccaagcagaatcgcacctcggatttcgacgccaactggaagttcatgctgtccgattccgtgcaggcgcaggatccggcgttcgacgattcggcctggcagcaggtcgacctgccgcatgactacagcatcacgcagaagtactcgcagagtaatgaggccgaaagcgcataccttcccggcggcaccggctggtaccgcaagtccttcaccatcgaccgggacctcgccggcaagcgcatcgccatcaacttcgacggcgtgtacatgaacgccaccgtctggttcaacggcgtcaagctcggcacccatccgtacggctactcgccgttctccttcgacctgaccggcaacgccaagttcggtggggagaacaccatcgtcgtcaaggtcgagaacaggctgccgtccagccgctggtactccggctccggcatctaccgcgacgtcaccctcaccgtcaccgacggcgtgcacgtcggcaataacggcgtggccatcaagaccccgagcctcgccacccaaaacggcggcaacgtgacgatgaacctcaccaccaaggtcgccaacgacaccaaggccgcggcgaacatcaccctcaagcagaccgtgttccccaagggaggcaagaccgacgccgccatcggcaccgtcaccaccgcatccaagtccatcgcggccggtgccagcgcggacgtgacctccacgatcaccgccgcctcgcccaagctgtggagcatcaagaacccgaacctgtacaccgtgcgcaccgaagtgctcaacggcggcaaggtgctcgacacttacgacaccgaatacggcttccgctggaccggcttcgatgcgaccagcggtttctcgctcaacggcgagaaagtcaagctcaagggcgtctcaatgcatcatgaccagggatcgctcggtgcggtcgccaaccgccgcgccatcgagcgccaggtcgagattctccagaagatgggcgtcaactcgatccgcaccacgcacaaccccgcagccaaggcgctgattgacgtctgcaacgagaagggcgtcctcgtggtcgaagaggtcttcgacatgtggaaccggtcgaagaacggcaacaccgaggattacggcaagtggttcggccaggccatcgccggtgacaacgccgtcctgggtggcgacaaggacgagacctgggccaagttcgacctgaccagcaccatcaaccgtgacaggaacgccccgtccgtcatcatgtggtcgctcggcaacgagatgatggaaggcatcagcggcagcgtctcgggcttcccggctacctccgccaagctggtcgcatggacgaaggccgcggacagcacccgcccgatgacctacggcgacaacaagatcaaggccaactggaacgagtcgaacaccatgggcgacaacctgaccgccaacggcggcgtggtcggcaccaactactccgacggcgcgaactacgacaagatccgcacgacccacccctcatgggccatctacggttccgagacggcgtccgccatcaacagccgaggcatctacaaccgcaccaccggcggcgcccagtcaagcgacaagcagctgaccagctatgacaattccgcagtcggctggggtgccgtcgccagctccgcctggtacgacgtggtccagcgcgatttcgtcgccggcacatacgtgtggaccggcttcgactatctcggcgaacccaccccgtggaacggcaccggctccggcgccgtgggctcctggccgtcgccgaagaactcgtacttcggcatcgtcgacaccgcaggattcccgaaggacacctattacttctatcagagccagtggaacgacgacgtgcacacgctgcacatcctccccgcatggaacgagaacgtcgtcgccaagggctccggcaacaacgtgccggtcgtcgtctacaccgacgcggccaaggtcaagctgtacttcacaccgaagggcagcaccgaaaagcgactgatcggagagaagtccttcaccaagaagaccaccgcggccggatacacctatcaggtctacgagggcgccgacaaggactccaccgcccacaagaacatgtacctgacctggaacgtgccgtgggccgagggcaccatctccgccgaagcatacgacgagaacaacaggctgatccccgaggggtccaccgagggcaacgcgtcggtgaccaccaccggcaaggccgcgaagcttaaagccgatgccgaccgcaagacgatcaccgcggacggcaaggacctgtcgtacatcgaggtcgacgtgaccgacgccaacggccatatcgtccccgatgccgccaaccgcgtcaccttcgacgtcaagggcgccggcaaactggtcggcgtcgacaacggcagctcgccggatcacgactcctatcaggccgacaaccgcaaggcgttcagcggcaaggtgctcgccatcgtccagtccaccaaggaggcgggcgagatcaccgtcaccgccaaggccgacggtctgcaatcatccacagtgaagatcgccaccaccgccgtccccggcaccagcaccgagaagacggtccgcagcttctactactcgcgcaactactacgtcaagaccggcaacaagccgattttgccgagtgatgtcgaggtgcgctactccgacggcacgtcggaccgtcagaacgtcacatgggacgcagtcagcgacgaccagatcgccaaggccggttcgttcagcgtggccggcacggtcgccgggcagaagatctccgtgcgcgtgacgatgatcgacgagatcggtgcgctgctcaactattcggccagcacaccggtcggcacgcccgccgtgctgcctggctcgcgtccggccgtgctgcccgacggcaccgtgaccagcgcgaacttcgccgtcgactggaccaagcccgctgacaccgtgtacaacacggccggcaccgtcaaggtccccggcaccgccaccgtcttcggcaaggagttcaaggtcaccgcgacgatccgcgtgcagcggtcgcaggtcaccatcggcagcagcgtctccggcaatgcgctgcgcctgactcagaacatccccgccgacaagcagtccgacacgctggacgccatcaaggacggctccacgaccgtcgacgccaataccggcggcggcgcgaacccgtcagcatggaccaactgggcgtactcgaaggccggccacaacaccgccgagatcaccttcgagtacgcgaccgagcagcagctcggccagattgtcatgtacttcttccgcgacagcaacgcggtgaggttccccgacgccggcaagacgaagatccagatctccgcggacggcaagaactggacggatctcgctgccacggagaccatcgcggcccaggagtcgtccgaccgagtcaagccgtacacctatgacttcgctccggtgggagccacgttcgtcaaggtcacggtcaccaacgccgacaccacaacccccagcggcgtggtctgcgccggcctgaccgagatcgagctgaagaccgcgaccagcaagttcgtcacgaacacgtccgccgcgctctcgtcgctgacggtgaacggcacgaaggtctccgactccgtgctcgccgccggctcctacaacacgcccgcgatcatcgcggacgtcaaagccgagggcgaaggcaacgccagcgtcaccgtgctgcccgcgcacgacaacgtgatccgcgtgatcaccgagtccgaggaccacgtcacgcgcaagaccttcactatcaacctgggcacggagcaggaattccccgcagactccgatgaacgcgactacccggccgccgacatgacggtcaccgcgggtagcgaacagacgtccggcaccgcgaccgaaggcccgaagaaattcgcggtcgacggcaacaccagcacgtactggcattccaactggacgcccaccaccgtgaacgacctgtggatcgccttcgagctccagaaacccaccaagctcgacgcgctgcgctacctgccgcgccccgcgggcagcaagaacggctccgtcaccgaatacaaggttcaggtcagcgatgacggcaccaactggaccgacgcgggctccggcacatggaccaccgattacggctggaagctcgccgagttcaatcagccggtgaccaccaagcacgtgcggctcaaggccgtccacacctatgcggattccggcaacgacaagttcatgtccgcctccgaaatccgcctgcgcaaggccgtcgacaccaccgacatcagcggcgcgaccgtgaccgtgcccgccaagctgaccgtcgaccgggtggacgccgaccatcccgccaccttcgccacgaaggacgtgacggtgacgttgggcgacgccacgctgcgctacggcgtggactacctgctcgactacgcgggcaacaccgccgtcggcaaggccacggtgaccgtgcgcggcatcgacaagtactccggcaccgtcgccaagacgttcaccatcgaactgaagaacgccccggcgccggaaccgacgctgacctcggtgagcgtcaagaccaagccttccaagctgacctacgtggtcggcgacgcgttcgacccggcaggactggtgctgcagctcaactatgacgacgacagcaccggcaccgtgacctggaacacgcagacggccggcgacttcacgttcaagcctgcgctcgacgcgaagctcaaggtcaccgacaagaccgtcaccgtcacctaccagggcaagtccgcggtcatcgacatcaccgtctcgcagcccgccccgaccgtcagcaagacggatctggacaaggccatcaaggcgatcgaggccaagaacccggattcatccaagtacacggccgactcgtggaagaccttcgcggacgccatggcgcatgccaaggccgtcatcgcggatgattccgccacccagcaggacatcgataacgcgctcaaggcgctgaccgacgcctacgccgggctgaccgagaagacgcccgaacccgcccccgtcagcaagtccgagctggacaagaagatcaaggcgatcgaggccgagaagctggacgggtcgaagtacacggccgagtcgtggaaggcgttcgagaccgccctggcgcatgccaaggccgtcatcgccagcgattccgccacccagcaggatgtggacgcggcccttggcgccctgacctccgctcgtgacggactgaccgagaagggcgaggtcaagcccgacccgaagcctgaaccgggcaccgtcgacaaggcggcgctggataaggcggtcaagaaggtcgaggccgagaagctggacgggtcgaagtacacggccgactcgtggaaggcgttcgagaccgccctggcgcatgccaaggccgtcatcggcaacgccaactccacgcagttcgacatcgacaacgcgctgtcgatgctcaacgacgcccgcgccgcgctcaaggagaagcccggccgcatcatcgccatcatcgatggcagcgcactgagcaagaccggcgcatccgtcgccgtcatcgcctccgtcgcggccgcgatgctggcggtcggtgccggcgtcatggcgttgcgccgcaagcgctcctga

SEQ ID NO: 26 - DNA, 465 bp, Bifidobacterium bifidum
  source 1..465: mol_type = genomic DNA; strain = CCOS 571
atgcattcgcgaccgatgttcccgattccggagacgcaggtcggcgacggctgggtcggcgacgtcgactacacgaagcaggccacgaagcaggacagcaatttcctcgtcgactgggtgcgcgtgtaccaatccgaggggcagccggtgacgcgtttcgacgacctggacggcgccgagagcggcgcgtaccggagcgcccccgcctcacggaccgaggggctgaccgcagtgagcaacggcgacgccgcctggcggaacaagaacaacttctactacggcggacagccgcggtatgagaccagccgttcccatgccgtgcccctgatgcagggccgattcgccctgcgcttgcggggcccacaaacactctgccgaatccggacgaatgcttttacccgcagcgagcgacccgttgcaggcaaggttgtgccgccgaatcggcgaaatcgcgcggcgccgtga

SEQ ID NO: 27 - DNA, 558 bp, Bifidobacterium bifidum
  source 1..558: mol_type = genomic DNA; strain = CCOS 571
atgatttcggcgagccgagagatacactccatcaacctgtaccgcaatgggtatgagaacgcgctgaacacgttcaagcgcatcaaggtcgaagtgtcctccaacgaggacttctccgacgcgaacgtgctgttcggcaccgccgacgtcgaggaaaccgccgcgaccaagctcgccgcgcagaccatcaacctgaccacgcccgtcaccgcgcggtacgtgcgcatctggcagaagggccattgcatccagaacacgaactcctcatggaagggatacggcaacggcgtgggcttgcgcgaaatcgaggtcatcgccaagctcaaggacggcgagaccctgccggacgcgcaggagacgcgcaacatcgcgctcggcaagttgccgtatgtgtacggcctcgacccgaccaacatcgcggcgatcagcgacggcaagcaggacgacaactacgccgtgcacaacagcaccggcgagcggtggctgcagttcgaatacaagaaccgctaccgtatccacgagggctcaagccgggcacctatccgctggtccgcatag

SEQ ID NO: 28 - DNA, 327 bp, Bifidobacterium bifidum
  source 1..327: mol_type = genomic DNA; strain = CCOS 571
atgagacaattattccgccgtatcacgaccatatccgccacatgcgcgttggccgtttcggttgcagccgcagtcgcggtggccgccgaacccaccacaggcaacatacttaccggcaaactgcccacgaccaacagcacgcatctgatcggcgacggcgacaccggattcggcccgaccgacagcaacatcaccaaaatcatcgccggagaagagaacggcagcgcggaaaacaacggatacgccagctgggacgacgtatacctgcaatatgatttcggcgagccgagagatacactccatcaacctgtaccgcaatgggtatga

SEQ ID NO: 29 - DNA, 5796 bp, Bifidobacterium bifidum
  source 1..5796: mol_type = genomic DNA; strain = CCOS 571
atgagaaaacgagtggtttcagtggcccttgcggttgcgctcgctgtcgccccactgggggtggtcagcacagcgtccgccgcgccgctttctgcaagtgatttgcaaactctcgcgctccgttcagcggctagctccaatgacgcgaacgccaacgatgtcgccaccgtcgcggacgatgctgccgtgaacggctggacgattgaccgcaacaccgccaaaggcggcgaaattcttgcagccggcactggcgattatgcgggatggactcatttcaagtccacctccgccaacggcaatgccaccagcagcagtggctatccggctgtcgccattagcggcaagacgattgacctgactcgggctggagagttctccatcaaggtcaagtccccgcaggctggttccgcgaaccgtttcggcttctacctcggctacaaggatccgggcaacgccctgttcctgggctatgacaaggggggctggttctggcagaagtatgtcggaggcaatggcgattggtacaacggcacacgtgtcgctgcccctgccgcgaacgctgaaatcaccgtcaacgtctcctggactgccgccaaggttgccacgctgactattgacggacagaaggcgttcgatgtggattactcctcgatgaccgcgctgaccgacaagctcgccatgaaggccggctcttactccggcaccagcgaagtcactgatgtctacttcaaaaacttcaccgtgggtgaagtcgccaagcacaacgtgaccggcaaggtcgtcgacgccagcggcgccgccatcgccggcgccgaagtcgtcaccggcaagaactccgccaccaccgcggcggacggcaccttcaccctcaccggcctcgccgccggcgactacacgctgaccgtgtccgccgaaggctacgatgacgccaccaagaccgtcaccgtcgccgatggcaatgcctcagtcggcaacatcacgctgaacaagtcggccgaagtcgctactgagacgctctccaccgccgccatggacgtgcgcgtgaagaagaacttcccgtccgtgtacgactacacgatgaagaagctcgacggcaagatcatgtacggccagccgaaggacgtgcgcgtcatcaccattaacggcaccgacgtgacgctgaaggactccgacgtgaccttcaagaaggtcagcgcgaccgaggcccagtacacgctgaacgtcaagagcggcgacaagatcaacgcggtcgtcaccgtgcagatcaaggtcgtcgacaatacgctcaagctcaacgtcaccaagatcgtgaacaaggcggatgacgccaagaccgaggccgaggagaacccggtccagacgattgcgttcccgaaccagagcctcatctccgtgcgctccggtcaggacggcgcccagttcaccggcgcccgcatgtcgtccgacactgccaggcccggtgacaccaacttcgacatcaccgccgacaccacggtcagcaatgcgaacgactacacctacggcttcgtctcgggcaatggcctgagcgccggcctgtggtccaactccgagcacgacggcaccaccgtcggcaacaccgtcgccggcggtgcgaggaacacccgcgtgctcacctccacccagaaggtcggcaaggccacctccttcggtctgggcactgcaccgtggtactaccaccgcgtcgtgaccgacacgaagaagaacacctacaccgtcgaagagaccgacatgccgaagatggccgtcgccatcgccggcgacgagaacgaggacggcacggtcaactgggaagatggcgcgatcgcataccgcgacatcatgaacaacccatacaagtccgaggaagtgcccgagctggtcgcatggcgcatcgccatgaacttcggctcccaggcgcagaacccgttcctgaccacgcttgacaacgtcaagaaggtcgccctcaacaccgacggcctcggccagtccgtgctgctcaagggctacggcaacgaaggccacgactccggccatccggactacggcgacatcaacacccgcgccggcggcgccgcggacatgaacaccctgatggagaagggcaccaagtacggcgcccgcttcggcgtgcacgtcaacgcgtccgaaatgtacccggaagccaaggccttcagcgaggacatggtgcgtcgcaactccagtggcggcctgagctacggctggaactggctggatcagggcatcggcatcgacggcatctacgatctcgcctccggcatgcgcaagtcccgcttcgctgacctcaagagcaaggtcggcgacaacatggacttcatctacctcgacgtgtggggcaacaacacctccggcgccgaggactcgtgggaaacccgcaagatgagccaaatgatcaaccagaacggctggcgtatgaccaccgaatggggcgccggcaacgagtatgacgcaaccttccagcactgggccgccgaccttacctacggtggttccgccatgaagggcgagaattctcaggtcatgcgcttcctgcgcaaccaccagaaggacagctgggtcggcgactacccgtcgtacggccaggccgcgaacgccccgctgctcggcggctacagcatgaaggacttcgaaggctggcagggacgcaacgactacgccgcgtacatcaggaacctgtacacgcacgacgtgtccaccaagttcatccagcacttcaaggtcgtccgctgggtcaacagcccgctcgacgccacctccgtgaaggatgcctccgtcaacaacggcaacgagcagatcacgctgaaggacgaccacggcaacgtcgtcgtcctgtcccgtggctccaacgacaccaacaacaccgcgtaccgcaaccgcaccatcacgctcaacggcatcaccgtggcgtccggcgcggtctccccgggcaacagcaacaccgtcaagggcactgagtcctacctgctgccgtggctgtgggacgtgaacaccggcaagctcgtcaagtcctccgatgagaagctgtaccactggaatacccagggcggcaccaccgagtggaccctgccgaaggattggcagaacctcgcctccgtcaaggtgtaccagctcaccgaccagggcaagaccaacgagaagaccgtcgccgtctccggcggcaagatctcgctgaccgccgaggccgagaccccgtacgtcgtgaccaagggctccgagaagcagatctccgtcaagtggagcgaaggcatgcacgtcgtggacgccggcttcaacggcggccagaacaccctcaaggacaactgggccgtctccggcaccggcaaggccgaggtcgaaggcaccaacaacgccatgctgcgcctgaccggtgacgtgaaggtctcccagaagctcaccgacctgaccgcgggcaagcgttacgcgatctacgtgggcgtggacaaccgcaccaactccccggccaagatcaccgtgaccaacggcaccaaggtcctcgccaccaacgagaccggcaagtcgatcgccaagaactacatcaaggcgtacggccacaacacgtactcgaacactgaaggcggcagcagctacttccagaacatgtacgtgtggttcgtggctccggaaagcggcgacgtcaaggtgacgctctcgcactccggcgcatgcgacaacaccgaccatgtgtacttcgatgacgtgcgcgtgcttgagaacggttacaagggcctgacgctcaacgctgacggcaccctgaagacgctcaccaacgacttcgaggacaatgcccagggcatctggccgttcgtggtctccggctccgaaggcgtcgaggacaaccgtatccacctgtccgagctgcatgacccgttcacgcaggccggctgggatgtcaagaagatggacgacgtgctcgacggcaagtggtccgtcaaggccaacggcctgatccagaagggcacgctgatctaccagacgatcccgcagaacgtcaagctggaaccgggcgagacctacaaggtgtcgttcaagtaccagtccggctccgacgacatctacgccatcgccaccggtgacggcgagtacaacgccagcaccgtcaagctcaccaacctgaagaaggccctgggcgaggacggcacggccgagttcgagatcaccggttccatcaccggcgacagctggttcggcatctactcgacctccaccgctcccgacctgcagaacacgtccgattccgccgccaacttcggtggctacaaggacttcgtcctcgatgacctgaaggtcgagcacgtggcatccgccgagcacaccaaggccgacgccgaggcgaagctcaaggaagtcaaggacacctacgacggcaagagcggcgactactccgccgaagtctggaccacgtacgtgaacacggtggccgagatcgaggcgctcatcgccaaggataagcccgactacaccaccgcctacaacaaggctgtggcgctggccgagtacatgaagaacgcaccgggcgacgactcgaacgatgcgtacgacgtggcgaccgacgcctacaccgtcgaggcgggcagccagcaggcactgtcgggcggcaatgagggcccggcaagcctcgcccaggacggcaatgccggcactcactggcacacctcctggagcgccaacgcagtgtccgccggtaccgcctggtatcagttcaacctgaacgagccgacgaccatcgacggtctgcgttacatggcccgcagtggcggcgcgaacgcgaacggcaagatcaagaagtacaagattacgctgacgctgtccgacggcaccaccaaggacgtcgtcaccaatggcacgttcaccacgaccagcggtgtctggcagaaggtcaagttcgacgcggtcaagaacgtcaccaaggtgcgcatcaccgctctcgaaaccgcgggacagtccgcgggcgaggtgaacacgtacgcctccgctgccgagctgcgtgtcaccacggttcgcgacgtgccgagcaccgaggtcaaggtcaacaagtgcgatctgcagaacctgtacgatgacgcttccgccctgaccgaggccacctacaccgccgacacctggaaggtgctcgtcgccaagcgcgacgccgccaagaaggtgctggacgacgagaacgccaccgcgcacgacgtggctctcgcctaccagaacctgaaggatgccatcgccgccctggaagagcgcgtggacacctccaagctcgccggcctggtcgccgatgccgagaagctcaaggaatccgcgtacaccaaggattcgtgggccgcgttcaagaaggctctggatgccgccaaggccgtcctgaacaatgcgaacgccaccaaggctgacgtcgatgccgcgtacaacgcgctgaacgccgcgatgaaggccctgaagcccgcctcctccaagccgactccgaacccggagacgaccgacaagagcaagttgcaggccaccatcgatcaggccaaggccctggatctgagcggttacacgaagaagtccgcccaggccgtgcgtgacgccctcgccaaggcccagagcgtcctcgccgatgacaacgccacccaggctgacatcgacgccgcgcagaaggccctcgctgacgcgatcgccgcactggagaaggccgatgccaacggcaacgccatctccaagacgggtgcgaacgtcgccgtcatcggcatggccggcatgatgctggtcgccgccgctggcgcggtgttcatcgcccgcaagcgcgccgagtga

SEQ ID NO: 30 - DNA, 2604 bp, Bifidobacterium bifidum
  source 1..2604: mol_type = genomic DNA; strain = CCOS 571
atggtttcgccacatcatcttttgaagattgcgactgcgctgtctgcggttgcgttgacggcttccgttgcggttacgccggcttacgcattgcaggatattgcgattgaggatgctgttgcacagagcggtcccgttactgcagataatggcgttgttgtgcagtctgacgatcagtctgacgatcagacgggcgaccagcagtcgcaagacggcatgcctgacaatccgaatgcgaaacttccggataccgtcagtgacgagatttccgacgatgccaccgtggtttcggaagacctggccgttacccccgaaggtgaggtcaagaatattgaaaccggcgaaaccgtgaccgatgcgacactggtcggtacgcaggatcagcagccggatccgctcgccaaaaccaacggcgaatccttcattccggtaagtgcggaagatgtcaaaaatgctgtagccgacgcgaacgtacagctctccaaattcgaaggcaatgaatacggcgcgcactggggcacttacaacaatacgaaagccttcttcgactaccagaacaacctgttcgtgcagcaagccaaaggcgtgatcgacgtttccgaatggcaaggcgacatcgactgggccaaagcgaaagccgacggcgtggaaggcgtgatcatccgcctcggctacggttggggcaacaatgctgacaggaaagcacaacgtaacatttccgaatgcaagcgacttggtatcccgttcggcatctactggtattcctacgcggacactccgtcgattgccaaagaagaaggtgccggcgtggtcgccaaactcaagcgattcggtgtgcgcgcgagcgacttggcgtaccccgtgtactacgacctggaaaagtggacgtggaaggggcatcagccgcccaccgatccgaacgtgtacagcgatatcgtcaacaattggtacggcgcgctgcagtccgccggatacaagaacctgggcgtctactcgtacaccagctacctgcaaggcccgctgaaacatgccgacatttacgccaaaaccacgtgggtggcgcagtacggcgctcgcatgggattcgactcgttcccgaccaacagtcgcggatggcaatacaccagctccggcaaagtgggcggcatcaggggcaatgtggatatgaatgcgtttgggaataaggagtatgtgaatggcgggagtagtaatgatttacaagcggccattgatgtacgcaaaatgactgcagttaccattccgaatggcaattattacatcaatgttcgttccaaagtcgcgtccagtgttgatgttcctggtggaagtgctgcggattccaccgccattcagttgtattccggcaacagttccaaggctcagcagttcacttttactaggcagtctgatgggagctacgagatcgtcaatgtgaattccggcaaggctttggacgtgcgtaatggtgttgctgagaataacgctgtagtgcaacagtattctcgaaataattcccaggctcagcgatggtttattcgtgattccggagcgggatcttatttgcagtccgctctcggtaattgggttctggatttgagtggcggaaatactgcgaatggtgctgcgattcggctgtatgcgccaaatggcactgcttcacagctgtttgttgtttcgtcaagcgatgccagtatagctactggagtgtcgatgattatcacttcggttgcgaataaaaaacttgtcacggatgtgacgggtgcgtcaacggcgaacggggcgcgagtccagcttgattcaagtaataatacgaacgcccagaaatatcgatttgaatcaatcggcaacggaacttacaagattatcaatgctaattcaggcaaggtgctggatgtggctggcggatccactgctgatggagcggcattgcagcaatatacgagtaacaatactgttgctcagcagtggacagtgcggaattacggcagcggcaggattgcgctggtgtcggtgaacgccaacaaagctgtcgatattccaggtggcaatgccgtgcagcaggctcagctgcagctttattcgccgaatggcactgttgctcagcagtggttggtcgcaaaggcgccgttgacgttgcgcgaacgtttgaacgaaaccgcagccaagcataggcaggatctgccggatggcacctacacgttcggatcgaagcttaatacgtccatgaagatggatgtaagcggcgcttcccgttcaaattatggaaacgtgcagatctgggcgggtaatggaaccaatgctcagaagtggaaagtgacgcatgattccaacggatacgtgaccttgaccagtgtgaactccggcaaagtgttggatgtgaatggcggcgtatctgccaacggaaccaacgtgcaacagtacgattccaatggaacatacgctcagaagtggatagcagtcaaaaactccgatggctcatacacgttccagtcggcgctcgcggaaaacaaagttcttgatgtttcgggtgcttctacctcgaatggtgccaatgtgcaactgtataccgcaaatggcacgaatgctcaaaagtgggtgaaatag

SEQ ID NO: 31 - DNA, 2337 bp, Bifidobacterium bifidum
  source 1..2337: mol_type = genomic DNA; strain = CCOS 571
atgtcattcgatctcattccggaaccgcaatccgtgacattgaaaggggatgccgccgattcggatgatgccggagctccggtacccgtcgcgttgccgctcgtaggccgcatctcggaagatcgcgacatcgatgacatcgccggcgttttcccgacccagcttgccgatgatatcgaagcggcgacgggcctgcgctgggacatcgccagcgatgtcatggtggccggactgcccgcgccaaatgccgcgattgcgcacccggttccgccacacgcttcatgctggaagtcgttcatcacactgagactcgaccccccatccctggagccgcaggaataccgcctgaccatcgcacgctccggcatagacgtcgtcggcggcgatacggaaggcgtgcgcaacggcgtgcagacccttcgccagatcatccgtcagtgcgcgccggcgctgcctcgactcgtcatcgcggacaaaccggcgtacaaggtccgcggctactatctcgacgccacgcgcggccgcgtccccacgctcgactggctgaaaacatgggccgaccggctgtgcctgtacaagtacaaccagcttcagctatacatcgagcacacgttcatgttcgacgatctgagcgagacttggcgcggcaccagcccgctcaagcccgccgacatcatcgcgttcgacgaatactgcgcgcggctcggcatcgagctggtgccgtcggtctcgactttcggacaccagtacatggcgatgcgcacgcgcgagctgcgccatctcggcgagtttcccgaggacgccgaccgtcggtacggtttcgtggagcggcagcgtcaccacacgctcaacatcaccgaaccggagtccctcgcgttctcgttcaagctgatcgatgcttacatgcagctgttccgcacacgcaagttcaacatctgcggcgatgagacgttcgacttgggtcgcggccgttccaagcccgaagccgaacgccgcggcgtggccgcgatgtacgccgacttcgtatcgcagctgtgccgtcacttgtctgagcgcggccgcgatccgatgttctggggcgacatcgctgtggagatgccgcagatcctcggcctgttgccggataacgtcacgctgctcaactggctgtacgcgccgggcatcggcgaagacaaggtacgcctcgtcgcgcaggccggggccccgcagtacgtgtgctcggcggtgtggtgctggaatgcgctgctgccgcgcctcgacgactcgtggaacaacatctcccgactcgcgcgctacggcgtgaagtacggtgcggtcgggtacctggtgaccgactggggtgattacgggcatgtcaacgacccgcgcatggcggtttccggcatgatattcggcgcgcagtgcgcgtggaatccgatggctcatatccagggtgaggccgggtgcggcgacggcgaagaaggctctgctgccggttatgccgatgccgccgccgatgtcgtccgcgagaacaaggccgccgcggacggggattccccggcgccgctcccgtcgagctcggaaagcgacgattacaccggtggcgcggccgacgcgattgcgggcgcgccggccggcggcgatgggtcgtgtgccgagatgtgccgtcgcgtggccgaggtcgaatacggcgaccgttcgggcggcatagtcgaggcactgcgagatgccgcctgtcgtgtggcgttcggctgggatgacatggtctggtactgcgagctggatgaaggcgacggacggatgaatcgcgatgccgcatcggcgatgcatctcggcgtgcatgggttcagcggggaatacgggcgggagtgggaggcccgactgctgggcagtgccgatcttgatgaggctcggcgcacgatgctgcaagggctgtcgccgcatatcgtccgcgcggccgaggcgaacgaggcgctgctgtgcgacgccatgcgactcggcgccgccgccggtcgcgcatcgcgtttgggtgcggctcggcgtgatgtgccggcgatgctggccgccatcgaggggcagcgctggttcaacttggtcggcttgtgccttgcgcggcggcatgacgtcatcacggtcgatgccggcgatatcgcccgcgcgtcggccggattgatcgaaccggacgcaggctcttccgcagggccggaagccgtgcaggatgtctcgattcgagtggcacgtggtctggagcgctggttcgagacctactgcgacttgtggcgttctgtcagcgcggaatcggaattggcccgcatcgcctccatcgtgtggcgctgtgccgacgccctgcgttcataa

SEQ ID NO: 32 - DNA, 915 bp, Bifidobacterium bifidum
  source 1..915: mol_type = genomic DNA; strain = CCOS 571
atggctgatagattcggagcttttctgcctcacgacacctcgggcgacgttgcccagctgcacggcatcggtctgcaaaaattcggcgacacctggtatgcatatggcgagaacaaggtaaacggcaatttgttccagggcgtgtgctgttatacaaccaccgatttcatcgcatggcgcagccacggcatcgtacttgatgtgcaagaagacggctccgccctcgccgctgaccgtatcggcgagcggccgaaggtgctgcattgccccgccaccggcaaatatgtcatgtatattcatgccgaaacgcccgactacggatacgcgcacatcggcgtcgccgtggccgacgccccaaccggcccgttcgcattccagaccaccatcacgtggcgtggctacctgagccgtgacatcggcgtcttccaggacgaggacggcagcggctacatcatgagcgaggaccgcgaccatggcacgcacatttaccgtctcgccgacgactacctcaccatcgtcgaggacgtcgcctgtgagcgtgccaccgactacccatacggcctggaatcgccgaccatcatcaagaaggacggcctgtactactggttcggctcgcaactgaccagctgggacaccaacgacaacaagtactccaccgccaccgacctgcacggcccgtggagcgagtggaagctgttcgcgccggagggtgcgaagacctacgattcgcaggtcgatatcgtggtgccgctggacgacgacccgtacaacagcgagcacttcctgttcatcggcgaccgctggcaggagcatgacctcggcaactcgccgatcgtacagatgccgatttccatcgccgacggcgtggcgtcgctgacgtggagcgacacctacgagggcaccacccaccgctga

SEQ ID NO: 33 - DNA, 1905 bp, Bifidobacterium bifidum
  source 1..1905: mol_type = genomic DNA; strain = CCOS 571
atgatgcaattcaccatgtccggaaccatgctgcgcttcgatgagacgacgctgcggttctcattcagccgcgacggtgcgacctggagcggctgcgacggcatagagccgcagctgacgcgcgaggatcgttcattctcgttcgccggcgccgctaccgtcacgcatgagcggatcgagaccggcaccggagtcggcgtgcgcagcgtgttcgccgggttcgcgggtgcggattacgcttttgagacgtatatctggatcgagcggtcgagcggcgacgtgctatgcgaatgggtgccactgcgcgagtgtggggccgagccgcgcatcgacagggtgctttggcctgcgccgctgtccttcgaccatgcggacgcgcatgacgtgacgctcatcacccacgagcagggcgtgatgataccgaataactggcctaccgaggtcggcactgatgcggtctccttcggcgggcggttcgagactgcgggcgggtacatgccctggttcgcgcagttgcgcagcgacgggcatgcgtacatcgccatctgcgagacgccatggaacgcaggatatgacatcgaccatccggccggcggaccgtacacgcacgtcggaatgtggttcgagccaagtctcggccgcatggattaccgtcgtgtcgtgcgatatcgtctcctcgaccacgccgaccataccgccgtctgcaagacgtaccgcgcttacgtcaacgaacgcggccggctgcgcacgctggcggagaaggcggcgcgcaacccgtcggtgcgtgacctgctcggccgttcatgggtgcatgtcggcatcaagacgaaggttcagccggactcgtcgttctatgatcccgcgcagcccggcaagaatgattcgctcgtcacgttcgcgcagcgcgaacggcagatgcgcacgctgcacgaaatgggtgccgggcggctgtacctgcatctggacggctgggcgcagcccggctacgacaacggccatcccgactatctgcccgcatgccgcgaggccggcggctggaagggcatgaaatcgctggtcgacgcctgtcatgagcagggagacctgttcggcacgcatgaccaataccgcgactattacttcgccgcgcgcacgttcgacccgcgcaatgcgatccgactggcggacggcacgatgcccgagcacgcgatgtgggccggcgggcgccaaacgtacctgtgcgccgagcttgcgccggattacgtgcgccgcaacttcagtgagatcgccacgcacggcatcgcgctggactgcgcgtatctggacgtgttcacctgcaatgaaggcgacgaatgctcgcatcccgagcaccgcatgacccgccgcggatgctacgagcgccgcgccgaatgcttcgaatacctgctggcgcacggcattctcacctcgtccgaggaggtgtccgactgggcggtgccgagcctggtgttctgccattacgcgccgtacgacttccagatgcgctcccccgacgcgcctcggcacggcatcccggtgccgttgtacaacctcgtctaccatgactgcgtgatccagccgtggatgatggatcgggtggccggtggcgacgactacatgctgtacgcgctgctcaacggcggtgcgccgtacctgatccgtgacgccgcatatgccggcatggacggtgatatgaatgccgcactgcgcgcccgtaccgagaatgacatcgagcggtgtgcggtcgtggccgggctgcaccggcgcgtcggcatgcaggagctcgtgcggcatgaccttgtcggcggcgacccgctggtgcagcgctcggtcttcgccgacggcaccgccgtgacgtgtgatttccacgcgcagacgtatgaggtagcagcgaacggttctcattaa

SEQ ID NO: 34 - DNA, 3417 bp, Bifidobacterium bifidum
  source 1..3417: mol_type = genomic DNA; strain = CCOS 571
atgcgagcgaatggtaattccacgcatgagattctaggcaaaatcgtgacggccatcgcgtcgatcgccatgaccgcggcgttcgcggttccggccatggcggccagcgacagcgacacggcggccgactccggcacgacccaacagtcgggccagtacaagatctacccccagccgcagaacaccgagtatggcgacggcagtctgattctgcgcgacaaggcgaacaccgtcgtggaaccgggaatcgattccgccaccaaggcccgtctgaacgaggccctgaagctcaagggcatcgagaccaccgcggccgacgccgtgcccgaaaccgcgtaccagctcaacgtgctggtcggcatcaacggatccaacggcgtggtcgacaagtacgccaagcagctgatcgccgacggcaccctgaaggtggacgacagcacgttctccaagaacgactcctatgtgcttgccgtccgtcagggcaacgcgaagacgcccgacacgatcctcgtgctcggccgcgacaccgattccgcgttctacggcctgactaccctgtaccagatcttccagcagctgccgggccgcgccgtcagcaacctgaccttcagcgactgggccgacgtcaagtcccgcggcttcatcgagggctattacggcagcccgtggagcaccaaggaccgcgtgaacctgatgacctggggcggctactacaagatgaacacctacgtgtacgcccccaaggacgatccgctgcaccgcaacaactggcgcggcctgtacaccgaagaccagatcgagaacgagatcaagccgcaggccgaggccggcaacaagtcgaaggtgcgtttcgtttacgcgctcgcgccgttccataacgacggcgaggctaggggcaagcacttccgcttcgacaccgaggagcactaccagaaggacctcaaggaactcaaggccaagtacatgcagaccattgacgccggcgtacgccagatcgcactgctcgccgacgactccaccgactggggtgcgcagtacggcaacgacaacacctacgtgcgcgtgctgaaggacctgaccgactggatccacgagctgcagcaggagaagaacgacgacggcaccgcgaagtacgagggcctcaaggacacgatcctctactgcccggccctgtacagctacaccggagccggtgacgcctggtacaaggacatcccatccaacgtgcagatcgtcatgaccggcggccgcaccttcggcgtggccagcaaggacttcgccgacacgttcaccaagaacaccggtcgcgccccgttcatgtggatcaactggccgtgctccgacatgaaccgcaacaccgcctaccagtacctggtcatgggaggccagaacaacttcctcaagcccggcgccacatacggcacctacgacggcatcatgctcaacccgatgcagcagtccgagccgagcaagcagggcatcttcatggccgccgactacagctggaacctgtggcagagcgaaaaggacggacaacagtcctgggaggactccttcagctacatcgaccacaactcgccgatcgcgtccaagggctcccgtggcctgcgtgacctcgccatgaacatgcgcatcctcaacgacggcggcatcgacggcgctcacaaggatgccgagtatgacgccgtcaacaagtggtggatcaacaatgagtccgtcgactacaccggcaagctcgacgtcaagggcgtcctgaccgaactcaagggcaagctggacggcggcaccgccaccgcggccgacttcagccaggcgctcaccgtctacaccacgctgcagcgcgccgcgaagaactaccgcgcgaaccccggcgacaagaacatgtttgaccagatcgaaccgtggatcagctactgggacgacctgaccgcctccgccatcgactacatcaccgccgccaagcaggcgctcgcgggtgacaccgagaccgcgaaggcgacctacgcaaccgcgaaggccgctttcgccaagtccgacacgcatacgatcgccgattactaccagcgcaacaagcccgcccgtggcggcctggtcatcgtgcgcccgaccgtgcaggcgctcgattccttcgtcaaggccaagaccagcggcagcgtcaccccgacgccctccgacgccacggtcagcacgaacggcgtgggtgccgcggcatggcatgagaacgtcgatccgaaggccgtcatcgacggtgacgacagcacgttcttctggatgcagtccgccggctgcgactgtgtcaaggccaacgccgccctcaccgtcacctacgccgaggcccgcaaggccaaggagttccgcttcatccaggcggaaaagggcggtgacacgatcgtcaacggcaagatcgaataccaggatgcagatggcaactggaccaagatcggcgacgtgaacggcaaccagaagcagatcttcacgctggactccgccgcgaccgtcaaggccgtgcgcatcaccaacttggcccagacctccaagtggtggaaggtgtacgacctgtccgccaccaagatcgacgaacccggtacggtgaccaaggacgcgctgaacgccaaggtcgccgaagccgagaaggtcgactccgccgactggaccaagtcgagccgcgaggctctcgccgacgcgattgccgccgccaaggccgtcgctgccgaccaggacgccacgcaggccaaggtcgacgccgccgtcgccgcgctcgaatcggcgatgaagggtgtcgaacggtacacggcgaagaccgccgaccagctgaaggccgagcacgtctccaacgacgatgccacctacaccgaggcgagctacaacaagtaccagtccgcgtacgacgacttcgccgccgcgctggccaacgccgacgatctggcgaaggccgacggcgaggcgctggaagcggcctacaccgccgcgaagtccgcgttgcgctacgaccagtccgcacgcgactacgcgcagctcgcgctgaacgacgccgagccttacgtgggcaaggcctccgagtacaccaaggacagctatgccaagttcaccgtcgcgtatgaggccctgagcaagcagctgaaggccgacccgaacggcgagggcgatcccgccacctacaccgcgctgcgcgctgcactcgacaaggccatcaagggcctggtcaagagcgacggcaccgagctggagcggcctggcgagaagcccggcgagaagcccggcgagaacaagcccggcgagaagccgggtgtcaacaagcccggcgccgacggcaagctgtccaacacgggcgccgacgtcttcggtctcatcgccgcgatgacgatgttggccgcggtgggcgtgacgatggccggcctgcgcaagcggatcggctga

SEQ ID NO: 35 - DNA, 5268 bp, Bifidobacterium bifidum
  source 1..5268: mol_type = genomic DNA; strain = CCOS 571
atgaaccgaagaatatctatgtggttgcgcggtcgtggctctccatggagacctgccgccgccgtgatcgccacggtatccatgatgctggcgacggtcatgggcccccatttcgcggggatgcgggcgcaggccgtcgagcccgtgcagaccaccaccatctcgcacaccaagatcgccggcgacggcaactacttcacgttcgccgacaacgcctgggatccgggcaacgacgtgcatacgtggtccaaggcgccatccgacagcctcccggcggaggacatctggtatacggtccgattcttcggcagcgcaatcgacgtctatgcaggcaagaacaggcccatgggcaaggtgaaatactatattgacggagcggaaaaaggcacgtacagcctgtacaacgcctccaacatcaacgagacgaaaatcgcgtcgttcaccggacttgacgaaggtgagcacgtgttcaaggcggtggccacaggcgagcgggacacgaattccacgaacgcgctgatcgactgcgccaaggtcgtcgtgacccatcagccgtacgtggtcaccggcgtcacactcgacacgacctcgatgacgctgggcgttggcgacagcaagcggatctcgtatacggtggcgcccgattacgcgaccatcgatgacatgacctacacgagcggcgacacgagtgtcgccacagtcggcgcagatggcacggtcaccgcggtcgcgccgggagccaccgccatcaccgtggcctcgactgcggccggcatcagcaagatcgtcgacgtcaccgtcgccaggatggcaccgaacctgaccggcggcatcgtggaccccgatacgcaatacacgcagaagcggttcgacgaggtgaaggccctcacgacgaacaaccgggcgctgaaagcatggaagaacgacaaggtcaacagcgagatctcgctcgccgcggtcggcaccaccgtctcgaacctgacggtcaccgcaaacgacctgacgtcgcagggcggcgacgtcatcgccaaaagcaacgtcacggccacgttcatcaagtccaccaaggcatacaacggcagctatctcggctacggagacccgaaccgcgaggtgcccgccgccaccgagaccaaccgctccgaaagcaacgacatcctctaccaaagcggcccgatcacggtgaaggccaatcaggtgcagaacatctgggtatcgttcgccataccgaaggacgccaaggccggcacctacaccaccacgctgaccgccaccgcagacgggatggaaacgccgctcacattcacctacaccatcgaggtgaaggccgctacgctgcccgatccggcagagtatgagaagaacttcgacgtggaactgtggcagtacccgtattcctccgcggaatactacggcgtcaccccgttctcggacgagcacctgcagatcctcagatcaagcatggaactgtacaagtccatcggcggccacgccatcacaaccaccatcaacgaggacgcatggtccggccagacctacagcgccaacgccatccactacccgtcgatggtgaagtggaccaagagcgggggcgggttcacctacgacttcaccgacttcgacaagtgggtgacgttcaacaagggactcggcatcggcgacaagatcgtgatatacagcatcgcgccctggcacggcaacttcacgtactgggagaacgggacgatgaagtccgagaaatacacggtcggatccgagcgctggaggagcgtgtggaccgacttcctgcgcaagctcatcgagcatctgatggacaagggctggttcgacgagtcgtacatcggcatcgacgagcgcggcttcagcgccgacgcgttcgacctgatcgactcgatacggaatatccacgacgtgccgttgaagaccgccggcgcgatggacggcttcgtcaacaagttcgacctggcgctgcgcgtcaccgacctgaacgtcggcgacaccgcggcggcggcccatcccaccgacttcacccggctgatcgaggcgcgtgaggcaaagggccttcgcaccacgctgtactcctgcaccgagcatgagcccggcaacttctcgctgtccgccccggtcgaaagctactggtcggtggtcaacgcgggcgagcagaccagcggcttcctgcgctgggcgtacgacgcatgggtggccgacccgctcaacgacgccacgcacaacgccttcgagcccggcgacccgttcctgatttatccgagcgagaagagcggagacaaggtgtccaagtcgtccgtgcgcctggagcgcatcgccgagggcgtgcgcgacgtgaacaagatcaggctcatggtcaccgagatcccctcgctgcaggccgatgccgacgcgatgtacgcgaagatcagaacgacggtgacgacctcgcactcgtatctgaccgccgcgcaggtcacgcagcttgccaatgagatgagcggattcaagggtgacctcgacacgctcaccgacaagtacatatccctcaaggcgcaaggcacaagcacggtggaatccgttgccatcgacggcggcgaccaggagatcatgctgggcacggcgaagcagctgaccgcgacactcaagccggcgaacctgctgaacgcgtccgtgacatggcgctcgtccaagaccggcgtcgccacggtatccgccaagggcgtggtgaccgccgccggcgtgggctccaccacgatcaccgccacgtccaaggccgacccgacgaaatcggcgagcatcaccctcaccgtgacgccacaggtcgtggcgcaggggctgcactactactcgttcgacgactcgaacgccaacgactcctggggcacccgcaacggcaccgccgacgccacggcgcaatacgttgacggcaagtccggcaaggcgctcaaggtcacggacggcaaaggcgtgaccctggccggcggcaacgacatcgcgaagacggatccgtggacgatcggctactgggtgcgttccgacgccgaactcaccggccgctcggccgtgatgacgagcgcggacggcaagtactcggccgacctcaagatggacgccgaccgcgaatctggcttccgtgtcggcaccgccagcggcgacgtgctcaccttccggtacgacttccagccgggcacgtggtattacatcgcgtggactcaggacaaggcagccggactgaccatgtacgtcaacggcacgaagatcggcgtgacgaacacgtggacgaagatgcacgatgtcgtcgccccgatcgacgtgatcggcggccccggcttcactggcctgatcgacgaggtgaagatctacaagcgcgtgctctccgacaccgagatcgcggccggcatgctgctgcccggtctcaacctcgccgagcatgagaccgatatgtacatcggcgggacgtacaccatcgtcgccaacctgcagggcggcgacggcgatggtacagtcacgttcgagtcgagcgacccgaccatcgccaaggtcgacgcctccggcaccgttaccggagtgtcccgcggaacggcggtcattaccgtccggggaggtggattcaccgacatggtgaccgtcaacgtgagccgcgaactcaccatcaagaacacgctgccgcagtacaagctcgaccagacgaaggtcacggacgtgcacaagtcactcgacacgtccaaccagtacttcggccagcccgacatgatccgcacgaagagcggccgtctgatcacctcgttcccgcaagggcacggcaagggaccgttgatcatgaagatcagcgacgacgacggcgccacctggacgcgcaagaccgacatccccgcgtcgtgggccggttcgcaggagacgccgaccctgtacgtgctcaacctggccgacggcaccgaacgcatcatgatgatcaccgcctgcccgggctggggtaccgactccgccggcaacaggtacggatggaacacatcgtattccgacgacaacggtgagacctggaccgaataccggcactggcagtccaaccgcacgtacgacaacgccaacaacgacgccatcgtcgccatggcgagtctcgtgcagctcaaggactccgacggcaacgacatccagaaatggatgggcgtctaccacaactacgcgtacgtcaatttcaggacgtacctcaccttcgacgagaacggcgacgagcagtggagcgaatcggagccgtatcttgcgcaatggcgcagcatcgaaagcgcataccagatgtgcgagatcggcatgttccgttctcccgacggcaagcggatcatcggtctggcgcgcagccagtcgcacaacaacccggcgacactgatctactccgatgatgagggtgagacgtggagcaagccgatggatctgcccggctcgctggcgggcgaacggcacaagattgcctacgatccgatttccggccgtctgctggtcacgttccgcgagatcaactacgatctcaacggcaacaaccggttcgacggcggcaacgactggaacgccggcgactgggtggcctgggtcggtacctacgaccagctcattaaccaggaggacggcgaataccgcatcctgctggccgaggactggacgagcaacgcgaagtcgggcgacaccggctacgcgggcgtagcggtgctcgacgacggtacgttcatcatggacacgtacggtcactgggacaaggaattctcgcagaactggcccggcggcgtcaccaccgaccgctgctatatcaagcaggcgaagttcaagctcggcgaggtcgaatacgcgaacggtctcatcgaccgcagcggactgaaggcggcaatcaagcgggcagaggcgctgaatgcggccgactacacggctgactcgtgggcgaagatggatgccgcagtgaaggccgcgaaggccggagacgccgacgattccctgcaacaggcgcaggttgacgcgctcgccgccgccatcgatgcggccatcgacgggttgaaggccaaggatgacggtgacaagcccggaccgggtgatggtggcaagcccggcgcgggtgacggcaagcctgacgacggcggcaagcccgatcccggcaagccgggcaagggcgacgagaccaagcccgataccggcaagaagggcgggctgagcgccaccggtgccggcatcgttccgatcgccgccgcggccctgacgctcatggccggtgcggcgctcgcactggccaagcgctga

SEQ ID NO: 36 - DNA, 4884 bp, Bifidobacterium bifidum
  source 1..4884: mol_type = genomic DNA; strain = CCOS 571
atgcgattgaggagagtgaaagcggccataggcagtgtcctggcagccgtgacactactgagtatgtcgctgaccggcgtcacggcggcgcaagccagcgatgacaatcttgcactgaatcagaccgtaacggccagttcatatgaagtggcgacgactgcgcccgagaaggccgtggacggcgatctcggcacacgctggggcaccgcgcagaacaaggccgcaaacgaatggatcgaggtcggcttgggcggcaccaagaccgtcaagcagatcaacatcgatttcgagcgcaaagatgccgatcagaacatcaccagttttaaagtcgagctgaagcagggcgacacctataccaaggtgtatcagaaggacacccgcgccaagcaacaggagatcatcctgcttgaccaggcacagcaggcgtccgccgtgaaggtgaccgtactgtccgccgacggcggcaccatgaactgggtcaacgtgggcatcaacgagatttccgtgtactccgcacccaaggagaccgtgctcgacacggccgacacgaaccacatgctcggcgccaccatgaccgcctccagcaacgagacggcgacgctcacccccgacaaggcgatcgaccagaaccgcaccggccgcaacaaccgttgggccagcggctacgagaccccgagcaacatctggctgaaggcggaattcccgcggctcaccgcggtcaaggacatccgcatctatttcttcgagcgtgacgtgaatccgaagcctaccaatgtccagagcttcgacctgtcctacaccgactccgaaggcaccgagcacacgctcaagtccggctacgcgatgacggcctccgggaccggttatgtcgctgacgtcgtcatccagctcgatcaggccgtcaacgcccgctcgctcaagctgtcgaacttcgcgatcaaaagctccgaatacaacaacgtgagcgtcgccgaatgggaggcgtattcgaacgatcaggccgagccgggcgccacgctcgacagcgtggtctccgacctcgaatccaaccacctgaccatcgagaccgacaccgacacgctggctctgcccactgtgcccgacggctacaccgtcaagttcaacggcgccgactacgagcagctcatcgccgccgacggcaccgtcaaccacccgctggtcgacaagaccgtgcaggtcgcctacgtcgtcaccgacaccgccaccggcaacaccaagacgacctccgacatcccctacgtcgtcaaaggcaccaaccagcagcaggaaggcaacaacgccaagccgaccatcatccccgaaatcgccgaatggcattcgaccagtgccgccaagctggcggcctccgccgtgacgaaggtcgtctacgacgatgattcgctcaaggccgtggtcgacgagttcgtcgccgactacaaggacttcaccggcatcaagctgaccgccaagaagggcgccgccgaagccggtgcgttcaacttcgtcaagaccgactccacggccgcgatcgcgcagctcggcgacgaaggctacacgatggacatccgggccgaccgcgtggtcgccaagtcgtccagcgtgaccggcaacatgtacgcgatgcagacgatcctgcagatgaccaagcaggacgccagcggcttcgtgatcggttcgatgcgcgactacccgcgcttcaccacccgcggactcctgctcgacgtggcccgcaagcccgtctcgctggagatgatgcgcgagatcacccgcacgatgcgctactacaagatgaacgacttccaggcgcatctgtccgacaactacatcttcctggagaactacggcaagggcgacaacgaggacgaggcgttcaaggcctatgacgcgttccgtctcgaatccagcctgaccaacgacaagggcgaatcgcccacggccgaggactactccatctccaagaagacgttcaagcagttcatccaggacgagcgcgccctcggcatgaacgtcgtgccggagatcgacgtgcccgcgcacgccaactccttcacgaagatctggcccgagctcatggtgaagggacgggtctccccgatcaacagcaaccgaccgctcatcgaccacctcgacgtgtccaagcccgagaccatcgccaagatcaaggagatcttcgacgactacaccaagggcgacgacccgacgttcgacagcgacacgaccgtgcacatcggtgccgacgagttcctctacaactacacggcataccgtaagttcatcaacgagatcgtcccctacatcaaggacacgaacaccgtgcgcatgtggggcggcctgacttggatcaacgaccacaagacggagatcaccaaggacgcgatcgagaacgtcgagatgaacctgtggtccaaggactgggccgacggcctccagatgtacaacatgggctacaagctgatcaacacgatcgacgactacggctacatggtgcccaacggcagctacggacgtgccaacgcgtacggcgacctgctgaacatcagccgcgtcttcgacagcttcgagcccaacaagatccgcagcagcggcggctaccaggccgtgccctccggcgacgaccagatgctcggcgccgcgttcgcgatctggagcgacaacatcgacaagagcgcatcgggtctgaccgaatccgacctgtactggcgcttcttcgacgcgatgccgttctacgccgagaagacctgggccgccaccggcaaggagaagggcaccgccgcgaaactcacggcgctggccgccaagcagggaaccggccctcgcaccaacccgtactaccaggccacttcgaagaacagcgtctacgagagctacgacttcaacgacgggctcgccgacgccagcggcaacggccgcgacctgaccatcggcgacggcagcaaggccgccgtcaaggaccagtcgctcaagctcgccggcggctccagctatgccacatccaagctcgacaagctcggcaacggcaacgagctgacgttcgacgtgacgctccagcaggctgccaagcccggagacatcctcttcgaggccgacgctccgtacggcacgcatgacatccgcgtcatggagaacggcaagctcggcttcacgcgagagctgtacaactactacttcgattacgagctgccggtcggcaagacggtcaccgtgaccatcaaggtggaccagcagaccacgaagctgtatgttgacggcgaattcgtgagcgacgcgaccggcaagtacatcgacaagggcatcgagaagaagaccggcatcaccgccgcgaccttcgcactgccgctgcagcgtatcggctcgaagacctctgcgatcaacggcgtcatcgacaacgtcatcgtcaagaagtccgaggccgagacagaccagtacaacaagtcctgctggaccggcacgaccaattccgagacccaatacaacgacacggaaggtctgctgcggtacgcgttcgacaacaacccgagcaccatctggcattccaactggaagggcgccacggacaagctgaccggttcgaactcgttctacgccgagatcgacatgtgccagaagtacacgatcaaccagttctccttcacgccgcgtaccagccaggacagcggacaggtgaccaaggccgatctgtacgtcaaggccaacgcgaacgacgagtggaagcaggtcgccacggatcaggtgttcgaggccagccgcgccaagaagaccttcatgttcgacgagcaggaagtgcggtacgtcaagttcgtggccaagtcctcgaacgatggctgggtcgcggtctccgagttcggcgtggcgaacaagccgtcctccaccgtgcgcgtattcgtcgcggccgatccggccgaaggcggtacggtgagcgtagccgccgagggcgaaaccggcaccgatacggccgtggacgtcgcatccggtgcctcggtgaccgccaaggcggtcacggcggatggctacaggttcagcggctggttcacgacggccagtgagacggcggtttccaccgatgccacgtacacgttcgcagccgacggcaacacggctctgaccgcgaagttcaccaaggactccacgccggatcccggcccgaagccgaccatcagctcgatcgccgtcaccaagccgaccgtcaccgattacaaggtcggcgacacgttcgacgccactggactggcggtcaccgccacaatgtcggacggcagcacgaagaccctgacggccggcgagtacacgctctcagccacgcaggacggtgccgcggtcgcgcttgacaaggcgttcgccaaggccggaaaggtcacggtcacggtcaccgccaacggcaagaccgccaccttcgacgtgaccgtgacggccaaggatcccgatccggaacccgcgacgctcaagtccatcaaggtcacgtccaagccggacaaggccacgtacaccgtggatgagacgttcgccaagaccggtctcgcggtgacgggcacctggtccgacggcaagacggcgctgctgaaggacggcgaatacaagctgtccgcggtcgacgccgacggcaagaccgtcgacttgaccaagccgttcacggccgcgggcgacgtcaccgtcacagtgacctccggcaagctcaccgactccttcacgatcaccgtcaaggcaaagaccgtgaccccgactcctggcgacaacaagccgggcgagaacaagcccggtgccgacaagccgaagccgaacacgcctgacgaggtcgccaagaccggtgcttcggtcacggcggtcgtcttcagcgccctgttgctgttgtccgcaggctacctgctggtgcgtaagcgcaggatctga

SEQ ID NO: 37 - DNA, 5883 bp, Bifidobacterium bifidum
  source 1..5883: mol_type = genomic DNA; strain = CCOS 571
atgcgcagcaaggcattaggaggcctgcttgccgcggctctgtcgttgagcccggcagtggccatcggcgtgcagacggcgtacgcggcaggcggcgatacggcggcgaccgaatacacgctgtaccccaagccgcattcgatccgttatgacagcggacagtacattcttcgcgacatcaacgtcatctatgacgatgacatcgatgaagccacccaggaccgactggacgaagtggcggcgctcaagaacctgaacgtcaccgaatccgacgccgcagtcagcggcaagaccaacgtctatgtgggcgtgaacggttccgatggcaaggccgagactgccatcgaaagcaagtacagcccggattccgccatcttcgacaagaccgactcatacttcctcaagtccgacaacggcacgatctccgtgctcggcaaagacaccgacgccagcttctacggcctgaccaccctgtaccaggtgctcggccagatcgatagcctgacgatccgcaacttcaccgtcaccgactacgccgacgtggtcagccgcggtttcatcgaaggctactacggcaacccgtggagcactcaggaccgcatcaacctgatgaagtggggcggctactacaagctcaactcgtacttctacgcccccaaggacgatccgaagcacaactcgcagtggcgtacgctgtacacgcaggacgagctcgacaccaagatcaagccgctcgccgatgccggcaacgcctccaagacgcgtttcgtgttcgctctgcacccgttcatgaacaacgcgattcggttcaactccgaggcgaactatcaggccgatctgaaggtgctgcaggacaagttcgcgcagaccatcggggtcggcgtacgccagatcgccatcctggccgatgacgcggcgaacgtgggtggtaacaattacaccaagctgctcaccgatatggtcgcgtggctcaaggaaatgaagaagacctacccggacctcaagaccacgctgccgttcgtcacgcaggaatacatgggcaacggcatgagctacttcgccaacttccccaaggaagtgcagatcgtcatgaccggcggacgcgtctggggcgaggtctcgcagaacttcaccgacacgttcacctcgaacgtcggccgcggcccctacatgtggatcaactggccgtgctccgacaactcgaagagccacctgatcatgggcggctacgacacgttcctgcatccgggcgtcgacccgtcgaagatccagggcatcgtgctcaacccgatgcagcagtccgagccgagcaaggtcggcatcttcggcaacgccacctactcgtggaacatctggcggagcaagagcgaggccgaccaggcatggcaggattcgttctcgttcgtcgaccacaactccgctgtgccgaccgccgcgtcgaacgcgctgcgtgagctgtccaagcacaccatcaaccagaacatggattcgcgcgtcaccgcgctgcaggagtccgtggacctggctcccgcactgaccgccgccaaggcgaagctggctgacggcaccatcaccgcccacgacctgaccgacatcaaggccgcgttcgtcacgctgcagaaggcagccaagacctaccgcgccaagggcgatgcgaagatgctcggcgacatcggcaaggactacaccgggcaggacgccaacgagcagatcgccccgtggatcgactgctgggatgacaccaccaaggccgcgctcgcctacatcgcgggtatcgaggccgcgctgaacggcgacacgtcctcgacgctcaaggagtactccgacgcgcagtccgcgttcgccgcgtccaagaagcacggcttctactacgtgaaccacaccgagtacgccgaagtcggcgtgcagcacatcgtgccgttcatcaaggcgatggactcctacctgtccaacaaggtgcagcaggaggccgacccgaacgtcgtgaccaagacgtacatcagcgacgtgttcaccacgccgacctccggctcgatcgaagacatcttcgacggcaaagacagcacggtcacggtgttccagaacccgaactacctgcacaagggcaactacgtcggcgtgaagttcagcaaggccaccacgctcaagagcatccgcttcgcgttcaacggcggcaagaaccacttctaccactccaagctgcagaccaccacggacggcgagaactggaccgacgtgccggatgcgacgttcgagcgcccgaagggctccgaggagccgatcaaagtcaccggtctgaacatcaccggcgtcaccggcgtgcgactgatcgccaccgccgacaacggcgatgacctgtggctcggcatcaagggcatcgacgtcaacaaggtcgagaaggagactctcgctccgtacacggcgactggcgtgcagctggagaacctcaacgccacctacagcagcaccaaggagcagatgatcgacggcaacccgtccacgatcacctacctcaaggacccgaacggcgacaagatcgcggccggcgcggccgtgatcgtcgatctgggctccagcaagccgatcggcgaggtcacgattaccggtcatgcctccagccccgacgaccgtccgagccagggcgtcgtcgaggtcagcgatgacaaggccacctggaccaagcttggtgacctgagagacgacgtcacctccacggtgagcggcaacgtgaccgggcgctacgtacgcatccgcaacaccgccgccaagaacgtgtggtggcgcgtggccgagatcaccgtgaccccgcccgagaccgccgatccgctcaagtcggtgtacacgaacaagacgcagcacgggtacaccgccacgctgggcgccaatacggcggagctgttcgacaaatcgcacacgatgcttgacggcggccagtacgccggactcgatctgctggcgatccgtgatctgaccgacgtcgagctgaacaccggtaacgcaaaccccagcctgcagatctccgacaacggactcgtctggaccactgtcgagccgggcaacctcacgggcaagaccgcccgctacgtgcgcgtgatcaatggcaccgacggagcggccttctcggtcaacaagctcaaggtcggcttttccacggtcggcaagttcggcaagctggtctcatccgacatccagaagcgcaacgattggggaaccgacacccgcgaaagcggcaacgcgttcgacggcgatatgaccacggtcatcaagttcgccggccagccgcgacagggcaacaccgcggtgttcgatctcggtcagccgatcgacatcacctcgctgcgcatctacacgcaggacacgcagtacgactacatccgcgacaccaaggtgcagatgtccgtcgatggcaagacctgggttgacgcgttcgagattggcgacggcgtgtccgacaccgacaccacgaccgcgttcggcgacataagcgacacgaacaagaagaccgattccaactacccgaacgtcttctactacggcaaggacgacatcgccaacggcaccggcatgcgctacctgcgtctgctgaccaccgccgactacccgcagcgtgcactcgcgttcaacgagttcatggtgaatcagggcgcctacgtctccaccgaggcgaacgcggcgttctccgccaccaaggtcgaggagcgcggccacgcgccgagcaacatgatcgacggcgacctgaccaccacgtacaagccgagtgcggccaatggctccctcacctacaagatcgatgacccgagcgacatcaagtcgttccgcatcgtgcagtcgggtgccgccagcggtgcgaccgtcaccggtacggtgtacgacgcggcgaccggaaccactgccggtgtgacggctcgtgcagccgcccgctccgcctccgaggtgacgttcggaacgctggaacaggcgatcaacgagttcaaggtgccggacggcaagcagctgctcagcgtcaagatcgcgtggggtaacgccatccccgagatctccgagttcatcacgctcgactcggccgacaccgacgccgacatcaccacggccaaggcggcgctcaaggagaagatcgacgcgaccgtcgacacttccgcctggaccgcgaacgccaagaccgcgtatgacgaggccaaggccaccgcccaggctgtgtacggcagcccgctggtcggcaaggcttcgatcgacgccgcagccagcgccgtgcaaagcgccatcgacgccgcgcagaccaaggccgacgccgccgcggtcgccgcgctgcgcaagctcgtggacgcagccgtcaccaacgacggccacttctacacgacagtgacgttcaccgcgtacaccgacgcgctcgacgaggtcaaggaagcgctgaaggacacgaacaacctgtccacagcagacgccgcaagcctcaagaaggccgtcgatgacgctctcgccgcgctgaaggactccacctaccagcgtgagctcgcgacgatcgccgtcgacagcttcgccacgatcgacgaggccgactacaccacgaactcctacgcggctttcaaggccgctaaggaggcgctcgacaccctgatcgccgccgacggcaccaagccggccacgttcaagaccaagaccgccgagtacaccaccgccaagaccgcgctggtgaacgtcgccgccctcaaggcccagatcgcgcacgagagcaactacgtggaggcgaactacaccgccgactcgtggaaggcgtactccgacgcgctcaaggccgcaaaggcggagctggtcaacggcaccaccgattcggtggccgccgcgctgaccgcgctgaccactgccgagaacgcgctggtgcgcaccacgacgccgcccgacaccaccgtgctcgacgagaccatcgccgacatggaaaaggttaacggcagcgagtacaccaccgacagctacaaggccctgaccgacgccatcgccaaggcgaagagcgacaaggccaagggggatgccggcctcaaccagcagaacatcgacgccatgaaggcggcgaaggaggccctggtctccaccgtcgaactcaaggcgaaggtcgccgaagccggcaaggttgactcgtccaagtacaccacggccagctacgaggcgctctccaagctgctcgccgcgaaggacgacaccacctccgacccggtcgtcaagggtctcgacacgctgtacaagtccggcaccgtcaaggagctcgccgagcgcgtcgccgccatcgacgccgcggtcgccaagctggatgcccgcgccaccggtgtcaaggactacgtggacggcatcaagctcaaggacaacgacaagggctactacaccgacgccagctacaaggcctactccgacgcctacaaggcgctgaagaagctggccgccgccggtgaaggcgaagtcgggattgccgagttcaccgaagccaaggaggcgttcgaagccgccgaggccaagcttgcctacaagtccgcggactacggcaagatcgatgacctgctcgccaaggtcccctcggacctgagcggctacaccgccgactcggtggcgaagttcgaggcggccaagaaggccgtgaagcgcggactgaccatcgaccagcagagcaaggtcgacgcgatggccgacgcgctcgaagccgccatcaagggcctgactctcaagtcgcaggcacccggtggcaacaagggcgacggaacccagagcggcactcataagggtgacgacatctccaagaccggcgccgacgtgcaggtgttcgcgatcatcatcgcgctcgccacgtgcgctggcctgggcgccgtggcctacgcccgtcgccgccgcgaggcgtga

SEQ ID NO: 38 - DNA, 5772 bp, Bifidobacterium bifidum
  source 1..5772: mol_type = genomic DNA; strain = CCOS 571
atggcgacactcatgtgcggcctgatcacatccggacgtctcgcgcgcgccgtggaaccggcgatcgccggcgtcacggcgagcggtgccgccgcggacggccatgctgcggccgccgcagtcgacggcgatccggcgacctactggcagtcacccgcagactcgtcgatgcaggactaccgccgcttcctcgacttcgacctgcacggcacatacagcatctcgcagatcgacatcaccaacctcgccggctcgtactaccactacgaggtctatctgtccaaggacggcgccgattacggcaaggtggcctacaagagcgatgacgcccccgcgaccggtgccgccgacacgcatcccatcgatgccaccgaagccgcatacgcgcgcatcagcgtcagctacaactccgccgcccagcaggtcaacctcgccgaagtcgctttccacggcaccaaggtctccgacgagcaggccagccccaaggccatctccgtcaccgacttcgactcaagctcgtggggccgggagtgggcccgcgtagagaccgacgccgactatgccgccgagaagaccgtcaccgaggtcagaaacctcgtcggccgcgtgatcggcgagcggtgggtcgacaagttcgacttccagctgcgcggcaaggccgacggcaaggacgtgttcgaaatctcagacgccggcgacgaccggatctccatccgcggcaacaacggcgtctcgctcgcatccggcctcaattattatctgcgccactggtgcaaggtcgactacaacccgctgttcggctcccagctgtccatgccggagagcctgcctgcagtcggccggaaaatcctcaaatacacgaactacgagtaccggtacgcgctcaacttctgcacctactcgtacacgatggcgttctggaactgggatgattacgagccgttcctcgactgggcggcgatgaacggcgtgaacctcatgctcgacatcgtcggacaggaggaggtgctgcgcgagacgctcacccaatacggctacagcgacgacgaggtgcgcgaatacctgtccgggccgggatactacgcatggttctacatgcagaacctgtactcggtcggaggccccctgcccgccgcatggttcgagcagcgcgtcgagctcggccgcaggatccacgaccgcatgcaggcgtacggcatcacgccggtcatccagggcttcggcggtcaggtgccggtcgacttccaggagaagaacccgacgtcggtggccgcgtcgtccggcacatggtccggtttcgaccgcccgtacatgatcaagacgtatctgaccgacgccgacaaggcggccggcaaggaggactacttccagaaggtcggcgacacgttctacaaagctcaggagaacgtgttcggcaaggtgtcgaactactacgcggtcgatccgttccatgagggcggcacgattccagacggcttcgacatcgtcgacatctaccgcaccgtgcagcgcaagatgctcgaccatgacccggcggccgtgtgggtgatgcagcagtggcagtggggcatcgacgagacgaagctctccgggctcgcggacaagggtcaggcgcttgtgctcgacctgcagagcgacctgcgctcgcaggccagcccgatggagaaccagggcgtgccgtgggtgtggaacatgttgcacaacttcggcggacgcatgggccttgacggagtgccggaggtgatctcccaggacatcacgaaagcctacaactccagcggttacatgcgcggcatcggcatcacgcccgaagccatcgacaattcgccgatcgtctacgagctgctgttcgacatgacctgggagcaggatccggtcgactatcgctcctggacgcaggagtatgccgagcgccggtacggcggaaccgacgggacgatcgagaaggcgtgggacatcctgcttgacaccgcctacaagcacacggacggcgaatactatcagggtgcgggcgaatccatcatcaacgcccgcccgtccgacaacaccatcggctcggcgtccacgtggggccacagcgacatagattacgacaagcggcagttcgagaaggccgcggccctgttcgagcaggcgtatgactcgtacaaggactcggccggcttccgctacgattacgtcgacgtgatgcggcaggtgctggccaacagcttccaggaataccagccgctcgccgggcaggcgtacaagtcgggcgatctggagacgttccggacactgtcgtcccggatgctggacatcatcaaggcgcaggacaagctgctttccagctcggacgacttcctcgtgggcgcatggatcgacgacgcccgcacgatgctcgacggcgcggacgattggaccgccgacctgttcgagctcaacgcgcgtgcgctcgtcaccacgtggggtctgaacaagaacggctcgctcatcgactattccaaccgccagtgggccgggctgaccggcgactactactatcggcggtggaagacgtacgtggacaaccggctgaacaaactggagcacggcaccgacttcaccgacccggactggttcgactacggctggcagtgggcgaaccgcaaaagcgacgaggacgggtatggtttcgcgaccgaagccgcggatgacgtcgaccagaaagcgctcggcaagatcatcctcgaccagtattcggtcaccgcgatggacgacgtcaccgacggtggcaccgcggtcgaacgcacgaacctggcgctgggacatgacgtgaccgacgaggacaccggcaccgtcgtccctgacgtgaccgacggcaacaccgacaccggctggacgcagacgggcaagaccgacgccacgctggtggtcgacctcgacggcacgtattcgatcaccggcgccggcatcaccctgcagcagatcgcggccgacttcccgctgcgctacgagatcgatgtctggaacggctcgggatgggtcgagataggccgcagcgaagccgatgccgtcagctccaagaacgaggtcgccgccgacatactcggctccaaggtgcgttggaagttgcattcgacgaacgggcgggacctgaccggcatctacgagctgtccgtctggggagccgcgcagccgcagcccgagtacacgaatctggcgcttggcggcgccgcaagcgccggccccagcgagcggcccgcgtcgaacggcaacgacggcgacgacggtacgctgtgggtcggcaacgggtcagacccgaactggtatcggatcgacctggcgtcggcgcagcgtgtggaccgcgtgcggctcgtgttcgagacggccggccgcctgttccagttcagggtcgtcgcggggctcgctgacggtaccgagcggacactgatcgacgagacgcagaaccagggcgccctcgatcaggtgtatgcggcgaatcttggcgaggaggtcgaacacgtcaccgtggagttcacggggtcggtcggcggcaccgcatggccggcgctcgccgaactcgaactgctgcaggaggccggcgacaccatcgaaggcgtgaacatcgccacctcggccgccatcacgtcgtcgcccaccaaggacgcgcccgagaacgccggcgcgctggtggacggcaaggcgaccgcatgggtgtcccgcgacggcgccacgccggcatggttccagctcgactacgccaaggcccgtgaggtcgacagcatacggctcaagttcgaggaaggccagcccgaccgcagcatgcagttcacgttgaaggtgatcgacgcgaacggcgacgaacatacggtcgccgaacgaaccgaagccgacctgagcaaacagcagggcatcgtgatggacgtgccggtcggcatgagcatcacacgcatccgcatggacatcgccgacgcccgcatcccctccagcggctcgcccgcatggccgctggtgtccgagatcgaggtctacgcgacgcccggcaacgtggctcgcgacgccaagacgacggcctcggacggctcgacgctcacggccgccgacctcgccaagctgacggacggagaccgcgacagcgcggccaccttgacggccacggccgacaagaccctgacattcacgctcgcgaaggccgcggacatcaacatgctcggcctgctggcctccggtaacagcgagccggtgcggttcaaggccgaataccgcgtggtgccgcaggacggcggcggtgcgggcgatggtgcagccgaccagtggaagacgctgaccgactattccggcaatgcgcagatgaagccggagatcgtggcccgccttgcacggcccgtctacaccgacgcggtacgcatcaccgtgctcaacgagaaccccgtcgccatcaacgagttgtacctgtatcaggcggatgcaggggcgtctctggaaagctacctctcgtccgtcgagaccgtgctcggcaagctcaccgtcggcgaatacgccggcaacgtcacccgggcggcgaagaccaagctcgaagtcgtgctggagcgggcgcgggcggcgctggacgccggactgacctcgcgcgaggccggccggtggacgacaaccgtcgaagacgcggtgtccgagttctaccgcaccggctacgtgtcgctcgaccgcaacgcgctgtatgtggcgatcgatgacgccgcggcgctgatcgcgtctctggacgcgcatggcctgcccgcgtcgtcggccgcattggccgaggcgagggcaagcgccaagcaggtctccgacgcctacggcacggtgacgcaacaggatctcgatgacgccgcggctgcgttgcggcggtcggccgacacggcgctcgcgcagcttgacgcgcaggagcgctatcaggtcgtgctggatgcggcggtcaagacgctggaggacgcgcagacggcgggcagcgtcggcgaatacgaggggcagcatccgcaaagcgccgcggacgctctgcgccaggccatcgacgacgccaaggccgcacacggtgcagccgccggagacgccgataaggtggacgccgcggcggacgcgctgcgccaggcgacgaccgcgttcaacgagtcaatcgtgcacatcgatgcggccgcgttggatgcggcgaagcgggcggcgaccggtctggtcgaatcgcagtacgacagggctgcgtgggcgacgatgcgtgaggctcttgcggccgccggagacaccgacatggcgcacatctcccaggccgacgtggacacccttgcggcacggctgaatgatgccatcgccgcgctgtccgacgcgcgtctggaccgtggcccgctgtctgacgccatcgcgtcggctcaggctctcaaggaaggcgattacactgccgaatcgtggaaggcgttcgccgaggcgttggccgccgcgcaggaggagtgcggacggcgatccaccacgcagggcgatctggacaaggccgtaaaggccctgactcaggctcgcgacgggctggagcgcaagcagcccggcggcggtgaggaaccaggaggtggcgaggatcccgagcagcctggcagcggcgggcagaccgagcgtcccggtggcgtcaagcccggcgccggcggcaacgccgatacctccaaacctggcacggcgggcacggaaaccctctcgaagacgggcgcccagacgctgctgctcgcagtgacggcgtgcatcatgcttgccgcgggattctccatcacgacggcgcgctcccgtcgccggtga

SEQ ID NO: 39 - DNA, 3339 bp, Bifidobacterium bifidum
  source 1..3339: mol_type = genomic DNA; strain = CCOS 571
atggagaaaagctccaatagacgtttcggtgtgcgtaccgttgcggccattgtagccggcctgatggtcggtggcatgtgcaccgcgatgacggccagcgccgccgacgatagtgcagccgggtacagtgccacggctcccgtgaatctcacccgcccggccaccgtcccgagcatggacggctggaccgacggcaccggcgcatggacgctgggtgaaggcacccgcgtcgtgtcgagcgatgcactggccgcgcgtgcgcagagcctggcatccgagctgaccaagttcaccgacgtggacatcaaggccgcgaccggttcagcgacgggcaaagacatctcgctcacgttggatgcatcgaagaaggccgaactgggtgatgagggattcaagctgtccatcggctcgaagggccttgaggtcatcggcgcgaccgacatcggcgtgttctacggcacccgttccgtctcgcagatgcttcgccagggccagctgaccctgcccgccggcacggtcgccacgaagccgaagtacaaggagcgcggcgctacgctgtgcgcctgccagatcaacatctccactgactggatcgaccgtttcctgtcggacatggccgacctgcgcctcaactacgtgctgctggagatgaagctcaagccggaagaggacaacaccaagaaggccgccacatggtcgtactacacgcgcgacgacgtcaagaagttcgtcaagaaggcgaataactacggcatcgacgtgattccggagattaactccccgggccacatgaacgtctggctggagaactacccggagtaccagcttgccgacaactcaggccggaaggatcccaacaagctcgacatctccaacccggaggccgtgaagttctacaagacgctgattgacgaatacgacggcgtattcaccacgaaatactggcacatgggcgccgacgagtacatgatcggcaccagcttcgacaactacagcaagttgaagacgttcgccgaaaagcagtacggagccggcgcgacaccgaatgatgcgttcaccggcttcatcaatgacatagataaatacgtcaaggccaagggcaagcagctgcgcatctggaacgacggcatcgtcaacaccaagaacgtctccctgaacaaggacatcgtcatcgaatactggtatggtgccggccgcaagccgcaggagctcgtccaggacggatacacgttgatgaacgcgacccaggccctgtactggtcccgttcggcgcaagtgtacaaggtcaacgccgcaagactgtacaacaacaactggaacgtcggaacctttgacggcggacgacagatcgacaagaactacgacaaactgaccggtgcgaaggtgtccatctggcccgacagctcgatctaccagaccgagaacgaggtcgagaaggagatcttcgacggcatgcgcttcatctcacagatgacctggtccgattcccgcccgtgggcgacgtggaacgatatgaaggcggacatcgacaagatcggctacccgctggacatccgcgaatacgactacaccccggtcgatgccggcatctacgacatcccccagttgaagtcgatctccaaaggtccgtgggagctcatcaccacgcctgacggctactaccagatgaaggatacagtgtccggaaagtgtcttgcgctcttcaccggaagcaagcatctcgatgtcgtcacccaggtcggcgcgacgccggaactgcgcaactgcgccgacgtgtccgtcggtcaggatcagcgcgacaccgccaatgagcgtaacacgcagaagtggcagatccgcgccgacaaggatggcaagtacaccatctccccggcgctgacgcagcagcgtctcgccatcgccaccggcaacgagcagaacatcgatctggagacccatcgtcccgccgctggcaccgtcgctcagttcccggctgacctggtcagcgacaacgcgctgttcacgctcaccggccacatgggcatgtccgccaccgtggactccaagaccgtgaacccggcctccccgtccaagatcaccgtcaaggtgagggccgcatccaacgcgaacaccggtgacgtcaccgtcaccccggtcgtgccggaaggctgggagatcaagcccggaagcgtcagcctcaagtcgattccggccggcaaggccgccatcgcctacttcaacgtggtcaacaccactgggacgggcgacgccaccgtgcagttcaagctcaccaacaccaagaccggtgaagagctcggcaccaccagcgtggcgctgaccggcagcctgaccaaggacgtggaagcaagcgactacgcggcgagctctcaggagaccaccggcgagcatgctcccgtgggcaacgccttcgacaagaacgcaaacacgttctggcacagcaagtactccaaccccagcgccaatcttccgcactggctggcgttcaaggcgtcgcccggcgagggcaacaagattgcggccatcacccacctgtaccgtcaggacaagctgaacggcccggccaagaacgtcgcagtgtacgtggtggcggccagcgacgccaacagcgttgccgatgtcaccaactggggtgaacccgtcgccacggctgagttcccgtacaccaaggaactgcagaccatcgcgctgccgaataccattccgtccggtgacgtgtacgtcaagttccagatcaacgacgcatggggcctcaccgaaaccagcgcaggcgtcacctgggcggctgtcgccgagctggctgcgacggccaaggccactccggtcgagctcaccgagcccgagcagccgaaggacaaccccgaggtgacggagactccggaggctacgggtgtgaccgtttctggcgatggtgttgccaatggcgcgctgtcgctgaagaagggcaccaccgcgcagctgaccgcgaaggtcgcgccggatgacgcggaccaggcggtgacgtgggcctccagcgatgacaaggtcgtgacggtcgacaagaccggcaaggtgaccgccgtcgcgaagggcgtggcgaaggtgaccgcgaccacggcgaacggcaagtccgcctctgtgaccgtcaccgtcaccgaggactctgaggtgcccggcccgactggcccgaccgagccgaccaaaccgggtacggagaagccgaccacgaagccgaccacgaagccgaacgacggcaagctcagcgccaccggcgccgacacggcggtgctcgccaccatcgcggctctgttcgcgctggccggcggtgcggtcgtcgcggttcgtcgccgcagcgtccgctga

SEQ ID NO: 40 - DNA, 2022 bp, Bifidobacterium breve
  source 1..2022: mol_type = genomic DNA; strain = CCOS 586
atgaacggtcaaacatctctgcaaggatggaccatcatccccatgccgcaaaccatgcagcataaagcgaacatcgcattgctgccaatgtgcggacgaatcaacgaagcgcgcgcagtcggtgacgatcggcacatacttgccgtgcagctcatcgacgacatccgcgcagccaccggattggaatgggatatcgccaccggcgaccgctggccgggattcatcaccttgacgacctttgacgacccccatgcacacccgtccggtgcatatacgctcgatgtcaccccggacggcgtgaccgtagcgggagcggatttcgagggtgtgcgtaacggcgtgcagactttgcgtcagctcatccgccaatgcggcgccgccctgccctgcctgcacatcgaggaccgacccgccttcgaaacgcgcggctactacctcgacgtcacccgcgggcgcgtccccaccctcgactggctcaaacactgggccgacaagctctgcctgtacaaatacaaccagctccagctctacatcgaacacaccttcgcgttcgactcgatgagcgagacctggcgcggttccagcccgctcaccccgcgcgacatcctcgaattcgacgactactgcgccgaacgcggcatcgagctggtcccgtcggtctccacgttcggacacctctacatggccctgcgcacgcaatccctgcgcgacctcggcgagttccccgaaaccgccgacgagccgttcggattcatcgaccgcatgcaccaccacacattgaacatcagcgacgaccgcgccttcgacctgtcatgccggcttatcgacgactacctgcagctgttccgctccgacaaattcaacatctgcgccgacgagaccttcgacctcggcaagggccggtccaagcccctcgccgaccgcatcggcgtcgcggccatgtacgccgactacgtcacccgcctgtgccgccacctcgaagcccggggcaggcggccgatgatgtggggcgacatcgccctcgaacaccccgagatcctcgaccggctccccgaaaccgtcaccctgctcaactggcagtacgaccctcaggtcacggacgagaagatccgcaccgtcgccgaatccggtgccaagcagatcgtatgcccggccgtatggtgctggaacgcgctcctgccgcgcatcgacgacgcctggaacaacatcacccgcatggcccgctacggcgcgcaataccatgcgcaaggcatgctcatcaccgactggggcgacttcggccacgtcaacgacccgcgcatggccatccccggcatgatcatcggcgcacaggaatcatggaacccgggacgaatcccggatgaggccgacatgctccgccgcatctcccgactcgaataccacgacgccagcggcgaactgcttgatatccttgctcgcgcaagccatgcggccagtttcgaatggaaccacctgatcacttggctggaacttgatgacggacaaggcggagtcaacaccggggtcctgcaaaccatcccgggactgctgccggaaaacgaacgaccggacgatgtgatccgttccctccagaacgaaagcaagacaccgtcacttgcggaatcccgacgaatgctgctccgctatctgaaacaccgcatcacgctcggcgaaaccgcagatcaccttctgcaggccagtacccgtcgaatctccgcgatcaccgcgaccgcaggaccgcggaacgcaggaaacgccgctgcattccgcatagccgtcgaggggcagcgactgctgaaccgggttggcctccgacttgcgtccgagaccgggatcactgacactttgcagccgaacaccacgtctcaacatgacgatgaggcgaaccttgctgaagcattggagatctggatggaggcgtatgcgacgcaatggagcacggtcagccgagactccgaactccgtcggctgcaagatacggtgtggaagttaacggaccatctgcgcttccaatccgtctga

SEQ ID NO: 41 - DNA, 525 bp, Bifidobacterium breve
  source 1..525: mol_type = genomic DNA; strain = CCOS 586
atgaaatatcccgacatcccgaacgtcaccaaggaatatctggacgaagtgcggcccgccatcaacgccggcggatcatgctcggccacggccggagccaccatcggcgcccttagcccgcccttggtcgtcgatggcaccagcatcgacatcgacgccagcggcgtgcccgtggcctcgatcaactcatacagcgtgggccaatgcacctggtgggccgccgcccgacggcaacagatcggcaaaccggtggacccctacatgggccacggctacatgtgggcggccagcgccgagaaacacggataccccaccggcggcacaatccaactgggcgacgtgatgagcttcgagcgaggagtgctgggagccagcggcgaatacgggcacgtcgccatcgtcgaggaaatccacgaggacggttccatcctcatcagcgagtccggcgtcgaccaacgccgcgcctggacacgcctgctgacacgcgagcaggccgagaaccccggcatcacctacatccactag

SEQ ID NO: 42 - DNA, 1851 bp, Bifidobacterium breve
  source 1..1851: mol_type = genomic DNA; strain = CCOS 586
atgagattgtcatcaggtgatcacgctgggcgtgcttctgctcggactcgacacaggaaatggctgcggtctggcttggcagcggtgctgtcgatggccacgctggcggcattggccgcaccggcaagcgccaccgacaccatgccgtggggaggccagtatgccgataccagccatccggagcagtctgcgggagccaaccagccatatcagcatggctataccggtctggatattctgaactggaacccggacgcggataaggactcggaatacctgcgctcccgtgtaccgttgcagacccgcatcgccgccaacgccgcgacgcagaaagacccgaacctgccggccgatacggagatgttcaaccttgctggcgactacggcaacgcgttcttcgagtcgttccacgacaacagcgtgttctcgcagtacctgttcaactactggcagtacacggattattacggtacctggcatggtcagccgactgcgaacgtgccaaagagcctctacgacgagaaggcccagtctgactggactcagaaatggtttgagttcggtacgttgaatctgccgaatgccgcatataccaatgtcgcgcacaagaatggcgcgaagtcgattgcgacgatcttctattctggcaacgaccgaggagaacagacctataaggacctgcttcaaggcaagcgtgccgacggcacctatccggtggctgacaagctggtggagattgccaagtactatggttttgatggttacttcgttaaccaggaatccagtgtgaattctgctgatgtgccggcctaccaagacttcatgaagcagatcatcgatcagggcatttatatccagtggtacgactccgccacatacccgaatggcggcgtcagctatcaaaacatgttcaacgatgccaattcgccgtgggtgcaggatccgaacaagggtaaaatctctgattcgatcttcctgaactactggttcagcggcaacatgctacaagattcagctgatcacgccaagagccttggcattgatccgaagtacgcggtattcgcgggtatcgaggccggacagaagaaatttggtagcatcgccagcaacgcgaattatatgaatgtcaaccttgacgctgacggcaagccctatgtgagccttgcggcacttggcaccgatttcgtctcccacgagttgggtgacgacaagaaggtctacccgaagtatcagaaccaggtattcgaccgcgaacgccgtctgtggactggttcgtccaccggtgagaagggcacgacggatatatctgatccgtatattgatgatggtacgagcagcgatagctggaagggtttcgcttcgcagatcgccgagcgctccgtcatcgacggtccggtcttctccacatcgttcaacaccggtcatggtctcgaatggcgagacaacggcgaacagaccagcaaccagcagtggggcaatatcaacctccaagacattctgccgacatggcagtggtggattgatgccgattccgacccgctgcaagccgacttcgactatggcaagaaatatgaagcggcaccacgctttaactacaccaaggtcggcggctacgaaggcggcgattcgctggtgttgagcggcaagctgtccagcgacaacaccgtccgtctctacaagaccgacttgagcgtcgctgccggctccaaggtcgaactgacctacaacaagctcaacagcgacgattccaagctgcaactcggcctcattttcgccgacgacaccaagactatagtgccggtggacatggagacggtggggcaagcaacggctggaagaccgcaactgtag

SEQ ID NO: 43 - DNA, 1884 bp, Bifidobacterium breve
  source 1..1884: mol_type = genomic DNA; strain = CCOS 586
atggaattcaccgtatccggcactaccgtgcgattcgatgaacgaacgatgcagttcgcttttacgcgcgacggtgccgaatggaatacctgcgctgatttcaagccgaccctgcaatgcacgcaaggcaccttcgcgttcgccgatgccacttccatcacccacgaacaacgcgaaaccggcacgggaaccggcattcgcagcatcttcaccggctttgggcacagcgcatactcctttgaaacctatgtatgggtggagcgcgcctccggcgatgtgctcttcgaatggattccgctcaatgagcaagggctgaccgtcaccggtgtgacgtggcctgcggcaatcgccttcgaccgtgacgactcccacgacgtgacgcttattacccacgaacagggcgtgatgattcccaacacatggcctaccgccgtgagcaccaaagatatcaccttcgacggccgcttcgaaacggctggcggttacatgccgtggttcgcgcagctccgcgcagacggacacgggtatatcgccatctgcgaaactccatggaatgccggttacggcatcgaccatcccagcaatggcccgtacacccatatcaatacttggtttgagccaagcctcggcacaatgaattatcgccgcgtggtacgctaccagttcctcgaccacgccgatcatacggcggtgtgcaaggcctatcgttcgtacgtcaacgaacgaggtcgcctgcgtacgctcgccgaaaaggcggcgcgcaatccctccgtgcgcgacctgattggtcgttcatgggtgcacgtcggcatcaaaaccaaagtgcagcccgactcgttctactatgacaaggaccaccccgaaaagaacgattcattggtcacctttgcgcagcgcgaaaagcaaatgcgaacattgcacagcatgggtgcaggccgactgtacatgcacttggatggttgggcacagcccggatacgacaacgcacaccccgactatttgccggcttgtcaggaggcaggcggctgggaaggcatgaagtcgctggtcgacgcctgccatgagcaaggcgatattttcggcacgcatgaccagtaccgcgattactacttcaccgcgcaaacctttgatgccaacaatgcgattcggctggcagacggtacgatgcccgaacacgcacgctgggcgggcggccaccagacctacctgtgcgccgagctcgcaccggactacgtacgccgcaatttcacccagattgccgcgcatggcatcaagctcgactgtgcatacttggatgttttcacctgcaacgaaggcgacgaatgctcgaaccccgaacaccgcatgacccgccgcgaatgctttgaccgccgcaccgaatgcttcgaatatctgctctcgcacggcatcctctcctcgtccgaagaggtatcggactgggcagtgccaagcctgatattctgccattacgcgccatacgacttccagatgcgctcacccaacgagccgcgccaaggtgtgccggtgccgctgtataacctcgtctaccatgattgcgttatcgaaccatggatgatggagcgtgtggtgaacggcgacgattacatgctgtacgccttgctgaacggcggagctccatacctgattcgcgatgccgcatacatcggcgttgacggcgacatggacgacgaacagcgcgctcgcacggaaaacgacatcgaacgctgccatacggtcgctgcattccatgagcgggtcggcatgcaggaactggttcgtcacgagttcgtggatgatgatccgctggtgcagcgttcggtattcgtggacggcactgcggtcacttgcgacttccatacgcaaacctatcgcatcaccgattga

SEQ ID NO: 44 - DNA, 714 bp, Bifidobacterium breve
  source 1..714: mol_type = genomic DNA; strain = CCOS 586
atgtcgaccgatcagttacgtcaatcggccagcgtatggggctcacagctggcagccgccggcattaacgtcgatcttgcacccgtgctgggcacggtagtggcgaatcgggccgccaacgcccccattggtgcgctctatcgcgatttcggactggacgcggcaggcaacgcggctcagggcacggcgtttgtgcagggcatggccgatgccggagtccaatccgccatcaaacattatccgggattgggcgcggtaaccggcaacacggatttcaccgccgacggcatcctggatacgaccaccaccttggatggcacggaaatcaaggcgtttgacaccaccatcgaccaggccgaccctgccatggtgatgatggcattggccacctatcaggccattgaccccaatgatcccgcggtgttttcctcaatcatcattgacgggcatcttcgcggcgatctgggatatgacggcgtagtgatttcggattcgatgtcggcagcggccgtgagcgcctacgacaccactcaattgggagtcaaactcgtggaagccggcggcgatttggcctgcatcggatacaccaactatgtcgcgccgattctggatggattgtctacgcgagcggcatcggatcccgccttcgccaatgaggtcactcagtcggcgattcgtgtgatgacgttgaaaatcaagatgggattggcctaa

SEQ ID NO: 45 - DNA, 402 bp, Bifidobacterium breve
  source 1..402: mol_type = genomic DNA; strain = CCOS 586
gtgtcgatcggcgaggacacggccggtgtgcggttgtgcggcgaagttcgcggcggcggtctgttcctgtatcctctgggagaagggcagattcgcaaaatctccgacgtgccgcccaccaccgataccgcgttgttcgacaaaggatatgaaggatccgcaagctaccgcatcccgtctttggtgactacgccctccggtgtgacgattgccggtgccgaccaacgtgtggtcatctccaacgatgccccaatgagatccatttcgttgtccgccgctccttggatagcggacagacgtggctgccgctgcaaaccgtcatcgcctttcccggtgatgggctggatggcgcgagcgtcatcgactcatgcatcgtctgtgatcgtgagacaggccgtgtga

SEQ ID NO: 46 - DNA, 1302 bp, Bifidobacterium breve
  source 1..1302: mol_type = genomic DNA; strain = CCOS 586
atgtccacctttccacagctggccacccatccgggcatgaacccggccgcggtcatccaaggcgaccgttggcgcatcggcatcatcaccgaatcgctcgtccgcctcgaatggcaggacaacggcaagttcgaagaccacgccacccaaatgatcgtcaatcgcgattggttgtcggatgacgcgaacggtgcggatggcgcgaaccgtgcggacggcacttcaaacccgccgaagttcacgaagaccgagcgcgacgggctgctcatcatcgatacgccggcgctgcgcctcacctacgacatgcagccgttcagtaaggaaggtctgagcatcgtcgtcaaaggcgtggcgaattcccagatgaatacgtggcactacggcgaagcccaggacggcaacctgcgcggcaccgcgcgtacgctggacgcggtcgacggtgagatcgaactggggctcggcgtgatctcccgcgacggctgggccgtgctcgacgattccgcatcaaacgtgatcgtcgaaggcgccgaagccgccaccgtcaagggcgaggccaacccattcggtatgtgggtcattccgcgcgagcaccccggcaaggatctgtatgtcttcggatacggccatcggtacatcgaagccgtgcaggacttctacaagctcaccggcccgacgccgctgctgccgcgcttcgcgctcggcaactggtggagccgctaccaccggtacacggaagccgagtatctggagcttgtcgatcgcttcgagcaggaaggcctgccgttcaccacggccgtcatcgacatggactggcatctggtcgacaatgtcgacccgaagtatggatccggatggaccggctacacgtggaaccgtgaattcttccccgaccccgagcgattccagcgcattctgcacgagcatggactgcgcacgacgctcaacgtccacccgcgcgacggcgttcgagcatttgaagacggctatgccaaagtcgccgaacacatgggcattgacccggcgggcggcgagccggtcgagttcgatctgaccagtccgcgattcatggaagcgtacttcgacctgcaccatgggctcgaaaccctgggcaccgatttctggtggctggactggcagcagggcggcgtgacccgccagaagggtcttgacccgctgtggatgctgaaccacatgcattatctcgactccgccgtgacgggcgctggccgctcacgttctcgcgatatgccggcccgggctcgcaccgttatccggtcgggttctccggcgacacagtcgtga

SEQ ID NO: 47 - DNA, 1017 bp, Bifidobacterium breve
  source 1..1017: mol_type = genomic DNA; strain = CCOS 586
gtgattgatgtttctgcctggcagggcaatatcaactggcaggccgtgaaaaactcaggcgtggaaggcgcaatcattcgcattggctacgggtgggacaacgggtttgacaagcaggcacttcgcaatatcagcgagtgcaggcgtttgggtattccgtttggcatatacctctattcgtacgcttatgacgccaataccggtgctgctgagggatccagtttagtgaacctgctgcagaaagccggtgtgagctcaagcgatttgggctatccggtgtattacgacttagagaggtggacctggacgggtcatgaggtgcccaacgaccctgggacgtatgacggcattgtcaatgcttggtatggcaggttgcaatccgctggatacaacaatcttgctgtgtattcatacacttcttatctgaatactgcgctcaatagtggcaatatccactctaagacccgttgggttgcgcagtatggttcctcgatgggctatacggcattccccaccaacgaccgtggctggcagtacaccagcaggggcagcgtgagcggcattagcggcaccgtggatttgaatgcgtttggtaatcaaactgccacgtcctcggctcctagcgttcctgtctaccgtgtctacaatcccaacagcggattgcaccattacacgatgaattacaacgaagtcataatgctggttggtaaggggtggcgatacgagaaaaccgcctttagagcgggccaatccggcatacctgtctaccgtgtctacaatccgaatgacggaaaccatctgttcacgatgaatagttacgagcgagacaatctggctcgtttggggtggcacgatgaaggcgtgtcttggtacgtgccttccggcggatccattaacgtataccgtttgtataacccgaacaatggtgagcatgttttcaccacggaatatggcgagtatgtgcttgttggcagggctggatggcatcaggaaggcgttgcctggacatctctgtga

SEQ ID NO: 48 - DNA, 375 bp, Bifidobacterium longum
  source 1..375: mol_type = genomic DNA; strain = CCOS 974
gtggggattccagttcgatccggcgggtatcagctcatcggagccaagaagaccttcatcgacgagatgaaccaatggatgaaaaccgaatccaacaagccgttcctgttcaccgaatacggtgcggacaccgatgccggtgtgcacaagctgccgagcgtgcaatggagtgaagagtaccaatgtgagtacctcgccatgcagcacgaagtgttcgacatgttcgaggcagtagtgggcgagcaggtatggaacctctgcgacttccaaaccggtgaaggcatcatgcgcgtcgacggcaataagaagcgtcttcactcgcgaccgtcagcccaaagccgccgcctatgtactcaaggagcgttgggagactaa

SEQ ID NO: 49 - DNA, 1203 bp, Bifidobacterium longum
  source 1..1203: mol_type = genomic DNA; strain = CCOS 974
gtggcgacaatatgtgctttggcagtggtgttggccggcgtggcagcctacggatggcatgtcggatggttcgccaaatccacatcaaacggcaacaccaccactccccagacctcccagacaagtgcgttaccacgcgccgacgttccttccccgaagaagaacgaaccggccgcacaagcccaacgtgcggtatccgccatgacacttgaggaacgtgtgggacagttagtgatggtgccgttgcttgccggttccgatccctcctcgttggcttcaacaatcgccgatgagcacattggctcggccattctcattggcaattggaataccggcgtcgatacggtcaagaccgcgaccgcgcaactccaaggatatgcgccggccggcaatcgtctcatcatcgccaccgatcaggagggtggtcaggtgcagcatctgactggtaccggtttcgacacaatgcccagtgccgttgagcagggcacaatgtccgctgacgcattgcgtcaatcggcaggcacgtggggttcgcaattagctgctgctggcataaatgtcgatttggcaccggtgctcggcacggtggtgggcgatcgtgcctcaaatactccgattggcgcgctggatcgtgatttcggattggatgcggccggcaacgccgaacatggcatcgccgttattgaaggtttgcgagacgctcaggtcggcgcggcggtcaaacattatcctgggttgggcgcggtaagcggcaataccgacttcaccaccgaaggcattctggatacgaccaccacgttggacggggccgaggcaggtgcctttgaccaggcgatcaccaagaccgatcccgcaatggcgatgatgtcgttggccacgtatcaatccatcgacccgaataatccggcggtgttttcttctacgattatcgacggtcatattcgcaacgccctgaagtacacgggcgtggtgatttcggattcgatgtctgccgaagcgttaagcagctacgatgtaagccaattgggagtaaagcttgtcgaggcgggcggcgacatgtcctgcatcggtcagacggattacgtaaagccaattgtggacggcctcaacgaacgggcgaagtccgatccggctttcgcttcgaaggtgacggcggcggcgactcgcgtaatggcattgaaaatcaagatgggactggcctga

SEQ ID NO: 50 - DNA, 1896 bp, Bifidobacterium longum
  source 1..1896: mol_type = genomic DNA; strain = CCOS 974
atggaattcaccgtatccggcactaccgtgcgattcgatgaacgaacaatgcagttcgcttttacgcgcgacggtgccgaatggaatacctgcgctgatttcaagccgaccctgcaatgcgcgcaaggcaccttcgcgttcgcggatgccacttccatcacccatgagcagcgtgaaaccggcacgggaaccggcattcgcagcatcttcaccggctttgggcacagcgcatactcctttgaaacctatgtatgggtggagcgtgcctcaggcgatgtgctcttcgaatggattccgctcaatgagcaaggcctgaacatcaccaatgtgacgtggcccgcagccatggacttcgattgcgccgacgaccatgacacgacactcatcacccatgagcaaggtgtgatgatccccaacacatggcctaccgccgtgagcaccaaagacatcgccttcgacggccgcttcgaaacggctggcggttacatgccgtggttcgcgcagctccgcgcagacggacacgggtatatcgccatctgcgaaactccatggaatgccggttacggcatcgaccatcccagcaatggcccgtacacccatatcaatacttggtttgagccaagcctcggcacaatgaattatcgccgcgtggtacgctaccagttcctcgaccacgccgatcacacggcggtgtgcaaggcctatcgttcgtacgtcaacgaacgaggtcgcctgcgtacgctcgccgaaaaggcggcgcgcaatccctccgtgcgtgacctgattggtcgctcgtgggtgcacatcggcatcaaaaccaaagtgcagcccgactcgtactactacgataaggaccaccccgagaaaaacgagtcgctggtcaccttcgcacagcgcgaaaagcaaatgcgaacactgcacggcatgggtgcaggccgactgtacatgcacttggatggttgggcacagcccggatacgacaacgcacaccccgactatctgccggcctgtcaggaggcaggcggctgggaaggcatgaagtcgctggtcgacgcctgccatgagcaaggcgatattttcggcacgcatgaccagtaccgcgattactacttcaccgcgcaaacctttgatgccaacaatgcgattcggctggcagacggtacgatgcccgaacacgcacgctgggcgggcggccgccagacctacctgtgcgccgagctcgcaccggactacgtgcgccgcaatttcacccagattgccgcgcatggcatcaaactcgactgtgcatacttggatgtcttcacctgcaacgaaggcgacgaatgctcgaaccccgaacaccgcatgacccgccgcgaatgctttgaccgccgcgccgagtgcttcgaatatctgctctcgcacggcatcctctcctcgtccgaagaggtatcggactgggcagtgccaagcctgatattctgccattacgcgccatacgacttccagatgcgctcacccaacgagccgcgccaaggtgtgccggtgccgctgtataacctcgtctaccatgattgcgttatcgagccgtggatgatggagcgtgtggtggacggcgacgattacatgctgtacgccttgctcaacggcggagctccatacctgattcgcgatgccgcatacatcggcgttgacggcgacatggacgacgaacagcgcgctcgcacggaaaacgacatcgaacgctgccatacggtcgctgcattccatgagcgggtcggcatgcaggaactggttcgtcacgagttcgtggatgatgatccgctggtgcagcgttcggtattcgcggacggcactgcggtcacttgcgacttccatacgcaaacctatcgcatcaccgactgcccgcatcactga

SEQ ID NO: 51 - DNA, 948 bp, Bifidobacterium longum
  source 1..948: mol_type = genomic DNA; strain = CCOS 974
gtgggaatcctcaacaaaggcaagccgaaacacggacacctgcaccggcgcgtgggcatgacgctgaccgcgcttgccgtcgcggtctccatggcgttcgccccggcggcgatggccgacatgcagggcgtggacatgagcaactggcagtgcggcgcggacgtgtacaacatgcaggccgatttcatcgtggtcggcaccacatggggcaccgggcaagtcaacaacaactgcttggtgtccggcgtcaacacggacgccaaccgcatgatcgcccaggcacaagcatccggcaagaaattcggtttgtatcactacgcgatgggaggcaacccggaggcggaagcccaattcttctatcgcaacacgtcgaactattggcgtcacggcatcgtggcgttggattgggagatggacgacaaccccgcatggggcgattgggattgggtacgccgattcacggcggagtgtgaacggctctcgggcggcgtcaagccgctgctctacaccggccccgtggccggcaccatccccggcgacatccgcgccaactacggtttgtggatcgcgcagtacgcgaacatgagcccgaccggctaccaggccaacccgtggatgctgggcgcgtacggcgaggccatgcgacagtacagcggtaccggcgtggtcaacacgtggagtcccatcgacctcaacatcttccgtggcgaaggctggcagtggatctgtacgccaaccccaccggctccacagccccggccccggcaacgcccgcgcccgtgcagccgagcactcccacggccaacaccaacacgggtggcatcagccacgtcatgcaatggggagaaaccatctggggactcgccgtcgcctatgatgcttggcccctgtccgcgtggcatacgccgagcggtgacatcaaccgctactacgtgggcgacgtcgtaa

SEQ ID NO: 52 - DNA, 2136 bp, Bifidobacterium longum
  source 1..2136: mol_type = genomic DNA; strain = CCOS 974
gtgcccacattcgaatataaggctgatgccgcaacgccgtgtctcaccctgattcccgctccggtgacattggaatacacgcatggcacggctatgatcgggtccttggtgacgatcgagaaacgaattcccgaatatgccgtcaccgaagatgccgatgagacctgggaaacgctgccaatcgagcagctgtccagcgaactcgagcgttactgcggcgtcgccgtccgtacgcgccgagtactcacagccacagatgaagccgacgccggtgccaatgccgccgaaaaagcgcgagatgcgggcgtgggtgcgggtgcgggcgcgggtgcgccggcagcgatgaacggcaccgtcatattgctgtgtgtggatgcgcgattggcgcatgacgaatacacgctggatgtgttcgcctcagataccatcgccgtgcgtggtggcagcgaaagcggattgcggtacggcatgcagacactgcggcagatgatcaggcaaacctcgcggaccttgccctgcctgcatatccaagacaagcctgcgttcgcggtgcgcgcctacagtcttgacgtgacgcgcgggcgtgtgccgacgatggcgttcctcacctggttcatcgaccagctggcgttgtataagtacaaccaattccagctgtacgtggaacatgccttcgctttcggcgagctcagcgaagcgtggcgcggcactgacccgctcacggccgacgacatcatgttcctggacgaatactgcgcacaccacggcatcgaactggtgccgtccttggccactttcgggcacatgtatatgaacctgcgcacccgtgaacatcgtggactgggagagttccccgaagacgccgaccgtccgttcagtttcatcgaacgaatggaacaccacacactgaacgcagccaaccccaagtcgcacgatttcgcctcgcgcctaatcgaggaatacgcgccgctgttccgctcgcggtccttcaatattggtggcgatgagactttcgacttgggccgtggccggtccgtacaggactcgccgggcgccagtcgcgatgagctgtatgccgatttcgtcaaagacctatgcagcacgcttgcccatcgtggtctacaaccgatgctgtgggcggatatcgcgctcgagaatccgcacaccatggacttgctgcccggcgacatcacgatgctcaactggatgtatgagccggatatcgacgaaagcaagatccagaccatcgcatcacaaggccgccgccaattcgtgtgccctgccgtgcgtgcgtggagccggttcttccccgactatgacggtgcctggctgaacacgtaccgcatggcggtggctgggctcaaatatggtgcggaaggcatggtggtcaccgattggggtgactatgggcatgtcaatgatccgcggttgagcgtgccgggactgtgctacggtgcgcaaaacgcgtggaacccggttgcaattgacgcatgcgaaatgaaccatcggatttctaatctggcgtatggcgacgaatccggttggcttatggattcactcgctcgcatcgactccgatggagtgtcgttcccctgggacctcgccgtgcaagtgctggaactggaatacggttccggcaccggcatgctgaatacggatgtggcatcgtatgtggaacggtcgtgcgggggagagctcatgtttgaccgtgcgctgggatgcgccgacgcgcgccgccgattgctcctgcggaaccatgcccgtctcgaacgacgccgggattgcgaccgggcgttgattgattgcggcagtgcggttgtcgcggtgctcgatggcttggcacgaggaggattgaacccggagctgctgtgggtgatgctggatgggcagcggttgttcaatagactgggtgaggaactgctggtattggctggcggcgaggacgcctgtgacaccaaggatgtgaccggccgtgcgctggatgcctcgcgccgcgcccgtcttgccgccgatttggagttgtggttcgaacggtaccgtgtgcagtggctgtccatcggccgttacgccgaattggcgcgtatcgcccatgtggtgtggtcgtttgcggacatattgcgtaggggagcgctatag

SEQ ID NO: 53 - DNA, 1905 bp, Bifidobacterium longum
  source 1..1905: mol_type = genomic DNA; strain = CCOS 974
atggtggaaaagaagcggctcgacggctcggatgcgaagggcgcgacgggtgcggcgcgtgcgacgggcatgctgcgtgcgttgcgtgcgcgtgcggcggcgtccggtgcggggcgcggcgtgaccgcggcggtgacgtccgtcgcggcggtggcgatgttgggcgcggtgtgcgtcactccggccagtgcgctcgcggatgtgaacgccgacgcggacgcgtccggcgtccggtccgcggcctccgcgaccgcctcggacggaacctcatccgacgcgcgctcgcaggcgcggtccgcgacttccgcgacgcgtccggtcgcgaacggtgtgacggtcgccgatgagacggctgccgacgcgatgccggacaatccggatgccgagctgccggacaaggtgtccgccgagatctccgatgacgcgatggtggtgtccgagcaatatgcggctacgccggaaggcgagttgaaggacatcgagacgggcgagacggtcaccgatccgaagatcgtgggcacggagagcaagcagcccgatccgttggcgaagacggatggcgagtcgttcattccggtgagtgccgcggatgtgaaggagaaggtcgctgcgaacggcggtgacgtgaacgcggtctcctcgaagacgcgcgcggccaacgcgtcggtgaagctcgccgcgttgcagaacaacgagtacggtgcgcactggggcacgtacaacggtacgcaggcgttcttcgacgcgcgcaacaacctgttcgcccagcaggcgaagggcgtgatcgacgtgtccgcatggcagaacacgatcgactggcaggcggtgaagaacgcgggcgtcgagggcgcgatcatccgtctgagctacggctggggcaatggcttcgacgtgcaggcgaagcgcaacatcagcgaatgcaagcgtctcggcatcccgttcggcgtgtacgtcttctcgtatgcggaaagtgcggccgacggcgcatccgagggtgcggacgtggtgaacctgctgcgccaggcaggcgtgaatccgggcgacctgagttacccggtgttctacgatctggagaactggacgtacacggggcacaagagcccaacgagtccgagcgtgtacgacggtatggtgaactcctggtacggcaaactgcaggcggccggttacaacaatctgagcgtctactcgtacacgagctacctgaacagcgcgctcaacagctccaacatccacggcaagacccgttgggtcgcccagtatggctccaccatgcagtacaccgcgttccccacgaacgaccgcggctggcagtacacgagcggcggctccatcaacggcatttccggtcgcgtggatatgaatgcgttcggcaactatcaattcactcccgcggttcagataacttgggtttatcgctctgaagatattgctgtcggagcatcggttgattacccttcatcggatattgattacaaatggcaatcgtataatttatccagcaaacgttggaaaaccatcaccgattggacaggtgccaattgggcgggttgggtggaccagttaggagactattggctacacgtggaagcacgtgactctcggactcgcaagattatcggatcgcagacaatcgctttcaggtatgcacccggtacaacgcgtgtggcggcaacatatgcgggatggcagaacaatcatgtgttgctgggggaaagttcaaataatgcggctgcccattatgagattaaaatctatgatgttagacgcagcaaatgggtgcaaggtttcaagggaccgtgggcgatttggaagcccaaaaagggtatatattggactcattatgaggtatatacctctgatgggagactcgcggacactaagacttatgcgttcggtgtctaa

SEQ ID NO: 54 - DNA, 174 bp, Lactococcus lactis
  source 1..174: mol_type = genomic DNA; strain = CCOS 949
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 55 - DNA, 2982 bp, Lactococcus lactis
  source 1..2982: mol_type = genomic DNA; strain = CCOS 949
atgataaaaagttcatttaaagctcaaccgtttttagtaagaaatacaattttatctccaaacgataaacggagttttactgaatatactcaagtcattgagactgtaagtaaaaataaagtttttttggaacagttactactagctaatcctaaactctatgatgttatgcagaaatataatgctggtctgttaaagaagaaaagggttaaaaaattatttgaatctatttacaagtattataagagaagttatttacgatcaactccatttggattatttagtgaaacttcaattggtgttttttcgaaaagttcacagtacaagttaatgggaaagactacaaagggtataagattggatactcagtggttgattcgcctagttcataaaatggaagtagatttctcaaaaaagttatcatttactagaaataatgcaaattataagtttggagatcgagtttttcaagtttataccataaatagtagtgagcttgaagaagtaaatattaaatatacgaatgtttatcaaattatttctgaattttgtgagaatgactatcaaaaatatgaagatatttgtgaaactgtaacgctttgctatggagacgaatatagagaactatcggaacaatatcttggcagtctgatagttaatcattatttgatctctaatttacaaaaagatttgttgtcagatttttcttggaacacttttttgactaaagttgaagcaatagatgaagataaaaaatatataattcctctgaaaaaagttcaaaagtttattcaagaatactcagaaatagaaattggtgaaggtattgagaaactgaaagaaatatatcaggaaatgtcacaaattcttgagaatgataattatattcaaattgatttaattagtgatagtgaaataaattttgatgttaaacaaaagcaacaattagaacatttagctgagtttttaggaaatacgacaaaatctgtaagaagaacatatttggatgactataaggataaatttatcgaaaaatatggtgtagatcaagaagtacaaataacagaattatttgattctacatttggcataggagctccatataattataatcatcctcgaaatgacttttatgagtccgaaccgagtactctatactattcagaagaggagagagaaaagtacctcagcatgtatgtagaagccgttaaaaatcataatgtaattaatcttgacgacttagagtctcattatcaaaaaatggacttagaaaagaaaagtgaacttcaagggttagaattatttttgaatttggcaaaggagtatgaaaaagatatttttattttaggggatatcgttggaaataataatttgggaggggcatcaggtagattttctgcactctctccggagttaacaagttatcatagaacgatagtagattctgtcgaaagagaaaatgagaataaagaaattacatcgtgtgaaatagtatttcttccagaaaatatcagacatgctaacgttatgcatacatcaattatgaggaggaaagtacttccattttttacaagtacaagtcacaatgaagttctgttaactaatatctatattggaatagacgaaaaagaaaaattttatgcacgagacatttcaactcaagaggtattgaaattctacattacaagcatgtacaataaaacgttattcagtaatgagctaagatttctttacgaaatttcattagatgacaagtttggtaatttaccttgggaacttatttacagagactttgattatattccacgtttagtatttgacgaaatagtaatatctcctgctaaatggaaaatttggggaagggatgtaaatagtaagatgacaataagagaacttattcaaagcaaagaaattcccaaagagttttatattgtcaatggagataataaagtttatttatcacaggaaaacccattggatatggaaattttagagtcggcgataaagaagagctcaaaaagaaaagattttatagagctacaagaatattttgaagatgaaaatatcataaataaaggagaaaaggggagagttgccgatgttgtagtgccttttattagaacgagagcattaggtaatgaagggagagcatttataagagagaaaagagtttcggttgaacggcgtgaaaaattgccctttaacgagtggctttatctaaagttgtacatttctataaatcgtcaaaatgaatttttactgtcgtatcttccagatattcagaaaatagtagcaaacctgggtggaaatctattcttcctaagatatactgatcctaaaccacatattagattgcgtataaaatgttcagatttatttttagcttacggatctattcttgaaatcttaaaaaggagtcggaaaaataggataatgtcaacttttgatatttctatttatgatcaagaagtagaaagatatggtggatttgatactttagagttatccgaagcaatattttgtgccgattctaaaattattcctaatttgcttacattgataaaagatactaataatgattggaaagtcgatgatgtatcaatcttggtgaattatttatatctgaaatgcttctttgagaatgataacaaaaggattcttaattttttgaatttagttagtcctaaaaaggttaaagaaaatgtcaatgaaaagattgaacattatcttaagcttctgaaagttaataatctaggtgaccaaattttttatgacaagaattttaaagaattaaagcatgccataaaaaatttatttttaaaaatgatagctcaagattttgaacttcagaaagtttattcaattattgacagtatcattcatgtccataataaccgactaattggtattgaacgagataaagagaaattaatttattacacacttcaaaggttgtttgtttcggaagaatacatgaaatga

SEQ ID NO: 56 - DNA, 1801 bp, Lactococcus lactis
  source 1..1801: mol_type = genomic DNA; strain = CCOS 949
atggatgaagtgaaagaattcacatcaaaacaattttttaatactttacttactcttccaagcaccttgaagttaatttttcagttggaaaaacgttatgcaatttatttaattgtgctaaatgctatcacagcttttgttccgttggctagtctttttatttatcaagatttaataaactctgtgctaggttcagggagacatcttatcaatattattatcatctattttattgttcaagtgataacaacagttctgggacagctggaaagttatgttagtggaaaatttgatatgcgactttcttacagtatcaatatgcgcctcatgaggactacctcatctcttgaattaagtgattatgagcaggctgatatgtataatatcatagaaaaagttactcaagacagcacttacaagccttttcagctatttaatgctatcattgttgtgctttcatcgtttatctcattgttatctagtctattttttattggaacatggaacattggggtagcaattttactccttattgttccagtattatctttggtactttttctcagagtgggacaattagagtttttaatccagtggcagagagcaagttctgaaagagaaacatggtatattgtatatttattgactcatgatttttcatttaaagaaatcaagttaaataatattagcaattacttcattcataaatttggaaaattaaagaaaggatttatcaaccaagatttagctattgctcgtaagaagacatatttcaatatttttcttgatttcattttgaatttgataaatattcttacgatatttgctatgatcctttcggtaagagcaggaaaacttcttataggtaatttggtaagtctcatacaagctatttctaaaatcaatacttattctcaaacaatgattcaaaatatttacatcatttataatactagtttgtttatggaacaactttttgagtttttaaagagagaaagtgtagttcacaaaaaaatagaagatactgaaatatgcaatcaacatataggaactgttaaagtaattaatttatcatatgtttaccctaattcgaatgcctttgcactaaagaatatcaatttatcctttgaaaaaggagaattaactgctattgtaggaaaaaatggttcagggaaaagtacactagtaaagataatttcaggattatatcaaccaactatgggaataatccaatacgacaaaatgagaagtagtttgatgcctgaggagttttatcagaaaaacatatcggtgctgttccaagattttgtgaagtatgagttaacgataagagagaatataggattgagtgatttgtcttctcaatgggaagatgagaaaattattaaagtactagataatttaggactcgattttttgaaaactaataatcaatatgtacttgatacgcagttaggaaattggtttcaagaagggcatcaactttcaggaggtcagtggcaaaaaattgcattagcaaggacattctttaagaaagcttcaatttatattttagatgaaccaagtgctgcactcgatcctgtagctgaaaaagaaatatttgattattttgttgctctttcggaaaataatatttcaattttcatttctcatagtttgaatgctgccagaaaagcaaataaaatcgtggttatgaaagatggacaggtcgaagatgttggaagtcatgatgtccttctgagaagatgtcaatactatcaagaactttattattcagagcaatatgaggataatgatgaat

SEQ ID NO: 57 - DNA, 1113 bp, Lactococcus lactis
  source 1..1113: mol_type = genomic DNA; strain = CCOS 949
atgttagcggagttaaaaaataaagataactcaaagatatatcagaaaaagatagacaattatattgaatatattgttagcaaactttcaacatatgggcttttaacaggatcactttattcgggagcagctggcattgcattaagtatcctacatttacgagaagatgacgaaaaatataagaatcttcttgatagcctaaatagatatatcgaatatttcgtcagagaaaaaattgaaggatttaatttggaaaacattactcctcctgattatgacgtgattgaaggtttatctgggatactttcctatctattattaatcaacgacgagcaatatgatgatttgaaaatactcattatcaattttttatcaaatctgactaaagaaaacaaaggactaatatcgctttacatcaaatcggagaatcagatgtctcaatcagaaagtgagatgtatccactaggctgtttgaatatgggattagcacatggacttgctggagtgggctgtatcttagcttatgcccacataaaaggatatagtaatgaagcctcgttgtcagctttgcaaaaaattatttttatttatgaaaagtttgaacttgaaaggaaaaaacagtttctatggaaagatggacttgtagcagatgaattaaaaaaagagaaagtaattagggaagcaagtttcattagagatgcatggtgctatggaggtccaggtattagtctgctatacttatacggaggattagcactggataatgactattttgtagataaagcagaaaaaatattagagtcagctatgcaaaggaaacttggtattgattcatatatgatttgccatggctattctggtttaatagaaatttgttctttatttaagcggctattaaatacaaaaaagtttgattcatacatggaagaatttaatgttaatagtgagcaaattcttgaagaatacggagatgaaagtggcacgggttttcttgaaggaataagtggctgtatactggtattatcgaaatttgaatattcaatcaattttacttattggagacaagcactgttactttttgacgattttttgaaaggagggaagaggaaatga

SEQ ID NO: 58 - DNA, 738 bp, Lactococcus lactis
  source 1..738: mol_type = genomic DNA; strain = CCOS 949
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 59 - DNA, 2049 bp, Lactococcus lactis
  source 1..2049: mol_type = genomic DNA; strain = CCOS 949
gtgaaaaaaatactaggtttcctttttatcgtttgttcgttgggtttatcagcaactgtgcatggggagacaacaaattcacaacagttactctcaaataatattaatacggaattaattaatcataattctaatgcaattttatcttcaacagagggatcaacgactgattcgattaatctaggggcgcagtcacctgcagtaaaatcgacaacaaggactgaattggatgtaactggtgctgctaaaactttattacagacatcagctgttcaaaaagaaatgaaagtttcgttgcaagaaactcaagttagttctgaattcagtaagagagatagcgttacaaataaagaagcagttccagtatctaaggatgagctacttgagcaaagtgaagtagtcgtttcaacatcatcgattcaaaaaaataaaatcctcgataataagaagaatagagctaacttcgttacttcctctccgcttattaaggaaaaaccatcaaattctaaagatgcatctggtgtaattgataattctgcttctcctctatcttatcgtaaagctaaggaagtggtatctcttagacaacctttaaaaaatcaaaaagtagaggcacaacctctattgataagtaattcttctgaaaagaaagcaagtgtttatacaaattcacatgatttttgggattatcagtgggatatgaaatatgtgacaaataatggagaaagctatgcgctctaccagccctcaaagaaaatttctgttggaattattgattcaggaatcatggaagaacatcctgatttgtcaaatagtttaggaaattattttaaaaatcttgttcctaagggagggtttgataatgaagaacctgatgaaactggaaatccaagtgatattgtcgacaaaatgggacacgggacggaagtcgcaggtcagattacagcaaatggtaatattttaggagtagcaccagggattactgtaaatatatacagagtatttggtgaaaatctttcgaaatcggaatgggtagctagagcaataagaagagctgcggatgatgggaacaaggtcatcaatataagtgctggacagtatcttatgatttcaggatcgtatgatgatggaacaaatgattatcaagagtatcttaattataagtcagcaataaattatgcaacagcaaaaggaagtattgttgtcgcagctcttggtaatgatagtttaaacatacaagataaccaaacaatgataaactttcttaagcgtttcagaagtataaaggttcctggaaaagttgtagatgcaccgagtgtatttgaggatgtaatagccgtaggtggaatagatggttatggtaatatttctgattttagtaatattggagcggatgcaatttatgctcctgctggcacaacggccaattttaaaaaatatgggcaagataaatttgtcagtcagggttattatttgaaagattggctttttacaactactaatactggctggtaccaatatgtttatggcaactcatttgctactcctaaagtatctggggcactggcattagtagttgataaatatggaataaagaatcctaaccaactaaaaaggtttcttctaatgaattctccagaagttaatgggaatagagtattgaatattgttgatttattgaatgggaaaaataaagcttttagcttagatacagataaaggtcaggatgatgctattaaccataaatcgatggagaatcttaaagagtctagggatacaatgaaacaggaacaagataaagaaattcaaagaaatacaaataacaatttttctatcaaaaatgattttcataacatttcaaaagaagtaatttcagttgattataatattaatcaaaaaatggctaataatcgaaattcgagaggtgctgtttctgtacgaagtcaagaaattttacctgttactggagatggagaagattttttaccggctttaggtatagtgtgtatctcaatccttggtatattgaaaagaaagactaaaaattga

SEQ ID NO: 60 - DNA, 687 bp, Lactococcus lactis
  source 1..687: mol_type = genomic DNA; strain = CCOS 949
gtgtataaaattttaatagttgatgatgatcaggaaattttaaaattaatgaagacagcattagaaatgagaaactatgaagttgcgacgcatcaaaacatttcacttcccttggatattactgattttcagggatttgatttgattttgttagatatcatgatgtcaaatattgaagggacagaaatttgtaaaaggattcgcagagaaatatcaactccaattatctttgttagtgcgaaagatacagaagaggatattataaacggcttaggtattggtggggatgactatattactaagccttttagccttaaacagttggttgcaaaagtggaagcaaatataaagcgagaggaacgcaataaacatgcagttcatgttttttcagagattcgtagagatttaggaccaattacattttatttagaagaaaggcgagtctgtgtcaatggtcaaacaattccactgacttgtcgtgaatacgatattcttgaattactatcacaacgaacttctaaagtttatacgagagaggatatttatgatgacgtatatgatgaatattctaatgcactttttcggtcaatctcggaatatatttatcagattaggagtaagtttgcaccatacgatattaatccgataaaaacggttcggggacttgggtatcagtggcatgggtaa

SEQ ID NO: 61 - DNA, 1345 bp, Lactococcus lactis
  source 1..1345: mol_type = genomic DNA; strain = CCOS 949
atgggtaaaaaatattcaatgcgtcgacggatatggcaagctgtcattgaaattatcataggtacttgtctacttatcctgttgttactgggcttgactttctttctacgacaaattggacaaatcagtggttcagaaactattcgtttatctttagattcagataatttaactatttctgatatcgaacgtgatatgaaacactacccatatgattatattatttttgacaatgatacaagtaaaattttgggaggacattatgtcaagtcggatgtacctagttttgtagcttcaaaacagtcttcacataatattacagaaggagaaattacttatacttattcaagcaataagcatttttcagttgttttaagacaaaacagtatgcctgaatttacaaatcatacgcttcgttcaatttcttataatcaatttacttaccttttcttttttcttggtgaaataatactcattattttttctgtctatcatctcattagagaattttctaagaattttcaagccgttcaaaagattgcattgaagatgggggaaataactacttttcctgaacaagaggaatcaaaaattattgaatttgatcaggttctgaataacttatattcgaaaagtaaggagttagctttccttattgaagcggagcgtcatgaaaaacatgatttatccttccaggttgctgcactttcacatgatgttaagacacctttaacagtattaaaaggaaatattgaactgctagagatgactgaagtaaatgaacaacaagctgattttattgagtcaatgaaaaatagtttgactgtttttgacaagtattttaacacaatgattagttatacaaaacttttgaatgatgaaaatgattacaaagcgataatctccctggaggattttttgatagatttatcagttgagttggaagagttgtcaacaacttatcaagtggattatcagctagttaaaaaaacagatttaaccactttttacggaaatacattagctttaagtcgagcacttatcaatatctttgttaatgcctgtcagtatgctaaagagggtgaaaaaatagtcagtttgagtatttatgatgatgaaaaatatctctattttgaaatctggaataatggtcatcctttttctgaacaagcaaaaaaaaatgctggaaaactatttttcacagaagatactggacgtagtgggaaacactatgggattggactatcttttgctcaaggtgtagctttaaaacatcaaggaaacttaattctcagtaatcctcaaaaaggtggggcagaagttatcctaaaaataaaaaagtaat

SEQ ID NO: 62 - DNA, 678 bp, Lactococcus lactis
  source 1..678: mol_type = genomic DNA; strain = CCOS 949
atgcaggtaaaaattcaaaatctttctaaaacatataaagaaaagcaggtgctacaagatatcagttttgatattaaatctggaacagtctgtggtttattaggagttaacggtgcaggaaaatcaactttgatgaaaattttgtttggtttaatttctgcagatactggaaaaattttttttgatggacaagaaaagacaaataatcaacttggagccttaatcgaggctccagcaatatatatgaatttatctgctttcgataatcttaaaactaaggctttgctttttggaatttcagataagagaattcatgaaactctagaagtgattggtttggcagaaacaggaaagaaaagagcaggaaaattctctttagggatgaaacaacgtttgggaattggtatggctattcttacagaacctcaatttttaattcttgatgaacctactaatggtttggatcctgatggtattgcggagttgttaaacttaatcttaaaacttaaagctaaaggtgtgacaatcttgatttctagtcatcagttgcacgaaataagtaaagtagctagtcaaattattattttgaacaaaggtaagattcgttataatcatgcgaacaataaagaagacgacattgaacagttattctttaagattgtgcatggaggaatgtga

SEQ ID NO: 63 - DNA, 729 bp, Lactococcus lactis
  source 1..729: mol_type = genomic DNA; strain = CCOS 949
atgaaaagaataatagcatcagaagcaataaaattaaaaaaatcaggaactcttagattggtattaattatcccttttgtgactctatttatagcatttcttatgggtggaatacagatttttagtgttttttcaatttattggtgggaaactggttttttattccttttgatgagtttgctttttctttatgatataaaatcagaggagcaagctggaaattttcaaaatgtgaaatggaaaaagctgagttggaaaattcatttggccaaaatgttgttgatttggctaagaggtatactagcgagcatagtcttgattattttgctttatttggttgcttttgtgtttcaaggtattgtagtggtggattttatgaaagtaagtgtggcattgattgctatattactagcagcttcttggaatttaccctttatatacttgattttcaagtggattaatacttacgtattgttagctgcgaataccttgatttgtttaattgttgccccttttgttgcacaaactccagtatggttcttgctaccatacacttatcactataaagttacagaaagtttgttaaatatcaaaccatcaggagatttgttaacagggaagataaatttcagtatttgggaagttttattaccatttggactttccatagttgtaacgataggagtttcgtatttacttaaaggagtgatagaacatgataagaagtga

SEQ ID NO: 64 - DNA, 645 bp, Lactococcus lactis
  source 1..645: mol_type = genomic DNA; strain = CCOS 949
atgataagaagtgaatgtctcaaattaaaaaatagcttagggttttatttagtttttctctttactttattagagcttttaacggttcctatttatttagcttttggaagaagtcatgtttcaatgactgatttatcgctcatgatttttttgttttttccgttactggttacaattttgtctattctaatctttgaacaggagagtctggccaatcgtttccaagaaataaatgtaaataaaaaaagtagcagaatttggttatcaaagctaatagtagtggatttccttttgttctttccatcagcaatgatctggataattacgggagtttcacaggcagtagggcaacaaggaatgatgatcgcaacagctagctggttgatggcaatttttcttaatcattttcatcttttattgacctttataatcaatcgaggagggagcatgattatcgcgattattgaaatattactcattatttttgccagtaataaagttttattagcagcttattggtgtcccattgctttacctgttaattttatgataactgggcggtgtgcttatctgatagctgccgtagggtggattgttttatccacaataattcttgtagcattatctaaaaaaaagattagataa

SEQ ID NO: 65 - DNA, 1320 bp, Lactococcus lactis
  source 1..1320: mol_type = genomic DNA; strain = CCOS 949
atgccagtttctcgtattaaagttaaaaatagacatttaaaaaagaaagccaaaaaaccactggctttttataaaccagcgaccaaatttgctggcgctgttctcattgcaggaactttgacgaccactcatgagcttttgctccaacaaacaagcccaatggttcaggctgcaaccaattccacagaagcttttattgaaagtattgctgcatcagcaaaaccagttgccgatagtaatggtctttatccttcagttatgattgctcaagctattttggaaagtaactggggttcaagccaactttctcgtgccccctattataatttattcggtattcaaggaacttatcaaggaaaaagcgttgtttttaaaactcaagaatatctcaatggtaaatgggtcacaaaagatatgccatttagggtttatccttcctttaatcaaagtttccaagacaatgcttatgttttaaaaactacaaactttggaaatggtccttattatgctaaagcttggcgagcaaacgcggcaacctatcaagctgcaaccgcagccttaacaggaaaatatgcaactgaccctaattatggtgcttccctgaatcgaattatttctcaatataatttgactcgttttgacggtgcttcttctgctggtacttctaattccggtggttcaacagctacaaataccaataataattcaaatacaagctcaaccacttatacagttaaatctggcgatacactttggggaatttcgcaaaaatatggaattagtgttgctcaaattcaaagcgcaaacaatcttaaaagtacagtcatctatattgggcaaaagcttgtattgacaacttcaagttcttcgtctaatacaaatagttcaacttcttcaggaaattctgccggaactacaacgcctactacttcggtcactcctgccaaaccagcttcacagacgacgattaaggttaaatctggtgatacgctttggggactctctgtcaaatataaaacgacgattgctcaactcaagagttggaatcatttgaattctgatacaattttcattggacaaaacttgattgtttcacaatctgccggttcttcaagttcttcaacaggttcaagctcagcctctacgagttcaacttctaactcttctgcagcttcaaatacctctatccataaggttgttaaaggagatacgctttggggactttcacaaaaatctggtagcccaattgcttcaattaaggcttggaatcatttatcaagtgataccattttaattggtcaatatcttcgtattaaataa

SEQ ID NO: 66 - DNA, 1044 bp, Lactococcus lactis
  source 1..1044: mol_type = genomic DNA; strain = CCOS 949
atgaaaaaggtgattaaacggaaggttatattatttagccttccttttttattgcttttattgccaattctagctttctttgctctatttgtaggttcaagtgatttatcttcagatacagatattaataccaatacaccacagcaacaaactgccaaagtgatttgggatagagttctaaaagaaggtggaacgaaagaaggggcagctgctttacttggtaataatcaagcagaaagtgaacttcaaccttcaattattcaatctaatgcaacctataatgaagcaaaagcaatggataccactttaggcggttatgctttcggtttggctcaatgggatagtggcaggagagtaaacttgctaaactatgcgaaaagtcagaaaaaatcttggacagatactaatcttcaagttgagttcatgtttgagcaagacggtacagattcaacgttacttaaacaattaatcaaaggaactaatgttaagcaaacgactgaagatattatgcgaaagtgggaacgtgcaggggcggttgatagccttccaaaacgtcaaggttttgcggaatattggtacacgttcatgacgactggtggtgatagtggaactggtggtggttcaggaattactccagatataccttcaggttggactttagataaaccaattaatacaagtggttatcttgcgacaagttatgagtataaacaatgtacgtggtttacatggaatcgagcccaagatttcggtattacttttggaatgtatatgggaaatggtgctgattggcaacatcaagcaggatatactgtaacgactactccaacacttcatagtgcggttagctttagtggaggtcaaacagtaggcggtcaatggactgctgacccacaatacggtcacgtggcctttgtggaaggaatacattcagacggttcagtcttgatttcacagtcaggaactggttttagtacagtttatactttccaagtattaacaaaagcacaagctagtcaattacattatgtgataggaaaataa

SEQ ID NO: 67 - DNA, 894 bp, Lactococcus lactis
  source 1..894: mol_type = genomic DNA; strain = CCOS 949
atgcagtataaaaaaaaagaaaacaaggttttggtaatttttgtactggtcgtaatttttattttgtgtggatgcatatatgtcttactagtcagatatacccatactgataaaataacgacgatcagttataagcagccaactaaaacatcggcttctaacggttatgtagagcaaaaaggtgaagaagctgctgtgggtagtataacacttgtagatgatgctggtgtaccagaatgggttaaagttccctcaaaggtaaatctagataaatttactgatttatctacgaataatatcactatttatcgaattaacaatccggaagtcttaaaaacagttaccaatcgtacagatcaacggatgaaaatgtcagaagttatagctaagtatcctaatgctttgattatgaatgcttccgcttttgatatgcagacaggacaagtagctggatttcaaattaataatgggaagttgattcaagactggagtccaggtacaacgactcaatatgcttttgttattaacaaagatggttcgtgcaaaatttatgattcaagtacacctgctttaactattattaaaaatggagggcaacaagcctatgattttggtactgcgattatccgtgatggtaaaattcaaccaagtgatggctcagtagattggaagattcatatttttattgcgaatgataaagataataatctctatgctattttgagtgatacaaatgcaggttatgataatataatgaaatcagtatcaaatttgaagctccaaaatatgttattacttgatagtggcggctcaagtcaactatctgtcaatggtaaaacgattgttgctagtcaagatgatcgagccgtaccggattatattgtgatgaaataa

SEQ ID NO: 68 - DNA, 1500 bp, Lactococcus lactis
  source 1..1500: mol_type = genomic DNA; strain = CCOS 949
ttgactaataatgataaaactaagcgcttaaaaaaggaaaaatttattgcgggaacagcgctgattttaggaatgacgacttttggcgtggctggccatgctgatggagtaactgttggaagtagttcagatgcatcatcaagcacaggtagctcaagttcatcatcaggtacagggggttcaagttcatcatcaagtacaggtagctcaagctcatcatcaaattcaaatgcttcaagttcatcgtcaagtacaggtaattcaagttcatcatcaaaaccaagtacttcaggttcgacatcaaaaccaagtaatccaagttcatcaacaaataataattcaactagtaattcctctacaacaactcaagcaccatcaacttcagtttctaccatagctccttcagcatcaacttctacagttgcaccctctaattattcacaggataatgcctactatcaaacatcaactgcagcacaaattcctaattcttcagcagattcagctccttctatttacgctggccctgttttgaaaacaattgaagcagctaagtcaattgataaaattgatacttctagtacagaagcctttattaaaagtattgcagaccgcgtgagaattttagctggtaagaataatttgtatgcatcaattatcttggcacaggcgattttagaatctggttctggtcaaagtaatatgagtcagcagtattttaatattttcaatattacaggtgcttatttaggaaaatcaatttcatttaagacggaagaattttcgggaaataatccttattatattgaacaaagttttagagtttatagtaattatgaccaagccttggatgattatattaatttaatgattaaagggacaacttggaactcagaaatttatgctggcgcatggaagtcacatgctaagacttaccaagaggcggctcaagcattgcaaggaatttttgctactgacccagcatatgcacaaaaattaattgaaattattcaagaatataaattagatgcttatgataatgttgatagtacaacacaagttgtagatagtaaaattcctgaaagtccattggcggcatcaaaattagataattcagcttatccagaatataatggggtagaatatccaggtgctgatagttatgcttttggtaattgtacacagtatgtttataaccgtatcattcaattaggtgggctcgttggaacacatatgggaaatggtggagaatggggaattaatgcccaagctcaaggttatttcacaacaactgttccaactgagggttatgctgttagcttcccaccaggagtagctggttcaagttcagaatatggtcacgtagcttttgttgaaaaggtttattcagataactctattctcgtttcagagatgaatgttaaaggtaataatattgtcagcgaacgccatatttctgctggtgtagctgctttggcaacttatattcaacctaaataa

SEQ ID NO: 69 - DNA, 780 bp, Lactococcus lactis
  source 1..780: mol_type = genomic DNA; strain = CCOS 949
atgtcaagtattgaaaatatgattgcttggatgcaagcacgaaagggtaaggtaacttactcaatgaccttgcgaatgggtcctagaagttatgactgcagctcgtcagtattctttgcaatgattgctggaggttttctgtcagaaggttcaatgggtaatactgaaaccttgtttggaatgtcaggaacgaagctgaaagaaatcagtcgtggagaggtccatcgtggcgatatcttcatctcaggcactccaggaggttcggctggttcagatgggcatactggtattttcctaagcaatggctcattcattcactgctcttatactcacaatggaattgcggttgatacgaatgatgcatacatgagtactcgcttgccacatcacttttatcggattgttggttcaggttcagcaaatactgacagcaagcctcaaatggttacattaaatgttgatggacagtttggaaatgcgactgctaaacgattgcaagaatactttgatacagctggtaaagatggagtaatcagtcaccagtacaaacaaacctttaatcaaaatatctatgctgcgcagtttgattcatcactgactggttcaaacgtggttaaagcattgcaaagattcttaggcattggccaagacggcttgtttggccaaggtacaattaaagcactacaaaaacatcttggaacgacgcaagacggaactatcagcccagtttctgattctgttagagaattgcaacggcgattgaatgcgaataaattataa

SEQ ID NO: 70 - DNA, 816 bp, Lactococcus lactis
  source 1..816: mol_type = genomic DNA; strain = CCOS 949
atgaaacctaaaatgagacgtaaacttaaaaaaatgggagcttttatctttacagcatccatgattgcaatgatcgccttacttggtttagtacgaattcgaccggtagaacctcctaaaaaagcagcaactgaaaaaattgttgtaaatcatattttggatgagaaggttttggatttgaataaaccagttgttgacctttcgggttggcaacgtccagaagatattgattacaacaccttaagtcagcatgtgattggtgcggttattcgtgtcaatggttcttatggtcatgctgataattcagcaagcaaggatggagaagatactgcctataaacagcacattaaagctttccaagagcggggaatcccgactgcggtttatgcttttgtgactggagaaaacacttctgaaatgaggaagcaagcaagagacttttatcgtcgagcaagcccttataagccgacctattattggcttgatgtcgaggtaaccaatatgaaaaatatgaaccaagggattgaggcttttcgctctgagttggagaaacaaggagctaaaaacattggtatttatgcccaagactggttcttgcgagataatcaaataaaagttgataaatttaaagcgatttggattgctgcctatggacgaaatacaggttattgggatgcttcaccagaaacgactttaagttataaaatgcaacaatttactgaccaaggaactttaccaggttactcgggaaatgttgatttgaatatggttaataaccaaaccaactataatgaattgtttaaaaatcaaaaataa

SEQ ID NO: 71 - DNA, 2613 bp, Lactococcus lactis
  source 1..2613: mol_type = genomic DNA; strain = CCOS 949
atggataaaagaagcaaagaaaactttaaagcttcgctcaatgtccagaaacgtgtctctgatggaattgatgcgaatgcctttagaaaacctcgacaagccacgcaattaagtaaacaggcgtttaaagaagggaaaaaaacactcaaaattcgccgaaaaaactatcgaaaattaaaagctggttcaggtttacaagttgtcgcagccaaaaagaaagttcttgaagcaaaagcagataaaaccgtttctaaagcggtttttaaaaatgcaaagaaatctgatccaacacgagcagctaatcaagcgaaaggctatgccaagaatcaagccaagcaaacagcagtcaaccacttagtgttatcgcctcttgaaaaagatgatactttaagagaaggttcagacctctatagaaagactcaacgtaccaaaagtgggttaaaactttctaaggatgttgtcacttcttccgctaaagcttcggttaatctttcaaaccatacttacggtttagccaatcgaagtgttaatattgttcgaggccgtgggtttgtcagaactccttctgaattggggttcagaaggcaagccgcaaaaagaatgcggaactttcaaaataagctgagtgccgcaagaaaggcaaaaaaggcggagcaaggattttctttgattcggtcaatcttaaaaggccaacaaaccctgagtcgtgcgtttactttaatcgtgacctccaaagggttactcgtcgttcttgctgttatgtttatattgtgtctcttgggaatccttaacatggcctcctctgttccaacaaagcaagatgactttcaactgacaaagtcttggacatatttcacaaaacttgatgcagacaattcagagaacggaaactctttttatacgcctttggatgatgtgatgttttatatgaatgatcaatttgaagattataatttacaagatcaagtgcctgtaggttcaaatggcgcagcacttcctaatcaaaattatgaacaatatcttacaggcctttggtcagccttaaatggctcttctcctgattataagctaacaacaatggaggctttggagacggataagcgttctaaatattatctttctcctgatgattattcagattttaaagaacgtgttaatgaagtcggttatgatagccttgatggtcaacttcagtttccttatcaaacagagagtttggttatcaatcgtagatatggctacgaacggaatggagataaaactgaactccacgcaagcattgatgttagttcagccccagggcaagatttaacttcaccaatgcacggaattgttaattctgtgactgaccctaatacattggtcatttctgaagctgaaaatgcacggttgacaattgttgggattaatagcggacggtttattggtggtgaaacagtagatgctggaacacttcttggcaaggccacaaagtcaagtcttaatatgacctatgaaaaatataatgaagatgataaaaaatgggaaaaagttaatccagcattttattttcctaaagtgacctatactcagtttacctctttagcttcagatagctttgatccaggaaaaagtgtttctgaacgtgcggaggccgtttataatttcttaaccaaacttggctataaaaaagaaggaatttgcgctatcttaggttcttttactgaagaatctcaaatcaatcctaagcgagctgaaggagattatttatctccaccagttggtgcttctggtaattcttgggatgatccggcttggttagctatgggtgggttggatatttacggaaaatatccaaatattcttcatagagggttggggttaggtcaatggacagatacatctgatggttctgaaagacacactttacttttaaattatgccaaagcaaaaaataaaaaatggtacgacttagaccttcagcttgattttatgctcaatggagatactccagggaatcaaaccatgtttaaaaataccgctagtaatgccgtctcttcttcaattccagaactgaccaactacttcttgacttactgggaaggaaatccaggtgacaagattcaggcacgtgtacaagccgcccaaaattggttcacttacttctcaaataacggtggctccgatgcggatatgagcgcttcatcaaaagaactctttgagaaatataaagacaaaattaaacctctcccttctaataaagaaacacaacaagggcaaggatggcctgggaatggctatgaacctgggaattgtacttggtatgtctttaatcgtcaagctcaaattggtcataatattaatggctatatgggtaatggtggccaatggggttataactatactaaaactccaggagctacaattgattctaaaccacaagtgggagatgcggtaagtttctctccaggtgttgcagggtctagttcagaatatgggcacgtggctcaagttgaagttgtcaatccagatggtacatttttagtttctgaaatgaacactttaggcctttattcaatgggctatcgaatgtttaaaccaggagcaggaatgacatttgttcattttaaataa

SEQ ID NO: 72 - DNA, 978 bp, Lactococcus lactis
  source 1..978: mol_type = genomic DNA; strain = CCOS 949
atggaaaaaggattattagtcgatattggacgaaaatattggtcaatagcagagttaaaaagattagttcttttacttcaagagcataagctgacacatttacaacttcatctcaatgaaaatgaaggatttgcgctaaattttactgacagccctgtcagtaaaaaatattcagaaaatatgctcaaagaattgaaagaatttgcaaaaacacacgaaatcactttgattcctgattttgatagtccaggtcacatgggctccttattagagcaaaatcctgaatttgcactacctgacagcaatcaacaagcggtagatgttactaatccagcagtcattgattggattatgggaataattgataagatagtcgatattttcccagattcagatacttttcatatcggggctgatgaatttattgattttcggcaaattgaaaaatatccttatttagtggagaaaactcgggaaaaatatggaaataaggcaagtggtttagagttttattatgactatgttaatcaattgaccgagcatttgcagaaaaaaggaaaacaagttcgtatttggaatgatggatttttacgaaaagaccttcagtcattggttcctttgaataaaaatgtggaggtttgttattggacaaactgggacaaagggatggcagaagtcaaagaatggttaactaagggttataccttaataaatttttgtgataatgacctttattatgttttaggtgaagaagcaggctattcttatccaacggctgaaaaactggaaagggaaggaaaaattcaaaaattttctggtcagcaatatttaaatcaagaagagatgaaagctgtcagaggaacttacttttcgatttgggctgacaatgctgcagctaagtctgtcagtgaaattttagacgatcttagcaaggtacttccggtatttatgaaaatttatggaggaaatgatgaataa

SEQ ID NO: 73 - DNA, 1641 bp, Lactococcus lactis
  source 1..1641: mol_type = genomic DNA; strain = CCOS 949
atgaaaaaatcgaaaaaattttttattagaatatgcctttctacaggaattttggcatctgcaactttactgacagcttgttttggaactccagcaaaacatgtgaaagctaattataaagtgacagcagaagcaattccagattaccaaagtaagaatgctcccatttcatcttattggatgcctgataaatttctagaatggtcagcggaaaatgacaaagatttagtttacaatcaatcccgagtgccacttaccaaaagaatttctcctgataaactaagtccatccaatcaaaatcagaacaaaaagacaaaaattgtcgccttgtcgatgatgaactcacaaacttctggaaatccttcgcggggcacgacaaaatttgagagctatacttttgattattggcaatatattgacacactggtttattggggtggctcatctggtgaaggaataattgtaactccgtcagcagatgttattgatgaagcacacagcaatggtgttccggtacttggaacaattttcttgccgcctaaagaatacggtggaaaagtagattgggtcaaaacaatgctcaaaaaagatgagcaaggacaatatccatttgccagtcaaatggttaaggttgccaagacttatggatttgagggttggtttatcaatgaagaaacccaagggctaaatgctgacgatgcagctaacatgaaagccttgattcaacaggtgaaaaaagaagattctagtcttcaaatcatgtggtatgatgccatgactaaagatggaaaagtggattggcaaaatcagttaaatgaccaaaatgcgacatttgtacaagataaagcagcagacgcgatgtttttaaatttctggtggactcaaaataatttggccgaccaaaaattacttgaaaaatcgaatctctatgctaaaaatcacaatattgacccttataatatttatgccggaatagatgtgcaagcgaaagacgtccaaactccagttaaatggaaccttttagaaaaaggaaatcaagccactcaaacatcaattggactctatgcagcaagcgctacctacactaacgcaagtaattgggatgattttcaaaatcgtgaatcagcattctgggtcaatcaaaaagcagaccctcgtcaagttgatcactctgttaatgaatcatggacaggactttccaaatatgttctggaaaaatcagcaataagcggtaatgaatttaatactaattttaatttaggaaatggttataactattttaaagctggtcaaaaaatctcagaaatggattggaacgaccgcagtttagcaggtattttaccatcttaccgctggattattgacaacgaaggaaaaaataaaataagtccaagcttcgactttgcaaatgcttataacggtggaaattcactgaaatttatggctgaacatttagatgcaggcaaaagctcaaacatcacattgtttgctagtgatttgaaaattgctatgggagcaaaattctctgttagtatgcgctcagaccaagcgcttaaagtttctgcaatcttagaactagcaaatggtcaaaaagttagcattgcaggagataaaagcctgactgagaattggtcaaaataa

SEQ ID NO: 74 - DNA, 630 bp, Lactococcus lactis
  source 1..630: mol_type = genomic DNA; strain = CCOS 949
atgaaatataaaactcgacgaagaaaaccaaatcgacgtcaatccttgttaaaacagctcatctttattttcctagttattttaggaatcttagtctataataaaatttttaataaccaagcacaaaagccgtctgaagcacaacttcaagaaattaatgaacataaattcatcaaagaaattgctccactcgctcaaaaatcacaaaaagaaagtcaagtactagcttctatcaccattgctcaggcctgtcttgagtctaattttggcaaaagcgaacttgcaagtaaatatcataacctttttggtgtcaaagcatctgatgatgtgccaaaggtttcgcttgctacgcaagaatatgaaaatggtcagtgggtcactgtccaaggtgttttccgtgtttatccaaattttgctgattcagtttcggcacacacccagcttttcctttacggaacaacatggaattctaaacaatatgcttcagttcttagtgcaactgattataaaactgctgctaaagccgttcaaaattctggctatgcaacagaccccacttatgctgacaaacttatcaatatgattgaaacttatcatttaaatcaatatgataaaagttcaagtatttaa

SEQ ID NO: 75 - DNA, 780 bp, Lactococcus lactis
  source 1..780: mol_type = genomic DNA; strain = CCOS 949
atgtcaagtattgaaaatatgattgcttggatgcaagcacgaaagggtaaggtaacttactcaatgaccttgcgaatgggtcctagaagttatgactgcagctcgtcagtattctttgcaatgattgctggaggttttctgtcagaaggttcaatgggtaatactgaaaccttgtttggaatgtcaggaacgaagctgaaagaaatcagtcgtggagaggtccagcgtggcgatatcttcatctcaggcactccaggaggttcagctggttcggacggacacacgggtattttcttaagtaacggttcattcattcactgttcttacactcacaatggaattgcggttgatacgaatgatgcatatatgagtactcgcttaccacatcacttttatcgaattgttggttcaggttctggaaatactgacaataaacctcaaatggttacattaaatgttgatggccagtttggtaatgcgactgctaaacgattacaagaatactttgatacggctggtaaagacggagtaatcagtcaccagtacaaacaaacctttaatcaaaatatttatgctgcacagtttgattcatcactgacaggctcaaaagtggtcaaagcattgcaaagattcttaggcattggccaagacggcttgtttgggcaagctacgattaaagccttacagaaacaccttggaacaacgcaagacggaactatcagcccagtttctgattctgttagagaattacaaagacgattgaatgcgaataaattataa

SEQ ID NO: 76 - DNA, 780 bp, Lactococcus lactis
  source 1..780: mol_type = genomic DNA; strain = CCOS 949
atgtcaagtattgaaaatatgattgcttggatgcaagcacgaaaaggtaaagttacctattcaatgacttcacgaatgggtccaaaaagttatgattgtagttcatcagttttctttgcaatgattgctggaggttttctgtcagaaggttcaatgggtaatactgaaaccttgtttggaatgtcaggaacaaagctgaaagaaatcagtcgaggagaagtccagcgtggcgatatcttcatttcaggtactccaggaggttcagctggttcggacggacacacgggtattttcttaagtaacggttcattcattcactgttcttacactcacaatggaattgcggttgatacgaatgatgcatatatgagtactcgcttaccacaccacttttatcgaatcgttggttctggttcaggaaagactgacagtaaacctcaaatgattacattgaatgttgacggtcaatttggtaatgcgacagctaaacgattgcaagaatactttgatacggctggtaaagacggagtaatcagtcaccagtataaacaaacctttaaccaaaatatttatgctgcacagtttgattcatcactgacaggctcaaacgtggtaaaagcattgcaaagattcctaggaattggccaagacggattatttggtcaaggaactatcaaagctttacagaagcatcttggaacaacgcaagacggaactatcagcccagtatctgattctgtgagagaattacaacggagattaaatgcgaataaattataa

SEQ ID NO: 77 - DNA, 1212 bp, Lactococcus lactis
  source 1..1212: mol_type = genomic DNA; strain = CCOS 949
atgactaagcgcttaaagaaaaaagcaaaaccagttgtttttctagctcttgttcttggttgtgcattaattttatcaggagttcttggtttggcgggacatttaaacgaacaagaatcgtctaaaacaaaaaagaaaagtcaattgtcagaaactaaaaggagttcaacttccccaaaaaatatggataaaaatattgagcagatgactttggaacaaaaagttggtcagttatttatgacaggaatgccagcaaataactataatcaggcaacttttgatgcgattgaaaagtatcaagtaggtagtattattttgactggtcgctctaatctcagtgtcccagaaatgaaagatattactgacaaactacaaggcttagaaccggctaatcgcaaactcttgatatcctgtgaccaagagggaggaaatgttcaagttctccaaggtcagggtttttcacagattcctgatggattaactcaaggaagttggacagctgataaattacaaaaggaatcacaaacttggggaagtgaactttacgcagctggagtgaattttgacttagcaccagttgctgaccaagttttatctgccgactttgctccacaaaatgctccaattggctattggagtcgccaatatgcttatgataaagcaagtatcataagccatgctcaagcatttacggaaggcatgaaagcagccaaggttttgacaactgctaaacattttccagggcttggagcggtgacaggcaatacagatatcagtgcaggtgttagagatgaccaaacgaattccaatagcgaatctgttcaaattttcaaagatttaatctcttctgggacaccttcgattatgaccgcaaccgctatttatgataaaattgaccctaatcttcccggtgctttttcatccaaaatggttgatggtttactacggaaacaattaggctttgatggtttggttattacagatgatttatcaaatgctgtccaagtacaatcatggacgcctggtcaaagggcagttttagcattgtcagcaggaaatgaccttgttttagcaaatgagcctacacaaattccagaaatgatctcagaagttttgcaaaaggtaaaagcagacccagactttgctaaaaaaatcagtcaatcagccacaagagtgattaaagtaaaagaagaaatgaaacttgtagaatag

SEQ ID NO: 78 - DNA, 939 bp, Lactococcus lactis
  source 1..939: mol_type = genomic DNA; strain = CCOS 949
atgattgcccaagggattttagaatcaagtggtggacaaagtgccttagcaagcaattataataatatttttggagttaaatacacttctggtacacctgtttatctaccaacacaagagtatttgaatggaacaatgacaaatgttgttgaacccttccaagcttatagctcagtttatgacgcatgtgttgcccaagctaaaatgttacgtgcttcatcatattattctggggcttggcgtgaaaatacaagttcttacttagatgcgacagcttggcttgaaggacgttatgccacggatccaacttatgcttctaaattgaatagcgtgatttctgaacttggtttaagtgtttatgaccaaggaggagaaatatcaggaggaactgctgttacaactagttcatcagcctcaacaaattcagctggcacatacaaagtacaagagggtgattcattatcagcaatcgctgctcaatatggtacaactgttgatgcacttgtgtcagcaaatagtttagaaaatgcgaacgatattcatgtaggagaagttttgcaagttgctggtgctagcacaactacaacaagtaccaatacaacttccaatgtatcgtcaagttctacttataccgtcaaatcaggagatagtttatattcgattgcggaacaatatggaatgactgtttcatcactgatgtcagccaatggaatttatgatgttaattcaatgcttcaagtaggacaagtattgcaagtaactgtaagtactagtgcaacaacttcaaacacaacgacttcaaacagttatacaattcaaaatggtgacagcatttattcaattgccacagcaaatggtatgacagctgaccaattagcagccctcaatggatttggaattaatgacatgattcatccaggacaaacaattagaatctaa

SEQ ID NO: 79 - DNA, 1041 bp, Lactococcus lactis
  source 1..1041: mol_type = genomic DNA; strain = CCOS 949
atggaaaaagcaattaagcggaaggttatattagttagccttccttttttgttgcttttattgccaatattagctttcttttcattatttgttggttcaaattcttcttcagatacagatattaatactaatacaccacagcaacaaactgccaaagtgatttgggatagagttctaaaagaaggtggaacgaaagaaggggcggctgctttacttggtaataatcaagcagaaagtgaacttcaaccttcaattattcaatctaatgcaacttataatgaagcaaaagcaatggataccactttaggcggttatgctttcggcttggctcaatgggatagtggcaggcgagtaaacttgctaaactatgcgaaaagtcagaaaaaatcttggacagatactaatcttcaagttgagttcatgtttgagcaagacggtacagattcaacgttacttaaacaattagtcaaaggaactaatgttaagcaaacgactgaagatattatgcgaaagtgggaacgtgcaggggcggttgatagccttccaaaacgtcaaggttttgcggaatattggtacacgttcatgacgactggtggtgatagtggaactggtggtggttcaggaattactccagatataccttctggttggactttagataaaccaattaatacaagtggttatcttgcgacaagttatgagtataaacaatgtacatggttcacatggaatcgagccaaagatttcggtatcacttttggaatgtatatgggaaatggtgctgattggcaacatcaagcaggatatactgtaacgacaactccgacactccatagtgcggttagctttagcggtggtcaaacagtaggcggtcaatggaacgcagaccctgtttacggtcacgtggcttttgtggaaggaatacactctgacggttcagtcttaatttcacagtcaggaactggttttagtgcggtttatactttccaagtgctgacaaaagcacaagccagtcaattacattacgtgataggaaaataa

SEQ ID NO: 80 - DNA, 2154 bp, Lactococcus lactis
  source 1..2154: mol_type = genomic DNA; strain = CCOS 949
atgaagataaacaaaattgatacccgtcatggtactgctaatcaatccactttttcacatgggaattgccttccttacactggttttccttggggaatgaattatttttccccttcaaccggtgctgctcgtggagcttggtggtttcatccagaagaccgtacctttgaaggctaccgaatcactcaccaacccagtccttggatgggagatttctcacatttgacaatgacgcctgttgcaggtcctttgcctgaaacttccttgtggcacacagtgagttcataccgtccagaagaatcaacttttaatccaacgcaactcaaaattactcaattacgttatcagattacatctcagttaattccatcaatgtatggtggaattctatcaatgaattatcaaaatttaagccttaaagataatggcttgatgcttcatctccccggttcttatgaacttcaacaacttgacgattacagcttagagctttcaattattaattttgcgggttgtgaagatgaaaattttactttctatctcaaatttactgctgaccaaccttttgttttaagtggaaatttatctgatgaagattcttctgttcgcttagatttcaaagcagccaagcaggttcaaatacattttgctacctcttttatttccaaggaacaagccgctcttaatctggagcgtgaacaagataattcagcagaaacatatttggaaaatgctgaatcagcttggaataaccttttttcacgtatcgaaattgagcatcataatcaacaagaagtttccactttctatcacaatctttatcgctcatttctctttccacaaactttttatgaatttgaccaagagcatcaaaaaatccattacgatacaagctctaaaactgtcaagaaaggcccgctctatacaaacaatggcttctgggatactttccgaacagtctatccgctttatagtctaatagccgttgacgagtacggtgatatgctcgaaggtttcctaaattcttatcgtgcgactggttttcttccaaaatggctttctcctgatgaacgtggtctaatgcccggtaccttgatagacgctgtcattgctgatgctgcaagcaaaaacattcgtcctgatttgatgcccgagtttttggaagcaatgaaaaaaggagccacaagtcaatctgaaaactctaattacggccgtcgtggcacaaaagattatctaaaacttggttatgttccgctcactcaccatgaatcagttaatcacactttagattacgcattttctgattattgtatttctcaagtggcgaaacaaaccggtgacaaagaaatttcagacttctatgcccatcaagcaaaagactatcaaaatatttttgattctgaaactggttttatgcgagcaaaagatgctgacggaaatttcagagctgactttttagatattcgttggggtcgtgattatgcagaaggttctgcttggcagacttcttggtcagttcttcatgattttgctggattgattaaactacatggctcaagagaaaattttgaaaacaaactcatcgagctttgcaaccaacgaccaaatttcaatgttgaaggatatggttttgaaattcacgaaatgagtgaaatggctgctatcgaatttggacaagtggctatctcaaatcaaccaagtttccattacccatacctttttagctacattggtaaaccttggatggctacgcctttaatcaaaaatttactgacagaaacatttaatgatagtccgaagggctatcctggtgacgaagataatggaacaatggcagcttggtatattttttcaagtttaggattctatccagtcacagctgcttctaatcaatatgtattaggaattccactctgggacaaagctagaatcaacctttcctctggtcagcaacttactattcttgcagaaccaaatgctccgcaacaagttttcgtcaaccaaatcacatttactgacaaaaaagtcaatgatacttttatcaagcacgaagaattgataaaaggcggaaccctaaaatttgatttaggaatcgtaccaaatccacttaaatatacgaacgaacaacttccttattcactgacagaaaattaa

SEQ ID NO: 81 - DNA, 1155 bp, Lactococcus lactis
  source 1..1155: mol_type = genomic DNA; strain = CCOS 949
ttggtcaaaataagttttgatgttaagaaatttgaaggtcaaacaatcaaaaaaatcggtttaagcataaaatctgatcaagcaatggattttaaagccattaatctaggagaaatgactttgaccaatggtcaaaaagtcgctccaatcgcactttcggatgcaaaagtaactgatgaagcttttgaagaagaaggaacagtaggcggttttagactttcttggaaatcagatgcaaataaaaataatttttctacttatgaaatctatcagttgaatgatgatggaagtaaagaatttttaggagcaagcaatatcaatgccttctttgttaatgccttaaaacgcggcaaaaatatcaattcaacaaaatttgaaattgtcccaattaataaggctggagaatctggacattcagttacgacctctgtgaaatggccagataattcattagctaaagcggcatttgtagcagataaaaccctagttacaattggtgaaaaagtaactttaatgaatcaatctaatctagcttccgtcaaatataaatgggatattgatggtgcaagtcccgctacttctacagaaaagaatcctcaagtaagttttgataaagcagggagttattccgtcaaattaacggtcatcaatgaaaaaggacaagaagattcagtcactcaaactgaactgattactgtaattgatcaaccagtagaattaacaaattttgcattaaatcaatccgttcaagtagacagtttcactaatgaatctgaatcaggaccaaaagcagttgatggaaaattaaataccaaatggtgtgccgttggcccaggtaaacacaatattacaattgacattggaaaatcagaaaaaatcaatcaagtcctgattgaccatgcgcaaaaaggaggagaatcgcctgacatgaatacttctgattacaccattgagatttcaaaggataatcaaaattggacagaagttgtcaacgttaagaaaaataaattgggagaaaccaaagattctttcaaacagacagaagcgcgttacgttagaatcacagccactaaaccaacacaaggagcagataccgctgttcgtttatatgaaatacaagtattaggacaaaaaaaagctgacaaagggctgtaa

SEQ ID NO: 82 - protein, 57 aa, Lactococcus lactis
  source 1..57: note = Strain CCOS 949
MSTKDFNLDLVSVSKKDSGASPRITSISLCTPGCKTGALMGCNMKTATCHCSIHVSK

SEQ ID NO: 83 - DNA, 1191 bp, Lactococcus lactis
  source 1..1191: mol_type = genomic DNA; strain = CCOS 949
tagtacttgtacactcccccagtcatcggtctttaccttaggaagcgccctccttgcggttaggcaacctacttcgggtactcccaactcccgtggtgtgacgggcggtgtgtacaaggcccgggaacgtattcaccgcggcgtgctgatccgcgattactagcgattccgacttcatgtaggcgagttgcagcctacaatccgaactgagaatggttttaagagattagctaaacatcactgtctcgcgactcgttgtaccatccattgtagcacgtgtgtagcccaggtcataaggggcatgatgatttgacgtcatccccaccttcctccggtttatcaccggcagtctcgttagagtgcccaacttaatgatggcaactaacaataggggttgcgctcgttgcgggacttaacccaacatctcacgacacgagctgacgacaaccatgcaccacctgtatcccgtgtcccgaaggaacttcctatctctaggaatagcacgagtatgtcaagacctggtaaggttcttcgcgttgcttcgaattaaaccacatgctccaccgcttgtgcgggcccccgtcaattcctttgagtttcaaccttgcggtcgtactccccaggcggagtgcttattgcgttagctgcgatacagagaacttatagctccctacatctagcactcatcgtttacggcgtggactaccagggtatctaatcctgtttgctccccacgctttcgagcctcagtgtcagttacaggccagagagccgctttcgccaccggtgttcctccatatatctacgcatttcaccgctacacatggaattccactctcctctcctgcactcaagtctaccagtttccaatgcatacaatggttgagccactgccttttacaccagacttaataaaccacctgcgctcgctttacgcccaataaatccggacaacgctcgggacctacgtattaccgcggctgctggcacgtagttagccgtccctttctgggtagttaccgtcacttgatgagctttccactctcaccaacgttcttctctaccaacagagttttacgatccgaaaaccttcttcactcacgcggcgttgctcggtcagactttcgtccattgccgaagattccctactgctgcctcccgtaagaatttgggccgtgtctcagtcccaat

SEQ ID NO: 84 - DNA, 1710 bp, Lactobacillus jensenii
  source 1..1710: mol_type = genomic DNA; strain = CCOS 962
atgactaaacaaactttgcaagctgaagctaatggtcgtaatggtaagattaagtttgatattaatattgataataacgaaataaaagatataaaggttaccaaatctagtgaaacaccagcaatatttaatcaggtttttgataaattaaaaaattccattatagaggagcaatcatttgatgtagacgctgtatcgggcgctacaattatgacaagtgctttacttgattctggaaaaaaggctttgaatcaggcaggagttacaccagtagctaaagagagcgataaaacacatcgggaagtaaacctagatgtagatgttgccgtcattggctcaggtgccgcaggattgattgcagcttgccgagctctttcaatgggaaaaaatgtagtagttcttgaaaagaatggttatttaggtggagctacaatattaaatggttcaaatgtagtagctacaggttctaacttggctcagcaattatttggtactgaggcacaagaagatagtagtaaacggctttttgctgatattactcgtgaatgccgaggtacaaattatcctgaattatcaaaagttttggttgaaaatattggtaaagctgttgattttattaaagagtttgcgggtttgacttatcaaaaagcagaaacacaaacaattgagcattcagttaatcggcaagtggaaatgccaagtgaaagctcatatgaattaattaaaaagatagcagctgcctttgaagaaaagggtggcaagattttacttgatgctcgtgttgaaaaaatcaattctgaaaatggtgtacctattagtctagttgctgaaggaaagcaccaaactacaaacgttaagttcaaatcacttattttagctgccggtggttggggtgccaaagactttaaagaaaagcgaacttcgattccttattatggtccaatgacttcaactggagattatttcttctttaacaagggattaaatttggctagtcgtaatttagattggtataaagtttatccacatgggttagaagttgaacctggaattgctaaacttacaacttattcaacaaaagaagcaagtgatatgggggcaatttttattaatcgggctggtaatagaattgtaaatgaatcagatccatacactcattttagagatgcaattgctgctcaaaaagatcagatagcttttgttcttatggatcaaagaatttggaacagattttatgaattaatgcttaaatatggttttactgctgatgaaattagtcattattttgcacttgatggcaaacaaagcccaattttagtaaaaggcactcttgaaaccgtagctaataaagctggtattaattttgaaaatttacagcatactttatctaattatcaaaattatgccaaaaatggcaaagatcctgagttcggacgtgaagcaaaatttatgcatgaatattcaggtgatacttactacgttattgaacaaaaacttcgcttctgtacaactttaggtgggtatgaaactaatagccaaatgcaattattaaataatgatatgaagccagtggctaattattatgcagctggtgaagtaattggtggtgccaatggtcatgactctatgccaagtatgatgaattcatggagttatgcttcaggctttttagctggaactaatgccagcgacaattgtaataatcgataa

SEQ ID NO: 85 - DNA, 2154 bp, Lactobacillus jensenii
  source 1..2154: mol_type = genomic DNA; strain = CCOS 962
atgaaaactaaaaaaatcactggcgttgctaccgcagctttactagcctccgttgctgtgccagttaccaacaatttagctaatattcagacttcctatactgctaaagcagtcactggtcaacagcaagcatttttaaatacagctatacctaatgctgaagcagcttctgctaggtatggaacgtatacttccgtcatgcttgcacaatcaatacttgaatctggttggggcgcttcacttctcgccacacaggctaataacctatttggtatgaaaggttcatataatggacaaacttattatactaatacttcagaatgggcctctggaacaggttattacaatataaatgctggttttagaaaatatccttcttgggcagcatcttttgaagataatggttacaaattaagaacaggtactaccgataatccaagtcgttatagaatggcttggattgaaaatgctgcaaactatcaagtggctacacagggcttgaaagatgggggttatgctacttcaccaacttatcctcaatcgttaaatagagtaatttcttcttacggtcttaatcaatacgatccgtctgtagataccacaactaaaacaatgagagttttatcaaatggaacagtttattccggtccagccgattctagtgtagttagtgcaactggtaatattactgctggtcaagtggttactgttgataaaacagttacttataaaaatggactaagttatatgcatattagcaatggttggattaatggttcattgttaaccggaagttcaactcaagctaccacaacagaaaaagcaggaacaacaaccgatgcaggtaatgcagcaattaaggtagtatacacgagtgcaattgcagaatggaaaaatccaggtagtggagtagtaggctacttacaaaagggaacgacccaaacagtagttggaaaaatccaagtaaatggagcatggtggtataagctatcatcaggtaattgggtaccaggggaatatgtatatgtcacaggtgcatcaagaataccaacaattgataacaatgtagtaaatcaaaatacaaaagtaaaaataaaatatatatcaggctatagcatagcagtatggtcaaatccagcaataggaacaacaggccaatatttaaaagacggaacagaagtacaaacgattggatatactacagctaatggcaagaaatggtataaattagcgaataatacgtggataccagcagaatatactgaagtagtaagttcatcaacagtggtaacgaatattactaatgaaagtaatacagtagcgataaattatccgcactacagtatagctgtatggagtgaaccaggtagaaactcaacaggaaagtacttatcagatgggacaaaagtccaaacagtaggttataccacagtaaatggtaagaaatggtataagctagcagataatacgtggataccggcagagtatactaaagtagtaagttcatcaacagtagtaacgaatattactaatgaaagtaatacagtagcgataaattatccgcactacagcatagctgtatggggtgaaccaggtaaaaactcaacaggaaagtacttatcagatggaacaaaagtccaaacagtaggttataccacagtaaatggcaagaaatggtataagctagcagataatacgtggataccagcagaatatacgaaagcagtaagttcaacggatactactagcggaaataatacagtaacagtaaattatccaggctatagcatagtagtatggagtgaaccaggcagaaactcaacaggaaagtacatatcggatggaacaagtgtaaaatattacgcaaccgcaaattataatggtcaaacatggtataagataggtgaaaaccaatgggtaccaggtcaatatgtaaaagtaaatgcatcaaataatagtggaagcgtgacagtaaaatacatatccggatatggtatagcaatatggtcaaatccaggcagaaattcaacaggaaagtatctggagaatggcacaacagtaaaatattttgagacccaaaattacaatggccaaacatggtataaaataggtgaaaaccaatgggtaccagcacaatatgttagtgtaaattaa

SEQ ID NO: 86 - DNA, 2328 bp, Lactobacillus jensenii
  source 1..2328: mol_type = genomic DNA; strain = CCOS 962
atggcagagaataatcatcgttcagcaagtaatactgcacctaagaattctcgaatggccaacttgcatcatgatagtggtcaacccaagagacgcctctggctacaaattcttaagtggttttcaattggtattttgctgatactcgtttctggcgtcggtttatttgcttattacgccaaagacgctccatcaattagtcaagatcaactacaaagtggtggttcaagtagtttctatactagtgatggtaaattccttctttcattaggttcagaaaaaagaacttatgttaagaattctgatattcctcaaactttgaaagatgcagtagtatctgtagaagataaaagattttatcaagaaaatttagggattgacccaatcagaattgttggttcaatcgtctcaaatgccaagtcaagtggtattgcagctggtggttcaagtatcacccagcaattagttaagttaactgttttctctaccgctgcttcgcaaagaactctaaagagaaaggctcaagaagcttggctggcaattagagttgagcatgactacaccaaaaatcaaattttggaatactacattaataaggtttacatgaactatggtgtatatggcatgggaacagctgccgattactactacggcaagtcactaaaagatcttgacttagcacaaacggcattaattgctgggatgcctaatgccccagttgcttatgatccttatacttacccaaaggcagctaaatatcgtcgtgatattgttttaaatgcaatgtatgctaatggtaagatcagtaaggctcaattaaaggcagccaaggctgaatcaattacacaaggccttaaaacccaacaaaatagttcagaatcttcaattagaaggattgatgatccttatattaaggaagcaatttcagaagttaaatccaagggttatgatccttataatgataacttaaagattactttgaacatcgatcaagatgctcaaaacaaactttatgaacttgcaaatggttcaagtattccattttcatcaagtaaaatgcaagttggtgcaaccatcattaatccaagcaacggtcacgttgtagcaataattggtggacgtaatttaccatctgttcaattgggacttgaccgggcagtacaaactggccgctcaactggttcatctattaaaccagttttagattatgcacccgcaattgaatatttgaactggtcaacagcacactaccttgaagatactaaatatgtataccctggtacaagcatccagctttatgactgggataacaaatatatgggtaaaatgacgatgcgctatgcccttgaacaatcaagaaatgttccagctgttaaaaccttagctaaagtaggtatgaagaaagcttcactatttgctaaaaagatgaacatttcagttggttcaaatcaaggtctttcagtagccatcggtgcaaatgcttcttcccttcaaatggctggtgcatatgccgcctttgcaaatgaaggtgtttactacaagccacaatttgtatctcaaattgaaacagctgacggtgtggttcactcctactcagcaactggaacacgagttatgaagaaatcaactgcctacatgattactgatatgcttaaaggtgttctaactgaaggttctggtactaatgcgagaactggtctttatgaagctggtaaaactggtactgttaaatattctgatgatgaattagtaaaatatccttcatacgcaaacactccaaaagatgcatggttcgttggttatactaagaaatattcaattgggatttggaccggttatgacaatttaagcgatggtacaattagtggtcaaggccaatatgcttcccaatatctttataagtatatgatgaagtacttaatgagtgataaggaaaacagcaattggactaagccaagcaatgttgttagaaaacgaattgttaaaggttctgacccacttgaagttacttcctcaaagaaaaattcaacctctgagttgttcctacgtggtcacacaccagatggctcaaacgaggattctgatgaaagttcttcatcatcaaattcttcaagttcttctaaagataacgaagtagtaactaataagagcagcagttcttcaagttcttcatctagtgaaggacatgaagatggctcaacttctaactcaagtagtagtcaatcaagcagtggtggcagtcctaacaacaatcaaggaacaactactaataatcaaacgaataacaacaataatactggccaaaacaatggcggcggtaataattaa

SEQ ID NO: 87 - DNA, 6270 bp, Lactobacillus jensenii
  source 1..6270: mol_type = genomic DNA; strain = CCOS 962
atgttttcaaaaaataatatgaaaatgcgcatgcaaaaggttgctgataaagagcaacaacattttgctttgcgtaaattaaatgttgggttggtatccgttttaatgggtactacattatttttcgtgggacaaacatcaatagctaaggctgatacactagaatctactaagactagtgtcactgaaattagttctagctctaattctaatcaagaaaagttagaacaagctaaggctgtaagttctcaagagcaaactacaagtactgaagaaaatgtagattcagcttcatcaagtcaatcaaaaagtgaaaacaattctagctctagtgcgaagtcagaagtaagtactcaagatagaactgatagcaatgacaatgctactactaatcaagtagatactacgcaagtatctactacttcattgaaagctgttcaatcttctaatcaagctgctaacttattagctgaaagcaaagttgaagattcaacaacagctaatgttagtgatttcgattcatttaactcagctttgatgaatcaaaatattactactattaacattaatagtgactttacaagtggtaactatggtacaaatgttgaacaccgtagagttgttcctcgtactttagtaattaatggtaatggtcatacagttgaccttggttcaattggttacttcatgaatccaggtggtaatcaagcagctgattggacagtagaaaatggtactttctattctaagagtgctgttggtccatttgctcttactggtgatcacggtactgataaggcttggcaggcaaccaatggtgcagtacaccgtatgacttacaaggatatgacagtttatgctggtggtggtgcttatgctgcaaatgctgaaattgatttagaaggtactaacaatttaaacgctaccgcttcatatcaatctaagttctcaaatggtactgtttatactgaaactggtggtaacagatcaggtgttgaaggtcaatggattattattaaggataatgctaatgtgactgttaacactgatcacggtcaagctatttcaactgaatactctgatggaaatcacagaattgaagttggtgaaaatgccactttaaatggtaactatttatataccacttacggtactccttatggtggtggtaagatagctgttctttcaaacggtgcaaatggtggtactgtaatcttccataagggttctaagtcacactttactatgccaaatcaaaatgtaactggctttggtggtaatggttttagtaactatggtactggttctgttctttacttaggtgcagctaatactactattgaaaaaggtgcagaagttacaattgataaccctaaagaagctaataacgataacagtatcttcattcgttcagacggaacacaattaaatatctttggtaacttaattatcaatgatggtgcgcgtgactatactattcgtgccgataaccgtgtaagcatcaatgtgggtcaaagtattgcagatgatgattacagtaatgataatggtaaattaattattaatcgtaacggtgacttctacttgaataacgatggtggtgctctctcaatgtggggaccaattaccattaatgttaattcaggagctgctttagaagtttactcaactggaattaataatgtatctggttctggtgcaaatcctgctttaatgagagttggtggaagcagtaaattaaacgtttataaacgtggtactttcattttaagtgatagtagtaatggtaaagttagtctagttgatgattacaatgcaaatggtaacttcaacttcgacaatactgcaaaagttatttttgatatttcaaacaacaataaccaaaatagtagaatttttgctttaaatggtactgtatatgcaactcatgacaaggtaagagctatgctcacaccaagttctaatattgctgatatgggtacctttaagacggttcaatttaatactagtggtcaaggcggtgtgactggggatgcattacactctgagtatcaaaaccaagcctacaataacattgttgcgattagagatgcgattagaactggtcaattacgttaccttgaattctcaactcgttcagtaaataatgcaagtattactggtgaaggacatggctttactgataatggtgcacaaattaagggttcagtagttgatgaatcaactaacactggtgcggtagccgttgcctacgacaaagataataataaagttggttcaggtaatgttgatgaccaaggtaactttacaattaacttagataagccgcttcttaacaaagaagaagttaaagttagaattgaaacccctcgtggtgctagtggttttgtttcaactaccgctcctttaggtccaactgctaagagtccaattactgttgctaaagatgataatttagcaggtcaagatgctagccaatacatcaccaataaagatgaaattgcaaacattaagccaggtactactgactatgaaccaacctttaagagtgcagcttggaagagtgttgatactaataccaagaagggtattattactgtaacttatgcagataacactactactgacttagacgtagacttaaatgtagtagacaagattactgatgctgacaagttcactccagaaggtggaacaattaaagttgataatggtcataaattagatggtaatgatgcttatgaagcaatcaagaaccacgatgatttaccagcagataaagcaggctacaactgggttgtagatgaaaatcatccagccgttgatactactaagcctggtgatcaaactggctatgttcaagtaacttacaatgatggcagtaaatcagatttagttcctgttactgttcatgtaattgctgataatgaaaaatacactgctattggtaagactcaaaacctccaaaaaggccaaagtgcaaatcctcaagactttatcgctaataaagatggagaagttagccaagatggcaagacctatactaagttgcctgatggtactaaatatgaatgggtagatggaggaattgatacttctactattggtccaaaacaagctcaagttaaggtaacttatccagatggaaccactcaaattgttccagtaaaggctaacgtatattctgatgctaacttatcaactgttgttggtaaggatattaatgccggatacaaagaagacttgactaaccgtgcaattgatgcaattgataaggataagtcaactaaccttccaactgatccatcagcttatacttgggtaaatggcgcacctgatacatcaaagacaggtgatattccagcgactgtaaaggtaacgtatccagatggttcatacaatactgttgatgtaactgttcatgttactagtgatgctgaaaagtattcaccagttccagagaccatttctgttccaaagggtactgacttatcaggtcgagcaaaagatggtattgctaatgctgataaagatgctaatggtaaggaaaagttaccagatggtacaaaatatgaatgggacggcggagttccaaatacttcagtaactaataataagtatggtcatgtaaaggtaacgtatccagatggtagttctacagtagttgaagtacctttaaatattaccgataataataagtcagatgctgaaaagcataatccacaagctaatatccttcatgcaacattgaatcaagatcttagttcaaatgattgggctaaaaaaggcattttaaatgctgatgagcaagatggagcaacatttagatggagaaagggttatgttcctgatacttctaaagaaggccaagtttggggtattgttgtagtaagctatccagattcatcaattaatgaagttcgagttccagttatcgtccaaagtgatgcaagtaaatacggtattgaaactcaagaaatcaatgttcatgaaggaaccgatattagttcagatgaatgggctagaaaaggtattttgaatgccgatcaagctggaaaacagaatgaacaattaccagatggaactagctacacttgggcaaatggtaatgttccagatactacaaagccaaataagaagacaggttatgtaactgtaactttcccagataatagtagcagaactgttccagtaatcgttaatgtaattggtgatggtagaagtgatgctgaaaagtatcaacttaaggctcatgatatttggacttatataaacgatactcctgttgcagaaaaggcagtttcaaatcttgacgaactaaaagatgttagttcaattacttgggcaactacaccagatgtttctaaggttggtaacgttccagcgattgtagttgtaacttataaagatggtacttctaatgctgctcctattaacattgaggttaagggattagcaaatgattataagcctgaaggtacaactatttatgcaggtttaaatgaagatataactaacagagctgcagatggtattgctaataaagatcaaatgccagtagcaaataaaccagaaggtactgttcacacaacttattcatggaaggataatataattcctgatactactaaacctggtaccaagtatggaattgtagaagttaacttcccagatggttcaactaaggatgttccagttgaagttaaggtaactagtctagcatctgattaccaaaataagattgatactaagcaaattattgctaagtacaagggtaatatacctcaagcctctgacggtattgctaacaaagatcaagcaactaaagaaggagacaaagacttcccaagtttagctgatgtgttagctcctaatggtattcaatggaagaagaactttgaacccgatttgagtaagccaggcttaacaagtggtgaagcaattcttacctttaaggatggttcaactgccgaagttacaattccggtattagttcaaactgatgctgatcgcaatactccagaaactcagactattaagacacttcctggacaaacagtaaatcctgaagatggtgtaattaatttacataagcctggtgaaaataacccacaattgccagatggaacgaaggtaacttttgataatcaatcagatgttgatgactttactaagcatggaatgccaggttctgataagagcttcgatgctactgtaacttatccagatggaacaactgacaagattaagttgccggtacacattacagctgataatgaagttaatactccaattactcaaggtattattacacctaaggatagtgtgccagatgccaataaaggtattgctaacttaaagaaggctactaccaaagaaggtaaaacttatcctgcattgccagagaacaccacggttgaatgggttaaccctggtcaaatgaagactgagcttgaaaatgcaaagggtggtacaaccaagaattacgatgctgtagtaatttatccagataaatcaactgaaattgtttcaattccagtaactgttgctactgatgcagatacttataaagtagtaacacaaccaatcgatttgaaggatagaaacttacctgacaacgctgatgatggaattactaaccttcataaaccagcagactttaagactccacaattgccagatggtacacatgcagaatggcaagataaggatgctgctcaagaagtagttaagaacttgaagccaggtgaaactgtaaaacttccagctactgttgtcttcccagatggttccaagaagggtgaaggcattgatgtaagtgttcacttacatggtcaatctgatgattataatattgaaacacaaccagtaaatactgataaagatggtaatttacctgaaaatgctgactctggtattaagaacttaggaaagttacctgaaggaactcacgcaagctggggtgatggtgcccaagatattgccaagaatttgaagctaggtgaaactaaggatgtaccagcaacagttgtcttcccagatggctcaaagaaggaaattacaattccagttcatagagaaggtcaatctgatggttatgatgttgaaccacaactagtaaatactgataaaaatggacaactacctaatgcaaaggaaggtattaagaacttagccgatttaccagaaggaacaaatcaacctgggcagatagagctcaagataaaattaataagactaagcctggtacagatacaactgctcaagtag

SEQ ID NO: 88 - DNA, 645 bp, Lactobacillus jensenii
  source 1..645: mol_type = genomic DNA; strain = CCOS 962
atggcacgttcaggatctcgagtaaaaaaaacaaccctaactttagttaaagtctttggagtattacttatcttagtagtggcgtttgtttgctttcggtattaccgtagacaagcaatcaataatgaattaattagacaagaacaactagcaagagaacaggctgcagctaaggaattaaagataaaaacagatttcataaaaaaaatagggccaattgctcaaaaagctgatcaaggctttgctcttttgccaagtattacaattgcccaagcttgtttagaaagtaactatggccaaagtgaactatctcaaaagtataataatttatttggcgtaaaaagtaatgatcccaatacttcaaaaattttatctactaaagaatatgttaatggcaaatggattacagtaaaagctagtttccaaatttatgattcatatgaatcttcaattcaagctcatgctagattatttcaaaatggtactacttggaacaaagatcaatatcaacatgttttagcagcaaaagattataaaacccaggctaaagcactagttacagatggttatgcaacagatcctgattatgcttccaagttaattaacttaattgaacaatttaatttaaataaatatgacaattaa

SEQ ID NO: 89 - protein, 558 aa, Lactobacillus gasseri
  source 1..558: note = Strain CCOS 960
MLAQAAVESAWGQSGLAQSPNNNLFGIKGSYNGQSVNMNTGEYGSNGYYTTNAGFRKYPSYTESFEDNGSLLRNQMGNYYSGTWVENSKNYAQATQNGLQGKYATAPNYAQTLNSVIAANGFDKYDPVTQVVNENRTVAQTTPIMSAPVDASVGTQVGTARTGQNVNVTKYITYNNGVKRAYIGTGWINALAFSPITTNTTTKQNTAANTNNQASQAVKTPVAQTQQVKSQAPAAPVKAATVKVKSAAEVKTPVQTTTLNVKTENKAAQPVKQLAVSLAVAPKKVEVKKNVVKAEPAKTTPVKTETTKTENKEVKTATPVVKTTDTVKKVETPAVKPVESVKKESTPVVKTTPVTVTKEAAKTTEAPVVKPKKVEVKEIKTTPVTTSAKTVVKPVQSYQSAVTTAANNNYGTQWISTNTAPKAASTVLIKVTKTVDVLSAPDGQKLDQQVEAGSEFVVVASKYYNGNLYYEISNGKWIMAKYTTQEAQITAKSGVLTINSKPDYGVPVWRVPGQDQIAGKFLKDGSSWRYFRVANVQGQTWYDLGGNQWVSAKSVLVR

SEQ ID NO: 90 - protein, 208 aa, Lactobacillus gasseri
  source 1..208: note = Strain CCOS 960
MPRRRRQKNIQYVIARAFAICFTLVVILSGFLYWRHEVAVNEQLRQAQLEKERALQSKERFIKVVAPIAQRADKPYGLFPSVTIAQACLESNFGQSELSKKYYNLFGVKGTDPNTSRELTTSEFVNDHWETVTGRFQIYNSYEESIQAHTRLFVNGTSWNKDQYQHVLAAKDYASQAQALETDGYATDPGYAKKLIDLIKEFNLTQYD

SEQ ID NO: 91 - protein, 513 aa, Lactobacillus gasseri
  source 1..513: note = Strain CCOS 960
MVITLLTLDLEKYPSYTESFEDNGSLLRNQMGNYYSGTWVENSKNYAQATQNGLQGKYATAPNYAQTLNSVIAANGFDKYDPVTQVVNENRTVAQTTPIMSAPVDASVGTQVGTARTGQNVNVTKYITYNNGVKRAYIGTGWINALAFSPITTNTTTKQNTAANTNNQASQAVKTPVAQTQQVKSQAPAAPVKAATVKVKSAAEVKTPVQTTTLNVKTENKAAQPVKQLAVSLAVAPKKVEVKKNVVKAEPAKTTPVKTETTKTENKEVKTATPVVKTTDTVKKVETPAVKPVESVKKESTPVVKTTPVTVTKEAAKTTEAPVVKPKKVEVKEIKTTPVTTSAKTVVKPVQSYQSAVTTAANNNYGTQWISTNTAPKAASTVLIKVTKTVDVLSAPDGQKLDQQVEAGSEFVVVASKYYNGNLYYEISNGKWIMAKYTTQEAQITAKSGVLTINSKPDYGVPVWRVPGQDQIAGKFLKDGSSWRYFRVANVQGQTWYDLGGNQWVSAKSVLVR

SEQ ID NO: 92 - protein, 104 aa, Lactobacillus gasseri
  source 1..104: note = Strain CCOS 960
MKKRTFTGIATAALITTAGISVTNNLKPENPLKTGEGTVQAATYQQEFLNKAIPAATTASSKYGTYTSVMLAQAAVESAWVNQVWHNRLIITYSVLRALTMGNQ

SEQ ID NO: 93 - protein, 772 aa, Lactobacillus plantarum
  source 1..772: note = CCOS 893
MLSTALLPMLSGKADTASTNQKPAAATKGNSAASAASQQVTLSAGSQTETTAAGATDQSVASDGAKTDDQAESTSTTTATTSATSRVTVRAASQVAKADSTELQSQSSASEAAKDNAAASATADSTTSAVDQLDKTAKASAATSQASHSTTNETAKASAAASQDSHVTTDQSSVTVTSEVAKSAASSAAPKQATEQAVAAKISPKIETAVAADAVQSSAMMARSTRAMTSQEIFLSQIKAGAISGWNKYQVLPSVTAAQAILESGWGQSQLATQGNNLFGIKGSYHGQSIYFPTQEWNGSQYITIQDAFRKYPNWSASVEDHGAFLVVNPRYSNLIGVTDYRRVASLLQQDGYATAPTYASSLISIIEYNKLHEWDQEALSGQASGGNDNNQVQPDQDVTPTSGTHKFTKTTTIHNAPDATSAVVGTYNAGETVNYNGKLTVGNATWLRYQSYSGVSRYVMISQTTTNDNNNQATVTPASGSYKFTAKTNIRSAASKTAQVVGTYNAGETVYYNGKITTGGTTWLRYLSYSGAQHYVAMSGDEVGSVAKPDVVATSGSYRFTKTTAIKSSPATSATTVGSYNAGDTVYYNGKVTTNGQTWLRYMSYSGAQHYVQISGESTSTNVDKPQVTPQSGSYRFTQTTAIKNTPAGNAPSVGTYSAGDTVYYNAKVTANGQTWLRYLSYSGAQHYVAISGNAATGNTTSKPVTNSQGAFRFVTTTNIRTAPSTRASVVGEYNPGETVYYNGTVQAEGYTWLRYLSRSGATHYVAKLEG

SEQ ID NO: 94 - protein, 212 aa, Lactobacillus plantarum
  source 1..212: note = Strain CCOS 893
MAGKRRKSRRKQRTKTQLFVKKGRLQWVNILLVLAAMVGMVWYIQHNWAVKSRVTATAPTTTHAAFIKKLVPAAQQLDQQYHVLASITLSQAILESDWGQSTNATENNNLFGVKSTSGRLMTTQEYYDGAYHTVKRRFAVYDSWHASLVDHAKKLAYGTTWDSQHYAAVIKATDYQTAAQALQTAGYATDPSYAQKLINIIQKYDLQRYDRK

SEQ ID NO: 95 - protein, 421 aa, Lactobacillus plantarum
  source 1..421: note = Strain CCOS 893
MSSVLGITLAKPVQGKADQTSSPSTTKVKAATSGATSEVSSISTITANSATNVSSATHQFSTATTTSQQASSATSNISTTTASKQISKSTTGKLNSNETATTSNSAATSVDSTAPYIPAKSNVSFTDNIKSTIANTPTTVAAPAIPSHSTQFIDDSAPITSPTPVTTNSIHVRPFTVHSSFSFKPGQFLRFTLPNVALLRDDVEQGVPNYVKNFFIAIKPGAMIGWSQYHILPSISGAQALLESGWGKSTLSVQGHNLFGIKGSYHGHSIEMPTTEYLNGEDVTIEATFRKYPDWATSIVDHGAFLNQNSRYRNLLGVKNYSTVAWDLQNDGYATAPNYATSLINAIQDYDLQEWDQEAFTGNTGSTTTTTGNHRSGTYTFIQNSNIRTEPSLSAPIIGVYYPGDVVNYTGQIKAEGYTWL

SEQ ID NO: 96 - protein, 258 aa, Lactobacillus plantarum
  source 1..258: note = CCOS 893
MVVPVDIWNQRGDASDQVTLKWPDNRKPKAAFTVNRTLAAPGNTIKFTNASSKNATSYKWEFDGATKTTSTAKNPTVTYRKAGTYNVTLTAKNKDGQRHVTMKKLITITPKAQGALTLLSKNAKTSASGYTNSSEAPKMAVDGKLDTKWCATGKAPHTLRLDLGRQTTVSAVKLAHAKAGGEGADMNTRAWTIQVSTDGKRYTDVARTYNNTQATSLNTFAATSARYVRLVVDKPTQVADTAVRIYEMDVLGLTQTLK

SEQ ID NO: 97 - protein, 94 aa, Lactobacillus plantarum
  source 1..94: note = CCOS 893
MRKDTASTIKLYTTDLDVTADTQVSLTAKASGKSTAKLVVTLKDGRTKTIAGDRTLSKHWTTVSYDVSQLTKKQSKALASKSVPPRLMLATAFN

SEQ ID NO: 98 - protein, 408 aa, Lactobacillus crispatus
  source 1..408: note = CCOS 961
MKKRLLTSFAAAAMLTSVAVPAVNTTMMNQASSQRVSAATADQTAFLNKAAKQAVKAAKKYGTLPSVMIAQAITESGWGKSGLAVNANNLFGMKADDSWTGETYTAKTREEDKNGKSYYITAKFRKYPSFEQSFEDNGSKLRNGVSWDPLRYKGTWIENASTYAAATKALTGTYATDSKYDKALNSHITSSNLTKYDPVTVNTTRTYTAGKDSSTYNWPTAPSVASVIGSVKAGEKVVVTKTITFHDGSSRMYIDGRGWVNGSVLDKSSSATKEPVTQAPKNVPAVSKNLMHNAYVYDQNGKKLKGKMYKTSDENGGKWINTYGTKTIKGKTYYRVGENEYIAAGNIDGSLRFLKKNAYVYNQYGNRDNNLKHKKNSQIATYGSAITINGKKYYKVGIRQYVKKSNFM

SEQ ID NO: 99 - protein, 215 aa, Lactobacillus crispatus
  source 1..215: note = CCOS 961
MARKRKSRVPKSVKTVVRVFVILFILMVAFVGFRYYRRYAIQSEQIQQAQLQKEQEAAKLLKQKKDFIKKIGPIAREVDKSYDLLPSITIAQACLESNYGQSDLSQKYNNLFGVKGSNPNTSAVMTTKEYVKNKWVTVKARFQIYDSYEASIRAHARLFQNGTTWNHDQYKHVLASKDYKTQAKALVTDGYATDPDYADKLINLIEQFDLEKYDK

SEQ ID NO: 100 - protein, 430 aa, Bifidobacterium bifidum
  source 1..430: note = CCOS 571
MRMQQIKHGGRRPRPSSAPTIIAAMLCVVALVAAGTAWWMLRPQQKDTLEHLAQPQSSSSTLNRDKAPTPKPQKSQERQKAAGDSPAAKASRAIATMSLDERAGQLIMAPMFAGGNPADLSALISTRHVGSVVLIGNWNNGTAAAKTAADALQSYAPSGNQLIVSTDQEGGQVQHLKGSGFDTMPSAVAQGQMSADTLRSSAKTWGGQLKQAGINVDLAPVLGTVQVKRSSNAPIGALNRDFGLDSNGNAQHGIAFVEGMRDAGVGATVKHYPGLGAVTGNTDFTTEGILDTTTTLDGEEIGAFNTTIKQAKPAMVMMSLATYQRIDSSAPAAFSSKIIDGTLRGSVGYDGVVISDSLSAAAVSGIATKDLGVRLVDAGGDLACIGDTSYVTPILDGIIARAQSDPAFAKKVTASATRVMTLKYQMGLAK

SEQ ID NO: 101 - protein, 1935 aa, Bifidobacterium bifidum
  source 1..1935: note = Strain CCOS 571
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTAGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDIDNALKALTDAYAGLTEKTPEPAPVSKSELDKKIKAIEAEKLDGSKYTAESWKAFETALAHAKAVIASDSATQQDVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGSALSKTGASVAVIASVAAAMLAVGAGVMALRRKRS

SEQ ID NO: 102 - protein, 154 aa, Bifidobacterium bifidum
  source 1..154: note = Strain CCOS 571
MHSRPMFPIPETQVGDGWVGDVDYTKQATKQDSNFLVDWVRVYQSEGQPVTRFDDLDGAESGAYRSAPASRTEGLTAVSNGDAAWRNKNNFYYGGQPRYETSRSHAVPLMQGRFALRLRGPQTLCRIRTNAFTRSERPVAGKVVPPNRRNRAAP

SEQ ID NO: 103 - protein, 185 aa, Bifidobacterium bifidum
  source 1..185: note = CCOS 571
MISASREIHSINLYRNGYENALNTFKRIKVEVSSNEDFSDANVLFGTADVEETAATKLAAQTINLTTPVTARYVRIWQKGHCIQNTNSSWKGYGNGVGLREIEVIAKLKDGETLPDAQETRNIALGKLPYVYGLDPTNIAAISDGKQDDNYAVHNSTGERWLQFEYKNRYRIHEGSSRAPIRWSA

SEQ ID NO: 104 - protein, 108 aa, Bifidobacterium bifidum
  source 1..108: note = Strain CCOS 571
MRQLFRRITTISATCALAVSVAAAVAVAAEPTTGNILTGKLPTTNSTHLIGDGDTGFGPTDSNITKIIAGEENGSAENNGYASWDDVYLQYDFGEPRDTLHQPVPQWV

SEQ ID NO: 105 - protein, 1931 aa, Bifidobacterium bifidum
  source 1..1931: note = Strain CCOS 571
MRKRVVSVALAVALAVAPLGVVSTASAAPLSASDLQTLALRSAASSNDANANDVATVADDAAVNGWTIDRNTAKGGEILAAGTGDYAGWTHFKSTSANGNATSSSGYPAVAISGKTIDLTRAGEFSIKVKSPQAGSANRFGFYLGYKDPGNALFLGYDKGGWFWQKYVGGNGDWYNGTRVAAPAANAEITVNVSWTAAKVATLTIDGQKAFDVDYSSMTALTDKLAMKAGSYSGTSEVTDVYFKNFTVGEVAKHNVTGKVVDASGAAIAGAEVVTGKNSATTAADGTFTLTGLAAGDYTLTVSAEGYDDATKTVTVADGNASVGNITLNKSAEVATETLSTAAMDVRVKKNFPSVYDYTMKKLDGKIMYGQPKDVRVITINGTDVTLKDSDVTFKKVSATEAQYTLNVKSGDKINAVVTVQIKVVDNTLKLNVTKIVNKADDAKTEAEENPVQTIAFPNQSLISVRSGQDGAQFTGARMSSDTARPGDTNFDITADTTVSNANDYTYGFVSGNGLSAGLWSNSEHDGTTVGNTVAGGARNTRVLTSTQKVGKATSFGLGTAPWYYHRVVTDTKKNTYTVEETDMPKMAVAIAGDENEDGTVNWEDGAIAYRDIMNNPYKSEEVPELVAWRIAMNFGSQAQNPFLTTLDNVKKVALNTDGLGQSVLLKGYGNEGHDSGHPDYGDINTRAGGAADMNTLMEKGTKYGARFGVHVNASEMYPEAKAFSEDMVRRNSSGGLSYGWNWLDQGIGIDGIYDLASGMRKSRFADLKSKVGDNMDFIYLDVWGNNTSGAEDSWETRKMSQMINQNGWRMTTEWGAGNEYDATFQHWAADLTYGGSAMKGENSQVMRFLRNHQKDSWVGDYPSYGQAANAPLLGGYSMKDFEGWQGRNDYAAYIRNLYTHDVSTKFIQHFKVVRWVNSPLDATSVKDASVNNGNEQITLKDDHGNVVVLSRGSNDTNNTAYRNRTITLNGITVASGAVSPGNSNTVKGTESYLLPWLWDVNTGKLVKSSDEKLYHWNTQGGTTEWTLPKDWQNLASVKVYQLTDQGKTNEKTVAVSGGKISLTAEAETPYVVTKGSEKQISVKWSEGMHVVDAGFNGGQNTLKDNWAVSGTGKAEVEGTNNAMLRLTGDVKVSQKLTDLTAGKRYAIYVGVDNRTNSPAKITVTNGTKVLATNETGKSIAKNYIKAYGHNTYSNTEGGSSYFQNMYVWFVAPESGDVKVTLSHSGACDNTDHVYFDDVRVLENGYKGLTLNADGTLKTLTNDFEDNAQGIWPFVVSGSEGVEDNRIHLSELHDPFTQAGWDVKKMDDVLDGKWSVKANGLIQKGTLIYQTIPQNVKLEPGETYKVSFKYQSGSDDIYAIATGDGEYNASTVKLTNLKKALGEDGTAEFEITGSITGDSWFGIYSTSTAPDLQNTSDSAANFGGYKDFVLDDLKVEHVASAEHTKADAEAKLKEVKDTYDGKSGDYSAEVWTTYVNTVAEIEALIAKDKPDYTTAYNKAVALAEYMKNAPGDDSNDAYDVATDAYTVEAGSQQALSGGNEGPASLAQDGNAGTHWHTSWSANAVSAGTAWYQFNLNEPTTIDGLRYMARSGGANANGKIKKYKITLTLSDGTTKDVVTNGTFTTTSGVWQKVKFDAVKNVTKVRITALETAGQSAGEVNTYASAAELRVTTVRDVPSTEVKVNKCDLQNLYDDASALTEATYTADTWKVLVAKRDAAKKVLDDENATAHDVALAYQNLKDAIAALEERVDTSKLAGLVADAEKLKESAYTKDSWAAFKKALDAAKAVLNNANATKADVDAAYNALNAAMKALKPASSKPTPNPETTDKSKLQATIDQAKALDLSGYTKKSAQAVRDALAKAQSVLADDNATQADIDAAQKALADAIAALEKADANGNAISKTGANVAVIGMAGMMLVAAAGAVFIARKRAE

SEQ ID NO: 106 - protein, 867 aa, Bifidobacterium bifidum
  source 1..867: note = Strain CCOS 571
MVSPHHLLKIATALSAVALTASVAVTPAYALQDIAIEDAVAQSGPVTADNGVVVQSDDQSDDQTGDQQSQDGMPDNPNAKLPDTVSDEISDDATVVSEDLAVTPEGEVKNIETGETVTDATLVGTQDQQPDPLAKTNGESFIPVSAEDVKNAVADANVQLSKFEGNEYGAHWGTYNNTKAFFDYQNNLFVQQAKGVIDVSEWQGDIDWAKAKADGVEGVIIRLGYGWGNNADRKAQRNISECKRLGIPFGIYWYSYADTPSIAKEEGAGVVAKLKRFGVRASDLAYPVYYDLEKWTWKGHQPPTDPNVYSDIVNNWYGALQSAGYKNLGVYSYTSYLQGPLKHADIYAKTTWVAQYGARMGFDSFPTNSRGWQYTSSGKVGGIRGNVDMNAFGNKEYVNGGSSNDLQAAIDVRKMTAVTIPNGNYYINVRSKVASSVDVPGGSAADSTAIQLYSGNSSKAQQFTFTRQSDGSYEIVNVNSGKALDVRNGVAENNAVVQQYSRNNSQAQRWFIRDSGAGSYLQSALGNWVLDLSGGNTANGAAIRLYAPNGTASQLFVVSSSDASIATGVSMIITSVANKKLVTDVTGASTANGARVQLDSSNNTNAQKYRFESIGNGTYKIINANSGKVLDVAGGSTADGAALQQYTSNNTVAQQWTVRNYGSGRIALVSVNANKAVDIPGGNAVQQAQLQLYSPNGTVAQQWLVAKAPLTLRERLNETAAKHRQDLPDGTYTFGSKLNTSMKMDVSGASRSNYGNVQIWAGNGTNAQKWKVTHDSNGYVTLTSVNSGKVLDVNGGVSANGTNVQQYDSNGTYAQKWIAVKNSDGSYTFQSALAENKVLDVSGASTSNGANVQLYTANGTNAQKWVK

SEQ ID NO: 107 - protein, 778 aa, Bifidobacterium bifidum
  source 1..778: note = Strain CCOS 571
MSFDLIPEPQSVTLKGDAADSDDAGAPVPVALPLVGRISEDRDIDDIAGVFPTQLADDIEAATGLRWDIASDVMVAGLPAPNAAIAHPVPPHASCWKSFITLRLDPPSLEPQEYRLTIARSGIDVVGGDTEGVRNGVQTLRQIIRQCAPALPRLVIADKPAYKVRGYYLDATRGRVPTLDWLKTWADRLCLYKYNQLQLYIEHTFMFDDLSETWRGTSPLKPADIIAFDEYCARLGIELVPSVSTFGHQYMAMRTRELRHLGEFPEDADRRYGFVERQRHHTLNITEPESLAFSFKLIDAYMQLFRTRKFNICGDETFDLGRGRSKPEAERRGVAAMYADFVSQLCRHLSERGRDPMFWGDIAVEMPQILGLLPDNVTLLNWLYAPGIGEDKVRLVAQAGAPQYVCSAVWCWNALLPRLDDSWNNISRLARYGVKYGAVGYLVTDWGDYGHVNDPRMAVSGMIFGAQCAWNPMAHIQGEAGCGDGEEGSAAGYADAAADVVRENKAAADGDSPAPLPSSSESDDYTGGAADAIAGAPAGGDGSCAEMCRRVAEVEYGDRSGGIVEALRDAACRVAFGWDDMVWYCELDEGDGRMNRDAASAMHLGVHGFSGEYGREWEARLLGSADLDEARRTMLQGLSPHIVRAAEANEALLCDAMRLGAAAGRASRLGAARRDVPAMLAAIEGQRWFNLVGLCLARRHDVITVDAGDIARASAGLIEPDAGSSAGPEAVQDVSIRVARGLERWFETYCDLWRSVSAESELARIASIVWRCADALRS

SEQ ID NO: 108 - protein, 304 aa, Bifidobacterium bifidum
  source 1..304: note = Strain CCOS 571
MADRFGAFLPHDTSGDVAQLHGIGLQKFGDTWYAYGENKVNGNLFQGVCCYTTTDFIAWRSHGIVLDVQEDGSALAADRIGERPKVLHCPATGKYVMYIHAETPDYGYAHIGVAVADAPTGPFAFQTTITWRGYLSRDIGVFQDEDGSGYIMSEDRDHGTHIYRLADDYLTIVEDVACERATDYPYGLESPTIIKKDGLYYWFGSQLTSWDTNDNKYSTATDLHGPWSEWKLFAPEGAKTYDSQVDIVVPLDDDPYNSEHFLFIGDRWQEHDLGNSPIVQMPISIADGVASLTWSDTYEGTTHR

SEQ ID NO: 109 - protein, 634 aa, Bifidobacterium bifidum
  source 1..634: note = Strain CCOS 571
MMQFTMSGTMLRFDETTLRFSFSRDGATWSGCDGIEPQLTREDRSFSFAGAATVTHERIETGTGVGVRSVFAGFAGADYAFETYIWIERSSGDVLCEWVPLRECGAEPRIDRVLWPAPLSFDHADAHDVTLITHEQGVMIPNNWPTEVGTDAVSFGGRFETAGGYMPWFAQLRSDGHAYIAICETPWNAGYDIDHPAGGPYTHVGMWFEPSLGRMDYRRVVRYRLLDHADHTAVCKTYRAYVNERGRLRTLAEKAARNPSVRDLLGRSWVHVGIKTKVQPDSSFYDPAQPGKNDSLVTFAQRERQMRTLHEMGAGRLYLHLDGWAQPGYDNGHPDYLPACREAGGWKGMKSLVDACHEQGDLFGTHDQYRDYYFAARTFDPRNAIRLADGTMPEHAMWAGGRQTYLCAELAPDYVRRNFSEIATHGIALDCAYLDVFTCNEGDECSHPEHRMTRRGCYERRAECFEYLLAHGILTSSEEVSDWAVPSLVFCHYAPYDFQMRSPDAPRHGIPVPLYNLVYHDCVIQPWMMDRVAGGDDYMLYALLNGGAPYLIRDAAYAGMDGDMNAALRARTENDIERCAVVAGLHRRVGMQELVRHDLVGGDPLVQRSVFADGTAVTCDFHAQTYEVAANGSH

SEQ ID NO: 110 - protein, 1138 aa, Bifidobacterium bifidum
  source 1..1138: note = Strain CCOS 571
MRANGNSTHEILGKIVTAIASIAMTAAFAVPAMAASDSDTAADSGTTQQSGQYKIYPQPQNTEYGDGSLILRDKANTVVEPGIDSATKARLNEALKLKGIETTAADAVPETAYQLNVLVGINGSNGVVDKYAKQLIADGTLKVDDSTFSKNDSYVLAVRQGNAKTPDTILVLGRDTDSAFYGLTTLYQIFQQLPGRAVSNLTFSDWADVKSRGFIEGYYGSPWSTKDRVNLMTWGGYYKMNTYVYAPKDDPLHRNNWRGLYTEDQIENEIKPQAEAGNKSKVRFVYALAPFHNDGEARGKHFRFDTEEHYQKDLKELKAKYMQTIDAGVRQIALLADDSTDWGAQYGNDNTYVRVLKDLTDWIHELQQEKNDDGTAKYEGLKDTILYCPALYSYTGAGDAWYKDIPSNVQIVMTGGRTFGVASKDFADTFTKNTGRAPFMWINWPCSDMNRNTAYQYLVMGGQNNFLKPGATYGTYDGIMLNPMQQSEPSKQGIFMAADYSWNLWQSEKDGQQSWEDSFSYIDHNSPIASKGSRGLRDLAMNMRILNDGGIDGAHKDAEYDAVNKWWINNESVDYTGKLDVKGVLTELKGKLDGGTATAADFSQALTVYTTLQRAAKNYRANPGDKNMFDQIEPWISYWDDLTASAIDYITAAKQALAGDTETAKATYATAKAAFAKSDTHTIADYYQRNKPARGGLVIVRPTVQALDSFVKAKTSGSVTPTPSDATVSTNGVGAAAWHENVDPKAVIDGDDSTFFWMQSAGCDCVKANAALTVTYAEARKAKEFRFIQAEKGGDTIVNGKIEYQDADGNWTKIGDVNGNQKQIFTLDSAATVKAVRITNLAQTSKWWKVYDLSATKIDEPGTVTKDALNAKVAEAEKVDSADWTKSSREALADAIAAAKAVAADQDATQAKVDAAVAALESAMKGVERYTAKTADQLKAEHVSNDDATYTEASYNKYQSAYDDFAAALANADDLAKADGEALEAAYTAAKSALRYDQSARDYAQLALNDAEPYVGKASEYTKDSYAKFTVAYEALSKQLKADPNGEGDPATYTALRAALDKAIKGLVKSDGTELERPGEKPGEKPGENKPGEKPGVNKPGADGKLSNTGADVFGLIAAMTMLAAVGVTMAGLRKRIG

SEQ ID NO: 111 - protein, 1755 aa, Bifidobacterium bifidum
  source 1..1755: note = Strain CCOS 571
MNRRISMWLRGRGSPWRPAAAVIATVSMMLATVMGPHFAGMRAQAVEPVQTTTISHTKIAGDGNYFTFADNAWDPGNDVHTWSKAPSDSLPAEDIWYTVRFFGSAIDVYAGKNRPMGKVKYYIDGAEKGTYSLYNASNINETKIASFTGLDEGEHVFKAVATGERDTNSTNALIDCAKVVVTHQPYVVTGVTLDTTSMTLGVGDSKRISYTVAPDYATIDDMTYTSGDTSVATVGADGTVTAVAPGATAITVASTAAGISKIVDVTVARMAPNLTGGIVDPDTQYTQKRFDEVKALTTNNRALKAWKNDKVNSEISLAAVGTTVSNLTVTANDLTSQGGDVIAKSNVTATFIKSTKAYNGSYLGYGDPNREVPAATETNRSESNDILYQSGPITVKANQVQNIWVSFAIPKDAKAGTYTTTLTATADGMETPLTFTYTIEVKAATLPDPAEYEKNFDVELWQYPYSSAEYYGVTPFSDEHLQILRSSMELYKSIGGHAITTTINEDAWSGQTYSANAIHYPSMVKWTKSGGGFTYDFTDFDKWVTFNKGLGIGDKIVIYSIAPWHGNFTYWENGTMKSEKYTVGSERWRSVWTDFLRKLIEHLMDKGWFDESYIGIDERGFSADAFDLIDSIRNIHDVPLKTAGAMDGFVNKFDLALRVTDLNVGDTAAAAHPTDFTRLIEAREAKGLRTTLYSCTEHEPGNFSLSAPVESYWSVVNAGEQTSGFLRWAYDAWVADPLNDATHNAFEPGDPFLIYPSEKSGDKVSKSSVRLERIAEGVRDVNKIRLMVTEIPSLQADADAMYAKIRTTVTTSHSYLTAAQVTQLANEMSGFKGDLDTLTDKYISLKAQGTSTVESVAIDGGDQEIMLGTAKQLTATLKPANLLNASVTWRSSKTGVATVSAKGVVTAAGVGSTTITATSKADPTKSASITLTVTPQVVAQGLHYYSFDDSNANDSWGTRNGTADATAQYVDGKSGKALKVTDGKGVTLAGGNDIAKTDPWTIGYWVRSDAELTGRSAVMTSADGKYSADLKMDADRESGFRVGTASGDVLTFRYDFQPGTWYYIAWTQDKAAGLTMYVNGTKIGVTNTWTKMHDVVAPIDVIGGPGFTGLIDEVKIYKRVLSDTEIAAGMLLPGLNLAEHETDMYIGGTYTIVANLQGGDGDGTVTFESSDPTIAKVDASGTVTGVSRGTAVITVRGGGFTDMVTVNVSRELTIKNTLPQYKLDQTKVTDVHKSLDTSNQYFGQPDMIRTKSGRLITSFPQGHGKGPLIMKISDDDGATWTRKTDIPASWAGSQETPTLYVLNLADGTERIMMITACPGWGTDSAGNRYGWNTSYSDDNGETWTEYRHWQSNRTYDNANNDAIVAMASLVQLKDSDGNDIQKWMGVYHNYAYVNFRTYLTFDENGDEQWSESEPYLAQWRSIESAYQMCEIGMFRSPDGKRIIGLARSQSHNNPATLIYSDDEGETWSKPMDLPGSLAGERHKIAYDPISGRLLVTFREINYDLNGNNRFDGGNDWNAGDWVAWVGTYDQLINQEDGEYRILLAEDWTSNAKSGDTGYAGVAVLDDGTFIMDTYGHWDKEFSQNWPGGVTTDRCYIKQAKFKLGEVEYANGLIDRSGLKAAIKRAEALNAADYTADSWAKMDAAVKAAKAGDADDSLQQAQVDALAAAIDAAIDGLKAKDDGDKPGPGDGGKPGAGDGKPDDGGKPDPGKPGKGDETKPDTGKKGGLSATGAGIVPIAAAALTLMAGAALALAKR

SEQ ID NO: 112 - protein, 1627 aa, Bifidobacterium bifidum
  source 1..1627: note = Strain CCOS 571
MRLRRVKAAIGSVLAAVTLLSMSLTGVTAAQASDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGTGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDASGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKIRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVTADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKATYTVDETFAKTGLAVTGTWSDGKTALLKDGEYKLSAVDADGKTVDLTKPFTAAGDVTVTVTSGKLTDSFTITVKAKTVTPTPGDNKPGENKPGADKPKPNTPDEVAKTGASVTAVVFSALLLLSAGYLLVRKRRI

SEQ ID NO: 113 - protein, 1960 aa, Bifidobacterium bifidum
  source 1..1960: note = Strain CCOS 571
MRSKALGGLLAAALSLSPAVAIGVQTAYAAGGDTAATEYTLYPKPHSIRYDSGQYILRDINVIYDDDIDEATQDRLDEVAALKNLNVTESDAAVSGKTNVYVGVNGSDGKAETAIESKYSPDSAIFDKTDSYFLKSDNGTISVLGKDTDASFYGLTTLYQVLGQIDSLTIRNFTVTDYADVVSRGFIEGYYGNPWSTQDRINLMKWGGYYKLNSYFYAPKDDPKHNSQWRTLYTQDELDTKIKPLADAGNASKTRFVFALHPFMNNAIRFNSEANYQADLKVLQDKFAQTIGVGVRQIAILADDAANVGGNNYTKLLTDMVAWLKEMKKTYPDLKTTLPFVTQEYMGNGMSYFANFPKEVQIVMTGGRVWGEVSQNFTDTFTSNVGRGPYMWINWPCSDNSKSHLIMGGYDTFLHPGVDPSKIQGIVLNPMQQSEPSKVGIFGNATYSWNIWRSKSEADQAWQDSFSFVDHNSAVPTAASNALRELSKHTINQNMDSRVTALQESVDLAPALTAAKAKLADGTITAHDLTDIKAAFVTLQKAAKTYRAKGDAKMLGDIGKDYTGQDANEQIAPWIDCWDDTTKAALAYIAGIEAALNGDTSSTLKEYSDAQSAFAASKKHGFYYVNHTEYAEVGVQHIVPFIKAMDSYLSNKVQQEADPNVVTKTYISDVFTTPTSGSIEDIFDGKDSTVTVFQNPNYLHKGNYVGVKFSKATTLKSIRFAFNGGKNHFYHSKLQTTTDGENWTDVPDATFERPKGSEEPIKVTGLNITGVTGVRLIATADNGDDLWLGIKGIDVNKVEKETLAPYTATGVQLENLNATYSSTKEQMIDGNPSTITYLKDPNGDKIAAGAAVIVDLGSSKPIGEVTITGHASSPDDRPSQGVVEVSDDKATWTKLGDLRDDVTSTVSGNVTGRYVRIRNTAAKNVWWRVAEITVTPPETADPLKSVYTNKTQHGYTATLGANTAELFDKSHTMLDGGQYAGLDLLAIRDLTDVELNTGNANPSLQISDNGLVWTTVEPGNLTGKTARYVRVINGTDGAAFSVNKLKVGFSTVGKFGKLVSSDIQKRNDWGTDTRESGNAFDGDMTTVIKFAGQPRQGNTAVFDLGQPIDITSLRIYTQDTQYDYIRDTKVQMSVDGKTWVDAFEIGDGVSDTDTTTAFGDISDTNKKTDSNYPNVFYYGKDDIANGTGMRYLRLLTTADYPQRALAFNEFMVNQGAYVSTEANAAFSATKVEERGHAPSNMIDGDLTTTYKPSAANGSLTYKIDDPSDIKSFRIVQSGAASGATVTGTVYDAATGTTAGVTARAAARSASEVTFGTLEQAINEFKVPDGKQLLSVKIAWGNAIPEISEFITLDSADTDADITTAKAALKEKIDATVDTSAWTANAKTAYDEAKATAQAVYGSPLVGKASIDAAASAVQSAIDAAQTKADAAAVAALRKLVDAAVTNDGHFYTTVTFTAYTDALDEVKEALKDTNNLSTADAASLKKAVDDALAALKDSTYQRELATIAVDSFATIDEADYTTNSYAAFKAAKEALDTLIAADGTKPATFKTKTAEYTTAKTALVNVAALKAQIAHESNYVEANYTADSWKAYSDALKAAKAELVNGTTDSVAAALTALTTAENALVRTTTPPDTTVLDETIADMEKVNGSEYTTDSYKALTDAIAKAKSDKAKGDAGLNQQNIDAMKAAKEALVSTVELKAKVAEAGKVDSSKYTTASYEALSKLLAAKDDTTSDPVVKGLDTLYKSGTVKELAERVAAIDAAVAKLDARATGVKDYVDGIKLKDNDKGYYTDASYKAYSDAYKALKKLAAAGEGEVGIAEFTEAKEAFEAAEAKLAYKSADYGKIDDLLAKVPSDLSGYTADSVAKFEAAKKAVKRGLTIDQQSKVDAMADALEAAIKGLTLKSQAPGGNKGDGTQSGTHKGDDISKTGADVQVFAIIIALATCAGLGAVAYARRRREA

SEQ ID NO: 114 - protein, 1923 aa, Bifidobacterium bifidum
  source 1..1923: note = Strain CCOS 571
MATLMCGLITSGRLARAVEPAIAGVTASGAAADGHAAAAAVDGDPATYWQSPADSSMQDYRRFLDFDLHGTYSISQIDITNLAGSYYHYEVYLSKDGADYGKVAYKSDDAPATGAADTHPIDATEAAYARISVSYNSAAQQVNLAEVAFHGTKVSDEQASPKAISVTDFDSSSWGREWARVETDADYAAEKTVTEVRNLVGRVIGERWVDKFDFQLRGKADGKDVFEISDAGDDRISIRGNNGVSLASGLNYYLRHWCKVDYNPLFGSQLSMPESLPAVGRKILKYTNYEYRYALNFCTYSYTMAFWNWDDYEPFLDWAAMNGVNLMLDIVGQEEVLRETLTQYGYSDDEVREYLSGPGYYAWFYMQNLYSVGGPLPAAWFEQRVELGRRIHDRMQAYGITPVIQGFGGQVPVDFQEKNPTSVAASSGTWSGFDRPYMIKTYLTDADKAAGKEDYFQKVGDTFYKAQENVFGKVSNYYAVDPFHEGGTIPDGFDIVDIYRTVQRKMLDHDPAAVWVMQQWQWGIDETKLSGLADKGQALVLDLQSDLRSQASPMENQGVPWVWNMLHNFGGRMGLDGVPEVISQDITKAYNSSGYMRGIGITPEAIDNSPIVYELLFDMTWEQDPVDYRSWTQEYAERRYGGTDGTIEKAWDILLDTAYKHTDGEYYQGAGESIINARPSDNTIGSASTWGHSDIDYDKRQFEKAAALFEQAYDSYKDSAGFRYDYVDVMRQVLANSFQEYQPLAGQAYKSGDLETFRTLSSRMLDIIKAQDKLLSSSDDFLVGAWIDDARTMLDGADDWTADLFELNARALVTTWGLNKNGSLIDYSNRQWAGLTGDYYYRRWKTYVDNRLNKLEHGTDFTDPDWFDYGWQWANRKSDEDGYGFATEAADDVDQKALGKIILDQYSVTAMDDVTDGGTAVERTNLALGHDVTDEDTGTVVPDVTDGNTDTGWTQTGKTDATLVVDLDGTYSITGAGITLQQIAADFPLRYEIDVWNGSGWVEIGRSEADAVSSKNEVAADILGSKVRWKLHSTNGRDLTGIYELSVWGAAQPQPEYTNLALGGAASAGPSERPASNGNDGDDGTLWVGNGSDPNWYRIDLASAQRVDRVRLVFETAGRLFQFRVVAGLADGTERTLIDETQNQGALDQVYAANLGEEVEHVTVEFTGSVGGTAWPALAELELLQEAGDTIEGVNIATSAAITSSPTKDAPENAGALVDGKATAWVSRDGATPAWFQLDYAKAREVDSIRLKFEEGQPDRSMQFTLKVIDANGDEHTVAERTEADLSKQQGIVMDVPVGMSITRIRMDIADARIPSSGSPAWPLVSEIEVYATPGNVARDAKTTASDGSTLTAADLAKLTDGDRDSAATLTATADKTLTFTLAKAADINMLGLLASGNSEPVRFKAEYRVVPQDGGGAGDGAADQWKTLTDYSGNAQMKPEIVARLARPVYTDAVRITVLNENPVAINELYLYQADAGASLESYLSSVETVLGKLTVGEYAGNVTRAAKTKLEVVLERARAALDAGLTSREAGRWTTTVEDAVSEFYRTGYVSLDRNALYVAIDDAAALIASLDAHGLPASSAALAEARASAKQVSDAYGTVTQQDLDDAAAALRRSADTALAQLDAQERYQVVLDAAVKTLEDAQTAGSVGEYEGQHPQSAADALRQAIDDAKAAHGAAAGDADKVDAAADALRQATTAFNESIVHIDAAALDAAKRAATGLVESQYDRAAWATMREALAAAGDTDMAHISQADVDTLAARLNDAIAALSDARLDRGPLSDAIASAQALKEGDYTAESWKAFAEALAAAQEECGRRSTTQGDLDKAVKALTQARDGLERKQPGGGEEPGGGEDPEQPGSGGQTERPGGVKPGAGGNADTSKPGTAGTETLSKTGAQTLLLAVTACIMLAAGFSITTARSRRR

SEQ ID NO: 115 - protein, 1112 aa, Bifidobacterium bifidum
  source 1..1112: note = Strain CCOS 571
MEKSSNRRFGVRTVAAIVAGLMVGGMCTAMTASAADDSAAGYSATAPVNLTRPATVPSMDGWTDGTGAWTLGEGTRVVSSDALAARAQSLASELTKFTDVDIKAATGSATGKDISLTLDASKKAELGDEGFKLSIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSIYQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGATPELRNCADVSVGQDQRDTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTEKPTTKPTTKPNDGKLSATGADTAVLATIAALFALAGGAVVAVRRRSVR

SEQ ID NO: 116 - protein, 673 aa, Bifidobacterium breve
  source 1..673: note = Strain CCOS 586
MNGQTSLQGWTIIPMPQTMQHKANIALLPMCGRINEARAVGDDRHILAVQLIDDIRAATGLEWDIATGDRWPGFITLTTFDDPHAHPSGAYTLDVTPDGVTVAGADFEGVRNGVQTLRQLIRQCGAALPCLHIEDRPAFETRGYYLDVTRGRVPTLDWLKHWADKLCLYKYNQLQLYIEHTFAFDSMSETWRGSSPLTPRDILEFDDYCAERGIELVPSVSTFGHLYMALRTQSLRDLGEFPETADEPFGFIDRMHHHTLNISDDRAFDLSCRLIDDYLQLFRSDKFNICADETFDLGKGRSKPLADRIGVAAMYADYVTRLCRHLEARGRRPMMWGDIALEHPEILDRLPETVTLLNWQYDPQVTDEKIRTVAESGAKQIVCPAVWCWNALLPRIDDAWNNITRMARYGAQYHAQGMLITDWGDFGHVNDPRMAIPGMIIGAQESWNPGRIPDEADMLRRISRLEYHDASGELLDILARASHAASFEWNHLITWLELDDGQGGVNTGVLQTIPGLLPENERPDDVIRSLQNESKTPSLAESRRMLLRYLKHRITLGETADHLLQASTRRISAITATAGPRNAGNAAAFRIAVEGQRLLNRVGLRLASETGITDTLQPNTTSQHDDEANLAEALEIWMEAYATQWSTVSRDSELRRLQDTVWKLTDHLRFQSV

SEQ ID NO: 117 - protein, 174 aa, Bifidobacterium breve
  source 1..174: note = Strain CCOS 586
MKYPDIPNVTKEYLDEVRPAINAGGSCSATAGATIGALSPPLVVDGTSIDIDASGVPVASINSYSVGQCTWWAAARRQQIGKPVDPYMGHGYMWAASAEKHGYPTGGTIQLGDVMSFERGVLGASGEYGHVAIVEEIHEDGSILISESGVDQRRAWTRLLTREQAENPGITYIH

SEQ ID NO: 118 - protein, 616 aa, Bifidobacterium breve
  source 1..616: note = Strain CCOS 586
MRLSSGDHAGRASARTRHRKWLRSGLAAVLSMATLAALAAPASATDTMPWGGQYADTSHPEQSAGANQPYQHGYTGLDILNWNPDADKDSEYLRSRVPLQTRIAANAATQKDPNLPADTEMFNLAGDYGNAFFESFHDNSVFSQYLFNYWQYTDYYGTWHGQPTANVPKSLYDEKAQSDWTQKWFEFGTLNLPNAAYTNVAHKNGAKSIATIFYSGNDRGEQTYKDLLQGKRADGTYPVADKLVEIAKYYGFDGYFVNQESSVNSADVPAYQDFMKQIIDQGIYIQWYDSATYPNGGVSYQNMFNDANSPWVQDPNKGKISDSIFLNYWFSGNMLQDSADHAKSLGIDPKYAVFAGIEAGQKKFGSIASNANYMNVNLDADGKPYVSLAALGTDFVSHELGDDKKVYPKYQNQVFDRERRLWTGSSTGEKGTTDISDPYIDDGTSSDSWKGFASQIAERSVIDGPVFSTSFNTGHGLEWRDNGEQTSNQQWGNINLQDILPTWQWWIDADSDPLQADFDYGKKYEAAPRFNYTKVGGYEGGDSLVLSGKLSSDNTVRLYKTDLSVAAGSKVELTYNKLNSDDSKLQLGLIFADDTKTIVPVDMETVGQATAGRPQL

SEQ ID NO: 119 - protein, 627 aa, Bifidobacterium breve
  source 1..627: note = Strain CCOS 586
MEFTVSGTTVRFDERTMQFAFTRDGAEWNTCADFKPTLQCTQGTFAFADATSITHEQRETGTGTGIRSIFTGFGHSAYSFETYVWVERASGDVLFEWIPLNEQGLTVTGVTWPAAIAFDRDDSHDVTLITHEQGVMIPNTWPTAVSTKDITFDGRFETAGGYMPWFAQLRADGHGYIAICETPWNAGYGIDHPSNGPYTHINTWFEPSLGTMNYRRVVRYQFLDHADHTAVCKAYRSYVNERGRLRTLAEKAARNPSVRDLIGRSWVHVGIKTKVQPDSFYYDKDHPEKNDSLVTFAQREKQMRTLHSMGAGRLYMHLDGWAQPGYDNAHPDYLPACQEAGGWEGMKSLVDACHEQGDIFGTHDQYRDYYFTAQTFDANNAIRLADGTMPEHARWAGGHQTYLCAELAPDYVRRNFTQIAAHGIKLDCAYLDVFTCNEGDECSNPEHRMTRRECFDRRTECFEYLLSHGILSSSEEVSDWAVPSLIFCHYAPYDFQMRSPNEPRQGVPVPLYNLVYHDCVIEPWMMERVVNGDDYMLYALLNGGAPYLIRDAAYIGVDGDMDDEQRARTENDIERCHTVAAFHERVGMQELVRHEFVDDDPLVQRSVFVDGTAVTCDFHTQTYRITD

SEQ ID NO: 120 - protein, 237 aa, Bifidobacterium breve
  source 1..237: note = Strain CCOS 586
MSTDQLRQSASVWGSQLAAAGINVDLAPVLGTVVANRAANAPIGALYRDFGLDAAGNAAQGTAFVQGMADAGVQSAIKHYPGLGAVTGNTDFTADGILDTTTTLDGTEIKAFDTTIDQADPAMVMMALATYQAIDPNDPAVFSSIIIDGHLRGDLGYDGVVISDSMSAAAVSAYDTTQLGVKLVEAGGDLACIGYTNYVAPILDGLSTRAASDPAFANEVTQSAIRVMTLKIKMGLA

SEQ ID NO: 121 - protein, 133 aa, Bifidobacterium breve
  source 1..133: note = Strain CCOS 586
MSIGEDTAGVRLCGEVRGGGLFLYPLGEGQIRKISDVPPTTDTALFDKGYEGSASYRIPSLVTTPSGVTIAGADQRVVISNDAPMRSISLSAAPWIADRRGCRCKPSSPFPVMGWMARASSTHASSVIVRQAV

SEQ ID NO: 122 - protein, 433 aa, Bifidobacterium breve
  source 1..433: note = Strain CCOS 586
MSTFPQLATHPGMNPAAVIQGDRWRIGIITESLVRLEWQDNGKFEDHATQMIVNRDWLSDDANGADGANRADGTSNPPKFTKTERDGLLIIDTPALRLTYDMQPFSKEGLSIVVKGVANSQMNTWHYGEAQDGNLRGTARTLDAVDGEIELGLGVISRDGWAVLDDSASNVIVEGAEAATVKGEANPFGMWVIPREHPGKDLYVFGYGHRYIEAVQDFYKLTGPTPLLPRFALGNWWSRYHRYTEAEYLELVDRFEQEGLPFTTAVIDMDWHLVDNVDPKYGSGWTGYTWNREFFPDPERFQRILHEHGLRTTLNVHPRDGVRAFEDGYAKVAEHMGIDPAGGEPVEFDLTSPRFMEAYFDLHHGLETLGTDFWWLDWQQGGVTRQKGLDPLWMLNHMHYLDSAVTGAGRSRSRDMPARARTVIRSGSPATQS

SEQ ID NO: 123 - protein, 338 aa, Bifidobacterium breve
  source 1..338: note = Strain CCOS 586
MIDVSAWQGNINWQAVKNSGVEGAIIRIGYGWDNGFDKQALRNISECRRLGIPFGIYLYSYAYDANTGAAEGSSLVNLLQKAGVSSSDLGYPVYYDLERWTWTGHEVPNDPGTYDGIVNAWYGRLQSAGYNNLAVYSYTSYLNTALNSGNIHSKTRWVAQYGSSMGYTAFPTNDRGWQYTSRGSVSGISGTVDLNAFGNQTATSSAPSVPVYRVYNPNSGLHHYTMNYNEVIMLVGKGWRYEKTAFRAGQSGIPVYRVYNPNDGNHLFTMNSYERDNLARLGWHDEGVSWYVPSGGSINVYRLYNPNNGEHVFTTEYGEYVLVGRAGWHQEGVAWTSL

SEQ ID NO: 124 - protein, 124 aa, Bifidobacterium longum
  source 1..124: note = Strain CCOS 974
MGIPVRSGGYQLIGAKKTFIDEMNQWMKTESNKPFLFTEYGADTDAGVHKLPSVQWSEEYQCEYLAMQHEVFDMFEAVVGEQVWNLCDFQTGEGIMRVDGNKKRLHSRPSAQSRRLCTQGALGD

SEQ ID NO: 125 - protein, 400 aa, Bifidobacterium longum
  source 1..400: note = Strain CCOS 974
MATICALAVVLAGVAAYGWHVGWFAKSTSNGNTTTPQTSQTSALPRADVPSPKKNEPAAQAQRAVSAMTLEERVGQLVMVPLLAGSDPSSLASTIADEHIGSAILIGNWNTGVDTVKTATAQLQGYAPAGNRLIIATDQEGGQVQHLTGTGFDTMPSAVEQGTMSADALRQSAGTWGSQLAAAGINVDLAPVLGTVVGDRASNTPIGALDRDFGLDAAGNAEHGIAVIEGLRDAQVGAAVKHYPGLGAVSGNTDFTTEGILDTTTTLDGAEAGAFDQAITKTDPAMAMMSLATYQSIDPNNPAVFSSTIIDGHIRNALKYTGVVISDSMSAEALSSYDVSQLGVKLVEAGGDMSCIGQTDYVKPIVDGLNERAKSDPAFASKVTAAATRVMALKIKMGLA

SEQ ID NO: 126 - protein, 631 aa, Bifidobacterium longum
  source 1..631: note = Strain CCOS 974
MEFTVSGTTVRFDERTMQFAFTRDGAEWNTCADFKPTLQCAQGTFAFADATSITHEQRETGTGTGIRSIFTGFGHSAYSFETYVWVERASGDVLFEWIPLNEQGLNITNVTWPAAMDFDCADDHDTTLITHEQGVMIPNTWPTAVSTKDIAFDGRFETAGGYMPWFAQLRADGHGYIAICETPWNAGYGIDHPSNGPYTHINTWFEPSLGTMNYRRVVRYQFLDHADHTAVCKAYRSYVNERGRLRTLAEKAARNPSVRDLIGRSWVHIGIKTKVQPDSYYYDKDHPEKNESLVTFAQREKQMRTLHGMGAGRLYMHLDGWAQPGYDNAHPDYLPACQEAGGWEGMKSLVDACHEQGDIFGTHDQYRDYYFTAQTFDANNAIRLADGTMPEHARWAGGRQTYLCAELAPDYVRRNFTQIAAHGIKLDCAYLDVFTCNEGDECSNPEHRMTRRECFDRRAECFEYLLSHGILSSSEEVSDWAVPSLIFCHYAPYDFQMRSPNEPRQGVPVPLYNLVYHDCVIEPWMMERVVDGDDYMLYALLNGGAPYLIRDAAYIGVDGDMDDEQRARTENDIERCHTVAAFHERVGMQELVRHEFVDDDPLVQRSVFADGTAVTCDFHTQTYRITDCPHH

SEQ ID NO: 127 - protein, 315 aa, Bifidobacterium longum
  source 1..315: note = Strain CCOS 974
MGILNKGKPKHGHLHRRVGMTLTALAVAVSMAFAPAAMADMQGVDMSNWQCGADVYNMQADFIVVGTTWGTGQVNNNCLVSGVNTDANRMIAQAQASGKKFGLYHYAMGGNPEAEAQFFYRNTSNYWRHGIVALDWEMDDNPAWGDWDWVRRFTAECERLSGGVKPLLYTGPVAGTIPGDIRANYGLWIAQYANMSPTGYQANPWMLGAYGEAMRQYSGTGVVNTWSPIDLNIFRGEGWQWICTPTPPAPQPRPRQRPRPCSRALPRPTPTRVASATSCNGEKPSGDSPSPMMLGPCPRGIRRAVTSTATTWATS

SEQ ID NO: 128 - protein, 711 aa, Bifidobacterium longum
  source 1..711: note = Strain CCOS 974
MPTFEYKADAATPCLTLIPAPVTLEYTHGTAMIGSLVTIEKRIPEYAVTEDADETWETLPIEQLSSELERYCGVAVRTRRVLTATDEADAGANAAEKARDAGVGAGAGAGAPAAMNGTVILLCVDARLAHDEYTLDVFASDTIAVRGGSESGLRYGMQTLRQMIRQTSRTLPCLHIQDKPAFAVRAYSLDVTRGRVPTMAFLTWFIDQLALYKYNQFQLYVEHAFAFGELSEAWRGTDPLTADDIMFLDEYCAHHGIELVPSLATFGHMYMNLRTREHRGLGEFPEDADRPFSFIERMEHHTLNAANPKSHDFASRLIEEYAPLFRSRSFNIGGDETFDLGRGRSVQDSPGASRDELYADFVKDLCSTLAHRGLQPMLWADIALENPHTMDLLPGDITMLNWMYEPDIDESKIQTIASQGRRQFVCPAVRAWSRFFPDYDGAWLNTYRMAVAGLKYGAEGMVVTDWGDYGHVNDPRLSVPGLCYGAQNAWNPVAIDACEMNHRISNLAYGDESGWLMDSLARIDSDGVSFPWDLAVQVLELEYGSGTGMLNTDVASYVERSCGGELMFDRALGCADARRRLLLRNHARLERRRDCDRALIDCGSAVVAVLDGLARGGLNPELLWVMLDGQRLFNRLGEELLVLAGGEDACDTKDVTGRALDASRRARLAADLELWFERYRVQWLSIGRYAELARIAHVVWSFADILRRGAL

SEQ ID NO: 129 - protein, 634 aa, Bifidobacterium longum
  source 1..634: note = Strain CCOS 974
MVEKKRLDGSDAKGATGAARATGMLRALRARAAASGAGRGVTAAVTSVAAVAMLGAVCVTPASALADVNADADASGVRSAASATASDGTSSDARSQARSATSATRPVANGVTVADETAADAMPDNPDAELPDKVSAEISDDAMVVSEQYAATPEGELKDIETGETVTDPKIVGTESKQPDPLAKTDGESFIPVSAADVKEKVAANGGDVNAVSSKTRAANASVKLAALQNNEYGAHWGTYNGTQAFFDARNNLFAQQAKGVIDVSAWQNTIDWQAVKNAGVEGAIIRLSYGWGNGFDVQAKRNISECKRLGIPFGVYVFSYAESAADGASEGADVVNLLRQAGVNPGDLSYPVFYDLENWTYTGHKSPTSPSVYDGMVNSWYGKLQAAGYNNLSVYSYTSYLNSALNSSNIHGKTRWVAQYGSTMQYTAFPTNDRGWQYTSGGSINGISGRVDMNAFGNYQFTPAVQITWVYRSEDIAVGASVDYPSSDIDYKWQSYNLSSKRWKTITDWTGANWAGWVDQLGDYWLHVEARDSRTRKIIGSQTIAFRYAPGTTRVAATYAGWQNNHVLLGESSNNAAAHYEIKIYDVRRSKWVQGFKGPWAIWKPKKGIYWTHYEVYTSDGRLADTKTYAFGV

SEQ ID NO: 130 - protein, 439 aa, Lactococcus lactis
  source 1..439: note = Strain CCOS 949
MPVSRIKVKNRHLKKKAKKPLAFYKPATKFAGAVLIAGTLTTTHELLLQQTSPMVQAATNSTEAFIESIAASAKPVADSNGLYPSVMIAQAILESNWGSSQLSRAPYYNLFGIQGTYQGKSVVFKTQEYLNGKWVTKDMPFRVYPSFNQSFQDNAYVLKTTNFGNGPYYAKAWRANAATYQAATAALTGKYATDPNYGASLNRIISQYNLTRFDGASSAGTSNSGGSTATNTNNNSNTSSTTYTVKSGDTLWGISQKYGISVAQIQSANNLKSTVIYIGQKLVLTTSSSSSNTNSSTSSGNSAGTTTPTTSVTPAKPASQTTIKVKSGDTLWGLSVKYKTTIAQLKSWNHLNSDTIFIGQNLIVSQSAGSSSSSTGSSSASTSSTSNSSAASNTSIHKVVKGDTLWGLSQKSGSPIASIKAWNHLSSDTILIGQYLRIK

SEQ ID NO: 131 - protein, 347 aa, Lactococcus lactis
  source 1..347: note = Strain CCOS 949
MKKVIKRKVILFSLPFLLLLLPILAFFALFVGSSDLSSDTDINTNTPQQQTAKVIWDRVLKEGGTKEGAAALLGNNQAESELQPSIIQSNATYNEAKAMDTTLGGYAFGLAQWDSGRRVNLLNYAKSQKKSWTDTNLQVEFMFEQDGTDSTLLKQLIKGTNVKQTTEDIMRKWERAGAVDSLPKRQGFAEYWYTFMTTGGDSGTGGGSGITPDIPSGWTLDKPINTSGYLATSYEYKQCTWFTWNRAQDFGITFGMYMGNGADWQHQAGYTVTTTPTLHSAVSFSGGQTVGGQWTADPQYGHVAFVEGIHSDGSVLISQSGTGFSTVYTFQVLTKAQASQLHYVIGK

SEQ ID NO: 132 - protein, 297 aa, Lactococcus lactis
  source 1..297: note = Strain CCOS 949
MQYKKKENKVLVIFVLVVIFILCGCIYVLLVRYTHTDKITTISYKQPTKTSASNGYVEQKGEEAAVGSITLVDDAGVPEWVKVPSKVNLDKFTDLSTNNITIYRINNPEVLKTVTNRTDQRMKMSEVIAKYPNALIMNASAFDMQTGQVAGFQINNGKLIQDWSPGTTTQYAFVINKDGSCKIYDSSTPALTIIKNGGQQAYDFGTAIIRDGKIQPSDGSVDWKIHIFIANDKDNNLYAILSDTNAGYDNIMKSVSNLKLQNMLLLDSGGSSQLSVNGKTIVASQDDRAVPDYIVMK

SEQ ID NO: 133 - protein, 499 aa, Lactococcus lactis
  source 1..499: note = Strain CCOS 949
MTNNDKTKRLKKEKFIAGTALILGMTTFGVAGHADGVTVGSSSDASSSTGSSSSSSGTGGSSSSSSTGSSSSSSNSNASSSSSSTGNSSSSSKPSTSGSTSKPSNPSSSTNNNSTSNSSTTTQAPSTSVSTIAPSASTSTVAPSNYSQDNAYYQTSTAAQIPNSSADSAPSIYAGPVLKTIEAAKSIDKIDTSSTEAFIKSIADRVRILAGKNNLYASIILAQAILESGSGQSNMSQQYFNIFNITGAYLGKSISFKTEEFSGNNPYYIEQSFRVYSNYDQALDDYINLMIKGTTWNSEIYAGAWKSHAKTYQEAAQALQGIFATDPAYAQKLIEIIQEYKLDAYDNVDSTTQVVDSKIPESPLAASKLDNSAYPEYNGVEYPGADSYAFGNCTQYVYNRIIQLGGLVGTHMGNGGEWGINAQAQGYFTTTVPTEGYAVSFPPGVAGSSSEYGHVAFVEKVYSDNSILVSEMNVKGNNIVSERHISAGVAALATYIQPK

SEQ ID NO: 134 - protein, 259 aa, Lactococcus lactis
  source 1..259: note = Strain CCOS 949
MSSIENMIAWMQARKGKVTYSMTLRMGPRSYDCSSSVFFAMIAGGFLSEGSMGNTETLFGMSGTKLKEISRGEVHRGDIFISGTPGGSAGSDGHTGIFLSNGSFIHCSYTHNGIAVDTNDAYMSTRLPHHFYRIVGSGSANTDSKPQMVTLNVDGQFGNATAKRLQEYFDTAGKDGVISHQYKQTFNQNIYAAQFDSSLTGSNVVKALQRFLGIGQDGLFGQGTIKALQKHLGTTQDGTISPVSDSVRELQRRLNANKL

SEQ ID NO: 135 - protein, 271 aa, Lactococcus lactis
  source 1..271: note = Strain CCOS 949
MKPKMRRKLKKMGAFIFTASMIAMIALLGLVRIRPVEPPKKAATEKIVVNHILDEKVLDLNKPVVDLSGWQRPEDIDYNTLSQHVIGAVIRVNGSYGHADNSASKDGEDTAYKQHIKAFQERGIPTAVYAFVTGENTSEMRKQARDFYRRASPYKPTYYWLDVEVTNMKNMNQGIEAFRSELEKQGAKNIGIYAQDWFLRDNQIKVDKFKAIWIAAYGRNTGYWDASPETTLSYKMQQFTDQGTLPGYSGNVDLNMVNNQTNYNELFKNQK

SEQ ID NO: 136 - protein, 870 aa, Lactococcus lactis
  source 1..870: note = Strain CCOS 949
MDKRSKENFKASLNVQKRVSDGIDANAFRKPRQATQLSKQAFKEGKKTLKIRRKNYRKLKAGSGLQVVAAKKKVLEAKADKTVSKAVFKNAKKSDPTRAANQAKGYAKNQAKQTAVNHLVLSPLEKDDTLREGSDLYRKTQRTKSGLKLSKDVVTSSAKASVNLSNHTYGLANRSVNIVRGRGFVRTPSELGFRRQAAKRMRNFQNKLSAARKAKKAEQGFSLIRSILKGQQTLSRAFTLIVTSKGLLVVLAVMFILCLLGILNMASSVPTKQDDFQLTKSWTYFTKLDADNSENGNSFYTPLDDVMFYMNDQFEDYNLQDQVPVGSNGAALPNQNYEQYLTGLWSALNGSSPDYKLTTMEALETDKRSKYYLSPDDYSDFKERVNEVGYDSLDGQLQFPYQTESLVINRRYGYERNGDKTELHASIDVSSAPGQDLTSPMHGIVNSVTDPNTLVISEAENARLTIVGINSGRFIGGETVDAGTLLGKATKSSLNMTYEKYNEDDKKWEKVNPAFYFPKVTYTQFTSLASDSFDPGKSVSERAEAVYNFLTKLGYKKEGICAILGSFTEESQINPKRAEGDYLSPPVGASGNSWDDPAWLAMGGLDIYGKYPNILHRGLGLGQWTDTSDGSERHTLLLNYAKAKNKKWYDLDLQLDFMLNGDTPGNQTMFKNTASNAVSSSIPELTNYFLTYWEGNPGDKIQARVQAAQNWFTYFSNNGGSDADMSASSKELFEKYKDKIKPLPSNKETQQGQGWPGNGYEPGNCTWYVFNRQAQIGHNINGYMGNGGQWGYNYTKTPGATIDSKPQVGDAVSFSPGVAGSSSEYGHVAQVEVVNPDGTFLVSEMNTLGLYSMGYRMFKPGAGMTFVHFK

SEQ ID NO: 137 - protein, 325 aa, Lactococcus lactis
  source 1..325: note = Strain CCOS 949
MEKGLLVDIGRKYWSIAELKRLVLLLQEHKLTHLQLHLNENEGFALNFTDSPVSKKYSENMLKELKEFAKTHEITLIPDFDSPGHMGSLLEQNPEFALPDSNQQAVDVTNPAVIDWIMGIIDKIVDIFPDSDTFHIGADEFIDFRQIEKYPYLVEKTREKYGNKASGLEFYYDYVNQLTEHLQKKGKQVRIWNDGFLRKDLQSLVPLNKNVEVCYWTNWDKGMAEVKEWLTKGYTLINFCDNDLYYVLGEEAGYSYPTAEKLEREGKIQKFSGQQYLNQEEMKAVRGTYFSIWADNAAAKSVSEILDDLSKVLPVFMKIYGGNDE

SEQ ID NO: 138 - protein, 546 aa, Lactococcus lactis
  source 1..546: note = Strain CCOS 949
MKKSKKFFIRICLSTGILASATLLTACFGTPAKHVKANYKVTAEAIPDYQSKNAPISSYWMPDKFLEWSAENDKDLVYNQSRVPLTKRISPDKLSPSNQNQNKKTKIVALSMMNSQTSGNPSRGTTKFESYTFDYWQYIDTLVYWGGSSGEGIIVTPSADVIDEAHSNGVPVLGTIFLPPKEYGGKVDWVKTMLKKDEQGQYPFASQMVKVAKTYGFEGWFINEETQGLNADDAANMKALIQQVKKEDSSLQIMWYDAMTKDGKVDWQNQLNDQNATFVQDKAADAMFLNFWWTQNNLADQKLLEKSNLYAKNHNIDPYNIYAGIDVQAKDVQTPVKWNLLEKGNQATQTSIGLYAASATYTNASNWDDFQNRESAFWVNQKADPRQVDHSVNESWTGLSKYVLEKSAISGNEFNTNFNLGNGYNYFKAGQKISEMDWNDRSLAGILPSYRWIIDNEGKNKISPSFDFANAYNGGNSLKFMAEHLDAGKSSNITLFASDLKIAMGAKFSVSMRSDQALKVSAILELANGQKVSIAGDKSLTENWSK

SEQ ID NO: 139 - protein, 209 aa, Lactococcus lactis
  source 1..209: note = Strain CCOS 949
MKYKTRRRKPNRRQSLLKQLIFIFLVILGILVYNKIFNNQAQKPSEAQLQEINEHKFIKEIAPLAQKSQKESQVLASITIAQACLESNFGKSELASKYHNLFGVKASDDVPKVSLATQEYENGQWVTVQGVFRVYPNFADSVSAHTQLFLYGTTWNSKQYASVLSATDYKTAAKAVQNSGYATDPTYADKLINMIETYHLNQYDKSSSI

SEQ ID NO: 140 - protein, 259 aa, Lactococcus lactis
  source 1..259: note = Strain CCOS 949
MSSIENMIAWMQARKGKVTYSMTLRMGPRSYDCSSSVFFAMIAGGFLSEGSMGNTETLFGMSGTKLKEISRGEVQRGDIFISGTPGGSAGSDGHTGIFLSNGSFIHCSYTHNGIAVDTNDAYMSTRLPHHFYRIVGSGSGNTDNKPQMVTLNVDGQFGNATAKRLQEYFDTAGKDGVISHQYKQTFNQNIYAAQFDSSLTGSKVVKALQRFLGIGQDGLFGQATIKALQKHLGTTQDGTISPVSDSVRELQRRLNANKL

SEQ ID NO: 141 - protein, 259 aa, Lactococcus lactis
  source 1..259: note = Strain CCOS 949
MSSIENMIAWMQARKGKVTYSMTSRMGPKSYDCSSSVFFAMIAGGFLSEGSMGNTETLFGMSGTKLKEISRGEVQRGDIFISGTPGGSAGSDGHTGIFLSNGSFIHCSYTHNGIAVDTNDAYMSTRLPHHFYRIVGSGSGKTDSKPQMITLNVDGQFGNATAKRLQEYFDTAGKDGVISHQYKQTFNQNIYAAQFDSSLTGSNVVKALQRFLGIGQDGLFGQGTIKALQKHLGTTQDGTISPVSDSVRELQRRLNANKL

SEQ ID NO: 142 - protein, 403 aa, Lactococcus lactis
  source 1..403: note = Strain CCOS 949
MTKRLKKKAKPVVFLALVLGCALILSGVLGLAGHLNEQESSKTKKKSQLSETKRSSTSPKNMDKNIEQMTLEQKVGQLFMTGMPANNYNQATFDAIEKYQVGSIILTGRSNLSVPEMKDITDKLQGLEPANRKLLISCDQEGGNVQVLQGQGFSQIPDGLTQGSWTADKLQKESQTWGSELYAAGVNFDLAPVADQVLSADFAPQNAPIGYWSRQYAYDKASIISHAQAFTEGMKAAKVLTTAKHFPGLGAVTGNTDISAGVRDDQTNSNSESVQIFKDLISSGTPSIMTATAIYDKIDPNLPGAFSSKMVDGLLRKQLGFDGLVITDDLSNAVQVQSWTPGQRAVLALSAGNDLVLANEPTQIPEMISEVLQKVKADPDFAKKISQSATRVIKVKEEMKLVE

SEQ ID NO: 143 - protein, 312 aa, Lactococcus lactis
  source 1..312: note = Strain CCOS 949
MIAQGILESSGGQSALASNYNNIFGVKYTSGTPVYLPTQEYLNGTMTNVVEPFQAYSSVYDACVAQAKMLRASSYYSGAWRENTSSYLDATAWLEGRYATDPTYASKLNSVISELGLSVYDQGGEISGGTAVTTSSSASTNSAGTYKVQEGDSLSAIAAQYGTTVDALVSANSLENANDIHVGEVLQVAGASTTTTSTNTTSNVSSSSTYTVKSGDSLYSIAEQYGMTVSSLMSANGIYDVNSMLQVGQVLQVTVSTSATTSNTTTSNSYTIQNGDSIYSIATANGMTADQLAALNGFGINDMIHPGQTIRI

SEQ ID NO: 144 - protein, 346 aa, Lactococcus lactis
  source 1..346: note = Strain CCOS 949
MEKAIKRKVILVSLPFLLLLLPILAFFSLFVGSNSSSDTDINTNTPQQQTAKVIWDRVLKEGGTKEGAAALLGNNQAESELQPSIIQSNATYNEAKAMDTTLGGYAFGLAQWDSGRRVNLLNYAKSQKKSWTDTNLQVEFMFEQDGTDSTLLKQLVKGTNVKQTTEDIMRKWERAGAVDSLPKRQGFAEYWYTFMTTGGDSGTGGGSGITPDIPSGWTLDKPINTSGYLATSYEYKQCTWFTWNRAKDFGITFGMYMGNGADWQHQAGYTVTTTPTLHSAVSFSGGQTVGGQWNADPVYGHVAFVEGIHSDGSVLISQSGTGFSAVYTFQVLTKAQASQLHYVIGK

SEQ ID NO: 145 - protein, 717 aa, Lactococcus lactis
  source 1..717: note = Strain CCOS 949
MKINKIDTRHGTANQSTFSHGNCLPYTGFPWGMNYFSPSTGAARGAWWFHPEDRTFEGYRITHQPSPWMGDFSHLTMTPVAGPLPETSLWHTVSSYRPEESTFNPTQLKITQLRYQITSQLIPSMYGGILSMNYQNLSLKDNGLMLHLPGSYELQQLDDYSLELSIINFAGCEDENFTFYLKFTADQPFVLSGNLSDEDSSVRLDFKAAKQVQIHFATSFISKEQAALNLEREQDNSAETYLENAESAWNNLFSRIEIEHHNQQEVSTFYHNLYRSFLFPQTFYEFDQEHQKIHYDTSSKTVKKGPLYTNNGFWDTFRTVYPLYSLIAVDEYGDMLEGFLNSYRATGFLPKWLSPDERGLMPGTLIDAVIADAASKNIRPDLMPEFLEAMKKGATSQSENSNYGRRGTKDYLKLGYVPLTHHESVNHTLDYAFSDYCISQVAKQTGDKEISDFYAHQAKDYQNIFDSETGFMRAKDADGNFRADFLDIRWGRDYAEGSAWQTSWSVLHDFAGLIKLHGSRENFENKLIELCNQRPNFNVEGYGFEIHEMSEMAAIEFGQVAISNQPSFHYPYLFSYIGKPWMATPLIKNLLTETFNDSPKGYPGDEDNGTMAAWYIFSSLGFYPVTAASNQYVLGIPLWDKARINLSSGQQLTILAEPNAPQQVFVNQITFTDKKVNDTFIKHEELIKGGTLKFDLGIVPNPLKYTNEQLPYSLTEN

SEQ ID NO: 146 - protein, 384 aa, Lactococcus lactis
  source 1..384: note = Strain CCOS 949
MVKISFDVKKFEGQTIKKIGLSIKSDQAMDFKAINLGEMTLTNGQKVAPIALSDAKVTDEAFEEEGTVGGFRLSWKSDANKNNFSTYEIYQLNDDGSKEFLGASNINAFFVNALKRGKNINSTKFEIVPINKAGESGHSVTTSVKWPDNSLAKAAFVADKTLVTIGEKVTLMNQSNLASVKYKWDIDGASPATSTEKNPQVSFDKAGSYSVKLTVINEKGQEDSVTQTELITVIDQPVELTNFALNQSVQVDSFTNESESGPKAVDGKLNTKWCAVGPGKHNITIDIGKSEKINQVLIDHAQKGGESPDMNTSDYTIEISKDNQNWTEVVNVKKNKLGETKDSFKQTEARYVRITATKPTQGADTAVRLYEIQVLGQKKADKGL

SEQ ID NO: 147 - protein, 569 aa, Lactobacillus jensenii
  source 1..569: note = Strain CCOS 962
MTKQTLQAEANGRNGKIKFDINIDNNEIKDIKVTKSSETPAIFNQVFDKLKNSIIEEQSFDVDAVSGATIMTSALLDSGKKALNQAGVTPVAKESDKTHREVNLDVDVAVIGSGAAGLIAACRALSMGKNVVVLEKNGYLGGATILNGSNVVATGSNLAQQLFGTEAQEDSSKRLFADITRECRGTNYPELSKVLVENIGKAVDFIKEFAGLTYQKAETQTIEHSVNRQVEMPSESSYELIKKIAAAFEEKGGKILLDARVEKINSENGVPISLVAEGKHQTTNVKFKSLILAAGGWGAKDFKEKRTSIPYYGPMTSTGDYFFFNKGLNLASRNLDWYKVYPHGLEVEPGIAKLTTYSTKEASDMGAIFINRAGNRIVNESDPYTHFRDAIAAQKDQIAFVLMDQRIWNRFYELMLKYGFTADEISHYFALDGKQSPILVKGTLETVANKAGINFENLQHTLSNYQNYAKNGKDPEFGREAKFMHEYSGDTYYVIEQKLRFCTTLGGYETNSQMQLLNNDMKPVANYYAAGEVIGGANGHDSMPSMMNSWSYASGFLAGTNASDNCNNR

SEQ ID NO: 148 - protein, 717 aa, Lactobacillus jensenii
  source 1..717: note = Strain CCOS 962
MKTKKITGVATAALLASVAVPVTNNLANIQTSYTAKAVTGQQQAFLNTAIPNAEAASARYGTYTSVMLAQSILESGWGASLLATQANNLFGMKGSYNGQTYYTNTSEWASGTGYYNINAGFRKYPSWAASFEDNGYKLRTGTTDNPSRYRMAWIENAANYQVATQGLKDGGYATSPTYPQSLNRVISSYGLNQYDPSVDTTTKTMRVLSNGTVYSGPADSSVVSATGNITAGQVVTVDKTVTYKNGLSYMHISNGWINGSLLTGSSTQATTTEKAGTTTDAGNAAIKVVYTSAIAEWKNPGSGVVGYLQKGTTQTVVGKIQVNGAWWYKLSSGNWVPGEYVYVTGASRIPTIDNNVVNQNTKVKIKYISGYSIAVWSNPAIGTTGQYLKDGTEVQTIGYTTANGKKWYKLANNTWIPAEYTEVVSSSTVVTNITNESNTVAINYPHYSIAVWSEPGRNSTGKYLSDGTKVQTVGYTTVNGKKWYKLADNTWIPAEYTKVVSSSTVVTNITNESNTVAINYPHYSIAVWGEPGKNSTGKYLSDGTKVQTVGYTTVNGKKWYKLADNTWIPAEYTKAVSSTDTTSGNNTVTVNYPGYSIVVWSEPGRNSTGKYISDGTSVKYYATANYNGQTWYKIGENQWVPGQYVKVNASNNSGSVTVKYISGYGIAIWSNPGRNSTGKYLENGTTVKYFETQNYNGQTWYKIGENQWVPAQYVSVN

SEQ ID NO: 149 - protein, 775 aa, Lactobacillus jensenii
  source 1..775: note = Strain CCOS 962
MAENNHRSASNTAPKNSRMANLHHDSGQPKRRLWLQILKWFSIGILLILVSGVGLFAYYAKDAPSISQDQLQSGGSSSFYTSDGKFLLSLGSEKRTYVKNSDIPQTLKDAVVSVEDKRFYQENLGIDPIRIVGSIVSNAKSSGIAAGGSSITQQLVKLTVFSTAASQRTLKRKAQEAWLAIRVEHDYTKNQILEYYINKVYMNYGVYGMGTAADYYYGKSLKDLDLAQTALIAGMPNAPVAYDPYTYPKAAKYRRDIVLNAMYANGKISKAQLKAAKAESITQGLKTQQNSSESSIRRIDDPYIKEAISEVKSKGYDPYNDNLKITLNIDQDAQNKLYELANGSSIPFSSSKMQVGATIINPSNGHVVAIIGGRNLPSVQLGLDRAVQTGRSTGSSIKPVLDYAPAIEYLNWSTAHYLEDTKYVYPGTSIQLYDWDNKYMGKMTMRYALEQSRNVPAVKTLAKVGMKKASLFAKKMNISVGSNQGLSVAIGANASSLQMAGAYAAFANEGVYYKPQFVSQIETADGVVHSYSATGTRVMKKSTAYMITDMLKGVLTEGSGTNARTGLYEAGKTGTVKYSDDELVKYPSYANTPKDAWFVGYTKKYSIGIWTGYDNLSDGTISGQGQYASQYLYKYMMKYLMSDKENSNWTKPSNVVRKRIVKGSDPLEVTSSKKNSTSELFLRGHTPDGSNEDSDESSSSSNSSSSSKDNEVVTNKSSSSSSSSSSEGHEDGSTSNSSSSQSSSGGSPNNNQGTTTNNQTNNNNNTGQNNGGGNN

SEQ ID NO: 150 - protein, 2089 aa, Lactobacillus jensenii
  source 1..2089: note = Strain CCOS 962
MFSKNNMKMRMQKVADKEQQHFALRKLNVGLVSVLMGTTLFFVGQTSIAKADTLESTKTSVTEISSSSNSNQEKLEQAKAVSSQEQTTSTEENVDSASSSQSKSENNSSSSAKSEVSTQDRTDSNDNATTNQVDTTQVSTTSLKAVQSSNQAANLLAESKVEDSTTANVSDFDSFNSALMNQNITTININSDFTSGNYGTNVEHRRVVPRTLVINGNGHTVDLGSIGYFMNPGGNQAADWTVENGTFYSKSAVGPFALTGDHGTDKAWQATNGAVHRMTYKDMTVYAGGGAYAANAEIDLEGTNNLNATASYQSKFSNGTVYTETGGNRSGVEGQWIIIKDNANVTVNTDHGQAISTEYSDGNHRIEVGENATLNGNYLYTTYGTPYGGGKIAVLSNGANGGTVIFHKGSKSHFTMPNQNVTGFGGNGFSNYGTGSVLYLGAANTTIEKGAEVTIDNPKEANNDNSIFIRSDGTQLNIFGNLIINDGARDYTIRADNRVSINVGQSIADDDYSNDNGKLIINRNGDFYLNNDGGALSMWGPITINVNSGAALEVYSTGINNVSGSGANPALMRVGGSSKLNVYKRGTFILSDSSNGKVSLVDDYNANGNFNFDNTAKVIFDISNNNNQNSRIFALNGTVYATHDKVRAMLTPSSNIADMGTFKTVQFNTSGQGGVTGDALHSEYQNQAYNNIVAIRDAIRTGQLRYLEFSTRSVNNASITGEGHGFTDNGAQIKGSVVDESTNTGAVAVAYDKDNNKVGSGNVDDQGNFTINLDKPLLNKEEVKVRIETPRGASGFVSTTAPLGPTAKSPITVAKDDNLAGQDASQYITNKDEIANIKPGTTDYEPTFKSAAWKSVDTNTKKGIITVTYADNTTTDLDVDLNVVDKITDADKFTPEGGTIKVDNGHKLDGNDAYEAIKNHDDLPADKAGYNWVVDENHPAVDTTKPGDQTGYVQVTYNDGSKSDLVPVTVHVIADNEKYTAIGKTQNLQKGQSANPQDFIANKDGEVSQDGKTYTKLPDGTKYEWVDGGIDTSTIGPKQAQVKVTYPDGTTQIVPVKANVYSDANLSTVVGKDINAGYKEDLTNRAIDAIDKDKSTNLPTDPSAYTWVNGAPDTSKTGDIPATVKVTYPDGSYNTVDVTVHVTSDAEKYSPVPETISVPKGTDLSGRAKDGIANADKDANGKEKLPDGTKYEWDGGVPNTSVTNNKYGHVKVTYPDGSSTVVEVPLNITDNNKSDAEKHNPQANILHATLNQDLSSNDWAKKGILNADEQDGATFRWRKGYVPDTSKEGQVWGIVVVSYPDSSINEVRVPVIVQSDASKYGIETQEINVHEGTDISSDEWARKGILNADQAGKQNEQLPDGTSYTWANGNVPDTTKPNKKTGYVTVTFPDNSSRTVPVIVNVIGDGRSDAEKYQLKAHDIWTYINDTPVAEKAVSNLDELKDVSSITWATTPDVSKVGNVPAIVVVTYKDGTSNAAPINIEVKGLANDYKPEGTTIYAGLNEDITNRAADGIANKDQMPVANKPEGTVHTTYSWKDNIIPDTTKPGTKYGIVEVNFPDGSTKDVPVEVKVTSLASDYQNKIDTKQIIAKYKGNIPQASDGIANKDQATKEGDKDFPSLADVLAPNGIQWKKNFEPDLSKPGLTSGEAILTFKDGSTAEVTIPVLVQTDADRNTPETQTIKTLPGQTVNPEDGVINLHKPGENNPQLPDGTKVTFDNQSDVDDFTKHGMPGSDKSFDATVTYPDGTTDKIKLPVHITADNEVNTPITQGIITPKDSVPDANKGIANLKKATTKEGKTYPALPENTTVEWVNPGQMKTELENAKGGTTKNYDAVVIYPDKSTEIVSIPVTVATDADTYKVVTQPIDLKDRNLPDNADDGITNLHKPADFKTPQLPDGTHAEWQDKDAAQEVVKNLKPGETVKLPATVVFPDGSKKGEGIDVSVHLHGQSDDYNIETQPVNTDKDGNLPENADSGIKNLGKLPEGTHASWGDGAQDIAKNLKLGETKDVPATVVFPDGSKKEITIPVHREGQSDGYDVEPQLVNTDKNGQLPNAKEGIKNLADLPEGTNQPGQIELKIKLIRLSLVQIQLLK

SEQ ID NO: 151 - protein, 214 aa, Lactobacillus jensenii
  source 1..214: note = Strain CCOS 962
MARSGSRVKKTTLTLVKVFGVLLILVVAFVCFRYYRRQAINNELIRQEQLAREQAAAKELKIKTDFIKKIGPIAQKADQGFALLPSITIAQACLESNYGQSELSQKYNNLFGVKSNDPNTSKILSTKEYVNGKWITVKASFQIYDSYESSIQAHARLFQNGTTWNKDQYQHVLAAKDYKTQAKALVTDGYATDPDYASKLINLIEQFNLNKYDN

SEQ ID NO: 152 - DNA, 1323 bp, Lactobacillus rhamnosus
  source 1..1323: mol_type = genomic DNA; strain = CCOS 965
atgaaacatatcggacaacttaagaaatggccgctggtgatcttattggcgctattgtttggcgttggtgccgcaacaaccagtgtgatggccaacacccaatacatgacagtcaaagccgagagcgtcaatgtacggctaggtcccggcttggcatatggcatcatgggtcaggtaaaatccggcaatgaattgaccattatcggttccaaaaattcctggtatcaagtgcgccttgccggcaataaaatcggttgggttgcttcatggttagtcgatcaaagcgaagccgcaaccacgagtgcgaaagttgccacggtgaatcaaccggttaacgttcgcgaatatgccagccaggatgccaagcagttaggaacgcttaatgccggcgatagtgtcaaagttgtgtatcaagaaggcgattggacccagattgcctataacaatacggcggcatggatcactagcagcagtgtccagctcactggtcagacaactaatctggcacaacctgcccaagccaatctgacgcaagccaaaagcggggccgcattgaaagtcaccaccaataccatgactaacctgcgcaatgcggcgggtatcaatgccccatccgtcgagaaactcgataaaggcactgaactgaccgttaccaaacaacaagacgattggtatcaagtcactgcccctgatggcaaatcgggttatgttgccagctggaccgtcaccgccccgaataatggtcaaacccaaaaagcggcaacgaaactatccgaagccacgattgttctggatcccggacatggtggtacagacacgggtgcgccagctaacaataatcatgattacgaaaaaacctataccttgaaaacggccgaactcgttgccaatgctttgcgggctgctggtgctaacgttatcatgacgcgaacgactgacacctttgtcgacctcgcgccacgtccgacaactgctaacaacgcccatgccgatgcatttatctccttccattttgactccagcccgtccaaaaactctgcctccgggttcaccacctattactacagcagcaaaaaggatctcgccttggctaaggcagttaacaacgcttttgatgacttaccactagaaaatcgtggggttgcatttggtaactatgaagttctacgcgataataaacagccggccatcctgaatgaaatgggttatatcaataacgacaaagactttaagtatattaaaaacccaacgtatcaatctaaaattgcgaccaatattgttaacggtttaaatgcttactttaaagccggtcatcatcaataa

SEQ ID NO: 153 - DNA, 636 bp, Lactobacillus rhamnosus
  source 1..636: mol_type = genomic DNA; strain = CCOS 965
ttggcgaaaaagcgccgacgttttaccttaaagaaggttccattggcggcttggctggtcatcgctttttttgcggttggttcaatgttggttgtcagctcgtattggtcgcaacggcaagccgaagtttcacagcgccaagcaattgtcgataagaaggcggctgaaaaagcgaagaaagaggcttttatcaagcgattggtgccgacagcccaggccatgcagaaacagtatggtgtcttaacgagcattacgctgtcccaagcgattcttgagtctgattggggaacgagcacgttagccaaggattaccataacttatttggcattaaagggacggatcctgccaccacgaaagtattacggacgaaggaatatgttaacgataaatggattaccgttaatggtcggtttcgggtttatagcgatgatgctgcttcgattcgtgatcatgccttgttattcgttaacggtaccgactggaatccgcaacagtatgcaacggtaagagccgcaaaagattataagactgctgcctcggctttgcagacggacggctatgccactgatccggattatccccagaagctgattcatttgatcgaagcatggaatctgacccagtatgacaattga

SEQ ID NO: 154 - DNA, 765 bp, Lactobacillus rhamnosus
  source 1..765: mol_type = genomic DNA; strain = CCOS 965
atggcgatgttaatggcagatgtaagcagttggcagccggattcggatagctggtttcgcaagttggctgatctgggtgtcaaggcggtggtcgtcaaactgacagaaggaacgacttaccagaatcctaaggcggcggcacagttggcggctgggcgcaggatggggatgcaggtgcacgcttatcactatgcccattttcataacagtgccgaagccgttgcagaaggacgtttcttcggcgtcacggcccgcgcgctgggcgtgtcgacgcaaagtgtgatggcagcggatgttgaagcacctgatttgaatggggagctgacggggctgacgaatgttttcattcagacggtcaaagcgctagggtatccacacacagacctgtatacaatggcgaattggatgaaaaccagacgctttgatcgcgtggcattgattcctaagaacctgtggattgcgagttatggcgttgatcagccgggcatcaataatgtcgggacctggcagtttaccaataactttcacggcttaggggtggatatgagttatgactttttcggtcactacaccacgcgcttgaccggtaacctcaatggcggcgtggcgcgcgttcccacgattcgctatcacacggttttggcaggcgaaagctggtgggcgattgcagatcgatatggcttgaatatgtacaagctcgcggccctcaatggtaagacgatctacagtgtgattcatccgggcgatcagctgcggttgaattaa

SEQ ID NO: 155 - DNA, 1323 bp, Lactobacillus paracasei
  source 1..1323: mol_type = genomic DNA; strain = CCOS 1201
atgaagaaaattggacaattaaaaaaatggccgctggtcgttctggtggcgctacttttcggcgttggtgttgcaacaaccagtgtaatggccaacacacaatacatgactgtcaaggcagataccgtcaacgtgcggttaggtcctggtttggcatattcaattatgggacaagtgaaatccggtaatgaactgtcgattatcggtgcgaaaaactcctggtatcaagtccgactagctggtaataaaatcggctgggttgcctcttggttggtggatcaaagtgaagcagcaacttcacaggccaaagtggcaacggttaatcaaccggttaacgtccgtgaatatgccagtcaaaatgcaaaacaactaggttcgctaaatgccggcgacagcgtcaaggtcgtctatcaagaaggcgcttggactcagattgcgtacaacaccacggctgcatggatcaccagcagcagcgttcagttaaccggccaaaccaccaatttagcccagcctgcgcaaacggcgttggcaaccgagaaaagtgcgccggcgctgaaggtaacaaccaatacgatgaccaaccttcgcaatgccgccggtatcaatgcgccttcggttgaaaagctagacaaaggcaccgaactgacggtctctaagcaacaagatgattggtatgcggtcacggcaccagatggcaaaactggctacgttgcgagctggaccgtcagcgcaccaaatgacgggcaaacgcaaaaagccgctacaaaactgtcagaggctacgattgtccttgatccaggtcacggcggctccgacactggtgcgattgccaatgatggcaccgactatgaaaaaacatacacgcttaagacggccaatttagttgctaacgttttacgcgcggctggtgccaatgtcattatgacccgtacaacggatactttcgttgatttggcacctcgaccaaatactgccaacaatgctcacgcggatgctttcatctctttccattttgactccagtccatccaagaattcagcatctgggatcaccacctattactacgacagcaaaaaagaccttgccttggccaagtccgtcaacagtgctttcagcggccttccgctggaaaaccgcggtgtcgcctttggtaatttcgaagtcctgcgtgataataaacaacctgctattctgaacgagatgggttacatcaacaacgataaagatttccgtcaaatcaaagatccgagctatcagtccaagattgctaccgatattgtaaatggcctaaacgcctactttaaggcaggcaatcatcagtga

SEQ ID NO: 156 - DNA, 765 bp, Lactobacillus paracasei
  source 1..765: mol_type = genomic DNA; strain = CCOS 1201
atggtgatattaatggcagatgtgagtagctggcagccggaattggacagctggtttcgcaagctggcggatgttggggtgaaggcggtggtggtaaagctgacggaaggcaccacctaccgcaatcctaaggccgcagcgcaattggcggcaggtcggcgaatggggatgcaggtgcatggttatcattacgcgcattatcacaacagcgctgatgccgtggcggaaggacggttcttcggaaccacagccaaggctttaggtttgtcaaccgagtcggtaatggcagcagatgtggaggatccgggtttatctggtgagttgacgggggtgacgaatgtttttctccaaactgtcaaagcgatcggatatcctcacacggatctatataccatggccagttggctgaccgcgcggcgctttgatcgggttgccttgattccgaaaaatctgtggcttgccagttacggcgtgaatcaaccgggcgttgataatgtcggcacatggcagttcacgaataactttcaggggctaggtgttgatatgagttacgacttttttggtcactatacgacgcgtctcactggcacactcaacggtggtgtcgcgcgcgtgccaacgattcggtttcataccgtgcagccaggggagagctggtgggcgattgcgcaccagtacggacatgacatggacaagctggctgcattgaatggcaagacgattttgagtgtgattcaccctggggatcagctgcgggtggaataa

SEQ ID NO: 157 - DNA, 738 bp, Lactobacillus paracasei
  source 1..738: mol_type = genomic DNA; strain = CCOS 1201
gtgcgcagtcgggtcatttatgctgatacctatcgcaaacgtcatcaaagacgcttatttttgctggccgttttaatcatcgctttgggcgcactgtttgtttggtggcatcagcgcaacccccgccctgatccgcagacctatcccgtgttgggcgtgcgtcttgaccaaaccgatggggtgcaggattttgacagtttgcgcagcagcaaagtcagttttgtctatctcaaagcgactgaaggcagcagctattttgatgataattttaatacgaattttaatcaggcagctggcagtcgactgagcatcggcatttaccatgtgttcagctttgaaacgacaccccaagcccaagctgaccaattcacgcgccaagtcggccaaaatatcggtgatttaccgattggcatttacttaagctattacactgaaaagaagccaagcacacaatggctgacaactcatttacaaacttttgtcacattggtgcagcaacattatcatcgccaagtgttgctgatgggtagcccaagtattttaaaagctgtccaaaaagtcgacccgcaagcaccacgctgggttgtaagcgacaagaagccgacaacgagcagtggtttctggcaatatacgtctggcgcccggttgccgaatgggccacacgcagattaccgtgctgccgtatttatgggggatcgggcggcctttttaaaactgtctcagcagatagtcaattaa

SEQ ID NO: 158 - protein, 440 aa, Lactobacillus rhamnosus
  source 1..440: note = CCOS 965
MKHIGQLKKWPLVILLALLFGVGAATTSVMANTQYMTVKAESVNVRLGPGLAYGIMGQVKSGNELTIIGSKNSWYQVRLAGNKIGWVASWLVDQSEAATTSAKVATVNQPVNVREYASQDAKQLGTLNAGDSVKVVYQEGDWTQIAYNNTAAWITSSSVQLTGQTTNLAQPAQANLTQAKSGAALKVTTNTMTNLRNAAGINAPSVEKLDKGTELTVTKQQDDWYQVTAPDGKSGYVASWTVTAPNNGQTQKAATKLSEATIVLDPGHGGTDTGAPANNNHDYEKTYTLKTAELVANALRAAGANVIMTRTTDTFVDLAPRPTTANNAHADAFISFHFDSSPSKNSASGFTTYYYSSKKDLALAKAVNNAFDDLPLENRGVAFGNYEVLRDNKQPAILNEMGYINNDKDFKYIKNPTYQSKIATNIVNGLNAYFKAGHHQ

SEQ ID NO: 159 - protein, 211 aa, Lactobacillus rhamnosus
  source 1..211: note = Strain CCOS 965
MAKKRRRFTLKKVPLAAWLVIAFFAVGSMLVVSSYWSQRQAEVSQRQAIVDKKAAEKAKKEAFIKRLVPTAQAMQKQYGVLTSITLSQAILESDWGTSTLAKDYHNLFGIKGTDPATTKVLRTKEYVNDKWITVNGRFRVYSDDAASIRDHALLFVNGTDWNPQQYATVRAAKDYKTAASALQTDGYATDPDYPQKLIHLIEAWNLTQYDN

SEQ ID NO: 160 - protein, 254 aa, Lactobacillus rhamnosus
  source 1..254: note = Strain CCOS 965
MAMLMADVSSWQPDSDSWFRKLADLGVKAVVVKLTEGTTYQNPKAAAQLAAGRRMGMQVHAYHYAHFHNSAEAVAEGRFFGVTARALGVSTQSVMAADVEAPDLNGELTGLTNVFIQTVKALGYPHTDLYTMANWMKTRRFDRVALIPKNLWIASYGVDQPGINNVGTWQFTNNFHGLGVDMSYDFFGHYTTRLTGNLNGGVARVPTIRYHTVLAGESWWAIADRYGLNMYKLAALNGKTIYSVIHPGDQLRLN

SEQ ID NO: 161 - protein, 440 aa, Lactobacillus paracasei
  source 1..440: note = Strain CCOS 1201
MKKIGQLKKWPLVVLVALLFGVGVATTSVMANTQYMTVKADTVNVRLGPGLAYSIMGQVKSGNELSIIGAKNSWYQVRLAGNKIGWVASWLVDQSEAATSQAKVATVNQPVNVREYASQNAKQLGSLNAGDSVKVVYQEGAWTQIAYNTTAAWITSSSVQLTGQTTNLAQPAQTALATEKSAPALKVTTNTMTNLRNAAGINAPSVEKLDKGTELTVSKQQDDWYAVTAPDGKTGYVASWTVSAPNDGQTQKAATKLSEATIVLDPGHGGSDTGAIANDGTDYEKTYTLKTANLVANVLRAAGANVIMTRTTDTFVDLAPRPNTANNAHADAFISFHFDSSPSKNSASGITTYYYDSKKDLALAKSVNSAFSGLPLENRGVAFGNFEVLRDNKQPAILNEMGYINNDKDFRQIKDPSYQSKIATDIVNGLNAYFKAGNHQ

SEQ ID NO: 162 - protein, 254 aa, Lactobacillus paracasei
  source 1..254: note = Strain CCOS 1201
MVILMADVSSWQPELDSWFRKLADVGVKAVVVKLTEGTTYRNPKAAAQLAAGRRMGMQVHGYHYAHYHNSADAVAEGRFFGTTAKALGLSTESVMAADVEDPGLSGELTGVTNVFLQTVKAIGYPHTDLYTMASWLTARRFDRVALIPKNLWLASYGVNQPGVDNVGTWQFTNNFQGLGVDMSYDFFGHYTTRLTGTLNGGVARVPTIRFHTVQPGESWWAIAHQYGHDMDKLAALNGKTILSVIHPGDQLRVE

SEQ ID NO: 163 - protein, 245 aa, Lactobacillus paracasei
  source 1..245: note = Strain CCOS 1201
MRSRVIYADTYRKRHQRRLFLLAVLIIALGALFVWWHQRNPRPDPQTYPVLGVRLDQTDGVQDFDSLRSSKVSFVYLKATEGSSYFDDNFNTNFNQAAGSRLSIGIYHVFSFETTPQAQADQFTRQVGQNIGDLPIGIYLSYYTEKKPSTQWLTTHLQTFVTLVQQHYHRQVLLMGSPSILKAVQKVDPQAPRWVVSDKKPTTSSGFWQYTSGARLPNGPHADYRAAVFMGDRAAFLKLSQQIVN